(19) United States
(12) Patent Application Publication
(10) Pub. No.: US 2024/0392292 A1
(43) Pub. Date: Nov. 28, 2024

(54) RNAi Agents for Inhibiting Expression of Coronavirus (CoV) Viral Genomes, Compositions Thereof, and Methods of Use

(71) Applicant: Arrowhead Pharmaceuticals, Inc., Pasadena, CA (US)

(72) Inventors: Christine I Wooddell, Madison, WI (US); Anthony Nicholas, Oregon, WI (US); Audra Winter, Lake Mills, WI (US); Casi Schienebeck, Deerfield, WI (US); Tao Pei, Middleton, WI (US)

(21) Appl. No.: 18/791,757

(22) Filed: Aug. 1, 2024

Related U.S. Application Data
(63) Continuation of application No. PCT/US2023/061856, filed on Feb. 2, 2023.
(60) Provisional application No. 63/306,045, filed on Feb. 2, 2022.
(60) Provisional application No. 63/376,297, filed on Sep. 20, 2022.

(51) Int. Cl. C12N 15/113; A61K 9/00; A61K 47/54; A61P 31/14

(52) U.S. Cl. CPC C12N 15/1131; A61K 9/007; A61K 47/545; A61P 31/14; C12N 2310/11; C12N 2310/14; C12N 2310/315; C12N 2310/321; C12N 2310/322; C12N 2310/334

(57) ABSTRACT

Described are RNAi agents, compositions that include RNAi agents, and methods for inhibition of coronavirus (CoV) viral genome. The CoV RNAi agents and RNAi agent conjugates disclosed herein inhibit the expression of a SARS-CoV-2 viral genome, and the targeted portions of the genome are conserved across a variety of known coronaviruses. Pharmaceutical compositions that include one or more CoV RNAi agents, optionally with one or more additional therapeutics, are also described. Delivery of the described CoV RNAi agents to pulmonary cells, in vivo, provides for inhibition of CoV viral genome expression, including SARS-CoV-2, which can provide a therapeutic benefit to subjects, including human subjects, for the treatment of various diseases including COVID-19.

### CROSS REFERENCE TO RELATED APPLICATIONS

This application is a continuation of PCT application PCT/US2023/061856 under 35 U.S.C. 111(a). This application claims the benefit of priority of PCT application PCT/US2023/061856, filed on Feb. 2, 2023, which claims the benefit of U.S. Provisional Patent Application Ser. No. 63/306,045, filed on Feb. 2, 2022, and U.S. Provisional Patent Application Ser. No. 63/376,297, filed on Sep. 20, 2022, the contents of each of which are incorporated herein by reference in their entirety.

### SEQUENCE LISTING

This application contains a Sequence Listing (in compliance with Standard ST26) which has been submitted in xml format and is hereby incorporated by reference in its entirety. The xml copy is named 30702-US1_SeqListing.xml, created Jul. 31, 2024, and is 4939 kb in size.

### FIELD OF THE INVENTION

The present disclosure relates to RNA interference (RNAi) agents, e.g., double stranded RNAi agents, for inhibition of coronavirus (“CoV”) viral genome expression, including severe acute respiratory syndrome coronavirus 2 (SARS-COV-2), compositions that include CoV RNAi agents, and methods of use thereof.

### BACKGROUND

Coronaviruses (CoVs) are a large family of single-stranded RNA viruses capable of infecting animals including humans, and causing respiratory, gastrointestinal, hepatic, and neurologic diseases (Weiss and Leibowitz, Adv Virus Res 81:85-164 (2011)). There currently exist six identified human coronaviruses: two alpha-CoVs (HCoVs-NL63 and HCoVs-229E), two beta-CoVs (HCoVs-OC43 and HCoVs-HKU1), severe acute respiratory syndrome-CoV (SARS-CoV), and Middle East respiratory syndrome-CoV (MERS-CoV) (Wu et al, Int J Infect Dis 94:44-48 (2020)). The symptoms of CoVs vary from mild ailments similar to what is caused by the common cold with a fever, sneezing, cough, sore throat, or runny nose, to very severe cases of pneumonia and even death.

In December 2019, a contagious disease caused by severe acute respiratory syndrome coronavirus 2 (SARS-CoV-2), named Coronavirus Disease 2019 (COVID-19) was identified in Wuhan, China. SARS-CoV-2 is a positive-sense single stranded RNA (+ssRNA) virus. COVID-19 subsequently spread worldwide causing a global pandemic.

While highly effective vaccines against SARS-CoV-2 have since been identified that are capable of reducing severe outcomes in most subjects, breakthrough infections of vaccinated individuals still occur and the duration and extent of protection provided by the vaccines appears to wane over time, necessitating recurring booster vaccinations. Further, while certain small molecules, antibodies, and other alternative treatments have been shown at least anecdotally to alleviate symptoms of symptomatic COVID-19 infected individuals, in many cases the mechanisms of action remain scientifically controversial and none has been accepted universally as a sufficient therapeutic. Moreover, it is unknown what extent any of these alternative treatments may be successful at treating future CoV-related diseases.

Utilizing RNA interference to silence a viral genome has been successfully employed in humans and animals against, for example, the hepatitis B virus (HBV), and it is plausible that a similar approach can inhibit SARS-CoV-2 replication. However, to date, others have failed to design an RNAi agent that can provide advantages over the existing vaccines and alternative treatment options for patients (see, e.g., https://investors.alnylam.com/press-release?id=25901, 3 Aug. 2021 Press Release Announcing Discontinuation of RNA interference ALN-COV program “based on availability of highly effective vaccines and alternative treatment options” (last visited 30 Jan. 2023)). Thus, there remains a need for a therapeutic that can silence viral genomes of SARS-CoV-2, and in particular an RNAi agent with the potential to inhibit the replication of other CoV genomes beyond SARS-CoV-2 that may arise in the future.

While in vitro screening of potentially active sequences that are complementary to a known gene or genome being targeted is routine, the lack of a reliable correlation between in vitro data and in vivo activity frequently renders this screening exercise incomplete and potentially misleading, as often the most potent RNAi agent sequences in vivo are not always the most active performers in vitro. (See, e.g., D. Pascut et al., Biosci Rep. 35(2) (2015) (“In other words, the siRNA-mRNA target features involved in siRNA efficacy extracted from data that have small sample size and unique experimental settings (i.e. a set of siRNA against the same target or a restrict number of targets) are likely to perform unsatisfactorily when applied on large datasets under different experimental settings. In vitro experiments could not accurately represent the dynamic setting encountered in vivo.”)). Further, such screening fails to account for potential and unintended off-target effects, which can only be confirmed by in vivo exploration and confirmation. (See, e.g., P. Kamola et al., PLOS Computational Biology 11(12) (2015) (“While high on-target knockdown is essential, it is important to address the problem of unintended off-target effects”).

To be useful as a therapeutic against SARS-CoV-2 and potentially other future CoV outbreaks, the CoV RNAi agent must be able to silence highly conserved sequences in essential RNAs. Thus, identifying a highly-specific and conserved nucleotide sequence for an RNAi agent against CoV genomes (and specifically including a SARS-CoV-2 genome) that is proven to be capable of being delivered in vivo to the lung tissues and can provide highly potent and durable genome knockdown with minimal off-target effects is a significant challenge, but is required for the discovery of a useful RNAi agent therapeutic against CoV.

### SUMMARY

There exists a need for novel RNA interference (RNAi) agents (termed RNAi agents, RNAi triggers, or triggers), e.g., double stranded RNAi agents, that are able to selectively and efficiently inhibit the expression of CoV viral genomes, including but not limited to selectively and efficiently inhibiting the expression and thus the replication of SARS-CoV-2. Further, there exists a need for compositions of novel CoV-specific RNAi agents for use as a therapeutic or medicament for the treatment of COVID-19 and/or diseases or disorders that can be mediated at least in part by a reduction in CoV viral genome expression.

The nucleotide sequences and chemical modifications of the CoV RNAi agents disclosed herein, as well as their combination with certain specific targeting ligands suitable for selectively and efficiently delivering the CoV RNAi agents to pulmonary cells in vivo, differ from those previously disclosed or known in the art. The CoV RNAi agents disclosed herein provide for highly potent and efficient in vivo inhibition of the expression of a SARS-CoV-2 genome, and because of the conserved nature of the RNAi agent antisense strand sequences disclosed herein, are expected to effectively inhibit numerous coronavirus genomes beyond SARS-CoV-2.

In general, the present disclosure features CoV RNAi agents that are specific to SARS-CoV-2 and target a portion of the genome that is conserved across other CoV genomes, compositions that include the disclosed CoV RNAi agents, and methods for inhibiting expression of a SARS-CoV-2 viral genome and/or other CoV genomes in vitro and/or in vivo, using the CoV RNAi agents and compositions that include CoV RNAi agents described herein. The CoV RNAi agents described herein are able to selectively and efficiently decrease expression of a SARS-CoV-2 viral genome and potentially other CoV genomes.

The described CoV RNAi agents can be used in methods for therapeutic treatment (including potentially preventative or prophylactic treatment) of symptoms or diseases related to CoV viral infection, including but not limited to COVID 19 and lung inflammation.

In one aspect, the disclosure features RNAi agents for inhibiting expression of a SARS-CoV-2 viral genome or another CoV viral genome, wherein the RNAi agent includes a sense strand (also referred to as a passenger strand) and an antisense strand (also referred to as a guide strand). The sense strand and the antisense strand can be partially, substantially, or fully complementary to each other. The length of the RNAi agent sense strands described herein each can be 15 to 49 nucleotides in length. The length of the RNAi agent antisense strands described herein each can be 18 to 49 nucleotides in length. In some embodiments, the sense and antisense strands are independently 18 to 26 nucleotides in length. The sense and antisense strands can be either the same length or different lengths. In some embodiments, the sense and antisense strands are independently 21 to 26 nucleotides in length. In some embodiments, the sense and antisense strands are independently 21 to 24 nucleotides in length. In some embodiments, both the sense strand and the antisense strand are 21 nucleotides in length. In some embodiments, the antisense strands are independently 18, 19, 20, 21, 22, 23, 24, 25, 26, 27, 28, 29, or 30 nucleotides in length. In some embodiments, the sense strands are independently 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 nucleotides in length. The RNAi agents described herein, upon delivery to a cell expressing SARS-CoV-2 such as a pulmonary cell, inhibit the expression of one or more SARS-CoV-2 viral genome variants in vivo and/or in vitro through the RNA-induced silencing complex (RISC)-mediated cleavage of the viral RNA genome and RNA transcripts.

The CoV RNAi agents disclosed herein are designed to target a SARS-CoV-2 viral genome (see, e.g., SEQ ID NO:1) in a region of the genome that is anticipated to be conserved across a variety of different coronaviruses. In some embodiments, the CoV RNAi agents disclosed herein are designed to target a portion of a SARS-CoV-2 viral genome having the sequence of any of the sequences disclosed in Table 1.

In another aspect, the disclosure features compositions, including pharmaceutical compositions, that include one or more of the disclosed CoV RNAi agents that are able to selectively and efficiently decrease expression of a SARS-CoV-2 viral genome or a different CoV viral genome. The compositions that include one or more CoV RNAi agents described herein can be administered to a subject, such as a human or animal subject, for the treatment (including potential prophylactic treatment or inhibition) of symptoms and diseases associated with coronavirus infection, including but not limited to COVID-19 and lung inflammation.

Examples of CoV RNAi agent sense strands and antisense strands that can be used in a CoV RNAi agent are provided in Tables 3, 4, 5, and 6. Examples of CoV RNAi agent duplexes are provided in Tables 7A, 7B, 8, 9, and 10. Examples of 19-nucleotide core stretch sequences that may consist of or may be included in the sense strands and antisense strands of certain CoV RNAi agents disclosed herein, are provided in Table 2.

In another aspect, the disclosure features methods for delivering CoV RNAi agents to pulmonary epithelial cells in a subject, such as a mammal, in vivo. Also described herein are compositions for use in such methods. In some embodiments, disclosed herein are methods for delivering CoV RNAi agents to pulmonary cells (epithelial cells (including alveolar type I and type II pneumocytes), mesenchymal cells (including smooth muscle cells and fibroblasts), immune cells (including macrophages and mast cells) and endothelial cells) to a subject in vivo. In some embodiments, the subject is a human subject.

The methods disclosed herein include the administration of one or more CoV RNAi agents to a subject, e.g., a human or animal subject, by any suitable means known in the art. The pharmaceutical compositions disclosed herein that include one or more CoV RNAi agents can be administered in a number of ways depending upon whether local or systemic treatment is desired. Administration can be, but is not limited to, for example, intravenous, intraarterial, subcutaneous, intraperitoneal, subdermal (e.g., via an implanted device), and intraparenchymal administration. In some embodiments, the pharmaceutical compositions described herein are administered by inhalation (such as dry powder inhalation or aerosol inhalation), intranasal administration, intratracheal administration, or oropharyngeal aspiration administration.

In some embodiments, it is desired that the CoV RNAi agents described herein inhibit the expression of a CoV viral genome in the pulmonary epithelium, for which the administration is by inhalation (e.g., by an inhaler device, such as a metered-dose inhaler, or a nebulizer such as a jet or vibrating mesh nebulizer, or a soft mist inhaler). In some embodiments, the viral genome being inhibited is SARS-CoV-2.

The CoV RNAi agents described herein can be delivered to target cells or tissues using any oligonucleotide delivery technology known in the art. In some embodiments, a CoV RNAi agent is delivered to cells or tissues by covalently linking the RNAi agent to a targeting group. In some embodiments, the targeting group can include a cell receptor ligand, such as an integrin targeting ligand. Integrins are a family of transmembrane receptors that facilitate cell-extracellular matrix (ECM) adhesion. In particular, integrin alpha-v-beta-6 (αvβ6) is an epithelial-specific integrin that is known to be a receptor for ECM proteins and the TGF-beta latency-associated peptide (LAP), and is expressed in various cells and tissues. Integrin αvβ6 is known to be highly upregulated in injured pulmonary epithelium. In some embodiments, the CoV RNAi agents described herein are linked to an integrin targeting ligand that has affinity for integrin αvβ6. As referred to herein, an “αvβ6 integrin targeting ligand” is a compound that has affinity for integrin αvβ6, which can be utilized as a ligand to facilitate the targeting and delivery of an RNAi agent to which it is attached to the desired cells and/or tissues (i.e., to cells expressing integrin αvβ6). In some embodiments, multiple αvβ6 integrin targeting ligands or clusters of αvβ6 integrin targeting ligands are linked to a CoV RNAi agent. In some embodiments, the CoV RNAi agent-αvβ6 integrin targeting ligand conjugates are selectively internalized by lung epithelial cells, either through receptor-mediated endocytosis or by other means.

Examples of targeting groups useful for delivering CoV RNAi agents that include αvβ6 integrin targeting ligands are disclosed, for example, in International Patent Application Publication No. WO 2018/085415 and International Patent Application Publication No. WO 2019/089765, the contents of each of which are incorporated by reference herein in their entirety.

A targeting group can be linked to the 3′ or 5′ end of a sense strand or an antisense strand of a CoV RNAi agent. In some embodiments, a targeting group is linked to the 3′ or 5′ end of the sense strand. In some embodiments, a targeting group is linked to the 5′ end of the sense strand. In some embodiments, a targeting group is linked internally to a nucleotide on the sense strand and/or the antisense strand of the RNAi agent. In some embodiments, a targeting group is linked to the RNAi agent via a linker.

In another aspect, the disclosure features compositions that include one or more CoV RNAi agents that have the duplex structures disclosed in Tables 7A, 7B, 8, 9, and 10.

The use of CoV RNAi agents provides methods for therapeutic (including prophylactic) treatment of diseases or disorders related to coronavirus infection, such as COVID-19 caused by SARS-CoV-2. The CoV RNAi agents disclosed herein can be used to treat various respiratory diseases and injury related to coronavirus infection. In some embodiments, the CoV RNAi agents disclosed herein can be used to treat or prevent a pulmonary inflammatory disease or condition.

### Definitions

As used herein, the terms “oligonucleotide” and “polynucleotide” mean a polymer of linked nucleosides each of which can be independently modified or unmodified.

As used herein, an “RNAi agent” (also referred to as an “RNAi trigger”) means a chemical composition of matter that contains an RNA or RNA-like (e.g., chemically modified RNA) oligonucleotide molecule that is capable of degrading RNA or inhibiting (e.g., degrades or inhibits under appropriate conditions) translation of viral RNA (including viral RNA and viral mRNA messenger RNA (mRNA) transcripts) of a target coronavirus in a sequence specific manner. As used herein, RNAi agents may operate through the RNA interference mechanism (i.e., inducing RNA interference through interaction with the RNA interference pathway machinery (RNA-induced silencing complex or RISC) of mammalian cells), or by any alternative mechanism(s) or pathway(s). While it is believed that RNAi agents, as that term is used herein, operate primarily through the RNA interference mechanism, the disclosed RNAi agents are not bound by or limited to any particular pathway or mechanism of action. RNAi agents disclosed herein are comprised of a sense strand and an antisense strand, and include, but are not limited to: small (or short) interfering RNAs (siRNAs), double stranded RNAs (dsRNA), micro RNAs (miRNAs), short hairpin RNAs (shRNA), and dicer substrates. The antisense strand of the RNAi agents described herein is at least partially complementary to the RNA being targeted (e.g., SARS-CoV-2 RNA). RNAi agents can include one or more modified nucleotides and/or one or more non-phosphodiester linkages.

As used herein, the terms “silence,” “reduce,” “inhibit,” “down-regulate,” or “knockdown” when referring to expression of a given viral genome, mean that the expression of the viral genome (viral genomic RNA or subgenomic RNA), as measured by the level of RNA transcribed from the gene or genome, the number of viral genomes, or the level of polypeptide, protein, or protein subunit translated from the viral RNA in a cell, group of cells, tissue, organ, or subject in which the gene or viral genome is transcribed, is reduced when the cell, group of cells, tissue, organ, or subject is treated with the RNAi agents described herein as compared to a second cell, group of cells, tissue, organ, or subject that has not or have not been so treated. Without being bound to any particular theory, it is believed that the CoV RNAi agents disclosed herein utilize the RNA interference mechanism to inhibit CoV viral transcripts thereby leading to a reduction in viral genome expression.

As used herein, the terms “sequence” and “nucleotide sequence” mean a succession or order of nucleobases or nucleotides, described with a succession of letters using standard nomenclature.

As used herein, a “base,” “nucleotide base,” or “nucleobase,” is a heterocyclic pyrimidine or purine compound that is a component of a nucleotide, and includes the primary purine bases adenine and guanine, and the primary pyrimidine bases cytosine, thymine, and uracil. A nucleobase may further be modified to include, without limitation, universal bases, hydrophobic bases, promiscuous bases, size-expanded bases, and fluorinated bases. (See, e.g., Modified Nucleosides in Biochemistry, Biotechnology and Medicine, Herdewijn, P. ed. Wiley-VCH, 2008). The synthesis of such modified nucleobases (including phosphoramidite compounds that include modified nucleobases) is known in the art.

As used herein, and unless otherwise indicated, the term “complementary,” when used to describe a first nucleobase or nucleotide sequence (e.g., RNAi agent sense strand or targeted RNA) in relation to a second nucleobase or nucleotide sequence (e.g., RNAi agent antisense strand or a single-stranded antisense oligonucleotide), means the ability of an oligonucleotide or polynucleotide including the first nucleotide sequence to hybridize (form base pair hydrogen bonds under mammalian physiological conditions (or otherwise suitable in vivo or in vitro conditions)) and form a duplex or double helical structure under certain standard conditions with an oligonucleotide that includes the second nucleotide sequence. The person of ordinary skill in the art would be able to select the set of conditions most appropriate for a hybridization test. Complementary sequences include Watson-Crick base pairs or non-Watson-Crick base pairs and include natural or modified nucleotides or nucleotide mimics, at least to the extent that the above hybridization requirements are fulfilled. Sequence identity or complementarity is independent of modification. For example, a and Af, as defined herein, are complementary to U (or T) and identical to A for the purposes of determining identity or complementarity.

As used herein, “perfectly complementary” or “fully complementary” means that in a hybridized pair of nucleobase or nucleotide sequence molecules, all (100%) of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “partially complementary” means that in a hybridized pair of nucleobase or nucleotide sequence molecules, at least 70%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, “substantially complementary” means that in a hybridized pair of nucleobase or nucleotide sequence molecules, at least 85%, but not all, of the bases in a contiguous sequence of a first oligonucleotide will hybridize with the same number of bases in a contiguous sequence of a second oligonucleotide. The contiguous sequence may comprise all or a part of a first or second nucleotide sequence.

As used herein, the terms “complementary,” “fully complementary,” “partially complementary,” and “substantially complementary” are used with respect to the nucleobase or nucleotide matching between the sense strand and the antisense strand of an RNAi agent, or between the antisense strand of an RNAi agent and a sequence of a CoV RNA, such as a SARS-CoV-2 RNA.

As used herein, the term “substantially identical” or “substantial identity,” as applied to a nucleic acid sequence means the nucleotide sequence (or a portion of a nucleotide sequence) has at least about 85% sequence identity or more, e.g., at least 90%, at least 95%, or at least 99% identity, compared to a reference sequence. Percentage of sequence identity is determined by comparing two optimally aligned sequences over a comparison window. The percentage is calculated by determining the number of positions at which the same type of nucleic acid base occurs in both sequences to yield the number of matched positions, dividing the number of matched positions by the total number of positions in the window of comparison and multiplying the result by 100 to yield the percentage of sequence identity. The inventions disclosed herein encompass nucleotide sequences substantially identical to those disclosed herein.

As used herein, the terms “treat,” “treatment,” and the like, mean the methods or steps taken to provide relief from or alleviation of the number, severity, and/or frequency of one or more symptoms of a disease in a subject. As used herein, “treat” and “treatment” may include the prevention, management, prophylactic treatment, and/or inhibition or reduction of the number, severity, and/or frequency of one or more symptoms of a disease in a subject.

As used herein, the phrases “symptoms and diseases associated with coronavirus infection” and a “coronavirus associate disease” refer to a symptom, disease, or disorder that is caused by or associated with a coronavirus infection. A “coronavirus infection” includes an infection with any coronavirus such as, for example, the two alpha-CoVs (HCoVs-NL63 and HCoVs-229E), the two beta-CoVs (HCoVs-OC43 and HCoVs-HKU1), severe acute respiratory syndrome-CoV (SARS-CoV), and Middle East respiratory syndrome-CoV (MERS-CoV). The symptoms of a coronavirus infection depend on the seriousness of the infection and the type of coronavirus.

As used herein, the phrase “introducing into a cell,” when referring to an RNAi agent, means functionally delivering the RNAi agent into a cell. The phrase “functional delivery,” means delivering the RNAi agent to the cell in a manner that enables the RNAi agent to have the expected biological activity, e.g., sequence-specific inhibition of gene or viral genome expression.

Unless stated otherwise, use of the symbol [symbol] as used herein means that any group or groups may be linked thereto that is in accordance with the scope of the inventions described herein.

As used herein, the term “isomers” refers to compounds that have identical molecular formulae, but that differ in the nature or the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images are termed “enantiomers,” or sometimes optical isomers. A carbon atom bonded to four non-identical substituents is termed a “chiral center.”

As used herein, unless specifically identified in a structure as having a particular conformation, for each structure in which asymmetric centers are present and thus give rise to enantiomers, diastereomers, or other stereoisomeric configurations, each structure disclosed herein is intended to represent all such possible isomers, including their optically pure and racemic forms. For example, the structures disclosed herein are intended to cover mixtures of diastereomers as well as single stereoisomers.

As used in a claim herein, the phrase “consisting of” excludes any element, step, or ingredient not specified in the claim. When used in a claim herein, the phrase “consisting essentially of” limits the scope of a claim to the specified materials or steps and those that do not materially affect the basic and novel characteristic(s) of the claimed invention.

The person of ordinary skill in the art would readily understand and appreciate that the compounds and compositions disclosed herein may have certain atoms (e.g., N, O, or S atoms) in a protonated or deprotonated state, depending upon the environment in which the compound or composition is placed. Accordingly, as used herein, the structures disclosed herein envisage that certain functional groups, such as, for example, OH, SH, or NH, may be protonated or deprotonated. The disclosure herein is intended to cover the disclosed compounds and compositions regardless of their state of protonation based on the environment (such as pH), as would be readily understood by the person of ordinary skill in the art. Correspondingly, compounds described herein with labile protons or basic atoms should also be understood to represent salt forms of the corresponding compound. The RNAi agents described herein may be in a free acid, free base, or salt form. Pharmaceutically acceptable salts of the RNAi agent compounds described herein should be understood to be within the scope of the invention.

As used herein, the term “linked” or “conjugated” when referring to the connection between two compounds or molecules means that two compounds or molecules are joined by a covalent bond. Unless stated, the terms “linked” and “conjugated” as used herein may refer to the connection between a first compound and a second compound either with or without any intervening atoms or groups of atoms.

As used herein, the term “including” is used to herein mean, and is used interchangeably with, the phrase “including but not limited to.” The term “or” is used herein to mean, and is used interchangeably with, the term “and/or,” unless the context clearly indicates otherwise.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other objects, features, aspects, and advantages of the invention will be apparent from the following detailed description, accompanying figures, and from the claims.

### BRIEF DESCRIPTION OF THE DRAWINGS

FIG. 1. Chemical structure representation of the tridentate αvβ6 epithelial cell targeting ligand referred to herein as Tri-SM6.1-αvβ6-(TA14).

FIG. 2. Chemical structure representation of the peptide αvβ6 epithelial cell targeting ligand referred to herein as αvβ6-pep1.

FIG. 3. Graph plotting the reduction of CoV genomic viral RNA in lung tissue on Day 3 post infection (see also Example 10).

FIG. 4. Graph plotting the reduction of CoV subgenomic RNA in lung tissue on Day 3 post infection. (see also Example 10).

FIG. 5. Bar graph showing reduction of total inflammation of the superior lobe of the lung on Day 7 post infection (see also Example 10).

FIG. 6. Bar graph showing reduction of alveolar inflammation on Day 7 post infection (see also Example 10).

FIG. 7. Bar graph showing plaque-forming units (PFU) reduction on Day 3 post infection (see also Example 10).

FIG. 8. Line graph showing body weight restoration (see also Example 10).

FIG. 9. Pre-SARS-CoV-2 infection, % hamster body weight change (see also Example 13).

FIG. 10. Post-SARS-CoV-2 infection, % hamster body weight change (see also Example 13).

FIG. 11. Viral titers in lung tissue at days 3 and 7 post infection expressed in PFU/ml homogenized tissue. Dotted line indicates the limit of detection (LOD) of the assay of 10 PFU/ml (see also Example 13).

FIG. 12. Viral titers in lungs at days 3 and 7 post infection expressed as PFU/gram of lung tissue, normalized to the weight of the tissue. Dotted line indicates the limit of detection (LOD) of the assay of 10 PFU/gram (see also Example 13).

FIG. 13. Viral genomic RNA copies in lung homogenates at days 3 and 7 post infection, normalized to 100 mg of lung tissue (see also Example 13).

FIG. 14. Viral subgenomic RNA copies in the lung homogenates at days 3 and 7 post infection, normalized to 100 mg of lung tissue (see also Example 13).

FIG. 15A. Pre-SARS-CoV-2 infection, % body weight change: Body weight at day −7 was used to calculate % body weight gain/loss in the pre-infection phase (see also Example 14).

FIG. 15B. Post-SARS-CoV-2 infection, % body weight change: Body weight at day 0 was used to calculate % body weight gain/loss in post infection phase (see also Example 14).

FIG. 16A. Viral genomic RNA copies in the lung, 3 days post infection (see also Example 14).

FIG. 16B. Viral subgenomic RNA copies in the lung, 3 days post infection (see also Example 14).

FIG. 17A. Viral genomic RNA copies in the lung, 7 days post infection (see also Example 14).

FIG. 17B. Viral subgenomic RNA copies in the lung, 7 days post infection (see also Example 14).

FIG. 18A. Viral titers in lungs at day 3 post infection expressed in PFU/ml (see also Example 14).

FIG. 18B. Viral titers in lungs at day 3 post infection expressed in PFU/gram of lung tissue (see also Example 14).

FIG. 19A. Pre-SARS-CoV-2 infection, % body weight change: Body weight at day −7 was used to calculate % body weight gain/loss in the pre-infection phase (see also Example 14).

FIG. 19B. Post-SARS-CoV-2 infection, % body weight change: Body weight at day 0 was used to calculate % body weight gain/loss in post infection phase (see also Example 14).

FIG. 20A. Viral genomic RNA copies in the lung, 3 days post infection (see also Example 14).

FIG. 20B. Viral subgenomic RNA copies in the lung, 3 days post infection (see also Example 14).

FIG. 21A. Viral genomic RNA copies in the lung, 7 days post infection (see also Example 14).

FIG. 21B. Viral subgenomic RNA copies in the lung, 7 days post infection (see also Example 14).

FIG. 21C. Viral titers determined by plaque assay in PFU/ml, 3 days post infection (see also Example 14).

FIG. 21D. Viral titers normalized to the weight of the tissue and expressed as PFU/gram of lung tissue, 3 days post infection (see also Example 14).

FIG. 22A. Bar graph showing reduction of total inflammation of the superior lobe section of the lung on Day 7 post infection (see also Example 14).

FIG. 22B. Bar graph showing reduction of inflammation of the alveolar lung area of the superior lobe section of the lung on Day 7 post infection (see also Example 14).

FIG. 23A. H&E-stained superior lobe of the right lung of uninfected control group hamsters that were naïve or received saline vehicle, lungs collected 3 days post infection (see also Example 14).

FIG. 23B. H&E-stained superior lobe of the right lung of hamsters that received AC001927 or saline control, lungs collected 3 days post infection (see also Example 14).

FIG. 23C. H&E-stained superior lobe of the right lung of hamsters that received AC002617 or saline control, lungs collected 3 days post infection (see also Example 14).

FIG. 23D. H&E-stained superior lobe of the right lung of hamsters that received AC002618 or saline control, lungs collected 3 days post infection (see also Example 14).

FIG. 23E. H&E-stained superior lobe of the right lung of hamsters that received AC002620 or saline control, lungs collected 3 days post infection (see also Example 14).

FIG. 23F. H&E-stained superior lobe of the right lung of hamsters that received AC002621 or saline control, lungs collected 3 days post infection (see also Example 14).

FIG. 24A. H&E-stained superior lobe of the right lung of hamsters that received AC001927 or saline control, lungs collected 7 days post infection (see also Example 14).

FIG. 24B. H&E-stained superior lobe of the right lung of hamsters that received AC002617 or saline control, lungs collected 7 days post infection (see also Example 14).

FIG. 24C. H&E-stained superior lobe of the right lung of hamsters that received AC002618 or saline control, lungs collected 7 days post infection (see also Example 14).

FIG. 24D. H&E-stained superior lobe of the right lung of hamsters that received AC002620 or saline control, lungs collected 7 days post infection (see also Example 14).

FIG. 24E. H&E-stained superior lobe of the right lung of hamsters that received AC002621 or saline control, lungs collected 7 days post infection (see also Example 14).

FIG. 25A. Group average hamster body weights (g) pre- and post-SARS-CoV-2 infection (see also Example 15).

FIG. 25B. Total pulmonary inflammation area of the experimental groups treated with the CoV RNAi agents (see also Example 15).

FIG. 25C. Viral genomic RNA copies in the lung, 3 days post infection (see also Example 15).

FIG. 25D. Viral subgenomic RNA copies in the lung, 3 days post infection (see also Example 15).

FIG. 25E. Viral genomic RNA copies in the lung, 7 days post infection (see also Example 15).

FIG. 25F. Viral subgenomic RNA copies in the lung, 7 days post infection (see also Example 15).

### DETAILED DESCRIPTION

### RNAi Agents

Described herein are RNAi agents for inhibiting expression of a CoV viral genome, including but not limited to SARS-CoV-2 (referred to herein as CoV RNAi agents or CoV RNAi triggers). Each CoV RNAi agent disclosed herein comprises a sense strand and an antisense strand. The sense strand can be 15 to 49 nucleotides in length, and the antisense strand can be 18 to 49 nucleotides in length. The sense and antisense strands can be either the same length or they can be different lengths. In some embodiments, the sense and antisense strands are each independently 18 to 27 nucleotides in length. In some embodiments, both the sense and antisense strands are each 19-26 nucleotides in length. In some embodiments, the sense and antisense strands are each 21-24 nucleotides in length. In some embodiments, the sense and antisense strands are each independently 19-21 nucleotides in length. In some embodiments, the sense strand is about 19 nucleotides in length while the antisense strand is about 21 nucleotides in length. In some embodiments, the sense strand is about 21 nucleotides in length while the antisense strand is about 23 nucleotides in length. In some embodiments, a sense strand is 23 nucleotides in length and an antisense strand is 21 nucleotides in length. In some embodiments, both the sense and antisense strands are each 21 nucleotides in length. In some embodiments, the RNAi agent sense strands are 15, 16, 17, 18, 19, 20, 21, 22, 23, 24, 25, 26, 27, 28, 29, 30, 31, 32, 33, 34, 35, 36, 37, 38, or 39 nucleotides in length. In some embodiments, the RNAi agent antisense strands are 18, 19, 20, 21, 22, 23, 24, 25, 26, 27, 28, 29, 30, 31, 32, 33, 34, 35, 36, 37, 38, or 39 nucleotides in length. In some embodiments, a double-stranded RNAi agent has a duplex length of about 16, 17, 18, 19, 20, 21, 22, 23 or 24 nucleotides.

Examples of nucleotide sequences used in forming CoV RNAi agents are provided in Tables 2, 3, 4, 5, 6, and 10. Examples of RNAi agent duplexes, that include the sense strand and antisense strand sequences in Tables 2, 3, 4, 5, 6, are shown in Tables 7A, 7B, 8, 9, and 10.

In some embodiments, the region of perfect, substantial, or partial complementarity between the sense strand and the antisense strand is 15-26 (e.g., 15, 16, 17, 18, 19, 20, 21, 22, 23, 24, 25, or 26) nucleotides in length and occurs at or near the 5′ end of the antisense strand (e.g., this region may be separated from the 5′ end of the antisense strand by 0, 1, 2, 3, or 4 nucleotides that are not perfectly, substantially, or partially complementary).

A sense strand of the CoV RNAi agents described herein includes at least 15 consecutive nucleotides that have at least 85% identity to a core stretch sequence (also referred to herein as a “core stretch” or “core sequence”) of the same number of nucleotides in a SARS-CoV-2 RNA (including all viral RNA as well as viral mRNA). In some embodiments, a sense strand core stretch sequence is 100% (perfectly) complementary or at least about 85% (substantially) complementary to a core stretch sequence in the antisense strand, and thus the sense strand core stretch sequence is typically perfectly identical or at least about 85% identical to a nucleotide sequence of the same length (sometimes referred to, e.g., as a target sequence) present in the SARS-CoV-2 RNA target, which as noted elsewhere is a target sequence that is known to be conserved across a variety of coronaviruses. In some embodiments, this sense strand core stretch is 15, 16, 17, 18, 19, 20, 21, 22, or 23 nucleotides in length. In some embodiments, this sense strand core stretch is 17 nucleotides in length. In some embodiments, this sense strand core stretch is 19 nucleotides in length.

An antisense strand of a CoV RNAi agent described herein includes at least 17 consecutive nucleotides that have at least 85% complementarity to a core stretch of the same number of nucleotides in a SARS-CoV-2 RNA or another CoV RNA being targeted, and at least 15 consecutive nucleotides that have at least 85% complementarity to a core stretch of the same number of nucleotides to a core stretch of the same number of nucleotides in the corresponding sense strand. In some embodiments, an antisense strand core stretch is 100% (perfectly) complementary or at least about 85% (substantially) complementary to a nucleotide sequence (e.g., target sequence) of the same length present in a SARS-CoV-2 RNA target. In some embodiments, this antisense strand core stretch is 15, 16, 17, 18, 19, 20, 21, 22, or 23 nucleotides in length. In some embodiments, this antisense strand core stretch is 19 nucleotides in length. In some embodiments, this antisense strand core stretch is 17 nucleotides in length. A sense strand core stretch sequence can be the same length as a corresponding antisense core sequence or it can be a different length.

The CoV RNAi agent sense and antisense strands anneal to form a duplex. A sense strand and an antisense strand of a CoV RNAi agent can be partially, substantially, or fully complementary to each other. Within the complementary duplex region, the sense strand core stretch sequence is at least 85% complementary or 100% complementary to the antisense core stretch sequence. In some embodiments, the sense strand core stretch sequence contains a sequence of at least 15, at least 16, at least 17, at least 18, at least 19, at least 20, at least 21, at least 22, or at least 23 nucleotides that is at least 85% or 100% complementary to a corresponding 15, 16, 17, 18, 19, 20, 21, 22, or 23 nucleotide sequence of the antisense strand core stretch sequence (i.e., the sense and antisense core stretch sequences of a CoV RNAi agent have a region of at least 15, at least 16, at least 17, at least 18, at least 19, at least 20, at least 21, at least 22, or at least 23 nucleotides that is at least 85% base paired or 100% base paired.)

In some embodiments, the antisense strand of a CoV RNAi agent disclosed herein differs by 0, 1, 2, or 3 nucleotides from any of the antisense strand sequences in Table 2 or Table 3. In some embodiments, the sense strand of a CoV RNAi agent disclosed herein differs by 0, 1, 2, or 3 nucleotides from any of the sense strand sequences in Table 2, Table 4, Table 5, Table 6, or Table 10.

In some embodiments, the sense strand and/or the antisense strand can optionally and independently contain an additional 1, 2, 3, 4, 5, or 6 nucleotides (extension) at the 3′ end, the 5′ end, or both the 3′ and 5′ ends of the core stretch sequences. The antisense strand additional nucleotides, if present, may or may not be complementary to the corresponding sequence in a SARS-CoV-2 RNA. The sense strand additional nucleotides, if present, may or may not be identical to the corresponding sequence in a SARS-CoV-2 RNA. The antisense strand additional nucleotides, if present, may or may not be complementary to the corresponding sense strand's additional nucleotides, if present.

As used herein, an extension comprises 1, 2, 3, 4, 5, or 6 nucleotides at the 5′ and/or 3′ end of the sense strand core stretch sequence and/or antisense strand core stretch sequence. The extension nucleotides on a sense strand may or may not be complementary to nucleotides, either core stretch sequence nucleotides or extension nucleotides, in the corresponding antisense strand. Conversely, the extension nucleotides on an antisense strand may or may not be complementary to nucleotides, either core stretch nucleotides or extension nucleotides, in the corresponding sense strand. In some embodiments, both the sense strand and the antisense strand of an RNAi agent contain 3′ and 5′ extensions. In some embodiments, one or more of the 3′ extension nucleotides of one strand base pairs with one or more 5′ extension nucleotides of the other strand. In other embodiments, one or more of 3′ extension nucleotides of one strand do not base pair with one or more 5′ extension nucleotides of the other strand. In some embodiments, a CoV RNAi agent has an antisense strand having a 3′ extension and a sense strand having a 5′ extension. In some embodiments, the extension nucleotide(s) are unpaired and form an overhang. As used herein, an “overhang” refers to a stretch of one or more unpaired nucleotides located at a terminal end of either the sense strand or the antisense strand that does not form part of the hybridized or duplexed portion of an RNAi agent disclosed herein.

In some embodiments, a CoV RNAi agent comprises an antisense strand having a 3′ extension of 1, 2, 3, 4, 5, or 6 nucleotides in length. In other embodiments, a CoV RNAi agent comprises an antisense strand having a 3′ extension of 1, 2, or 3 nucleotides in length. In some embodiments, one or more of the antisense strand extension nucleotides comprise nucleotides that are complementary to the corresponding SARS-CoV-2 RNA sequence. In some embodiments, one or more of the antisense strand extension nucleotides comprise nucleotides that are not complementary to the corresponding SARS-CoV-2 RNA sequence.

In some embodiments, a CoV RNAi agent comprises a sense strand having a 3′ extension of 1, 2, 3, 4, or 5 nucleotides in length. In some embodiments, one or more of the sense strand extension nucleotides comprises adenosine, uracil, or thymidine nucleotides, AT dinucleotide, or nucleotides that correspond to or are the identical to nucleotides in a SARS-CoV-2 RNA sequence. In some embodiments, the 3′ sense strand extension includes or consists of one of the following sequences, but is not limited to: T, UT, TT, UU, UUT, TTT, or TTTT (each listed 5′ to 3′).

A sense strand can have a 3′ extension and/or a 5′ extension. In some embodiments, a CoV RNAi agent comprises a sense strand having a 5′ extension of 1, 2, 3, 4, 5, or 6 nucleotides in length. In some embodiments, one or more of the sense strand extension nucleotides comprise nucleotides that correspond to or are identical to nucleotides in a SARS-CoV-2 RNA sequence.

Examples of sequences used in forming CoV RNAi agents are provided in Tables 2, 3, 4, 5, 6, and 10. In some embodiments, a CoV RNAi agent antisense strand includes a sequence of any of the sequences in Tables 2, 3, or 10. In certain embodiments, a CoV RNAi agent antisense strand comprises or consists of any one of the modified sequences in Table 3. In some embodiments, a CoV RNAi agent antisense strand includes the sequence of nucleotides (from 5′ end→3′ end) 1-17, 2-15, 2-17, 1-18, 2-18, 1-19, 2-19, 1-20, 2-20, 1-21, or 2-21, of any of the sequences in Tables 2 or 3. In some embodiments, a CoV RNAi agent sense strand includes the sequence of any of the sequences in Tables 2, 4, 5, or 6. In some embodiments, a CoV RNAi agent sense strand includes the sequence of nucleotides (from 5′ end→3′ end) 1-18, 1-19, 1-20, 1-21, 2-19, 2-20, 2-21, 3-20, 3-21, or 4-21 of any of the sequences in Tables 2, 4, 5, or 6. In certain embodiments, a CoV RNAi agent sense strand comprises or consists of a modified sequence of any one of the modified sequences in Table 4, 5, 6, or 10.

In some embodiments, the sense and antisense strands of the RNAi agents described herein contain the same number of nucleotides. In some embodiments, the sense and antisense strands of the RNAi agents described herein contain different numbers of nucleotides. In some embodiments, the sense strand 5′ end and the antisense strand 3′ end of an RNAi agent form a blunt end. In some embodiments, the sense strand 3′ end and the antisense strand 5′ end of an RNAi agent form a blunt end. In some embodiments, both ends of an RNAi agent form blunt ends. In some embodiments, neither end of an RNAi agent is blunt-ended. As used herein a “blunt end” refers to an end of a double stranded RNAi agent in which the terminal nucleotides of the two annealed strands are complementary (form a complementary base-pair).

In some embodiments, the sense strand 5′ end and the antisense strand 3′ end of an RNAi agent form a frayed end. In some embodiments, the sense strand 3′ end and the antisense strand 5′ end of an RNAi agent form a frayed end. In some embodiments, both ends of an RNAi agent form a frayed end. In some embodiments, neither end of an RNAi agent is a frayed end. As used herein a frayed end refers to an end of a double stranded RNAi agent in which the terminal nucleotides of the two annealed strands form a pair (i.e., do not form an overhang) but are not complementary (i.e. form a non-complementary pair). In some embodiments, one or more unpaired nucleotides at the end of one strand of a double stranded RNAi agent form an overhang. The unpaired nucleotides may be on the sense strand or the antisense strand, creating either 3′ or 5′ overhangs. In some embodiments, the RNAi agent contains: a blunt end and a frayed end, a blunt end and 5′ overhang end, a blunt end and a 3′ overhang end, a frayed end and a 5′ overhang end, a frayed end and a 3′ overhang end, two 5′ overhang ends, two 3′ overhang ends, a 5′ overhang end and a 3′ overhang end, two frayed ends, or two blunt ends. Typically, when present, overhangs are located at the 3′ terminal ends of the sense strand, the antisense strand, or both the sense strand and the antisense strand.

The CoV RNAi agents disclosed herein may also be comprised of one or more modified nucleotides. In some embodiments, substantially all of the nucleotides of the sense strand and substantially all of the nucleotides of the antisense strand of the CoV RNAi agent are modified nucleotides. The CoV RNAi agents disclosed herein may further be comprised of one or more modified internucleoside linkages, e.g., one or more phosphorothioate or phosphorodithioate linkages. In some embodiments, a CoV RNAi agent contains one or more modified nucleotides and one or more modified internucleoside linkages. In some embodiments, a 2′-modified nucleotide is combined with modified internucleoside linkage.

In some embodiments, a CoV RNAi agent is prepared or provided as a salt, mixed salt, or a free-acid. In some embodiments, a CoV RNAi agent is prepared as a pharmaceutically acceptable salt. In some embodiments, a CoV RNAi agent is prepared as a pharmaceutically acceptable sodium salt. Such forms that are well known in the art are within the scope of the inventions disclosed herein.

### Modified Nucleotides

Modified nucleotides, when used in various oligonucleotide constructs, can preserve activity of the compound in cells while at the same time increasing the serum stability of these compounds, and can also minimize the possibility of activating interferon activity in humans upon administration of the oligonucleotide construct.

In some embodiments, a CoV RNAi agent contains one or more modified nucleotides. As used herein, a “modified nucleotide” is a nucleotide other than a ribonucleotide (2′-hydroxyl nucleotide). In some embodiments, at least 50% (e.g., at least 60%, at least 70%, at least 80%, at least 90%, at least 95%, at least 97%, at least 98%, at least 99%, or 100%) of the nucleotides are modified nucleotides. As used herein, modified nucleotides can include, but are not limited to, deoxyribonucleotides, nucleotide mimics, abasic nucleotides, 2′-modified nucleotides, inverted nucleotides, modified nucleobase-comprising nucleotides, bridged nucleotides, peptide nucleic acids (PNAs), 2′,3′-seco nucleotide mimics (unlocked nucleobase analogues), locked nucleotides, 3′-O-methoxy (2′ internucleoside linked) nucleotides, 2′-F-Arabino nucleotides, 5′-Me, 2′-fluoro nucleotide, morpholino nucleotides, vinyl phosphonate deoxyribonucleotides, vinyl phosphonate containing nucleotides, and cyclopropyl phosphonate containing nucleotides. 2′-modified nucleotides (i.e., a nucleotide with a group other than a hydroxyl group at the 2′ position of the five-membered sugar ring) include, but are not limited to, 2′-O-methyl nucleotides (also referred to as 2′-methoxy nucleotides), 2′-fluoro nucleotides (also referred to herein as 2′-deoxy-2′-fluoro nucleotides), 2′-deoxy nucleotides, 2′-methoxyethyl (2′-O-2-methoxylethyl) nucleotides (also referred to as 2′-MOE), 2′-amino nucleotides, and 2′-alkyl nucleotides. It is not necessary for all positions in a given compound to be uniformly modified. Conversely, more than one modification can be incorporated in a single CoV RNAi agent or even in a single nucleotide thereof. The CoV RNAi agent sense strands and antisense strands can be synthesized and/or modified by methods known in the art. Modification at one nucleotide is independent of modification at another nucleotide.

Modified nucleobases include synthetic and natural nucleobases, such as 5-substituted pyrimidines, 6-azapyrimidines and N-2, N-6 and 0-6 substituted purines, (e.g., 2-aminopropyladenine, 5-propynyluracil, or 5-propynylcytosine), 5-methylcytosine (5-me-C), 5-hydroxymethyl cytosine, inosine, xanthine, hypoxanthine, 2-aminoadenine, 6-alkyl (e.g., 6-methyl, 6-ethyl, 6-isopropyl, or 6-n-butyl) derivatives of adenine and guanine, 2-alkyl (e.g., 2-methyl, 2-ethyl, 2-isopropyl, or 2-n-butyl) and other alkyl derivatives of adenine and guanine, 2-thiouracil, 2-thiothymine, 2-thiocytosine, 5-halouracil, cytosine, 5-propynyl uracil, 5-propynyl cytosine, 6-azo uracil, 6-azo cytosine, 6-azo thymine, 5-uracil (pseudouracil), 4-thiouracil, 8-halo, 8-amino, 8-sulflydryl, 8-thioalkyl, 8-hydroxyl and other 8-substituted adenines and guanines, 5-halo (e.g., 5-bromo), 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

In some embodiments, the 5′ and/or 3′ end of the antisense strand can include abasic residues (Ab), which can also be referred to as an “abasic site” or “abasic nucleotide.” An abasic residue (Ab) is a nucleotide or nucleoside that lacks a nucleobase at the 1′ position of the sugar moiety. (See, e.g., U.S. Pat. No. 5,998,203). In some embodiments, an abasic residue can be placed internally in a nucleotide sequence. In some embodiments, Ab or AbAb can be added to the 3′ end of the antisense strand. In some embodiments, the 5′ end of the sense strand can include one or more additional abasic residues (e.g., (Ab) or (AbAb)). In some embodiments, UUAb, UAb, or Ab are added to the 3′ end of the sense strand. In some embodiments, an abasic (deoxyribose) residue can be replaced with a ribitol (abasic ribose) residue.

In some embodiments, all or substantially all of the nucleotides of an RNAi agent are modified nucleotides. As used herein, an RNAi agent wherein substantially all of the nucleotides present are modified nucleotides is an RNAi agent having four or fewer (i.e., 0, 1, 2, 3, or 4) nucleotides in both the sense strand and the antisense strand being ribonucleotides (i.e., unmodified). As used herein, a sense strand wherein substantially all of the nucleotides present are modified nucleotides is a sense strand having two or fewer (i.e., 0, 1, or 2) nucleotides in the sense strand being unmodified ribonucleotides. As used herein, an antisense strand wherein substantially all of the nucleotides present are modified nucleotides is an antisense strand having two or fewer (i.e., 0, 1, or 2) nucleotides in the antisense strand being unmodified ribonucleotides. In some embodiments, one or more nucleotides of an RNAi agent is an unmodified ribonucleotide. Chemical structures for certain modified nucleotides are set forth in Table 11 herein.

### Modified Internucleoside Linkages

In some embodiments, one or more nucleotides of a CoV RNAi agent are linked by non-standard linkages or backbones (i.e., modified intemucleoside linkages or modified backbones). Modified intemucleoside linkages or backbones include, but are not limited to, phosphorothioate groups (represented herein as a lower case “s”), chiral phosphorothioates, thiophosphates, phosphorodithioates, phosphotriesters, aminoalkyl-phosphotriesters, alkyl phosphonates (e.g., methyl phosphonates or 3′-alkylene phosphonates), chiral phosphonates, phosphinates, phosphoramidates (e.g., 3′-amino phosphoramidate, aminoalkylphosphoramidates, or thionophosphoramidates), thionoalkyl-phosphonates, thionoalkylphosphotriesters, morpholino linkages, boranophosphates having normal 3′-5′ linkages, 2′-5′ linked analogs of boranophosphates, or boranophosphates having inverted polarity wherein the adjacent pairs of nucleoside units are linked 3′-5′ to 5′-3′ or 2′-5′ to 5′-2′. In some embodiments, a modified intemucleoside linkage or backbone lacks a phosphorus atom. Modified intemucleoside linkages lacking a phosphorus atom include, but are not limited to, short chain alkyl or cycloalkyl inter-sugar linkages, mixed heteroatom and alkyl or cycloalkyl inter-sugar linkages, or one or more short chain heteroatomic or heterocyclic inter-sugar linkages. In some embodiments, modified intemucleoside backbones include, but are not limited to, siloxane backbones, sulfide backbones, sulfoxide backbones, sulfone backbones, formacetyl and thioformacetyl backbones, methylene formacetyl and thioformacetyl backbones, alkene-containing backbones, sulfamate backbones, methyleneimino and methylenehydrazino backbones, sulfonate and sulfonamide backbones, amide backbones, and other backbones having mixed N, O, S, and CH2 components.

In some embodiments, a sense strand of a CoV RNAi agent can contain 1, 2, 3, 4, 5, or 6 phosphorothioate linkages, an antisense strand of a CoV RNAi agent can contain 1, 2, 3, 4, 5, or 6 phosphorothioate linkages, or both the sense strand and the antisense strand independently can contain 1, 2, 3, 4, 5, or 6 phosphorothioate linkages. In some embodiments, a sense strand of a CoV RNAi agent can contain 1, 2, 3, or 4 phosphorothioate linkages, an antisense strand of a CoV RNAi agent can contain 1, 2, 3, or 4 phosphorothioate linkages, or both the sense strand and the antisense strand independently can contain 1, 2, 3, or 4 phosphorothioate linkages.

In some embodiments, a CoV RNAi agent sense strand contains at least two phosphorothioate intemucleoside linkages. In some embodiments, the phosphorothioate intemucleoside linkages are between the nucleotides at positions 1-3 from the 3′ end of the sense strand. In some embodiments, one phosphorothioate intemucleoside linkage is at the 5′ end of the sense strand nucleotide sequence, and another phosphorothioate linkage is at the 3′ end of the sense strand nucleotide sequence. In some embodiments, two phosphorothioate intemucleoside linkage are located at the 5′ end of the sense strand, and another phosphorothioate linkage is at the 3′ end of the sense strand. In some embodiments, the sense strand does not include any phosphorothioate intemucleoside linkages between the nucleotides, but contains one, two, or three phosphorothioate linkages between the terminal nucleotides on both the 5′ and 3′ ends and the optionally present inverted abasic residue terminal caps. In some embodiments, the targeting ligand is linked to the sense strand via a phosphorothioate linkage.

In some embodiments, a CoV RNAi agent antisense strand contains four phosphorothioate intemucleoside linkages. In some embodiments, the four phosphorothioate intemucleoside linkages are between the nucleotides at positions 1-3 from the 5′ end of the antisense strand and between the nucleotides at positions 19-21, 20-22, 21-23, 22-24, 23-25, or 24-26 from the 5′ end. In some embodiments, three phosphorothioate intemucleoside linkages are located between positions 1-4 from the 5′ end of the antisense strand, and a fourth phosphorothioate intemucleoside linkage is located between positions 20-21 from the 5′ end of the antisense strand. In some embodiments, a CoV RNAi agent contains at least three or four phosphorothioate intemucleoside linkages in the antisense strand.

### Capping Residues or Moieties

In some embodiments, the sense strand may include one or more capping residues or moieties, sometimes referred to in the art as a “cap,” a “terminal cap,” or a “capping residue.” As used herein, a “capping residue” is a non-nucleotide compound or other moiety that can be incorporated at one or more termini of a nucleotide sequence of an RNAi agent disclosed herein. A capping residue can provide the RNAi agent, in some instances, with certain beneficial properties, such as, for example, protection against exonuclease degradation. In some embodiments, inverted abasic residues (invAb) (also referred to in the art as “inverted abasic sites”) are added as capping residues (see Table 11). (See, e.g., F. Czauderna, Nucleic Acids Res., 2003, 31(11), 2705-16). Capping residues are generally known in the art, and include, for example, inverted abasic residues as well as carbon chains such as a terminal C₃H₇ (propyl), C₆H₁₃ (hexyl), or C₁₂H₂₅ (dodecyl) groups. In some embodiments, a capping residue is present at either the 5′ terminal end, the 3′ terminal end, or both the 5′ and 3′ terminal ends of the sense strand. In some embodiments, the 5′ end and/or the 3′ end of the sense strand may include more than one inverted abasic deoxyribose moiety as a capping residue.

In some embodiments, one or more inverted abasic residues (invAb) are added to the 3′ end of the sense strand. In some embodiments, one or more inverted abasic residues (invAb) are added to the 5′ end of the sense strand. In some embodiments, one or more inverted abasic residues or inverted abasic sites are inserted between the targeting ligand and the nucleotide sequence of the sense strand of the RNAi agent. In some embodiments, the inclusion of one or more inverted abasic residues or inverted abasic sites at or near the terminal end or terminal ends of the sense strand of an RNAi agent allows for enhanced activity or other desired properties of an RNAi agent.

In some embodiments, one or more inverted abasic residues (invAb) are added to the 5′ end of the sense strand. In some embodiments, one or more inverted abasic residues can be inserted between the targeting ligand and the nucleotide sequence of the sense strand of the RNAi agent. The inverted abasic residues may be linked via phosphate, phosphorothioate (e.g., shown herein as (invAb)s)), or other internucleoside linkages. In some embodiments, the inclusion of one or more inverted abasic residues at or near the terminal end or terminal ends of the sense strand of an RNAi agent may allow for enhanced activity or other desired properties of an RNAi agent. In some embodiments, an inverted abasic (deoxyribose) residue can be replaced with an inverted ribitol (abasic ribose) residue. In some embodiments, the 3′ end of the antisense strand core stretch sequence, or the 3′ end of the antisense strand sequence, may include an inverted abasic residue. The chemical structures for inverted abasic deoxyribose residues are shown in Table 11 below.

### CoV RNAi Agents

The CoV RNAi agents disclosed herein are designed to target specific positions on a SARS-CoV-2 viral genome (e.g., SEQ ID NO:1 (NC_045512.2), and these specific targeted positions were selected because they also had sequences believed to be conserved across various other CoV genomes. As defined herein, an antisense strand sequence is designed to target a SARS-CoV-2 viral genome at a given position on the genome when the 5′ terminal nucleobase of the antisense strand is aligned with a position that is 21 nucleotides downstream (towards the 3′ end) from the position on the genome when base pairing to the gene or viral genome. For example, as illustrated in Tables 1 and 2 herein, an antisense strand sequence designed to target a SARS-CoV-2 genome at position 29150 requires that when base pairing to the genome, the 5′ terminal nucleobase of the antisense strand is aligned with position 29170 of a SARS-CoV-2 genome.

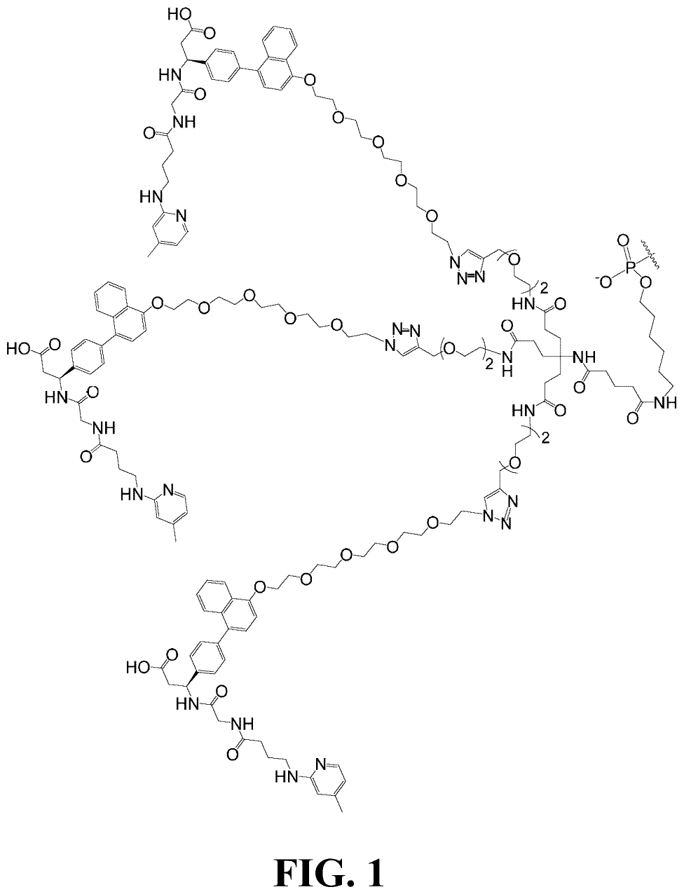

As provided herein, a CoV RNAi agent does not require that the nucleobase at position 1 (5′→3′) of the antisense strand be complementary to the viral genome, provided that there is at least 85% complementarity (e.g., at least 85, 86, 87, 88, 89, 90, 91, 92, 93, 94, 95, 96, 97, 98, 99, or 100% complementarity) of the antisense strand and the viral genome across a core stretch sequence of at least 17 consecutive nucleotides. For example, for a CoV RNAi agent disclosed herein that is designed to target position 29150 of a SARS-CoV-2 viral genome, the 5′ terminal nucleobase of the antisense strand of the of the CoV RNAi agent must be aligned with position 29170 of the genome; however, the 5′ terminal nucleobase of the antisense strand may be, but is not required to be, complementary to position 29170 of a SARS-CoV-2 viral genome, provided that there is at least 85% complementarity (e.g., at least 85, 86, 87, 88, 89, 90, 91, 92, 93, 94, 95, 96, 97, 98, 99, or 100% complementarity) of the antisense strand and the viral genome transcript across a core stretch sequence of at least 17 consecutive nucleotides. As shown by, among other things, the various examples disclosed herein, the specific site of binding of the genome by the antisense strand of the CoV RNAi agent (e.g., whether the CoV RNAi agent is designed to target a SARS-CoV-2 viral genome at position 29150, at position 4156, at position 6412, at position 4917, or at some other position) is an important factor to the level of inhibition achieved by the CoV RNAi agent. (See, e.g., Kamola et al., The siRNA Non-seed Region and Its Target Sequences are Auxiliary Determinants of Off-Target Effects, PLOS Computational Biology, 11(12), FIG. 1 (2015)).

In some embodiments, the CoV RNAi agents disclosed herein target a SARS-CoV-2 viral genome at or near the positions of the SARS-CoV-2 sequence shown in Table 1. In some embodiments, the antisense strand of a CoV RNAi agent disclosed herein includes a core stretch sequence that is fully, substantially, or at least partially complementary to a target SARS-CoV-2 19-mer sequence disclosed in Table 1.

TABLE 1
SARS-CoV-2 19-mer Target Sequences (taken from severe acute respiratory
syndrome coronavirus 2 isolate Wuhan-Hu-1, complete genome (NC_045512.2)
(SEQ ID NO: 1))

| | | | Targeted Viral |
|---|---|---|---|
| | SARS-CoV-2 19-mer | Corresponding | Genome Position |
| SEQ ID | Target Sequences | Positions of Sequence | (as referred to |
| No. | (5′→3') | on SEQ ID NO: 1 | herein) |
| 2 | CACACGUCCAACUCAGUUU | 300-318 | 298 |
| 3 | UUCUUAAGAGUGCUUAUGA | 3654-3672 | 3652 |
| 4 | UCUUAAGAGUGCUUAUGAA | 3655-1673 | 3653 |
| 5 | AUUAAUGGCAAUCUUCAUC | 4040-4058 | 4038 |
| 6 | CUGUGGUUAUACCUACUAA | 4158-4176 | 4156 |
| 7 | ACCUUUGACAAUCUUAAGA | 4919-4937 | 4917 |
| 8 | CAAUCUUAAGACACUUCUU | 4927-4945 | 4925 |
| 9 | AAUCUUAAGACACUUCUUU | 4928-4946 | 4926 |
| 10 | UGGGCACACUUUCUUAUGA | 5577-5595 | 5575 |
| 11 | AGAAAUUGACCCUAAGUUG | 5935-5953 | 5933 |
| 12 | AAGAAGUAGUGGAAAAUCC | 6411-6429 | 6409 |
| 13 | AAGUAGUGGAAAAUCCUAC | 6414-6432 | 6412 |
| 14 | UGCUUACGUUAAUACGUUU | 8041-8059 | 8039 |
| 15 | UAGACAAUGUCUUAUCUAC | 8142-8160 | 8140 |
| 16 | CAUGUGGUAGUGUUGGUUU | 10485-10503 | 10483 |
| 17 | UUUUGAUGUUGUUAGACAA | 10933-10951 | 10931 |
| 18 | GUAAUGCUUUAGAUCAAGC | 11436-11454 | 11434 |
| 19 | AGCUAUGACCCAAAUGUAU | 12286-12304 | 12284 |
| 20 | CAUGGUACCACAUAUAUCA | 13767-13785 | 13765 |
| 21 | AUGGUACCACAUAUAUCAC | 13768-13786 | 13766 |
| 22 | ACUGACAUUAGAUAAUCAA | 14052-14070 | 14050 |
| 23 | UACAUAAUCAGGAUGUAAA | 14501-14519 | 14499 |
| 24 | AUAAUCAGGAUGUAAACUU | 14504-14522 | 14502 |
| 25 | UAAUCAGGAUGUAAACUUA | 14505-14523 | 14503 |
| 26 | AUGUAAACUUACAUAGCUC | 14513-14531 | 14511 |
| 27 | UAAGGCUAGACUUUAUUAU | 14970-14988 | 14968 |
| 28 | GUGUCUCUAUCUGUAGUAC | 15116-15134 | 15114 |
| 29 | CUCUAUCUGUAGUACUAUG | 15120-15138 | 15118 |
| 30 | UCUAUCUGUAGUACUAUGA | 15121-15139 | 15119 |
| 31 | UAUCUGUAGUACUAUGACC | 15123-15141 | 15121 |
| 32 | AUACAAUGCUAGUUAAACA | 15887-15905 | 15885 |
| 33 | UACAAUGCUAGUUAAACAG | 15888-15906 | 15886 |
| 34 | CUAGCAAUGUUGCAAAUUA | 17024-17042 | 17022 |
| 35 | GCAUAAUGUCUGAUAGAGA | 17957-17975 | 17955 |
| 36 | AGACUCAUCUCUAUGAUGG | 18196-18214 | 18194 |
| 37 | GGAGUCACAUUAAUUGGAG | 20110-20128 | 20108 |
| 38 | UCGAUUCAGAUCUUAAUGA | 20945-20963 | 20943 |
| 39 | AUGAGUACGAACUUAUGUA | 26231-26249 | 26229 |
| 40 | GAACUUAUGUACUCAUUCG | 26239-26257 | 26237 |
| 41 | AACUUAUGUACUCAUUCGU | 26240-26258 | 26238 |
| 42 | UAUGUACUCAUUCGUUUCG | 26244-26262 | 26242 |
| 43 | AGUUAAUAGCGUACUUCUU | 26283-26301 | 26281 |
| 44 | UUGCUUUCGUGGUAUUCUU | 26306-26324 | 26304 |
| 45 | ACACUAGCCAUCCUUACUG | 26332-26350 | 26330 |
| 46 | ACUGCUGCAAUAUUGUUAA | 26369-26387 | 26367 |
| 47 | GCUGCAAUAUUGUUAACGU | 26372-26390 | 26370 |
| 48 | CUGCAAUAUUGUUAACGUG | 26373-26391 | 26371 |
| 49 | UGAUCUUCUGGUCUAAACG | 26457-26475 | 26455 |
| 50 | UCACGAACGCUUUCUUAUU | 27039-27057 | 27037 |
| 51 | AACGCUUUCUUAUUACAAA | 27044-27062 | 27042 |
| 52 | CAGUAAGUGACAACAGAUG | 27186-27204 | 27184 |
| 53 | GUAAGUGACAACAGAUGUU | 27188-27206 | 27186 |
| 54 | AGGAAGACCUUAAAUUCCC | 28455-28473 | 28453 |
| 55 | CCAAUUAACACCAAUAGCA | 28490-28508 | 28488 |
| 56 | UCCAGAUGACCAAAUUGGC | 28510-28528 | 28508 |
| 57 | CCAAGAUGGUAUUUCUACU | 28589-28607 | 28587 |
| 58 | AGAUGGUAUUUCUACUACC | 28592-28610 | 28590 |
| 59 | GAAUACACCAAAAGAUCAC | 28690-28708 | 28688 |
| 60 | GUCACUAAGAAAUCUGCUG | 29009-29027 | 29007 |
| 61 | ACUAAAGCAUACAAUGUAA | 29066-29084 | 29064 |
| 62 | AGACAAGGAACUGAUUACA | 29150-29168 | 29148 |
| 63 | ACAAGGAACUGAUUACAAA | 29152-29170 | 29150 |
| 64 | AACUGAUUACAAACAUUGG | 29158-29176 | 29156 |
| 65 | CGCAAAUUGCACAAUUUGC | 29178-29196 | 29176 |
| 66 | AAUUGGAUGACAAAGAUCC | 29286-29304 | 29284 |
| 67 | UGAAUAAGCAUAUUGACGC | 29331-29349 | 29329 |
| 68 | GACGCAUACAAAACAUUCC | 29345-29363 | 29343 |
| 69 | CACAUAGCAAUCUUUAAUC | 29668-29686 | 29666 |

SARS-CoV-2 severe acute respiratory syndrome coronavirus 2 isolate Wuhan-Hu-1, complete genome (NC_045512.2) (SEQ ID NO: 1), viral genome transcript (29903 bases):

| | |
|---|---|
| 1 | attaaaggtt tataccttcc caggtaacaa accaaccaac tttcgatctc ttgtagatct |
| 61 | gttctctaaa cgaactttaa aatctgtgtg gctgtcactc ggctgcatgc ttagtgcact |
| 121 | cacgcagtat aattaataac taattactgt cgttgacagg acacgagtaa ctcgtctatc |
| 181 | ttctgcaggc tgcttacggt ttcgtccgtg ttgcagccga tcatcagcac atctaggttt |
| 241 | cgtccgggtg tgaccgaaag gtaagatgga gagccttgtc cctggtttca acgagaaaac |
| 301 | acacgtccaa ctcagtttgc ctgttttaca ggttcgcgac gtgctcgtac gtggctttgg |
| 361 | agactccgtg gaggaggtct tatcagaggc acgtcaacat cttaaagatg gcacttgtgg |
| 421 | cttagtagaa gttgaaaaag gcgttttgcc tcaacttgaa cagccctatg tgttcatcaa |
| 481 | acgttcggat gctcgaactg cacctcatgg tcatgttatg gttgagctgg tagcagaact |
| 541 | cgaaggcatt cagtacggtc gtagtggtga gacacttggt gtccttgtcc ctcatgtggg |
| 601 | cgaaatacca gtggcttacc gcaaggttct tcttcgtaag aacggtaata aaggagctgg |
| 661 | tggccatagt tacggcgccg atctaaagtc atttgactta ggcgacgagc ttggcactga |
| 721 | tccttatgaa gattttcaag aaaactggaa cactaaacat agcagtggtg ttacccgtga |
| 781 | actcatgcgt gagcttaacg gaggggcata cactcgctat gtcgataaca acttctgtgg |
| 841 | ccctgatggc taccctcttg agtgcattaa agaccttcta gcacgtgctg gtaaagcttc |
| 901 | atgcactttg tccgaacaac tggactttat tgacactaag aggggtgtat actgctgccg |
| 961 | tgaacatgag catgaaattg cttggtacac ggaacgttct gaaaagagct atgaattgca |
| 1021 | gacacctttt gaaattaaat tggcaaagaa atttgacacc ttcaatgggg aatgtccaaa |
| 1081 | ttttgtattt cccttaaatt ccataatcaa gactattcaa ccaagggttg aaaagaaaaa |
| 1141 | gcttgatggc tttatgggta gaattcgatc tgtctatcca gttgcgtcac caaatgaatg |
| 1201 | caaccaaatg tgcctttcaa ctctcatgaa gtgtgatcat tgtggtgaaa cttcatggca |
| 1261 | gacgggcgat tttgttaaag ccacttgcga attttgtggc actgagaatt tgactaaaga |
| 1321 | aggtgccact acttgtggtt acttacccca aaatgctgtt gttaaaattt attgtccagc |
| 1381 | atgtcacaat tcagaagtag gacctgagca tagtcttgcc gaataccata atgaatctgg |
| 1441 | cttgaaaacc attcttcgta agggtggtcg cactattgcc tttggaggct gtgtgttctc |
| 1501 | ttatgttggt tgccataaca agtgtgccta ttgggttcca cgtgctagcg ctaacatagg |
| 1561 | ttgtaaccat acaggtgttg ttggagaagg ttccgaaggt cttaatgaca accttcttga |
| 1621 | aatactccaa aaagagaaag tcaacatcaa tattgttggt gactttaaac ttaatgaaga |
| 1681 | gatcgccatt attttggcat ctttttctgc ttccacaagt gcttttgtgg aaactgtgaa |
| 1741 | aggtttggat tataaagcat tcaaacaaat tgttgaatcc tgtggtaatt ttaaagttac |
| 1801 | aaaaggaaaa gctaaaaaag gtgcctggaa tattggtgaa cagaaatcaa tactgagtcc |
| 1861 | tctttatgca tttgcatcag aggctgctcg tgttgtacga tcaattttct cccgcactct |
| 1921 | tgaaactgct caaaattctg tgcgtgtttt acagaaggcc gctataacaa tactagatgg |
| 1981 | aatttcacag tattcactga gactcattga tgctatgatg ttcacatctg atttggctac |
| 2041 | taacaatcta gttgtaatgg cctacattac aggtggtgtt gttcagttga cttcgcagtg |
| 2101 | gctaactaac atctttggca ctgtttatga aaaactcaaa cccgtccttg attggcttga |
| 2161 | agagaagttt aaggaaggtg tagagtttct tagagacggt tgggaaattg ttaaatttat |
| 2221 | ctcaacctgt gcttgtgaaa ttgtcggtgg acaaattgtc acctgtgcaa aggaaattaa |
| 2281 | ggagagtgtt cagacattct ttaagcttgt aaataaattt ttggctttgt gtgctgactc |
| 2341 | tatcattatt ggtggagcta aacttaaagc cttgaattta ggtgaaacat ttgtcacgca |
| 2401 | ctcaaaggga ttgtacagaa agtgtgttaa atccagagaa gaaactggcc tactcatgcc |
| 2461 | tctaaaagcc ccaaaagaaa ttatcttctt agagggagaa acacttccca cagaagtgtt |
| 2521 | aacagaggaa gttgtcttga aaactggtga tttacaacca ttagaacaac ctactagtga |
| 2581 | agctgttgaa gctccattgg ttggtacacc agtttgtatt aacgggctta tgttgctcga |
| 2641 | aatcaaagac acagaaaagt actgtgccct tgcacctaat atgatggtaa caaacaatac |
| 2701 | cttcacactc aaaggcggtg caccaacaaa ggttactttt ggtgatgaca ctgtgataga |
| 2761 | agtgcaaggt tacaagagtg tgaatatcac ttttgaactt gatgaaagga ttgataaagt |
| 2821 | acttaatgag aagtgctctg cctatacagt tgaactcggt acagaagtaa atgagttcgc |
| 2881 | ctgtgttgtg gcagatgctg tcataaaaac tttgcaacca gtatctgaat tacttacacc |
| 2941 | actgggcatt gatttagatg agtggagtat ggctacatac tacttatttg atgagtctgg |
| 3001 | tgagtttaaa ttggcttcac atatgtattg ttctttctac cctccagatg aggatgaaga |
| 3061 | agaaggtgat tgtgaagaag aagagtttga gccatcaact caatatgagt atggtactga |
| 3121 | agatgattac caaggtaaac ctttggaatt tggtgccact tctgctgctc ttcaacctga |
| 3181 | agaagagcaa gaagaagatt ggttagatga tgatagtcaa caaactgttg gtcaacaaga |
| 3241 | cggcagtgag gacaatcaga caactactat tcaaacaatt gttgaggttc aacctcaatt |
| 3301 | agagatggaa cttacaccag ttgttcagac tattgaagtg aatagtttta gtggttattt |
| 3361 | aaaacttact gacaatgtat acattaaaaa tgcagacatt gtggaagaag ctaaaaaggt |
| 3421 | aaaaccaaca gtggttgtta atgcagccaa tgtttacctt aaacatggag gaggtgttgc |
| 3481 | aggagcctta aataaggcta ctaacaatgc catgcaagtt gaatctgatg attacatagc |
| 3541 | tactaatgga ccacttaaag tgggtggtag ttgtgtttta agcggacaca atcttgctaa |
| 3601 | acactgtctt catgttgtcg gcccaaatgt taacaaaggt gaagacattc aacttcttaa |
| 3661 | gagtgcttat gaaaatttta atcagcacga agttctactt gcaccattat tatcagctgg |
| 3721 | tatttttggt gctgacccta tacattcttt aagagtttgt gtagatactg ttcgcacaaa |
| 3781 | tgtctactta gctgtctttg ataaaaatct ctatgacaaa cttgtttcaa gctttttgga |
| 3841 | aatgaagagt gaaaagcaag ttgaacaaaa gatcgctgag attcctaaag aggaagttaa |
| 3901 | gccatttata actgaaagta aaccttcagt tgaacagaga aaacaagatg ataagaaaat |
| 3961 | caaagcttgt gttgaagaag ttacaacaac tctggaagaa actaagttcc tcacagaaaa |
| 4021 | cttgttactt tatattgaca ttaatggcaa tcttcatcca gattctgcca ctcttgttag |
| 4081 | tgacattgac atcactttct taaagaaaga tgctccatat atagtgggtg atgttgttca |
| 4141 | agagggtgtt ttaactgctg tggttatacc tactaaaaag gctggtggca ctactgaaat |
| 4201 | gctagcgaaa gctttgagaa aagtgccaac agacaattat ataaccactt acccgggtca |
| 4261 | gggtttaaat ggttacactg tagaggaggc aaagacagtg cttaaaaagt gtaaaagtgc |
| 4321 | cttttacatt ctaccatcta ttatctctaa tgagaagcaa gaaattcttg gaactgtttc |
| 4381 | ttggaatttg cgagaaatgc ttgcacatgc agaagaaaca cgcaaattaa tgcctgtctg |
| 4441 | tgtggaaact aaagccatag tttcaactat acagcgtaaa tataagggta ttaaaataca |
| 4501 | agagggtgtg gttgattatg gtgctagatt ttacttttac accagtaaaa caactgtagc |
| 4561 | gtcacttatc aacacactta acgatctaaa tgaaactctt gttacaatgc cacttggcta |
| 4621 | tgtaacacat ggcttaaatt tggaagaagc tgctcggtat atgagatctc tcaaagtgcc |
| 4681 | agctacagtt tctgtttctt cacctgatgc tgttacagcg tataatggtt atcttacttc |
| 4741 | ttcttctaaa acacctgaag aacattttat tgaaaccatc tcacttgctg gttcctataa |
| 4801 | agattggtcc tattctggac aatctacaca actaggtata gaatttctta agagaggtga |
| 4861 | taaaagtgta tattacacta gtaatcctac cacattccac ctagatggtg aagttatcac |
| 4921 | ctttgacaat cttaagacac ttctttcttt gagagaagtg aggactatta aggtgtttac |
| 4981 | aacagtagac aacattaacc tccacacgca agttgtggac atgtcaatga catatggaca |
| 5041 | acagtttggt ccaacttatt tggatggagc tgatgttact aaaataaaac ctcataattc |
| 5101 | acatgaaggt aaaacatttt atgttttacc taatgatgac actctacgtg ttgaggcttt |
| 5161 | tgagtactac cacacaactg atcctagttt tctgggtagg tacatgtcag cattaaatca |
| 5221 | cactaaaaag tggaaatacc cacaagttaa tggtttaact tctattaaat gggcagataa |
| 5281 | caactgttat cttgccactg cattgttaac actccaacaa atagagttga agtttaatcc |
| 5341 | acctgctcta caagatgctt attacagagc aagggctggt gaagctgcta acttttgtgc |
| 5401 | acttatctta gcctactgta ataagacagt aggtgagtta ggtgatgtta gagaaacaat |
| 5461 | gagttacttg tttcaacatg ccaatttaga ttcttgcaaa agagtcttga acgtggtgtg |
| 5521 | taaaacttgt ggacaacagc agacaaccct taagggtgta gaagctgtta tgtacatggg |
| 5581 | cacactttct tatgaacaat ttaagaaagg tgttcagata ccttgtacgt gtggtaaaca |
| 5641 | agctacaaaa tatctagtac aacaggagtc accttttgtt atgatgtcag caccacctgc |
| 5701 | tcagtatgaa cttaagcatg gtacatttac ttgtgctagt gagtacactg gtaattacca |
| 5761 | gtgtggtcac tataaacata taacttctaa agaaactttg tattgcatag acggtgcttt |
| 5821 | acttacaaag tcctcagaat acaaaggtcc tattacggat gttttctaca aagaaaacag |
| 5881 | ttacacaaca accataaaac cagttactta taaattggat ggtgttgttt gtacagaaat |
| 5941 | tgaccctaag ttggacaatt attataagaa agacaattct tatttcacag agcaaccaat |
| 6001 | tgatcttgta ccaaaccaac catatccaaa cgcaagcttc gataatttta agtttgtatg |
| 6061 | tgataatatc aaatttgctg atgatttaaa ccagttaact ggttataaga aacctgcttc |
| 6121 | aagagagctt aaagttacat ttttccctga cttaaatggt gatgtggtgg ctattgatta |
| 6181 | taaacactac acaccctctt ttaagaaagg agctaaattg ttacataaac ctattgtttg |
| 6241 | gcatgttaac aatgcaacta ataaagccac gtataaacca aatacctggt gtatacgttg |
| 6301 | tctttggagc acaaaaccag ttgaaacatc aaattcgttt gatgtactga agtcagagga |
| 6361 | cgcgcaggga atggataatc ttgcctgcga agatctaaaa ccagtctctg aagaagtagt |
| 6421 | ggaaaatcct accatacaga aagacgttct tgagtgtaat gtgaaaacta ccgaagttgt |
| 6481 | aggagacatt atacttaaac cagcaaataa tagtttaaaa attacagaag aggttggcca |
| 6541 | cacagatcta atggctgctt atgtagacaa ttctagtctt actattaaga aacctaatga |
| 6601 | attatctaga gtattaggtt tgaaaaccct tgctactcat ggtttagctg ctgttaatag |
| 6661 | tgtcccttgg gatactatag ctaattatgc taagcctttt cttaacaaag ttgttagtac |
| 6721 | aactactaac atagttacac ggtgtttaaa ccgtgtttgt actaattata tgccttattt |
| 6781 | ctttacttta ttgctacaat tgtgtacttt tactagaagt acaaattcta gaattaaagc |
| 6841 | atctatgccg actactatag caaagaatac tgttaagagt gtcggtaaat tttgtctaga |
| 6901 | ggcttcattt aattatttga agtcacctaa tttttctaaa ctgataaata ttataatttg |
| 6961 | gtttttacta ttaagtgttt gcctaggttc tttaatctac tcaaccgctg ctttaggtgt |
| 7021 | tttaatgtct aatttaggca tgccttctta ctgtactggt tacagagaag gctatttgaa |
| 7081 | ctctactaat gtcactattg caacctactg tactggttct ataccttgta gtgtttgtct |
| 7141 | tagtggttta gattctttag acacctatcc ttctttagaa actatacaaa ttaccatttc |
| 7201 | atcttttaaa tgggatttaa ctgcttttgg cttagttgca gagtggtttt tggcatatat |
| 7261 | tcttttcact aggtttttct atgtacttgg attggctgca atcatgcaat tgtttttcag |
| 7321 | ctattttgca gtacatttta ttagtaattc ttggcttatg tggttaataa ttaatcttgt |
| 7381 | acaaatggcc ccgatttcag ctatggttag aatgtacatc ttctttgcat cattttatta |
| 7441 | tgtatggaaa agttatgtgc atgttgtaga cggttgtaat tcatcaactt gtatgatgtg |
| 7501 | ttacaaacgt aatagagcaa caagagtcga atgtacaact attgttaatg gtgttagaag |
| 7561 | gtccttttat gtctatgcta atggaggtaa aggcttttgc aaactacaca attggaattg |
| 7621 | tgttaattgt gatacattct gtgctggtag tacatttatt agtgatgaag ttgcgagaga |
| 7681 | cttgtcacta cagtttaaaa gaccaataaa tcctactgac cagtcttctt acatcgttga |
| 7741 | tagtgttaca gtgaagaatg gttccatcca tctttacttt gataaagctg gtcaaaagac |
| 7801 | ttatgaaaga cattctctct ctcattttgt taacttagac aacctgagag ctaataacac |
| 7861 | taaaggttca ttgcctatta atgttatagt ttttgatggt aaatcaaaat gtgaagaatc |
| 7921 | atctgcaaaa tcagcgtctg tttactacag tcagcttatg tgtcaaccta tactgttact |
| 7981 | agatcaggca ttagtgtctg atgttggtga tagtgcggaa gttgcagtta aaatgtttga |
| 8041 | tgcttacgtt aatacgtttt catcaacttt taacgtacca atggaaaaac tcaaaacact |
| 8101 | agttgcaact gcagaagctg aacttgcaaa gaatgtgtcc ttagacaatg tcttatctac |
| 8161 | ttttatttca gcagctcggc aagggtttgt tgattcagat gtagaaacta aagatgttgt |
| 8221 | tgaatgtctt aaattgtcac atcaatctga catagaagtt actggcgata gttgtaataa |
| 8281 | ctatatgctc acctataaca aagttgaaaa catgacaccc cgtgaccttg gtgcttgtat |
| 8341 | tgactgtagt gcgcgtcata ttaatgcgca ggtagcaaaa agtcacaaca ttgctttgat |
| 8401 | atggaacgtt aaagatttca tgtcattgtc tgaacaacta cgaaaacaaa tacgtagtgc |
| 8461 | tgctaaaaag aataacttac cttttaagtt gacatgtgca actactagac aagttgttaa |
| 8521 | tgttgtaaca acaaagatag cacttaaggg tggtaaaatt gttaataatt ggttgaagca |
| 8581 | gttaattaaa gttacacttg tgttcctttt tgttgctgct attttctatt taataacacc |
| 8641 | tgttcatgtc atgtctaaac atactgactt ttcaagtgaa atcataggat acaaggctat |
| 8701 | tgatggtggt gtcactcgtg acatagcatc tacagatact tgttttgcta acaaacatgc |
| 8761 | tgattttgac acatggttta gccagcgtgg tggtagttat actaatgaca aagcttgccc |
| 8821 | attgattgct gcagtcataa caagagaagt gggttttgtc gtgcctggtt tgcctggcac |
| 8881 | gatattacgc acaactaatg gtgacttttt gcatttctta cctagagttt ttagtgcagt |
| 8941 | tggtaacatc tgttacacac catcaaaact tatagagtac actgactttg caacatcagc |
| 9001 | ttgtgttttg gctgctgaat gtacaatttt taaagatgct tctggtaagc cagtaccata |
| 9061 | ttgttatgat accaatgtac tagaaggttc tgttgcttat gaaagtttac gccctgacac |
| 9121 | acgttatgtg ctcatggatg gctctattat tcaatttcct aacacctacc ttgaaggttc |
| 9181 | tgttagagtg gtaacaactt ttgattctga gtactgtagg cacggcactt gtgaaagatc |
| 9241 | agaagctggt gtttgtgtat ctactagtgg tagatgggta cttaacaatg attattacag |
| 9301 | atctttacca ggagttttct gtggtgtaga tgctgtaaat ttacttacta atatgtttac |
| 9361 | accactaatt caacctattg gtgctttgga catatcagca tctatagtag ctggtggtat |
| 9421 | tgtagctatc gtagtaacat gccttgccta ctattttatg aggtttagaa gagcttttgg |
| 9481 | tgaatacagt catgtagttg cctttaatac tttactattc cttatgtcat tcactgtact |
| 9541 | ctgtttaaca ccagtttact cattcttacc tggtgtttat tctgttattt acttgtactt |
| 9601 | gacattttat cttactaatg atgtttcttt tttagcacat attcagtgga tggttatgtt |
| 9661 | cacaccttta gtacctttct ggataacaat tgcttatatc atttgtattt ccacaaagca |
| 9721 | tttctattgg ttctttagta attacctaaa gagacgtgta gtctttaatg gtgtttcctt |
| 9781 | tagtactttt gaagaagctg cgctgtgcac ctttttgtta aataaagaaa tgtatctaaa |
| 9841 | gttgcgtagt gatgtgctat tacctcttac gcaatataat agatacttag ctctttataa |
| 9901 | taagtacaag tattttagtg gagcaatgga tacaactagc tacagagaag ctgcttgttg |
| 9961 | tcatctcgca aaggctctca atgacttcag taactcaggt tctgatgttc tttaccaacc |
| 10021 | accacaaacc tctatcacct cagctgtttt gcagagtggt tttagaaaaa tggcattccc |
| 10081 | atctggtaaa gttgagggtt gtatggtaca agtaacttgt ggtacaacta cacttaacgg |
| 10141 | tctttggctt gatgacgtag tttactgtcc aagacatgtg atctgcacct ctgaagacat |
| 10201 | gcttaaccct aattatgaag atttactcat tcgtaagtct aatcataatt tcttggtaca |
| 10261 | ggctggtaat gttcaactca gggttattgg acattctatg caaaattgtg tacttaagct |
| 10321 | taaggttgat acagccaatc ctaagacacc taagtataag tttgttcgca ttcaaccagg |
| 10381 | acagactttt tcagtgttag cttgttacaa tggttcacca tctggtgttt accaatgtgc |
| 10441 | tatgaggccc aatttcacta ttaagggttc attccttaat ggttcatgtg gtagtgttgg |
| 10501 | ttttaacata gattatgact gtgtctcttt ttgttacatg caccatatgg aattaccaac |
| 10561 | tggagttcat gctggcacag acttagaagg taacttttat ggaccttttg ttgacaggca |
| 10621 | aacagcacaa gcagctggta cggacacaac tattacagtt aatgttttag cttggttgta |
| 10681 | cgctgctgtt ataaatggag acaggtggtt tctcaatcga tttaccacaa ctcttaatga |
| 10741 | ctttaacctt gtggctatga agtacaatta tgaacctcta acacaagacc atgttgacat |
| 10801 | actaggacct ctttctgctc aaactggaat tgccgtttta gatatgtgtg cttcattaaa |
| 10861 | agaattactg caaaatggta tgaatggacg taccatattg ggtagtgctt tattagaaga |
| 10921 | tgaatttaca ccttttgatg ttgttagaca atgctcaggt gttactttcc aaagtgcagt |
| 10981 | gaaaagaaca atcaagggta cacaccactg gttgttactc acaattttga cttcactttt |
| 11041 | agttttagtc cagagtactc aatggtcttt gttctttttt ttgtatgaaa atgccttttt |
| 11101 | accttttgct atgggtatta ttgctatgtc tgcttttgca atgatgtttg tcaaacataa |
| 11161 | gcatgcattt ctctgtttgt ttttgttacc ttctcttgcc actgtagctt attttaatat |
| 11221 | ggtctatatg cctgctagtt gggtgatgcg tattatgaca tggttggata tggttgatac |
| 11281 | tagtttgtct ggttttaagc taaaagactg tgttatgtat gcatcagctg tagtgttact |
| 11341 | aatccttatg acagcaagaa ctgtgtatga tgatggtgct aggagagtgt ggacacttat |
| 11401 | gaatgtcttg acactcgttt ataaagttta ttatggtaat gctttagatc aagccatttc |
| 11461 | catgtgggct cttataatct ctgttacttc taactactca ggtgtagtta caactgtcat |
| 11521 | gtttttggcc agaggtattg tttttatgtg tgttgagtat tgccctattt tcttcataac |
| 11581 | tggtaataca cttcagtgta taatgctagt ttattgtttc ttaggctatt tttgtacttg |
| 11641 | ttactttggc ctcttttgtt tactcaaccg ctactttaga ctgactcttg gtgtttatga |
| 11701 | ttacttagtt tctacacagg agtttagata tatgaattca cagggactac tcccacccaa |
| 11761 | gaatagcata gatgccttca aactcaacat taaattgttg ggtgttggtg gcaaaccttg |
| 11821 | tatcaaagta gccactgtac agtctaaaat gtcagatgta aagtgcacat cagtagtctt |
| 11881 | actctcagtt ttgcaacaac tcagagtaga atcatcatct aaattgtggg ctcaatgtgt |
| 11941 | ccagttacac aatgacattc tcttagctaa agatactact gaagcctttg aaaaaatggt |
| 12001 | ttcactactt tctgttttgc tttccatgca gggtgctgta gacataaaca agctttgtga |
| 12061 | agaaatgctg gacaacaggg caaccttaca agctatagcc tcagagttta gttcccttcc |
| 12121 | atcatatgca gcttttgcta ctgctcaaga agcttatgag caggctgttg ctaatggtga |
| 12181 | ttctgaagtt gttcttaaaa agttgaagaa gtctttgaat gtggctaaat ctgaatttga |
| 12241 | ccgtgatgca gccatgcaac gtaagttgga aaagatggct gatcaagcta tgacccaaat |
| 12301 | gtataaacag gctagatctg aggacaagag ggcaaaagtt actagtgcta tgcagacaat |
| 12361 | gcttttcact atgcttagaa agttggataa tgatgcactc aacaacatta tcaacaatgc |
| 12421 | aagagatggt tgtgttccct tgaacataat acctcttaca acagcagcca aactaatggt |
| 12481 | tgtcatacca gactataaca catataaaaa tacgtgtgat ggtacaacat ttacttatgc |
| 12541 | atcagcattg tgggaaatcc aacaggttgt agatgcagat agtaaaattg ttcaacttag |
| 12601 | tgaaattagt atggacaatt cacctaattt agcatggcct cttattgtaa cagctttaag |
| 12661 | ggccaattct gctgtcaaat tacagaataa tgagcttagt cctgttgcac tacgacagat |
| 12721 | gtcttgtgct gccggtacta cacaaactgc ttgcactgat gacaatgcgt tagcttacta |
| 12781 | caacacaaca aagggaggta ggtttgtact tgcactgtta tccgatttac aggatttgaa |
| 12841 | atgggctaga ttccctaaga gtgatggaac tggtactatc tatacagaac tggaaccacc |
| 12901 | ttgtaggttt gttacagaca cacctaaagg tcctaaagtg aagtatttat actttattaa |
| 12961 | aggattaaac aacctaaata gaggtatggt acttggtagt ttagctgcca cagtacgtct |
| 13021 | acaagctggt aatgcaacag aagtgcctgc caattcaact gtattatctt tctgtgcttt |
| 13081 | tgctgtagat gctgctaaag cttacaaaga ttatctagct agtgggggac aaccaatcac |
| 13141 | taattgtgtt aagatgttgt gtacacacac tggtactggt caggcaataa cagttacacc |
| 13201 | ggaagccaat atggatcaag aatcctttgg tggtgcatcg tgttgtctgt actgccgttg |
| 13261 | ccacatagat catccaaatc ctaaaggatt ttgtgactta aaaggtaagt atgtacaaat |
| 13321 | acctacaact tgtgctaatg accctgtggg ttttacactt aaaaacacag tctgtaccgt |
| 13381 | ctgcggtatg tggaaaggtt atggctgtag ttgtgatcaa ctccgcgaac ccatgcttca |
| 13441 | gtcagctgat gcacaatcgt ttttaaacgg gtttgcggtg taagtgcagc ccgtcttaca |
| 13501 | ccgtgcggca caggcactag tactgatgtc gtatacaggg cttttgacat ctacaatgat |
| 13561 | aaagtagctg gttttgctaa attcctaaaa actaattgtt gtcgcttcca agaaaaggac |
| 13621 | gaagatgaca atttaattga ttcttacttt gtagttaaga gacacacttt ctctaactac |
| 13681 | caacatgaag aaacaattta taatttactt aaggattgtc cagctgttgc taaacatgac |
| 13741 | ttctttaagt ttagaataga cggtgacatg gtaccacata tatcacgtca acgtcttact |
| 13801 | aaatacacaa tggcagacct cgtctatgct ttaaggcatt ttgatgaagg taattgtgac |
| 13861 | acattaaaag aaatacttgt cacatacaat tgttgtgatg atgattattt caataaaaag |
| 13921 | gactggtatg attttgtaga aaacccagat atattacgcg tatacgccaa cttaggtgaa |
| 13981 | cgtgtacgcc aagctttgtt aaaaacagta caattctgtg atgccatgcg aaatgctggt |
| 14041 | attgttggtg tactgacatt agataatcaa gatctcaatg gtaactggta tgatttcggt |
| 14101 | gatttcatac aaaccacgcc aggtagtgga gttcctgttg tagattctta ttattcattg |
| 14161 | ttaatgccta tattaacctt gaccagggct ttaactgcag agtcacatgt tgacactgac |
| 14221 | ttaacaaagc cttacattaa gtgggatttg ttaaaatatg acttcacgga agagaggtta |
| 14281 | aaactctttg accgttattt taaatattgg gatcagacat accacccaaa ttgtgttaac |
| 14341 | tgtttggatg acagatgcat tctgcattgt gcaaacttta atgttttatt ctctacagtg |
| 14401 | ttcccaccta caagttttgg accactagtg agaaaaatat ttgttgatgg tgttccattt |
| 14461 | gtagtttcaa ctggatacca cttcagagag ctaggtgttg tacataatca ggatgtaaac |
| 14521 | ttacatagct ctagacttag ttttaaggaa ttacttgtgt atgctgctga ccctgctatg |
| 14581 | cacgctgctt ctggtaatct attactagat aaacgcacta cgtgcttttc agtagctgca |
| 14641 | cttactaaca atgttgcttt tcaaactgtc aaacccggta attttaacaa agacttctat |
| 14701 | gactttgctg tgtctaaggg tttctttaag gaaggaagtt ctgttgaatt aaaacacttc |
| 14761 | ttctttgctc aggatggtaa tgctgctatc agcgattatg actactatcg ttataatcta |
| 14821 | ccaacaatgt gtgatatcag acaactacta tttgtagttg aagttgttga taagtacttt |
| 14881 | gattgttacg atggtggctg tattaatgct aaccaagtca tcgtcaacaa cctagacaaa |
| 14941 | tcagctggtt ttccatttaa taaatggggt aaggctagac tttattatga ttcaatgagt |
| 15001 | tatgaggatc aagatgcact tttcgcatat acaaaacgta atgtcatccc tactataact |
| 15061 | caaatgaatc ttaagtatgc cattagtgca aagaatagag ctcgcaccgt agctggtgtc |
| 15121 | tctatctgta gtactatgac caatagacag tttcatcaaa aattattgaa atcaatagcc |
| 15181 | gccactagag gagctactgt agtaattgga acaagcaaat tctatggtgg ttggcacaac |
| 15241 | atgttaaaaa ctgtttatag tgatgtagaa aaccctcacc ttatgggttg ggattatcct |
| 15301 | aaatgtgata gagccatgcc taacatgctt agaattatgg cctcacttgt tcttgctcgc |
| 15361 | aaacatacaa cgtgttgtag cttgtcacac cgtttctata gattagctaa tgagtgtgct |
| 15421 | caagtattga gtgaaatggt catgtgtggc ggttcactat atgttaaacc aggtggaacc |
| 15481 | tcatcaggag atgccacaac tgcttatgct aatagtgttt ttaacatttg tcaagctgtc |
| 15541 | acggccaatg ttaatgcact tttatctact gatggtaaca aaattgccga taagtatgtc |
| 15601 | cgcaatttac aacacagact ttatgagtgt ctctatagaa atagagatgt tgacacagac |
| 15661 | tttgtgaatg agttttacgc atatttgcgt aaacatttct caatgatgat actctctgac |
| 15721 | gatgctgttg tgtgtttcaa tagcacttat gcatctcaag gtctagtggc tagcataaag |
| 15781 | aactttaagt cagttcttta ttatcaaaac aatgttttta tgtctgaagc aaaatgttgg |
| 15841 | actgagactg accttactaa aggacctcat gaattttgct ctcaacatac aatgctagtt |
| 15901 | aaacagggtg atgattatgt gtaccttcct tacccagatc catcaagaat cctaggggcc |
| 15961 | ggctgttttg tagatgatat cgtaaaaaca gatggtacac ttatgattga acggttcgtg |
| 16021 | tctttagcta tagatgctta cccacttact aaacatccta atcaggagta tgctgatgtc |
| 16081 | tttcatttgt acttacaata cataagaaag ctacatgatg agttaacagg acacatgtta |
| 16141 | gacatgtatt ctgttatgct tactaatgat aacacttcaa ggtattggga acctgagttt |
| 16201 | tatgaggcta tgtacacacc gcatacagtc ttacaggctg ttggggcttg tgttctttgc |
| 16261 | aattcacaga cttcattaag atgtggtgct tgcatacgta gaccattctt atgttgtaaa |
| 16321 | tgctgttacg accatgtcat atcaacatca cataaattag tcttgtctgt taatccgtat |
| 16381 | gtttgcaatg ctccaggttg tgatgtcaca gatgtgactc aactttactt aggaggtatg |
| 16441 | agctattatt gtaaatcaca taaaccaccc attagttttc cattgtgtgc taatggacaa |
| 16501 | gtttttggtt tatataaaaa tacatgtgtt ggtagcgata atgttactga ctttaatgca |
| 16561 | attgcaacat gtgactggac aaatgctggt gattacattt tagctaacac ctgtactgaa |
| 16621 | agactcaagc tttttgcagc agaaacgctc aaagctactg aggagacatt taaactgtct |
| 16681 | tatggtattg ctactgtacg tgaagtgctg tctgacagag aattacatct ttcatgggaa |
| 16741 | gttggtaaac ctagaccacc acttaaccga aattatgtct ttactggtta tcgtgtaact |
| 16801 | aaaaacagta aagtacaaat aggagagtac acctttgaaa aaggtgacta tggtgatgct |
| 16861 | gttgtttacc gaggtacaac aacttacaaa ttaaatgttg gtgattattt tgtgctgaca |
| 16921 | tcacatacag taatgccatt aagtgcacct acactagtgc cacaagagca ctatgttaga |
| 16981 | attactggct tatacccaac actcaatatc tcagatgagt tttctagcaa tgttgcaaat |
| 17041 | tatcaaaagg ttggtatgca aaagtattct acactccagg gaccacctgg tactggtaag |
| 17101 | agtcattttg ctattggcct agctctctac tacccttctg ctcgcatagt gtatacagct |
| 17161 | tgctctcatg ccgctgttga tgcactatgt gagaaggcat taaaatattt gcctatagat |
| 17221 | aaatgtagta gaattatacc tgcacgtgct cgtgtagagt gttttgataa attcaaagtg |
| 17281 | aattcaacat tagaacagta tgtcttttgt actgtaaatg cattgcctga gacgacagca |
| 17341 | gatatagttg tctttgatga aatttcaatg gccacaaatt atgatttgag tgttgtcaat |
| 17401 | gccagattac gtgctaagca ctatgtgtac attggcgacc ctgctcaatt acctgcacca |
| 17461 | cgcacattgc taactaaggg cacactagaa ccagaatatt tcaattcagt gtgtagactt |
| 17521 | atgaaaacta taggtccaga catgttcctc ggaacttgtc ggcgttgtcc tgctgaaatt |
| 17581 | gttgacactg tgagtgcttt ggtttatgat aataagctta aagcacataa agacaaatca |
| 17641 | gctcaatgct ttaaaatgtt ttataagggt gttatcacgc atgatgtttc atctgcaatt |
| 17701 | aacaggccac aaataggcgt ggtaagagaa ttccttacac gtaaccctgc ttggagaaaa |
| 17761 | gctgtcttta tttcacctta taattcacag aatgctgtag cctcaaagat tttgggacta |
| 17821 | ccaactcaaa ctgttgattc atcacagggc tcagaatatg actatgtcat attcactcaa |
| 17881 | accactgaaa cagctcactc ttgtaatgta aacagattta atgttgctat taccagagca |
| 17941 | aaagtaggca tactttgcat aatgtctgat agagaccttt atgacaagtt gcaatttaca |
| 18001 | agtcttgaaa ttccacgtag gaatgtggca actttacaag ctgaaaatgt aacaggactc |
| 18061 | tttaaagatt gtagtaaggt aatcactggg ttacatccta cacaggcacc tacacacctc |
| 18121 | agtgttgaca ctaaattcaa aactgaaggt ttatgtgttg acatacctgg catacctaag |
| 18181 | gacatgacct atagaagact catctctatg atgggtttta aaatgaatta tcaagttaat |
| 18241 | ggttacccta acatgtttat cacccgcgaa gaagctataa gacatgtacg tgcatggatt |
| 18301 | ggcttcgatg tcgaggggtg tcatgctact agagaagctg ttggtaccaa tttaccttta |
| 18361 | cagctaggtt tttctacagg tgttaaccta gttgctgtac ctacaggtta tgttgataca |
| 18421 | cctaataata cagatttttc cagagttagt gctaaaccac cgcctggaga tcaatttaaa |
| 18481 | cacctcatac cacttatgta caaaggactt ccttggaatg tagtgcgtat aaagattgta |
| 18541 | caaatgttaa gtgacacact taaaaatctc tctgacagag tcgtatttgt cttatgggca |
| 18601 | catggctttg agttgacatc tatgaagtat tttgtgaaaa taggacctga gcgcacctgt |
| 18661 | tgtctatgtg atagacgtgc cacatgcttt tccactgctt cagacactta tgcctgttgg |
| 18721 | catcattcta ttggatttga ttacgtctat aatccgttta tgattgatgt tcaacaatgg |
| 18781 | ggttttacag gtaacctaca aagcaaccat gatctgtatt gtcaagtcca tggtaatgca |
| 18841 | catgtagcta gttgtgatgc aatcatgact aggtgtctag ctgtccacga gtgctttgtt |
| 18901 | aagcgtgttg actggactat tgaatatcct ataattggtg atgaactgaa gattaatgcg |
| 18961 | gcttgtagaa aggttcaaca catggttgtt aaagctgcat tattagcaga caaattccca |
| 19021 | gttcttcacg acattggtaa ccctaaagct attaagtgtg tacctcaagc tgatgtagaa |
| 19081 | tggaagttct atgatgcaca gccttgtagt gacaaagctt ataaaataga agaattattc |
| 19141 | tattcttatg ccacacattc tgacaaattc acagatggtg tatgcctatt ttggaattgc |
| 19201 | aatgtcgata gatatcctgc taattccatt gtttgtagat ttgacactag agtgctatct |
| 19261 | aaccttaact tgcctggttg tgatggtggc agtttgtatg taaataaaca tgcattccac |
| 19321 | acaccagctt ttgataaaag tgcttttgtt aatttaaaac aattaccatt tttctattac |
| 19381 | tctgacagtc catgtgagtc tcatggaaaa caagtagtgt cagatataga ttatgtacca |
| 19441 | ctaaagtctg ctacgtgtat aacacgttgc aatttaggtg gtgctgtctg tagacatcat |
| 19501 | gctaatgagt acagattgta tctcgatgct tataacatga tgatctcagc tggctttagc |
| 19561 | ttgtgggttt acaaacaatt tgatacttat aacctctgga acacttttac aagacttcag |
| 19621 | agtttagaaa atgtggcttt taatgttgta aataagggac actttgatgg acaacagggt |
| 19681 | gaagtaccag tttctatcat taataacact gtttacacaa aagttgatgg tgttgatgta |
| 19741 | gaattgtttg aaaataaaac aacattacct gttaatgtag catttgagct ttgggctaag |
| 19801 | cgcaacatta aaccagtacc agaggtgaaa atactcaata atttgggtgt ggacattgct |
| 19861 | gctaatactg tgatctggga ctacaaaaga gatgctccag cacatatatc tactattggt |
| 19921 | gtttgttcta tgactgacat agccaagaaa ccaactgaaa cgatttgtgc accactcact |
| 19981 | gtcttttttg atggtagagt tgatggtcaa gtagacttat ttagaaatgc ccgtaatggt |
| 20041 | gttcttatta cagaaggtag tgttaaaggt ttacaaccat ctgtaggtcc caaacaagct |
| 20101 | agtcttaatg gagtcacatt aattggagaa gccgtaaaaa cacagttcaa ttattataag |
| 20161 | aaagttgatg gtgttgtcca acaattacct gaaacttact ttactcagag tagaaattta |
| 20221 | caagaattta aacccaggag tcaaatggaa attgatttct tagaattagc tatggatgaa |
| 20281 | ttcattgaac ggtataaatt agaaggctat gccttcgaac atatcgttta tggagatttt |
| 20341 | agtcatagtc agttaggtgg tttacatcta ctgattggac tagctaaacg ttttaaggaa |
| 20401 | tcaccttttg aattagaaga ttttattcct atggacagta cagttaaaaa ctatttcata |
| 20461 | acagatgcgc aaacaggttc atctaagtgt gtgtgttctg ttattgattt attacttgat |
| 20521 | gattttgttg aaataataaa atcccaagat ttatctgtag tttctaaggt tgtcaaagtg |
| 20581 | actattgact atacagaaat ttcatttatg ctttggtgta aagatggcca tgtagaaaca |
| 20641 | ttttacccaa aattacaatc tagtcaagcg tggcaaccgg gtgttgctat gcctaatctt |
| 20701 | tacaaaatgc aaagaatgct attagaaaag tgtgaccttc aaaattatgg tgatagtgca |
| 20761 | acattaccta aaggcataat gatgaatgtc gcaaaatata ctcaactgtg tcaatattta |
| 20821 | aacacattaa cattagctgt accctataat atgagagtta tacattttgg tgctggttct |
| 20881 | gataaaggag ttgcaccagg tacagctgtt ttaagacagt ggttgcctac gggtacgctg |
| 20941 | cttgtcgatt cagatcttaa tgactttgtc tctgatgcag attcaacttt gattggtgat |
| 21001 | tgtgcaactg tacatacagc taataaatgg gatctcatta ttagtgatat gtacgaccct |
| 21061 | aagactaaaa atgttacaaa agaaaatgac tctaaagagg gttttttcac ttacatttgt |
| 21121 | gggtttatac aacaaaagct agctcttgga ggttccgtgg ctataaagat aacagaacat |
| 21181 | tcttggaatg ctgatcttta taagctcatg ggacacttcg catggtggac agcctttgtt |
| 21241 | actaatgtga atgcgtcatc atctgaagca tttttaattg gatgtaatta tcttggcaaa |
| 21301 | ccacgcgaac aaatagatgg ttatgtcatg catgcaaatt acatattttg gaggaataca |
| 21361 | aatccaattc agttgtcttc ctattcttta tttgacatga gtaaatttcc ccttaaatta |
| 21421 | aggggtactg ctgttatgtc tttaaaagaa ggtcaaatca atgatatgat tttatctctt |
| 21481 | cttagtaaag gtagacttat aattagagaa aacaacagag ttgttatttc tagtgatgtt |
| 21541 | cttgttaaca actaaacgaa caatgtttgt ttttcttgtt ttattgccac tagtctctag |
| 21601 | tcagtgtgtt aatcttacaa ccagaactca attaccccct gcatacacta attctttcac |
| 21661 | acgtggtgtt tattaccctg acaaagtttt cagatcctca gttttacatt caactcagga |
| 21721 | cttgttctta cctttctttt ccaatgttac ttggttccat gctatacatg tctctgggac |
| 21781 | caatggtact aagaggtttg ataaccctgt cctaccattt aatgatggtg tttattttgc |
| 21841 | ttccactgag aagtctaaca taataagagg ctggattttt ggtactactt tagattcgaa |
| 21901 | gacccagtcc ctacttattg ttaataacgc tactaatgtt gttattaaag tctgtgaatt |
| 21961 | tcaattttgt aatgatccat ttttgggtgt ttattaccac aaaaacaaca aaagttggat |
| 22021 | ggaaagtgag ttcagagttt attctagtgc gaataattgc acttttgaat atgtctctca |
| 22081 | gccttttctt atggaccttg aaggaaaaca gggtaatttc aaaaatctta gggaatttgt |
| 22141 | gtttaagaat attgatggtt attttaaaat atattctaag cacacgccta ttaatttagt |
| 22201 | gcgtgatctc cctcagggtt tttcggcttt agaaccattg gtagatttgc caataggtat |
| 22261 | taacatcact aggtttcaaa ctttacttgc tttacataga agttatttga ctcctggtga |
| 22321 | ttcttcttca ggttggacag ctggtgctgc agcttattat gtgggttatc ttcaacctag |
| 22381 | gacttttcta ttaaaatata atgaaaatgg aaccattaca gatgctgtag actgtgcact |
| 22441 | tgaccctctc tcagaaacaa agtgtacgtt gaaatccttc actgtagaaa aaggaatcta |
| 22501 | tcaaacttct aactttagag tccaaccaac agaatctatt gttagatttc ctaatattac |
| 22561 | aaacttgtgc ccttttggtg aagtttttaa cgccaccaga tttgcatctg tttatgcttg |
| 22621 | gaacaggaag agaatcagca actgtgttgc tgattattct gtcctatata attccgcatc |
| 22681 | attttccact tttaagtgtt atggagtgtc tcctactaaa ttaaatgatc tctgctttac |
| 22741 | taatgtctat gcagattcat ttgtaattag aggtgatgaa gtcagacaaa tcgctccagg |
| 22801 | gcaaactgga aagattgctg attataatta taaattacca gatgatttta caggctgcgt |
| 22861 | tatagcttgg aattctaaca atcttgattc taaggttggt ggtaattata attacctgta |
| 22921 | tagattgttt aggaagtcta atctcaaacc ttttgagaga gatatttcaa ctgaaatcta |
| 22981 | tcaggccggt agcacacctt gtaatggtgt tgaaggtttt aattgttact ttcctttaca |
| 23041 | atcatatggt ttccaaccca ctaatggtgt tggttaccaa ccatacagag tagtagtact |
| 23101 | ttcttttgaa cttctacatg caccagcaac tgtttgtgga cctaaaaagt ctactaattt |
| 23161 | ggttaaaaac aaatgtgtca atttcaactt caatggttta acaggcacag gtgttcttac |
| 23221 | tgagtctaac aaaaagtttc tgcctttcca acaatttggc agagacattg ctgacactac |
| 23281 | tgatgctgtc cgtgatccac agacacttga gattcttgac attacaccat gttcttttgg |
| 23341 | tggtgtcagt gttataacac caggaacaaa tacttctaac caggttgctg ttctttatca |
| 23401 | ggatgttaac tgcacagaag tccctgttgc tattcatgca gatcaactta ctcctacttg |
| 23461 | gcgtgtttat tctacaggtt ctaatgtttt tcaaacacgt gcaggctgtt taataggggc |
| 23521 | tgaacatgtc aacaactcat atgagtgtga catacccatt ggtgcaggta tatgcgctag |
| 23581 | ttatcagact cagactaatt ctcctcggcg ggcacgtagt gtagctagtc aatccatcat |
| 23641 | tgcctacact atgtcacttg gtgcagaaaa ttcagttgct tactctaata actctattgc |
| 23701 | catacccaca aattttacta ttagtgttac cacagaaatt ctaccagtgt ctatgaccaa |
| 23761 | gacatcagta gattgtacaa tgtacatttg tggtgattca actgaatgca gcaatctttt |
| 23821 | gttgcaatat ggcagttttt gtacacaatt aaaccgtgct ttaactggaa tagctgttga |
| 23881 | acaagacaaa aacacccaag aagtttttgc acaagtcaaa caaatttaca aaacaccacc |
| 23941 | aattaaagat tttggtggtt ttaatttttc acaaatatta ccagatccat caaaaccaag |
| 24001 | caagaggtca tttattgaag atctactttt caacaaagtg acacttgcag atgctggctt |
| 24061 | catcaaacaa tatggtgatt gccttggtga tattgctgct agagacctca tttgtgcaca |
| 24121 | aaagtttaac ggccttactg ttttgccacc tttgctcaca gatgaaatga ttgctcaata |
| 24181 | cacttctgca ctgttagcgg gtacaatcac ttctggttgg acctttggtg caggtgctgc |
| 24241 | attacaaata ccatttgcta tgcaaatggc ttataggttt aatggtattg gagttacaca |
| 24301 | gaatgttctc tatgagaacc aaaaattgat tgccaaccaa tttaatagtg ctattggcaa |
| 24361 | aattcaagac tcactttctt ccacagcaag tgcacttgga aaacttcaag atgtggtcaa |
| 24421 | ccaaaatgca caagctttaa acacgcttgt taaacaactt agctccaatt ttggtgcaat |
| 24481 | ttcaagtgtt ttaaatgata tcctttcacg tcttgacaaa gttgaggctg aagtgcaaat |
| 24541 | tgataggttg atcacaggca gacttcaaag tttgcagaca tatgtgactc aacaattaat |
| 24601 | tagagctgca gaaatcagag cttctgctaa tcttgctgct actaaaatgt cagagtgtgt |
| 24661 | acttggacaa tcaaaaagag ttgatttttg tggaaagggc tatcatctta tgtccttccc |
| 24721 | tcagtcagca cctcatggtg tagtcttctt gcatgtgact tatgtccctg cacaagaaaa |
| 24781 | gaacttcaca actgctcctg ccatttgtca tgatggaaaa gcacactttc ctcgtgaagg |
| 24841 | tgtctttgtt tcaaatggca cacactggtt tgtaacacaa aggaattttt atgaaccaca |
| 24901 | aatcattact acagacaaca catttgtgtc tggtaactgt gatgttgtaa taggaattgt |
| 24961 | caacaacaca gtttatgatc ctttgcaacc tgaattagac tcattcaagg aggagttaga |
| 25021 | taaatatttt aagaatcata catcaccaga tgttgattta ggtgacatct ctggcattaa |
| 25081 | tgcttcagtt gtaaacattc aaaaagaaat tgaccgcctc aatgaggttg ccaagaattt |
| 25141 | aaatgaatct ctcatcgatc tccaagaact tggaaagtat gagcagtata taaaatggcc |
| 25201 | atggtacatt tggctaggtt ttatagctgg cttgattgcc atagtaatgg tgacaattat |
| 25261 | gctttgctgt atgaccagtt gctgtagttg tctcaagggc tgttgttctt gtggatcctg |
| 25321 | ctgcaaattt gatgaagacg actctgagcc agtgctcaaa ggagtcaaat tacattacac |
| 25381 | ataaacgaac ttatggattt gtttatgaga atcttcacaa ttggaactgt aactttgaag |
| 25441 | caaggtgaaa tcaaggatgc tactccttca gattttgttc gcgctactgc aacgataccg |
| 25501 | atacaagcct cactcccttt cggatggctt attgttggcg ttgcacttct tgctgttttt |
| 25561 | cagagcgctt ccaaaatcat aaccctcaaa aagagatggc aactagcact ctccaagggt |
| 25621 | gttcactttg tttgcaactt gctgttgttg tttgtaacag tttactcaca ccttttgctc |
| 25681 | gttgctgctg gccttgaagc cccttttctc tatctttatg ctttagtcta cttcttgcag |
| 25741 | agtataaact ttgtaagaat aataatgagg ctttggcttt gctggaaatg ccgttccaaa |
| 25801 | aacccattac tttatgatgc caactatttt ctttgctggc atactaattg ttacgactat |
| 25861 | tgtatacctt acaatagtgt aacttcttca attgtcatta cttcaggtga tggcacaaca |
| 25921 | agtcctattt ctgaacatga ctaccagatt ggtggttata ctgaaaaatg ggaatctgga |
| 25981 | gtaaaagact gtgttgtatt acacagttac ttcacttcag actattacca gctgtactca |
| 26041 | actcaattga gtacagacac tggtgttgaa catgttacct tcttcatcta caataaaatt |
| 26101 | gttgatgagc ctgaagaaca tgtccaaatt cacacaatcg acggttcatc cggagttgtt |
| 26161 | aatccagtaa tggaaccaat ttatgatgaa ccgacgacga ctactagcgt gcctttgtaa |
| 26221 | gcacaagctg atgagtacga acttatgtac tcattcgttt cggaagagac aggtacgtta |
| 26281 | atagttaata gcgtacttct ttttcttgct ttcgtggtat tcttgctagt tacactagcc |
| 26341 | atccttactg cgcttcgatt gtgtgcgtac tgctgcaata ttgttaacgt gagtcttgta |
| 26401 | aaaccttctt tttacgttta ctctcgtgtt aaaaatctga attcttctag agttcctgat |
| 26461 | cttctggtct aaacgaacta aatattatat tagtttttct gtttggaact ttaattttag |
| 26521 | ccatggcaga ttccaacggt actattaccg ttgaagagct taaaaagctc cttgaacaat |
| 26581 | ggaacctagt aataggtttc ctattcctta catggatttg tcttctacaa tttgcctatg |
| 26641 | ccaacaggaa taggtttttg tatataatta agttaatttt cctctggctg ttatggccag |
| 26701 | taactttagc ttgttttgtg cttgctgctg tttacagaat aaattggatc accggtggaa |
| 26761 | ttgctatcgc aatggcttgt cttgtaggct tgatgtggct cagctacttc attgcttctt |
| 26821 | tcagactgtt tgcgcgtacg cgttccatgt ggtcattcaa tccagaaact aacattcttc |
| 26881 | tcaacgtgcc actccatggc actattctga ccagaccgct tctagaaagt gaactcgtaa |
| 26941 | tcggagctgt gatccttcgt ggacatcttc gtattgctgg acaccatcta ggacgctgtg |
| 27001 | acatcaagga cctgcctaaa gaaatcactg ttgctacatc acgaacgctt tcttattaca |
| 27061 | aattgggagc ttcgcagcgt gtagcaggtg actcaggttt tgctgcatac agtcgctaca |
| 27121 | ggattggcaa ctataaatta aacacagacc attccagtag cagtgacaat attgctttgc |
| 27181 | ttgtacagta agtgacaaca gatgtttcat ctcgttgact ttcaggttac tatagcagag |
| 27241 | atattactaa ttattatgag gacttttaaa gtttccattt ggaatcttga ttacatcata |
| 27301 | aacctcataa ttaaaaattt atctaagtca ctaactgaga ataaatattc tcaattagat |
| 27361 | gaagagcaac caatggagat tgattaaacg aacatgaaaa ttattctttt cttggcactg |
| 27421 | ataacactcg ctacttgtga gctttatcac taccaagagt gtgttagagg tacaacagta |
| 27481 | cttttaaaag aaccttgctc ttctggaaca tacgagggca attcaccatt tcatcctcta |
| 27541 | gctgataaca aatttgcact gacttgcttt agcactcaat ttgcttttgc ttgtcctgac |
| 27601 | ggcgtaaaac acgtctatca gttacgtgcc agatcagttt cacctaaact gttcatcaga |
| 27661 | caagaggaag ttcaagaact ttactctcca atttttctta ttgttgcggc aatagtgttt |
| 27721 | ataacacttt gcttcacact caaaagaaag acagaatgat tgaactttca ttaattgact |
| 27781 | tctatttgtg ctttttagcc tttctgctat tccttgtttt aattatgctt attatctttt |
| 27841 | ggttctcact tgaactgcaa gatcataatg aaacttgtca cgcctaaacg aacatgaaat |
| 27901 | ttcttgtttt cttaggaatc atcacaactg tagctgcatt tcaccaagaa tgtagtttac |
| 27961 | agtcatgtac tcaacatcaa ccatatgtag ttgatgaccc gtgtcctatt cacttctatt |
| 28021 | ctaaatggta tattagagta ggagctagaa aatcagcacc tttaattgaa ttgtgcgtgg |
| 28081 | atgaggctgg ttctaaatca cccattcagt acatcgatat cggtaattat acagtttcct |
| 28141 | gtttaccttt tacaattaat tgccaggaac ctaaattggg tagtcttgta gtgcgttgtt |
| 28201 | cgttctatga agacttttta gagtatcatg acgttcgtgt tgttttagat ttcatctaaa |
| 28261 | cgaacaaact aaaatgtctg ataatggacc ccaaaatcag cgaaatgcac cccgcattac |
| 28321 | gtttggtgga ccctcagatt caactggcag taaccagaat ggagaacgca gtggggcgcg |
| 28381 | atcaaaacaa cgtcggcccc aaggtttacc caataatact gcgtcttggt tcaccgctct |
| 28441 | cactcaacat ggcaaggaag accttaaatt ccctcgagga caaggcgttc caattaacac |
| 28501 | caatagcagt ccagatgacc aaattggcta ctaccgaaga gctaccagac gaattcgtgg |
| 28561 | tggtgacggt aaaatgaaag atctcagtcc aagatggtat ttctactacc taggaactgg |
| 28621 | gccagaagct ggacttccct atggtgctaa caaagacggc atcatatggg ttgcaactga |
| 28681 | gggagccttg aatacaccaa aagatcacat tggcacccgc aatcctgcta acaatgctgc |
| 28741 | aatcgtgcta caacttcctc aaggaacaac attgccaaaa ggcttctacg cagaagggag |
| 28801 | cagaggcggc agtcaagcct cttctcgttc ctcatcacgt agtcgcaaca gttcaagaaa |
| 28861 | ttcaactcca ggcagcagta ggggaacttc tcctgctaga atggctggca atggcggtga |
| 28921 | tgctgctctt gctttgctgc tgcttgacag attgaaccag cttgagagca aaatgtctgg |
| 28981 | taaaggccaa caacaacaag gccaaactgt cactaagaaa tctgctgctg aggcttctaa |
| 29041 | gaagcctcgg caaaaacgta ctgccactaa agcatacaat gtaacacaag ctttcggcag |
| 29101 | acgtggtcca gaacaaaccc aaggaaattt tggggaccag gaactaatca gacaaggaac |
| 29161 | tgattacaaa cattggccgc aaattgcaca atttgccccc agcgcttcag cgttcttcgg |
| 29221 | aatgtcgcgc attggcatgg aagtcacacc ttcgggaacg tggttgacct acacaggtgc |
| 29281 | catcaaattg gatgacaaag atccaaattt caaagatcaa gtcattttgc tgaataagca |
| 29341 | tattgacgca tacaaaacat tcccaccaac agagcctaaa aaggacaaaa agaagaaggc |
| 29401 | tgatgaaact caagccttac cgcagagaca gaagaaacag caaactgtga ctcttcttcc |
| 29461 | tgctgcagat ttggatgatt tctccaaaca attgcaacaa tccatgagca gtgctgactc |
| 29521 | aactcaggcc taaactcatg cagaccacac aaggcagatg ggctatataa acgttttcgc |
| 29581 | ttttccgttt acgatatata gtctactctt gtgcagaatg aattctcgta actacatagc |
| 29641 | acaagtagat gtagttaact ttaatctcac atagcaatct ttaatcagtg tgtaacatta |
| 29701 | gggaggactt gaaagagcca ccacattttc accgaggcca cgcggagtac gatcgagtgt |
| 29761 | acagtgaaca atgctaggga gagctgccta tatggaagag ccctaatgtg taaaattaat |
| 29821 | tttagtagtg ctatccccat gtgattttaa tagcttctta ggagaatgac aaaaaaaaaa |
| 29881 | aaaaaaaaaa aaaaaaaaaa aaa |

In some embodiments, a CoV RNAi agent includes an antisense strand wherein position 19 of the antisense strand (5′→3′) is capable of forming a base pair with position 1 of a 19-mer target sequence disclosed in Table 1. In some embodiments, a CoV RNAi agent includes an antisense strand wherein position 1 of the antisense strand (5′→3′) is capable of forming a base pair with position 19 of a 19-mer target sequence disclosed in Table 1.

In some embodiments, a CoV RNAi agent includes an antisense strand wherein position 2 of the antisense strand (5′→3′) is capable of forming a base pair with position 18 of a 19-mer target sequence disclosed in Table 1. In some embodiments, a CoV RNAi agent includes an antisense strand wherein positions 2 through 18 of the antisense strand (5′→3′) are capable of forming base pairs with each of the respective complementary bases located at positions 18 through 2 of the 19-mer target sequence disclosed in Table 1.

For the RNAi agents disclosed herein, the nucleotide at position 1 of the antisense strand (from 5′ end→3′ end) can be perfectly complementary to a SAR_S-CoV-2 viral genome (or other coronavirus genome being targeted), or can be non-complementary to a SARS-CoV-2 viral genome (or other coronavirus genome being targeted). In some embodiments, the nucleotide at position 1 of the antisense strand (from 5′ end→3′ end) is a U, A, or dT. In some embodiments, the nucleotide at position 1 of the antisense strand (from 5′ end→3′ end) forms an A:U or U:A base pair with the sense strand.

In some embodiments, a CoV RNAi agent antisense strand comprises the sequence of nucleotides (from 5′ end→3′ end) 2-18 or 2-19 of any of the antisense strand sequences in Table 2 or Table 3. In some embodiments, a CoV RNAi agent sense strand comprises the sequence of nucleotides (from 5′ end→3′ end) 1-17, 1-18, or 2-18 of any of the sense strand sequences in Table 2, Table 4, Table 5, or Table 6.

In some embodiments, a CoV RNAi agent is comprised of (i) an antisense strand comprising the sequence of nucleotides (from 5′ end→3′ end) 2-18 or 2-19 of any of the antisense strand sequences in Table 2 or Table 3, and (ii) a sense strand comprising the sequence of nucleotides (from 5′ end→3′ end) 1-17 or 1-18 of any of the sense strand sequences in Table 2, Table 4, Table 5, or Table 6.

In some embodiments, the CoV RNAi agents include core 19-mer nucleotide sequences shown in the following Table 2.

TABLE 2
CoV RNAi agent Antisense Strand and Sense Strand Core Stretch Base Sequences
(N = any nucleobase)

| | | | | Corresponding | |
|---|---|---|---|---|---|
| | Antisense Strand Base | | Sense Strand Base | Positions of | Targeted |
| | Sequence (5′→3′) | | Sequence (5′→3′) | Identified | Viral |
| SEQ ID | (Shown as an Unmodified | SEQ ID | (Shown as an Unmodified | Sequence on | Genome |
| NO: | Nucleotide Sequence) | NO: | Nucleotide Sequence) | SEQ ID NO: 1 | Position |
| 70 | AAACUGAGUUGGACGUGUG | 373 | CACACGUCCAACUCAGUUU | 300-318 | 298 |
| 71 | UAACUGAGUUGGACGUGUG | 374 | CACACGUCCAACUCAGUUA | 300-318 | 298 |
| 72 | NAACUGAGUUGGACGUGUG | 375 | CACACGUCCAACUCAGUUN | 300-318 | 298 |
| 73 | NAACUGAGUUGGACGUGUN | 376 | NACACGUCCAACUCAGUUN | 300-318 | 298 |
| 74 | UCAUAAGCACUCUUAAGAA | 377 | UUCUUAAGAGUGCUUAUGA | 3654-3672 | 3652 |
| 75 | ACAUAAGCACUCUUAAGAA | 378 | UUCUUAAGAGUGCUUAUGU | 3654-3672 | 3652 |
| 76 | NCAUAAGCACUCUUAAGAA | 379 | UUCUUAAGAGUGCUUAUGN | 3654-3672 | 3652 |
| 77 | NCAUAAGCACUCUUAAGAN | 380 | NUCUUAAGAGUGCUUAUGN | 3654-3672 | 3652 |
| 78 | UUCAUAAGCACUCUUAAGA | 381 | UCUUAAGAGUGCUUAUGAA | 3655-1673 | 3653 |
| 79 | AUCAUAAGCACUCUUAAGA | 382 | UCUUAAGAGUGCUUAUGAU | 3655-1673 | 3653 |
| 80 | NUCAUAAGCACUCUUAAGA | 383 | UCUUAAGAGUGCUUAUGAN | 3655-1673 | 3653 |
| 81 | NUCAUAAGCACUCUUAAGN | 384 | NCUUAAGAGUGCUUAUGAN | 3655-1673 | 3653 |
| 82 | GAUGAAGAUUGCCAUUAAU | 385 | AUUAAUGGCAAUCUUCAUC | 4040-4058 | 4038 |
| 83 | UAUGAAGAUUGCCAUUAAU | 386 | AUUAAUGGCAAUCUUCAUA | 4040-4058 | 4038 |
| 84 | AAUGAAGAUUGCCAUUAAU | 387 | AUUAAUGGCAAUCUUCAUU | 4040-4058 | 4038 |
| 85 | NAUGAAGAUUGCCAUUAAU | 388 | AUUAAUGGCAAUCUUCAUN | 4040-4058 | 4038 |
| 86 | NAUGAAGAUUGCCAUUAAN | 389 | NUUAAUGGCAAUCUUCAUN | 4040-4058 | 4038 |
| 87 | UUAGUAGGUAUAACCACAG | 390 | CUGUGGUUAUACCUACUAA | 4158-4176 | 4156 |
| 88 | AUAGUAGGUAUAACCACAG | 391 | CUGUGGUUAUACCUACUAU | 4158-4176 | 4156 |
| 89 | NUAGUAGGUAUAACCACAG | 392 | CUGUGGUUAUACCUACUAN | 4158-4176 | 4156 |
| 90 | NUAGUAGGUAUAACCACAN | 393 | NUGUGGUUAUACCUACUAN | 4158-4176 | 4156 |
| 91 | UCUUAAGAUUGUCAAAGGU | 394 | ACCUUUGACAAUCUUAAGA | 4919-4937 | 4917 |
| 92 | ACUUAAGAUUGUCAAAGGU | 395 | ACCUUUGACAAUCUUAAGU | 4919-4937 | 4917 |
| 93 | NCUUAAGAUUGUCAAAGGU | 396 | ACCUUUGACAAUCUUAAGN | 4919-4937 | 4917 |
| 94 | NCUUAAGAUUGUCAAAGGN | 397 | NCCUUUGACAAUCUUAAGN | 4919-4937 | 4917 |
| 95 | AAGAAGUGUCUUAAGAUUG | 398 | CAAUCUUAAGACACUUCUU | 4927-4945 | 4925 |
| 96 | UAGAAGUGUCUUAAGAUUG | 399 | CAAUCUUAAGACACUUCUA | 4927-4945 | 4925 |
| 97 | NAGAAGUGUCUUAAGAUUG | 400 | CAAUCUUAAGACACUUCUN | 4927-4945 | 4925 |
| 98 | NAGAAGUGUCUUAAGAUUN | 401 | NAAUCUUAAGACACUUCUN | 4927-4945 | 4925 |
| 99 | AAAGAAGUGUCUUAAGAUU | 402 | AAUCUUAAGACACUUCUUU | 4928-4946 | 4926 |
| 100 | UAAGAAGUGUCUUAAGAUU | 403 | AAUCUUAAGACACUUCUUA | 4928-4946 | 4926 |
| 101 | NAAGAAGUGUCUUAAGAUU | 404 | AAUCUUAAGACACUUCUUN | 4928-4946 | 4926 |
| 102 | NAAGAAGUGUCUUAAGAUN | 405 | NAUCUUAAGACACUUCUUN | 4928-4946 | 4926 |
| 103 | UCAUAAGAAAGUGUGCCCA | 406 | UGGGCACACUUUCUUAUGA | 5577-5595 | 5575 |
| 104 | ACAUAAGAAAGUGUGCCCA | 407 | UGGGCACACUUUCUUAUGU | 5577-5595 | 5575 |
| 105 | NCAUAAGAAAGUGUGCCCA | 408 | UGGGCACACUUUCUUAUGN | 5577-5595 | 5575 |
| 106 | NCAUAAGAAAGUGUGCCCN | 409 | NGGGCACACUUUCUUAUGN | 5577-5595 | 5575 |
| 107 | CAACUUAGGGUCAAUUUCU | 410 | AGAAAUUGACCCUAAGUUG | 5935-5953 | 5933 |
| 108 | UAACUUAGGGUCAAUUUCU | 411 | AGAAAUUGACCCUAAGUUA | 5935-5953 | 5933 |
| 109 | AAACUUAGGGUCAAUUUCU | 412 | AGAAAUUGACCCUAAGUUU | 5935-5953 | 5933 |
| 110 | NAACUUAGGGUCAAUUUCU | 413 | AGAAAUUGACCCUAAGUUN | 5935-5953 | 5933 |
| 111 | NAACUUAGGGUCAAUUUCN | 414 | NGAAAUUGACCCUAAGUUN | 5935-5953 | 5933 |
| 112 | GGAUUUUCCACUACUUCUU | 415 | AAGAAGUAGUGGAAAAUCC | 6411-6429 | 6409 |
| 113 | UGAUUUUCCACUACUUCUU | 416 | AAGAAGUAGUGGAAAAUCA | 6411-6429 | 6409 |
| 114 | AGAUUUUCCACUACUUCUU | 417 | AAGAAGUAGUGGAAAAUCU | 6411-6429 | 6409 |
| 115 | NGAUUUUCCACUACUUCUU | 418 | AAGAAGUAGUGGAAAAUCN | 6411-6429 | 6409 |
| 116 | NGAUUUUCCACUACUUCUN | 419 | NAGAAGUAGUGGAAAAUCN | 6411-6429 | 6409 |
| 117 | GUAGGAUUUUCCACUACUU | 420 | AAGUAGUGGAAAAUCCUAC | 6414-6432 | 6412 |
| 118 | UUAGGAUUUUCCACUACUU | 421 | AAGUAGUGGAAAAUCCUAA | 6414-6432 | 6412 |
| 119 | AUAGGAUUUUCCACUACUU | 422 | AAGUAGUGGAAAAUCCUAU | 6414-6432 | 6412 |
| 120 | NUAGGAUUUUCCACUACUU | 423 | AAGUAGUGGAAAAUCCUAN | 6414-6432 | 6412 |
| 121 | NUAGGAUUUUCCACUACUN | 424 | NAGUAGUGGAAAAUCCUAN | 6414-6432 | 6412 |
| 122 | AAACGUAUUAACGUAAGCA | 425 | UGCUUACGUUAAUACGUUU | 8041-8059 | 8039 |
| 123 | UAACGUAUUAACGUAAGCA | 426 | UGCUUACGUUAAUACGUUA | 8041-8059 | 8039 |
| 124 | NAACGUAUUAACGUAAGCA | 427 | UGCUUACGUUAAUACGUUN | 8041-8059 | 8039 |
| 125 | NAACGUAUUAACGUAAGCN | 428 | NGCUUACGUUAAUACGUUN | 8041-8059 | 8039 |
| 126 | GUAGAUAAGACAUUGUCUA | 429 | UAGACAAUGUCUUAUCUAC | 8142-8160 | 8140 |
| 127 | UUAGAUAAGACAUUGUCUA | 430 | UAGACAAUGUCUUAUCUAA | 8142-8160 | 8140 |
| 128 | AUAGAUAAGACAUUGUCUA | 431 | UAGACAAUGUCUUAUCUAU | 8142-8160 | 8140 |
| 129 | NUAGAUAAGACAUUGUCUA | 432 | UAGACAAUGUCUUAUCUAN | 8142-8160 | 8140 |
| 130 | NUAGAUAAGACAUUGUCUN | 433 | NAGACAAUGUCUUAUCUAN | 8142-8160 | 8140 |
| 131 | AAACCAACACUACCACAUG | 434 | CAUGUGGUAGUGUUGGUUU | 10485-10503 | 10483 |
| 132 | UAACCAACACUACCACAUG | 435 | CAUGUGGUAGUGUUGGUUA | 10485-10503 | 10483 |
| 133 | NAACCAACACUACCACAUG | 436 | CAUGUGGUAGUGUUGGUUN | 10485-10503 | 10483 |
| 134 | NAACCAACACUACCACAUN | 437 | NAUGUGGUAGUGUUGGUUN | 10485-10503 | 10483 |
| 135 | UUGUCUAACAACAUCAAAA | 438 | UUUUGAUGUUGUUAGACAA | 10933-10951 | 10931 |
| 136 | AUGUCUAACAACAUCAAAA | 439 | UUUUGAUGUUGUUAGACAU | 10933-10951 | 10931 |
| 137 | NUGUCUAACAACAUCAAAA | 440 | UUUUGAUGUUGUUAGACAN | 10933-10951 | 10931 |
| 138 | NUGUCUAACAACAUCAAAN | 441 | NUUUGAUGUUGUUAGACAN | 10933-10951 | 10931 |
| 139 | GCUUGAUCUAAAGCAUUAC | 442 | GUAAUGCUUUAGAUCAAGC | 11436-11454 | 11434 |
| 140 | UCUUGAUCUAAAGCAUUAC | 443 | GUAAUGCUUUAGAUCAAGA | 11436-11454 | 11434 |
| 141 | ACUUGAUCUAAAGCAUUAC | 444 | GUAAUGCUUUAGAUCAAGU | 11436-11454 | 11434 |
| 142 | NCUUGAUCUAAAGCAUUAC | 445 | GUAAUGCUUUAGAUCAAGN | 11436-11454 | 11434 |
| 143 | NCUUGAUCUAAAGCAUUAN | 446 | NUAAUGCUUUAGAUCAAGN | 11436-11454 | 11434 |
| 144 | AUACAUUUGGGUCAUAGCU | 447 | AGCUAUGACCCAAAUGUAU | 12286-12304 | 12284 |
| 145 | UUACAUUUGGGUCAUAGCU | 448 | AGCUAUGACCCAAAUGUAA | 12286-12304 | 12284 |
| 146 | NUACAUUUGGGUCAUAGCU | 449 | AGCUAUGACCCAAAUGUAN | 12286-12304 | 12284 |
| 147 | NUACAUUUGGGUCAUAGCN | 450 | NGCUAUGACCCAAAUGUAN | 12286-12304 | 12284 |
| 148 | UGAUAUAUGUGGUACCAUG | 451 | CAUGGUACCACAUAUAUCA | 13767-13785 | 13765 |
| 149 | AGAUAUAUGUGGUACCAUG | 452 | CAUGGUACCACAUAUAUCU | 13767-13785 | 13765 |
| 150 | NGAUAUAUGUGGUACCAUG | 453 | CAUGGUACCACAUAUAUCN | 13767-13785 | 13765 |
| 151 | NGAUAUAUGUGGUACCAUN | 454 | NAUGGUACCACAUAUAUCN | 13767-13785 | 13765 |
| 152 | GUGAUAUAUGUGGUACCAU | 455 | AUGGUACCACAUAUAUCAC | 13768-13786 | 13766 |
| 153 | UUGAUAUAUGUGGUACCAU | 456 | AUGGUACCACAUAUAUCAA | 13768-13786 | 13766 |
| 154 | AUGAUAUAUGUGGUACCAU | 457 | AUGGUACCACAUAUAUCAU | 13768-13786 | 13766 |
| 155 | NUGAUAUAUGUGGUACCAU | 458 | AUGGUACCACAUAUAUCAN | 13768-13786 | 13766 |
| 156 | NUGAUAUAUGUGGUACCAN | 459 | NUGGUACCACAUAUAUCAN | 13768-13786 | 13766 |
| 157 | UUGAUUAUCUAAUGUCAGU | 460 | ACUGACAUUAGAUAAUCAA | 14052-14070 | 14050 |
| 158 | AUGAUUAUCUAAUGUCAGU | 461 | ACUGACAUUAGAUAAUCAU | 14052-14070 | 14050 |
| 159 | NUGAUUAUCUAAUGUCAGU | 462 | ACUGACAUUAGAUAAUCAN | 14052-14070 | 14050 |
| 160 | NUGAUUAUCUAAUGUCAGN | 463 | NCUGACAUUAGAUAAUCAN | 14052-14070 | 14050 |
| 161 | UUUACAUCCUGAUUAUGUA | 464 | UACAUAAUCAGGAUGUAAA | 14501-14519 | 14499 |
| 162 | AUUACAUCCUGAUUAUGUA | 465 | UACAUAAUCAGGAUGUAAU | 14501-14519 | 14499 |
| 163 | NUUACAUCCUGAUUAUGUA | 466 | UACAUAAUCAGGAUGUAAN | 14501-14519 | 14499 |
| 164 | NUUACAUCCUGAUUAUGUN | 467 | NACAUAAUCAGGAUGUAAN | 14501-14519 | 14499 |
| 165 | AAGUUUACAUCCUGAUUAU | 468 | AUAAUCAGGAUGUAAACUU | 14504-14522 | 14502 |
| 166 | UAGUUUACAUCCUGAUUAU | 469 | AUAAUCAGGAUGUAAACUA | 14504-14522 | 14502 |
| 167 | NAGUUUACAUCCUGAUUAU | 470 | AUAAUCAGGAUGUAAACUN | 14504-14522 | 14502 |
| 168 | NAGUUUACAUCCUGAUUAN | 471 | NUAAUCAGGAUGUAAACUN | 14504-14522 | 14502 |
| 169 | UAAGUUUACAUCCUGAUUA | 472 | UAAUCAGGAUGUAAACUUA | 14505-14523 | 14503 |
| 170 | AAAGUUUACAUCCUGAUUA | 473 | UAAUCAGGAUGUAAACUUU | 14505-14523 | 14503 |
| 171 | NAAGUUUACAUCCUGAUUA | 474 | UAAUCAGGAUGUAAACUUN | 14505-14523 | 14503 |
| 172 | NAAGUUUACAUCCUGAUUN | 475 | NAAUCAGGAUGUAAACUUN | 14505-14523 | 14503 |
| 173 | GAGCUAUGUAAGUUUACAU | 476 | AUGUAAACUUACAUAGCUC | 14513-14531 | 14511 |
| 174 | UAGCUAUGUAAGUUUACAU | 477 | AUGUAAACUUACAUAGCUA | 14513-14531 | 14511 |
| 175 | AAGCUAUGUAAGUUUACAU | 478 | AUGUAAACUUACAUAGCUU | 14513-14531 | 14511 |
| 176 | NAGCUAUGUAAGUUUACAU | 479 | AUGUAAACUUACAUAGCUN | 14513-14531 | 14511 |
| 177 | NAGCUAUGUAAGUUUACAN | 480 | NUGUAAACUUACAUAGCUN | 14513-14531 | 14511 |
| 178 | AUAAUAAAGUCUAGCCUUA | 481 | UAAGGCUAGACUUUAUUAU | 14970-14988 | 14968 |
| 179 | UUAAUAAAGUCUAGCCUUA | 482 | UAAGGCUAGACUUUAUUAA | 14970-14988 | 14968 |
| 180 | NUAAUAAAGUCUAGCCUUA | 483 | UAAGGCUAGACUUUAUUAN | 14970-14988 | 14968 |
| 181 | NUAAUAAAGUCUAGCCUUN | 484 | NAAGGCUAGACUUUAUUAN | 14970-14988 | 14968 |
| 182 | GUACUACAGAUAGAGACAC | 485 | GUGUCUCUAUCUGUAGUAC | 15116-15134 | 15114 |
| 183 | UUACUACAGAUAGAGACAC | 486 | GUGUCUCUAUCUGUAGUAA | 15116-15134 | 15114 |
| 184 | AUACUACAGAUAGAGACAC | 487 | GUGUCUCUAUCUGUAGUAU | 15116-15134 | 15114 |
| 185 | NUACUACAGAUAGAGACAC | 488 | GUGUCUCUAUCUGUAGUAN | 15116-15134 | 15114 |
| 186 | NUACUACAGAUAGAGACAN | 489 | NUGUCUCUAUCUGUAGUAN | 15116-15134 | 15114 |
| 187 | CAUAGUACUACAGAUAGAG | 490 | CUCUAUCUGUAGUACUAUG | 15120-15138 | 15118 |
| 188 | UAUAGUACUACAGAUAGAG | 491 | CUCUAUCUGUAGUACUAUA | 15120-15138 | 15118 |
| 189 | AAUAGUACUACAGAUAGAG | 492 | CUCUAUCUGUAGUACUAUU | 15120-15138 | 15118 |
| 190 | NAUAGUACUACAGAUAGAG | 493 | CUCUAUCUGUAGUACUAUN | 15120-15138 | 15118 |
| 191 | NAUAGUACUACAGAUAGAN | 494 | NUCUAUCUGUAGUACUAUN | 15120-15138 | 15118 |
| 192 | UCAUAGUACUACAGAUAGA | 495 | UCUAUCUGUAGUACUAUGA | 15121-15139 | 15119 |
| 193 | ACAUAGUACUACAGAUAGA | 496 | UCUAUCUGUAGUACUAUGU | 15121-15139 | 15119 |
| 194 | NCAUAGUACUACAGAUAGA | 497 | UCUAUCUGUAGUACUAUGN | 15121-15139 | 15119 |
| 195 | NCAUAGUACUACAGAUAGN | 498 | NCUAUCUGUAGUACUAUGN | 15121-15139 | 15119 |
| 196 | GGUCAUAGUACUACAGAUA | 499 | UAUCUGUAGUACUAUGACC | 15123-15141 | 15121 |
| 197 | UGUCAUAGUACUACAGAUA | 500 | UAUCUGUAGUACUAUGACA | 15123-15141 | 15121 |
| 198 | AGUCAUAGUACUACAGAUA | 501 | UAUCUGUAGUACUAUGACU | 15123-15141 | 15121 |
| 199 | NGUCAUAGUACUACAGAUA | 502 | UAUCUGUAGUACUAUGACN | 15123-15141 | 15121 |
| 200 | NGUCAUAGUACUACAGAUN | 503 | NAUCUGUAGUACUAUGACN | 15123-15141 | 15121 |
| 201 | UGUUUAACUAGCAUUGUAU | 504 | AUACAAUGCUAGUUAAACA | 15887-15905 | 15885 |
| 202 | AGUUUAACUAGCAUUGUAU | 505 | AUACAAUGCUAGUUAAACU | 15887-15905 | 15885 |
| 203 | NGUUUAACUAGCAUUGUAU | 506 | AUACAAUGCUAGUUAAACN | 15887-15905 | 15885 |
| 204 | NGUUUAACUAGCAUUGUAN | 507 | NUACAAUGCUAGUUAAACN | 15887-15905 | 15885 |
| 205 | CUGUUUAACUAGCAUUGUA | 508 | UACAAUGCUAGUUAAACAG | 15888-15906 | 15886 |
| 206 | UUGUUUAACUAGCAUUGUA | 509 | UACAAUGCUAGUUAAACAA | 15888-15906 | 15886 |
| 207 | AUGUUUAACUAGCAUUGUA | 510 | UACAAUGCUAGUUAAACAU | 15888-15906 | 15886 |
| 208 | NUGUUUAACUAGCAUUGUA | 511 | UACAAUGCUAGUUAAACAN | 15888-15906 | 15886 |
| 209 | NUGUUUAACUAGCAUUGUN | 512 | NACAAUGCUAGUUAAACAN | 15888-15906 | 15886 |
| 210 | UAAUUUGCAACAUUGCUAG | 513 | CUAGCAAUGUUGCAAAUUA | 17024-17042 | 17022 |
| 211 | AAAUUUGCAACAUUGCUAG | 514 | CUAGCAAUGUUGCAAAUUU | 17024-17042 | 17022 |
| 212 | NAAUUUGCAACAUUGCUAG | 515 | CUAGCAAUGUUGCAAAUUN | 17024-17042 | 17022 |
| 213 | NAAUUUGCAACAUUGCUAN | 516 | NUAGCAAUGUUGCAAAUUN | 17024-17042 | 17022 |
| 214 | UCUCUAUCAGACAUUAUGC | 517 | GCAUAAUGUCUGAUAGAGA | 17957-17975 | 17955 |
| 215 | ACUCUAUCAGACAUUAUGC | 518 | GCAUAAUGUCUGAUAGAGU | 17957-17975 | 17955 |
| 216 | NCUCUAUCAGACAUUAUGC | 519 | GCAUAAUGUCUGAUAGAGN | 17957-17975 | 17955 |
| 217 | NCUCUAUCAGACAUUAUGN | 520 | NCAUAAUGUCUGAUAGAGN | 17957-17975 | 17955 |
| 218 | CCAUCAUAGAGAUGAGUCU | 521 | AGACUCAUCUCUAUGAUGG | 18196-18214 | 18194 |
| 219 | UCAUCAUAGAGAUGAGUCU | 522 | AGACUCAUCUCUAUGAUGA | 18196-18214 | 18194 |
| 220 | ACAUCAUAGAGAUGAGUCU | 523 | AGACUCAUCUCUAUGAUGU | 18196-18214 | 18194 |
| 221 | NCAUCAUAGAGAUGAGUCU | 524 | AGACUCAUCUCUAUGAUGN | 18196-18214 | 18194 |
| 222 | NCAUCAUAGAGAUGAGUCN | 525 | NGACUCAUCUCUAUGAUGN | 18196-18214 | 18194 |
| 223 | CUCCAAUUAAUGUGACUCC | 526 | GGAGUCACAUUAAUUGGAG | 20110-20128 | 20108 |
| 224 | UUCCAAUUAAUGUGACUCC | 527 | GGAGUCACAUUAAUUGGAA | 20110-20128 | 20108 |
| 225 | AUCCAAUUAAUGUGACUCC | 528 | GGAGUCACAUUAAUUGGAU | 20110-20128 | 20108 |
| 226 | NUCCAAUUAAUGUGACUCC | 529 | GGAGUCACAUUAAUUGGAN | 20110-20128 | 20108 |
| 227 | NUCCAAUUAAUGUGACUCN | 530 | NGAGUCACAUUAAUUGGAN | 20110-20128 | 20108 |
| 228 | UCAUUAAGAUCUGAAUCGA | 531 | UCGAUUCAGAUCUUAAUGA | 20945-20963 | 20943 |
| 229 | ACAUUAAGAUCUGAAUCGA | 532 | UCGAUUCAGAUCUUAAUGU | 20945-20963 | 20943 |
| 230 | NCAUUAAGAUCUGAAUCGA | 533 | UCGAUUCAGAUCUUAAUGN | 20945-20963 | 20943 |
| 231 | NCAUUAAGAUCUGAAUCGN | 534 | NCGAUUCAGAUCUUAAUGN | 20945-20963 | 20943 |
| 232 | UACAUAAGUUCGUACUCAU | 535 | AUGAGUACGAACUUAUGUA | 26231-26249 | 26229 |
| 233 | AACAUAAGUUCGUACUCAU | 536 | AUGAGUACGAACUUAUGUU | 26231-26249 | 26229 |
| 234 | NACAUAAGUUCGUACUCAU | 537 | AUGAGUACGAACUUAUGUN | 26231-26249 | 26229 |
| 235 | NACAUAAGUUCGUACUCAN | 538 | NUGAGUACGAACUUAUGUN | 26231-26249 | 26229 |
| 236 | CGAAUGAGUACAUAAGUUC | 539 | GAACUUAUGUACUCAUUCG | 26239-26257 | 26237 |
| 237 | UGAAUGAGUACAUAAGUUC | 540 | GAACUUAUGUACUCAUUCA | 26239-26257 | 26237 |
| 238 | AGAAUGAGUACAUAAGUUC | 541 | GAACUUAUGUACUCAUUCU | 26239-26257 | 26237 |
| 239 | NGAAUGAGUACAUAAGUUC | 542 | GAACUUAUGUACUCAUUCN | 26239-26257 | 26237 |
| 240 | NGAAUGAGUACAUAAGUUN | 543 | NAACUUAUGUACUCAUUCN | 26239-26257 | 26237 |
| 241 | ACGAAUGAGUACAUAAGUU | 544 | AACUUAUGUACUCAUUCGU | 26240-26258 | 26238 |
| 242 | UCGAAUGAGUACAUAAGUU | 545 | AACUUAUGUACUCAUUCGA | 26240-26258 | 26238 |
| 243 | NCGAAUGAGUACAUAAGUU | 546 | AACUUAUGUACUCAUUCGN | 26240-26258 | 26238 |
| 244 | NCGAAUGAGUACAUAAGUN | 547 | NACUUAUGUACUCAUUCGN | 26240-26258 | 26238 |
| 245 | CGAAACGAAUGAGUACAUA | 548 | UAUGUACUCAUUCGUUUCG | 26244-26262 | 26242 |
| 246 | UGAAACGAAUGAGUACAUA | 549 | UAUGUACUCAUUCGUUUCA | 26244-26262 | 26242 |
| 247 | AGAAACGAAUGAGUACAUA | 550 | UAUGUACUCAUUCGUUUCU | 26244-26262 | 26242 |
| 248 | NGAAACGAAUGAGUACAUA | 551 | UAUGUACUCAUUCGUUUCN | 26244-26262 | 26242 |
| 249 | NGAAACGAAUGAGUACAUN | 552 | NAUGUACUCAUUCGUUUCN | 26244-26262 | 26242 |
| 250 | AAGAAGUACGCUAUUAACU | 553 | AGUUAAUAGCGUACUUCUU | 26283-26301 | 26281 |
| 251 | UAGAAGUACGCUAUUAACU | 554 | AGUUAAUAGCGUACUUCUA | 26283-26301 | 26281 |
| 252 | NAGAAGUACGCUAUUAACU | 555 | AGUUAAUAGCGUACUUCUN | 26283-26301 | 26281 |
| 253 | NAGAAGUACGCUAUUAACN | 556 | NGUUAAUAGCGUACUUCUN | 26283-26301 | 26281 |
| 254 | AAGAAUACCACGAAAGCAA | 557 | UUGCUUUCGUGGUAUUCUU | 26306-26324 | 26304 |
| 255 | UAGAAUACCACGAAAGCAA | 558 | UUGCUUUCGUGGUAUUCUA | 26306-26324 | 26304 |
| 256 | NAGAAUACCACGAAAGCAA | 559 | UUGCUUUCGUGGUAUUCUN | 26306-26324 | 26304 |
| 257 | NAGAAUACCACGAAAGCAN | 560 | NUGCUUUCGUGGUAUUCUN | 26306-26324 | 26304 |
| 258 | CAGUAAGGAUGGCUAGUGU | 561 | ACACUAGCCAUCCUUACUG | 26332-26350 | 26330 |
| 259 | UAGUAAGGAUGGCUAGUGU | 562 | ACACUAGCCAUCCUUACUA | 26332-26350 | 26330 |
| 260 | AAGUAAGGAUGGCUAGUGU | 563 | ACACUAGCCAUCCUUACUU | 26332-26350 | 26330 |
| 261 | NAGUAAGGAUGGCUAGUGU | 564 | ACACUAGCCAUCCUUACUN | 26332-26350 | 26330 |
| 262 | NAGUAAGGAUGGCUAGUGN | 565 | NCACUAGCCAUCCUUACUN | 26332-26350 | 26330 |
| 263 | UUAACAAUAUUGCAGCAGU | 566 | ACUGCUGCAAUAUUGUUAA | 26369-26387 | 26367 |
| 264 | AUAACAAUAUUGCAGCAGU | 567 | ACUGCUGCAAUAUUGUUAU | 26369-26387 | 26367 |
| 265 | NUAACAAUAUUGCAGCAGU | 568 | ACUGCUGCAAUAUUGUUAN | 26369-26387 | 26367 |
| 266 | NUAACAAUAUUGCAGCAGN | 569 | NCUGCUGCAAUAUUGUUAN | 26369-26387 | 26367 |
| 267 | ACGUUAACAAUAUUGCAGC | 570 | GCUGCAAUAUUGUUAACGU | 26372-26390 | 26370 |
| 268 | UCGUUAACAAUAUUGCAGC | 571 | GCUGCAAUAUUGUUAACGA | 26372-26390 | 26370 |
| 269 | NCGUUAACAAUAUUGCAGC | 572 | GCUGCAAUAUUGUUAACGN | 26372-26390 | 26370 |
| 270 | NCGUUAACAAUAUUGCAGN | 573 | NCUGCAAUAUUGUUAACGN | 26372-26390 | 26370 |
| 271 | CACGUUAACAAUAUUGCAG | 574 | CUGCAAUAUUGUUAACGUG | 26373-26391 | 26371 |
| 272 | UACGUUAACAAUAUUGCAG | 575 | CUGCAAUAUUGUUAACGUA | 26373-26391 | 26371 |
| 273 | AACGUUAACAAUAUUGCAG | 576 | CUGCAAUAUUGUUAACGUU | 26373-26391 | 26371 |
| 274 | NACGUUAACAAUAUUGCAG | 577 | CUGCAAUAUUGUUAACGUN | 26373-26391 | 26371 |
| 275 | NACGUUAACAAUAUUGCAN | 578 | NUGCAAUAUUGUUAACGUN | 26373-26391 | 26371 |
| 276 | CGUUUAGACCAGAAGAUCA | 579 | UGAUCUUCUGGUCUAAACG | 26457-26475 | 26455 |
| 277 | UGUUUAGACCAGAAGAUCA | 580 | UGAUCUUCUGGUCUAAACA | 26457-26475 | 26455 |
| 278 | AGUUUAGACCAGAAGAUCA | 581 | UGAUCUUCUGGUCUAAACU | 26457-26475 | 26455 |
| 279 | NGUUUAGACCAGAAGAUCA | 582 | UGAUCUUCUGGUCUAAACN | 26457-26475 | 26455 |
| 280 | NGUUUAGACCAGAAGAUCN | 583 | NGAUCUUCUGGUCUAAACN | 26457-26475 | 26455 |
| 281 | AAUAAGAAAGCGUUCGUGA | 584 | UCACGAACGCUUUCUUAUU | 27039-27057 | 27037 |
| 282 | UAUAAGAAAGCGUUCGUGA | 585 | UCACGAACGCUUUCUUAUA | 27039-27057 | 27037 |
| 283 | NAUAAGAAAGCGUUCGUGA | 586 | UCACGAACGCUUUCUUAUN | 27039-27057 | 27037 |
| 284 | NAUAAGAAAGCGUUCGUGN | 587 | NCACGAACGCUUUCUUAUN | 27039-27057 | 27037 |
| 285 | UUUGUAAUAAGAAAGCGUU | 588 | AACGCUUUCUUAUUACAAA | 27044-27062 | 27042 |
| 286 | AUUGUAAUAAGAAAGCGUU | 589 | AACGCUUUCUUAUUACAAU | 27044-27062 | 27042 |
| 287 | NUUGUAAUAAGAAAGCGUU | 590 | AACGCUUUCUUAUUACAAN | 27044-27062 | 27042 |
| 288 | NUUGUAAUAAGAAAGCGUN | 591 | NACGCUUUCUUAUUACAAN | 27044-27062 | 27042 |
| 289 | CAUCUGUUGUCACUUACUG | 592 | CAGUAAGUGACAACAGAUG | 27186-27204 | 27184 |
| 290 | UAUCUGUUGUCACUUACUG | 593 | CAGUAAGUGACAACAGAUA | 27186-27204 | 27184 |
| 291 | AAUCUGUUGUCACUUACUG | 594 | CAGUAAGUGACAACAGAUU | 27186-27204 | 27184 |
| 292 | NAUCUGUUGUCACUUACUG | 595 | CAGUAAGUGACAACAGAUN | 27186-27204 | 27184 |
| 293 | NAUCUGUUGUCACUUACUN | 596 | NAGUAAGUGACAACAGAUN | 27186-27204 | 27184 |
| 294 | AACAUCUGUUGUCACUUAC | 597 | GUAAGUGACAACAGAUGUU | 27188-27206 | 27186 |
| 295 | UACAUCUGUUGUCACUUAC | 598 | GUAAGUGACAACAGAUGUA | 27188-27206 | 27186 |
| 296 | NACAUCUGUUGUCACUUAC | 599 | GUAAGUGACAACAGAUGUN | 27188-27206 | 27186 |
| 297 | NACAUCUGUUGUCACUUAN | 600 | NUAAGUGACAACAGAUGUN | 27188-27206 | 27186 |
| 298 | GGGAAUUUAAGGUCUUCCU | 601 | AGGAAGACCUUAAAUUCCC | 28455-28473 | 28453 |
| 299 | UGGAAUUUAAGGUCUUCCU | 602 | AGGAAGACCUUAAAUUCCA | 28455-28473 | 28453 |
| 300 | AGGAAUUUAAGGUCUUCCU | 603 | AGGAAGACCUUAAAUUCCU | 28455-28473 | 28453 |
| 301 | NGGAAUUUAAGGUCUUCCU | 604 | AGGAAGACCUUAAAUUCCN | 28455-28473 | 28453 |
| 302 | NGGAAUUUAAGGUCUUCCN | 605 | NGGAAGACCUUAAAUUCCN | 28455-28473 | 28453 |
| 303 | UGCUAUUGGUGUUAAUUGG | 606 | CCAAUUAACACCAAUAGCA | 28490-28508 | 28488 |
| 304 | AGCUAUUGGUGUUAAUUGG | 607 | CCAAUUAACACCAAUAGCU | 28490-28508 | 28488 |
| 305 | NGCUAUUGGUGUUAAUUGG | 608 | CCAAUUAACACCAAUAGCN | 28490-28508 | 28488 |
| 306 | NGCUAUUGGUGUUAAUUGN | 609 | NCAAUUAACACCAAUAGCN | 28490-28508 | 28488 |
| 307 | GCCAAUUUGGUCAUCUGGA | 610 | UCCAGAUGACCAAAUUGGC | 28510-28528 | 28508 |
| 308 | UCCAAUUUGGUCAUCUGGA | 611 | UCCAGAUGACCAAAUUGGA | 28510-28528 | 28508 |
| 309 | ACCAAUUUGGUCAUCUGGA | 612 | UCCAGAUGACCAAAUUGGU | 28510-28528 | 28508 |
| 310 | NCCAAUUUGGUCAUCUGGA | 613 | UCCAGAUGACCAAAUUGGN | 28510-28528 | 28508 |
| 311 | NCCAAUUUGGUCAUCUGGN | 614 | NCCAGAUGACCAAAUUGGN | 28510-28528 | 28508 |
| 312 | AGUAGAAAUACCAUCUUGG | 615 | CCAAGAUGGUAUUUCUACU | 28589-28607 | 28587 |
| 313 | UGUAGAAAUACCAUCUUGG | 616 | CCAAGAUGGUAUUUCUACA | 28589-28607 | 28587 |
| 314 | NGUAGAAAUACCAUCUUGG | 617 | CCAAGAUGGUAUUUCUACN | 28589-28607 | 28587 |
| 315 | NGUAGAAAUACCAUCUUGN | 618 | NCAAGAUGGUAUUUCUACN | 28589-28607 | 28587 |
| 316 | GGUAGUAGAAAUACCAUCU | 619 | AGAUGGUAUUUCUACUACC | 28592-28610 | 28590 |
| 317 | UGUAGUAGAAAUACCAUCU | 620 | AGAUGGUAUUUCUACUACA | 28592-28610 | 28590 |
| 318 | AGUAGUAGAAAUACCAUCU | 621 | AGAUGGUAUUUCUACUACU | 28592-28610 | 28590 |
| 319 | NGUAGUAGAAAUACCAUCU | 622 | AGAUGGUAUUUCUACUACN | 28592-28610 | 28590 |
| 320 | NGUAGUAGAAAUACCAUCN | 623 | NGAUGGUAUUUCUACUACN | 28592-28610 | 28590 |
| 321 | GUGAUCUUUUGGUGUAUUC | 624 | GAAUACACCAAAAGAUCAC | 28690-28708 | 28688 |
| 322 | UUGAUCUUUUGGUGUAUUC | 625 | GAAUACACCAAAAGAUCAA | 28690-28708 | 28688 |
| 323 | AUGAUCUUUUGGUGUAUUC | 626 | GAAUACACCAAAAGAUCAU | 28690-28708 | 28688 |
| 324 | NUGAUCUUUUGGUGUAUUC | 627 | GAAUACACCAAAAGAUCAN | 28690-28708 | 28688 |
| 325 | NUGAUCUUUUGGUGUAUUN | 628 | NAAUACACCAAAAGAUCAN | 28690-28708 | 28688 |
| 326 | CAGCAGAUUUCUUAGUGAC | 629 | GUCACUAAGAAAUCUGCUG | 29009-29027 | 29007 |
| 327 | UAGCAGAUUUCUUAGUGAC | 630 | GUCACUAAGAAAUCUGCUA | 29009-29027 | 29007 |
| 328 | AAGCAGAUUUCUUAGUGAC | 631 | GUCACUAAGAAAUCUGCUU | 29009-29027 | 29007 |
| 329 | NAGCAGAUUUCUUAGUGAC | 632 | GUCACUAAGAAAUCUGCUN | 29009-29027 | 29007 |
| 330 | NAGCAGAUUUCUUAGUGAN | 633 | NUCACUAAGAAAUCUGCUN | 29009-29027 | 29007 |
| 331 | UUACAUUGUAUGCUUUAGU | 634 | ACUAAAGCAUACAAUGUAA | 29066-29084 | 29064 |
| 332 | AUACAUUGUAUGCUUUAGU | 635 | ACUAAAGCAUACAAUGUAU | 29066-29084 | 29064 |
| 333 | NUACAUUGUAUGCUUUAGU | 636 | ACUAAAGCAUACAAUGUAN | 29066-29084 | 29064 |
| 334 | NUACAUUGUAUGCUUUAGN | 637 | NCUAAAGCAUACAAUGUAN | 29066-29084 | 29064 |
| 335 | UGUAAUCAGUUCCUUGUCU | 638 | AGACAAGGAACUGAUUACA | 29150-29168 | 29148 |
| 336 | AGUAAUCAGUUCCUUGUCU | 639 | AGACAAGGAACUGAUUACU | 29150-29168 | 29148 |
| 337 | NGUAAUCAGUUCCUUGUCU | 640 | AGACAAGGAACUGAUUACN | 29150-29168 | 29148 |
| 338 | NGUAAUCAGUUCCUUGUCN | 641 | NGACAAGGAACUGAUUACN | 29150-29168 | 29148 |
| 339 | UUUGUAAUCAGUUCCUUGU | 642 | ACAAGGAACUGAUUACAAA | 29152-29170 | 29150 |
| 340 | AUUGUAAUCAGUUCCUUGU | 643 | ACAAGGAACUGAUUACAAU | 29152-29170 | 29150 |
| 341 | NUUGUAAUCAGUUCCUUGU | 644 | ACAAGGAACUGAUUACAAN | 29152-29170 | 29150 |
| 342 | NUUGUAAUCAGUUCCUUGN | 645 | NCAAGGAACUGAUUACAAN | 29152-29170 | 29150 |
| 343 | CCAAUGUUUGUAAUCAGUU | 646 | AACUGAUUACAAACAUUGG | 29158-29176 | 29156 |
| 344 | UCAAUGUUUGUAAUCAGUU | 647 | AACUGAUUACAAACAUUGA | 29158-29176 | 29156 |
| 345 | ACAAUGUUUGUAAUCAGUU | 648 | AACUGAUUACAAACAUUGU | 29158-29176 | 29156 |
| 346 | NCAAUGUUUGUAAUCAGUU | 649 | AACUGAUUACAAACAUUGN | 29158-29176 | 29156 |
| 347 | NCAAUGUUUGUAAUCAGUN | 650 | NACUGAUUACAAACAUUGN | 29158-29176 | 29156 |
| 348 | GCAAAUUGUGCAAUUUGCG | 651 | CGCAAAUUGCACAAUUUGC | 29178-29196 | 29176 |
| 349 | UCAAAUUGUGCAAUUUGCG | 652 | CGCAAAUUGCACAAUUUGA | 29178-29196 | 29176 |
| 350 | ACAAAUUGUGCAAUUUGCG | 653 | CGCAAAUUGCACAAUUUGU | 29178-29196 | 29176 |
| 351 | NCAAAUUGUGCAAUUUGCG | 654 | CGCAAAUUGCACAAUUUGN | 29178-29196 | 29176 |
| 352 | NCAAAUUGUGCAAUUUGCN | 655 | NGCAAAUUGCACAAUUUGN | 29178-29196 | 29176 |
| 353 | GGAUCUUUGUCAUCCAAUU | 656 | AAUUGGAUGACAAAGAUCC | 29286-29304 | 29284 |
| 354 | UGAUCUUUGUCAUCCAAUU | 657 | AAUUGGAUGACAAAGAUCA | 29286-29304 | 29284 |
| 355 | AGAUCUUUGUCAUCCAAUU | 658 | AAUUGGAUGACAAAGAUCU | 29286-29304 | 29284 |
| 356 | NGAUCUUUGUCAUCCAAUU | 659 | AAUUGGAUGACAAAGAUCN | 29286-29304 | 29284 |
| 357 | NGAUCUUUGUCAUCCAAUN | 660 | NAUUGGAUGACAAAGAUCN | 29286-29304 | 29284 |
| 358 | GCGUCAAUAUGCUUAUUCA | 661 | UGAAUAAGCAUAUUGACGC | 29331-29349 | 29329 |
| 359 | UCGUCAAUAUGCUUAUUCA | 662 | UGAAUAAGCAUAUUGACGA | 29331-29349 | 29329 |
| 360 | ACGUCAAUAUGCUUAUUCA | 663 | UGAAUAAGCAUAUUGACGU | 29331-29349 | 29329 |
| 361 | NCGUCAAUAUGCUUAUUCA | 664 | UGAAUAAGCAUAUUGACGN | 29331-29349 | 29329 |
| 362 | NCGUCAAUAUGCUUAUUCN | 665 | NGAAUAAGCAUAUUGACGN | 29331-29349 | 29329 |
| 363 | GGAAUGUUUUGUAUGCGUC | 666 | GACGCAUACAAAACAUUCC | 29345-29363 | 29343 |
| 364 | UGAAUGUUUUGUAUGCGUC | 667 | GACGCAUACAAAACAUUCA | 29345-29363 | 29343 |
| 365 | AGAAUGUUUUGUAUGCGUC | 668 | GACGCAUACAAAACAUUCU | 29345-29363 | 29343 |
| 366 | NGAAUGUUUUGUAUGCGUC | 669 | GACGCAUACAAAACAUUCN | 29345-29363 | 29343 |
| 367 | NGAAUGUUUUGUAUGCGUN | 670 | NACGCAUACAAAACAUUCN | 29345-29363 | 29343 |
| 368 | GAUUAAAGAUUGCUAUGUG | 671 | CACAUAGCAAUCUUUAAUC | 29668-29686 | 29666 |
| 369 | UAUUAAAGAUUGCUAUGUG | 672 | CACAUAGCAAUCUUUAAUA | 29668-29686 | 29666 |
| 370 | AAUUAAAGAUUGCUAUGUG | 673 | CACAUAGCAAUCUUUAAUU | 29668-29686 | 29666 |
| 371 | NAUUAAAGAUUGCUAUGUG | 674 | CACAUAGCAAUCUUUAAUN | 29668-29686 | 29666 |
| 372 | NAUUAAAGAUUGCUAUGUN | 675 | NACAUAGCAAUCUUUAAUN | 29668-29686 | 29666 |

The CoV RNAi agent sense strands and antisense strands that comprise or consist of the nucleotide sequences in Table 2 can be modified nucleotides or unmodified nucleotides. In some embodiments, the CoV RNAi agents having the sense and antisense strand sequences that comprise or consist of any of the nucleotide sequences in Table 2 are all or substantially all modified nucleotides.

In some embodiments, the antisense strand of a CoV RNAi agent disclosed herein differs by 0, 1, 2, or 3 nucleotides from any of the antisense strand sequences in Table 2. In some embodiments, the sense strand of a CoV RNAi agent disclosed herein differs by 0, 1, 2, or 3 nucleotides from any of the sense strand sequences in Table 2.

As used herein, each N listed in a sequence disclosed in Table 2 may be independently selected from any and all nucleobases (including those found on both modified and unmodified nucleotides). In some embodiments, an N nucleotide listed in a sequence disclosed in Table 2 has a nucleobase that is complementary to the N nucleotide at the corresponding position on the other strand. In some embodiments, an N nucleotide listed in a sequence disclosed in Table 2 has a nucleobase that is not complementary to the N nucleotide at the corresponding position on the other strand. In some embodiments, an N nucleotide listed in a sequence disclosed in Table 2 has a nucleobase that is the same as the N nucleotide at the corresponding position on the other strand. In some embodiments, an N nucleotide listed in a sequence disclosed in Table 2 has a nucleobase that is different from the N nucleotide at the corresponding position on the other strand.

Certain modified CoV RNAi agent sense and antisense strands are provided in Table 3, Table 4, Table 5, Table 6, and Table 10. Certain modified CoV RNAi agent antisense strands, as well as their underlying unmodified nucleobase sequences, are provided in Table 3. Certain modified CoV RNAi agent sense strands, as well as their underlying unmodified nucleobase sequences, are provided in Tables 4, 5, and 6. In forming CoV RNAi agents, each of the nucleotides in each of the underlying base sequences listed in Tables 3, 4, 5, and 6, as well as in Table 2, above, can be a modified nucleotide.

The CoV RNAi agents described herein are formed by annealing an antisense strand with a sense strand. A sense strand containing a sequence listed in Table 2, Table 4, Table 5, or Table 6 can be hybridized to any antisense strand containing a sequence listed in Table 2 or Table 3, provided the two sequences have a region of at least 85% complementarity over a contiguous 16, 17, 18, 19, 20, or 21 nucleotide sequence.

In some embodiments, a CoV RNAi agent antisense strand comprises a nucleotide sequence of any of the sequences in Table 2 or Table 3.

In some embodiments, a CoV RNAi agent comprises or consists of a duplex having the nucleobase sequences of the sense strand and the antisense strand of any of the sequences in Table 2, Table 3, Table 4, Table 5, Table 6, or Table 10.

Examples of antisense strands containing modified nucleotides are provided in Table 3. Examples of sense strands containing modified nucleotides are provided in Tables 4, 5 and 6.

As used in Tables 3, 4, 5, 6, and 10, the following notations are used to indicate modified nucleotides, targeting groups, and linking groups:
A=adenosine-3′-phosphate
C=cytidine-3′-phosphate
G=guanosine-3′-phosphate
U=uridine-3′-phosphate
I=inosine-3′-phosphate
a=2′-O-methyladenosine-3′-phosphate
as =2′-O-methyladenosine-3′-phosphorothioate
c=2′-O-methylcytidine-3′-phosphate
cs=2′-O-methylcytidine-3′-phosphorothioate
g=2′-O-methylguanosine-3′-phosphate
gs=2′-O-methylguanosine-3′-phosphorothioate
i=2′-O-methylinosine-3′-phosphate
is =2′-O-methylinosine-3′-phosphorothioate
t=2′-O-methyl-5-methyluridine-3′-phosphate
ts=2′-O-methyl-5-methyluridine-3′-phosphorothioate
u=2′-O-methyluridine-3′-phosphate
us=2′-O-methyluridine-3′-phosphorothioate
Af=2′-fluoroadenosine-3′-phosphate
Afs=2′-fluoroadenosine-3′-phosporothioate
Cf=2′-fluorocytidine-3′-phosphate
Cfs=2′-fluorocytidine-3′-phosphorothioate
Gf=2′-fluoroguanosine-3′-phosphate
Gfs=2′-fluoroguanosine-3′-phosphorothioate
Tf=2′-fluoro-5′-methyluridine-3′-phosphate
Tfs=2′-fluoro-5′-methyluridine-3′-phosphorothioate
Uf=2′-fluorouridine-3′-phosphate
Ufs=2′-fluorouridine-3′-phosphorothioate
dT=2′-deoxythymidine-3′-phosphate
A_{UNA}=2′,3′-seco-adenosine-3′-phosphate
A_{UNAS}=2′,3′-seco-adenosine-3′-phosphorothioate
C_{UNA}=2′,3′-seco-cytidine-3′-phosphate
C_{UNA}s=2′,3′-seco-cytidine-3′-phosphorothioate
G_{UNA}=2′,3′-seco-guanosine-3′-phosphate
G_{UNA}s=2′,3′-seco-guanosine-3′-phosphorothioate
U_{UNA}=2′,3′-seco-uridine-3′-phosphate
U_{UNA}s=2′,3′-seco-uridine-3′-phosphorothioate
a_2N=see Table 11
a_2Ns=see Table 11
(invAb)=inverted abasic deoxyribonucleotide-5′-phosphate, see Table 11
(invAb)s=inverted abasic deoxyribonucleotide-5′-phosphorothioate, see Table 11
s=phosphorothioate linkage
p=terminal phosphate (as synthesized)
vpdN=vinyl phosphonate deoxyribonucleotide
cPrpa=5′-cyclopropyl phosphonate-2′-O-methyladenosine-3′-phosphate (see Table 11)
cPrpas=5′-cyclopropyl phosphonate-2′-O-methyladenosine-3′-phosphorothioate (see Table 11)
cPrpu=5′-cyclopropyl phosphonate-2′-O-methyluridine-3′-phosphate (see Table 11) cPrpus=5′-cyclopropyl phosphonate-2′-O-methyluridine-3′-phosphorothioate (see Table 11)
(Alk-SS-C6)=see Table 11
(C6-SS-Alk)=see Table 11
(C6-SS-C6)=see Table 11
(6-SS-6)=see Table 11
(C6-SS-Alk-Me)=see Table 11
(NH2-C6)=see Table 11
(TriAlkl4)=see Table 11
(TriAlkl4)s=see Table 11
-C6-=see Table 11
-C6s-=see Table 11
-L6-C6-=see Table 11
-L6-C6s-=see Table 11
-Alk-cyHex-=see Table 11
-Alk-cyHexs-=see Table 11
-(TA14)=see Table 11 (structure of (TriAlkl4)s after conjugation)
(TA14)s=see Table 11 (structure of (TriAlkl4)s after conjugation)

As the person of ordinary skill in the art would readily understand, unless otherwise indicated by the sequence (such as, for example, by a phosphorothioate linkage “s”), when present in an oligonucleotide, the nucleotide monomers are mutually linked by 5′-3′-phosphodiester bonds. As the person of ordinary skill in the art would clearly understand, the inclusion of a phosphorothioate linkage as shown in the modified nucleotide sequences disclosed herein replaces the phosphodiester linkage typically present in oligonucleotides. Further, the person of ordinary skill in the art would readily understand that the terminal nucleotide at the 3′ end of a given oligonucleotide sequence would typically have a hydroxyl (—OH) group at the respective 3′ position of the given monomer instead of a phosphate moiety ex vivo. Additionally, for the embodiments disclosed herein, when viewing the respective strand 5′→3′, the inverted abasic residues are inserted such that the 3′ position of the deoxyribose is linked at the 3′ end of the preceding monomer on the respective strand (see, e.g., Table 11). Moreover, as the person of ordinary skill would readily understand and appreciate, while the phosphorothioate chemical structures depicted herein typically show the anion on the sulfur atom, the inventions disclosed herein encompass all phosphorothioate tautomers (e.g., where the sulfur atom has a double-bond and the anion is on an oxygen atom). Unless expressly indicated otherwise herein, such understandings of the person of ordinary skill in the art are used when describing the CoV RNAi agents and compositions of CoV RNAi agents disclosed herein.

Certain examples of targeting groups and linking groups used with the CoV RNAi agents disclosed herein are included in the chemical structures provided below in Table 11. Each sense strand and/or antisense strand can have any targeting groups or linking groups listed herein, as well as other targeting or linking groups, conjugated to the 5′ and/or 3′ end of the sequence.

TABLE 3
CoV RNAi agent Antisense Strand Sequences

| | | | Underlying Base | |
|---|---|---|---|---|
| | | | Sequence (5′ → 3′) | |
| AS | | SEQ ID | (Shown as an Unmodified | SEQ ID |
| Strand ID | Modified Antisense Strand (5′ → 3′) | NO. | Nucleotide Sequence) | NO. |
| AM11997-AS | asAfsasCfuGfaGfuUfgGfaCfgUfgUfgUfsc | 676 | AAACUGAGUUGGACGUGUGUC | 1060 |
| AM11999-AS | usGfsasUfuUfuCfcAfcUfaCfuUfcUfuCfsc | 677 | UGAUUUUCCACUACUUCUUCC | 1061 |
| AM12001-AS | usUfsasGfgAfuUfuUfcCfaCfuAfcUfuCfsc | 678 | UUAGGAUUUUCCACUACUUCC | 1062 |
| AM12003-AS | asAfsasCfcAfaCfaCfuAfcCfaCfaUfgAfsc | 679 | AAACCAACACUACCACAUGAC | 1063 |
| AM12005-AS | asUfsasCfaUfuUfgGfgUfcAfuAfgCfuUfsg | 680 | AUACAUUUGGGUCAUAGCUUG | 1064 |
| AM12007-AS | usGfsasUfaUfaUfgUfgGfuAfcCfaUfgUfsc | 681 | UGAUAUAUGUGGUACCAUGUC | 1065 |
| AM12009-AS | usUfsgsAfuAfuAfuGfuGfgUfaCfcAfuGfsc | 682 | UUGAUAUAUGUGGUACCAUGC | 1066 |
| AM12011-AS | usUfsusAfcAfuCfcUfgAfuUfaUfgUfaCfsc | 683 | UUUACAUCCUGAUUAUGUACC | 1067 |
| AM12013-AS | asAfsgsUfuUfaCfaUfcCfuGfaUfuAfuGfsc | 684 | AAGUUUACAUCCUGAUUAUGC | 1068 |
| AM12015-AS | usAfsasGfuUfuAfcAfuCfcUfgAfuUfaUfsg | 685 | UAAGUUUACAUCCUGAUUAUG | 1069 |
| AM12017-AS | usAfsgsCfuAfuGfuAfaGfuUfuAfcAfuCfsc | 686 | UAGCUAUGUAAGUUUACAUCC | 1070 |
| AM12019-AS | asUfsasAfuAfaAfgUfcUfaGfcCfuUfaCfsc | 687 | AUAAUAAAGUCUAGCCUUACC | 1071 |
| AM12021-AS | usUfsasCfuAfcAfgAfuAfgAfgAfcAfcCfsa | 688 | UUACUACAGAUAGAGACACCA | 1072 |
| AM12023-AS | usAfsusAfgUfaCfuAfcAfgAfuAfgAfgAfsc | 689 | UAUAGUACUACAGAUAGAGAC | 1073 |
| AM12025-AS | usCfsasUfaGfuAfcUfaCfaGfaUfaGfaGfsc | 690 | UCAUAGUACUACAGAUAGAGC | 1074 |
| AM12027-AS | usGfsusCfaUfaGfuAfcUfaCfaGfaUfaGfsc | 691 | UGUCAUAGUACUACAGAUAGC | 1075 |
| AM12029-AS | usAfsasUfuUfgCfaAfcAfuUfgCfuAfgAfsc | 692 | UAAUUUGCAACAUUGCUAGAC | 1076 |
| AM12031-AS | usCfsasUfcAfuAfgAfgAfuGfaGfuCfuUfsc | 693 | UCAUCAUAGAGAUGAGUCUUC | 1077 |
| AM12033-AS | usUfscsCfaAfuUfaAfuGfuGfaCfuCfcAfsc | 694 | UUCCAAUUAAUGUGACUCCAC | 1078 |
| AM12035-AS | usAfscsAfuAfaGfuUfcGfuAfcUfcAfuCfsc | 695 | UACAUAAGUUCGUACUCAUCC | 1079 |
| AM12037-AS | usGfsasAfuGfaGfuAfcAfuAfaGfuUfcGfsu | 696 | UGAAUGAGUACAUAAGUUCGU | 1080 |
| AM12039-AS | asCfsgsAfaUfgAfgUfaCfaUfaAfgUfuCfsg | 697 | ACGAAUGAGUACAUAAGUUCG | 1081 |
| AM12041-AS | usGfsasAfaCfgAfaUfgAfgUfaCfaUfaAfsg | 698 | UGAAACGAAUGAGUACAUAAG | 1082 |
| AM12044-AS | asAfsgsAfaGfuAfcGfcUfaUfuAfaCfuAfsc | 699 | AAGAAGUACGCUAUUAACUAC | 1083 |
| AM12046-AS | asAfsgsAfaUfaCfcAfcGfaAfaGfcAfaGfsc | 700 | AAGAAUACCACGAAAGCAAGC | 1084 |
| AM12048-AS | usAfsgsUfaAfgGfaUfgGfcUfaGfuGfuAfsc | 701 | UAGUAAGGAUGGCUAGUGUAC | 1085 |
| AM12050-AS | usUfsasAfcAfaUfaUfuGfcAfgCfaGfuAfsc | 702 | UUAACAAUAUUGCAGCAGUAC | 1086 |
| AM12052-AS | asCfsgsUfuAfaCfaAfuAfuUfgCfaGfcAfsg | 703 | ACGUUAACAAUAUUGCAGCAG | 1087 |
| AM12054-AS | usAfscsGfuUfaAfcAfaUfaUfuGfcAfgCfsa | 704 | UACGUUAACAAUAUUGCAGCA | 1088 |
| AM12057-AS | usGfsusUfuAfgAfcCfaGfaAfgAfuCfaGfsg | 705 | UGUUUAGACCAGAAGAUCAGG | 1089 |
| AM12059-AS | asAfsusAfaGfaAfaGfcGfuUfcGfuGfaUfsg | 706 | AAUAAGAAAGCGUUCGUGAUG | 1090 |
| AM12062-AS | usUfsusGfuAfaUfaAfgAfaAfgCfgUfuCfsg | 707 | UUUGUAAUAAGAAAGCGUUCG | 1091 |
| AM12064-AS | usAfsusCfuGfuUfgUfcAfcUfuAfcUfgUfsc | 708 | UAUCUGUUGUCACUUACUGUC | 1092 |
| AM12066-AS | asAfscsAfuCfuGfuUfgUfcAfcUfuAfcUfsg | 709 | AACAUCUGUUGUCACUUACUG | 1093 |
| AM12068-AS | usGfsasUfcUfuUfgUfcAfuCfcAfaUfuUfsg | 710 | UGAUCUUUGUCAUCCAAUUUG | 1094 |
| AM12070-AS | usAfsusUfaAfaGfaUfuGfcUfaUfgUfgAfsg | 711 | UAUUAAAGAUUGCUAUGUGAG | 1095 |
| AM12469-AS | usUfsgsUfcUfaAfcAfaCfaUfcAfaAfaGfsg | 712 | UUGUCUAACAACAUCAAAAGG | 1096 |
| AM12471-AS | usCfsusUfgAfuCfuAfaAfgCfaUfuAfcCfsa | 713 | UCUUGAUCUAAAGCAUUACCA | 1097 |
| AM12473-AS | usGfsusUfuAfaCfuAfgCfaUfuGfuAfuGfsc | 714 | UGUUUAACUAGCAUUGUAUGC | 1098 |
| AM12475-AS | usUfsgsUfuUfaAfcUfaGfcAfuUfgUfaUfsg | 715 | UUGUUUAACUAGCAUUGUAUG | 1099 |
| AM12477-AS | usCfsusCfuAfuCfaGfaCfaUfuAfuGfcAfsc | 716 | UCUCUAUCAGACAUUAUGCAC | 1100 |
| AM12479-AS | usCfsasUfuAfaGfaUfcUfgAfaUfcGfaCfsc | 717 | UCAUUAAGAUCUGAAUCGACC | 1101 |
| AM12481-AS | usCfscsAfaUfuUfgGfuCfaUfcUfgGfaCfsc | 718 | UCCAAUUUGGUCAUCUGGACC | 1102 |
| AM12483-AS | usCfsasAfaUfuGfuGfcAfaUfuUfgCfgGfsc | 719 | UCAAAUUGUGCAAUUUGCGGC | 1103 |
| AM12485-AS | usGfsasAfuGfuUfuUfgUfaUfgCfgUfcAfsc | 720 | UGAAUGUUUUGUAUGCGUCAC | 1104 |
| AM12563-AS | usGfsgsAfaUfuUfaAfgGfuCfuUfcCfuUfsg | 721 | UGGAAUUUAAGGUCUUCCUUG | 1105 |
| AM12565-AS | usGfscsUfaUfuGfgUfgUfuAfaUfuGfgAfsc | 722 | UGCUAUUGGUGUUAAUUGGAC | 1106 |
| AM12567-AS | asGfsusAfgAfaAfuAfcCfaUfcUfuGfgAfsc | 723 | AGUAGAAAUACCAUCUUGGAC | 1107 |
| AM12569-AS | usGfsusAfgUfaGfaAfaUfaCfcAfuCfuUfsg | 724 | UGUAGUAGAAAUACCAUCUUG | 1108 |
| AM12571-AS | usUfsgsAfuCfuUfuUfgGfuGfuAfuUfcAfsc | 725 | UUGAUCUUUUGGUGUAUUCAC | 1109 |
| AM12573-AS | usAfsgsCfaGfaUfuUfcUfuAfgUfgAfcAfsg | 726 | UAGCAGAUUUCUUAGUGACAG | 1110 |
| AM12575-AS | usUfsasCfaUfuGfuAfuGfcUfuUfaGfuGfsg | 727 | UUACAUUGUAUGCUUUAGUGG | 1111 |
| AM12577-AS | usGfsusAfaUfcAfgUfuCfcUfuGfuCfuGfsc | 728 | UGUAAUCAGUUCCUUGUCUGC | 1112 |
| AM12579-AS | usUfsusGfuAfaUfcAfgUfuCfcUfuGfuCfsc | 729 | UUUGUAAUCAGUUCCUUGUCC | 1113 |
| AM12581-AS | usCfsasAfuGfuUfuGfuAfaUfcAfgUfuCfsc | 730 | UCAAUGUUUGUAAUCAGUUCC | 1114 |
| AM12583-AS | usCfsgsUfcAfaUfaUfgCfuUfaUfuCfaGfsc | 731 | UCGUCAAUAUGCUUAUUCAGC | 1115 |
| AM13120-AS | usCfsasUfaAfgCfaCfuCfuUfaAfgAfaGfsc | 732 | UCAUAAGCACUCUUAAGAAGC | 1116 |
| AM13122-AS | usUfsasGfaUfaAfgAfcAfuUfgUfcUfaAfsg | 733 | UUAGAUAAGACAUUGUCUAAG | 1117 |
| AM13124-AS | usAfsusGfaAfgAfuUfgCfcAfuUfaAfuGfsc | 734 | UAUGAAGAUUGCCAUUAAUGC | 1118 |
| AM13126-AS | asAfsasCfgUfaUfuAfaCfgUfaAfgCfaUfsc | 735 | AAACGUAUUAACGUAAGCAUC | 1119 |
| AM13128-AS | usCfsusUfaAfgAfuUfgUfcAfaAfgGfuGfsc | 736 | UCUUAAGAUUGUCAAAGGUGC | 1120 |
| AM13160-AS | usUfsgsAfuUfaUfcUfaAfuGfuCfaGfuAfsc | 737 | UUGAUUAUCUAAUGUCAGUAC | 1121 |
| AM13543-AS | usUfsasguagguauAfaCfcAfcagcsa | 738 | UUAGUAGGUAUAACCACAGCA | 1122 |
| AM13545-AS | asAfsasGfaagugucUfuAfaGfauugsc | 739 | AAAGAAGUGUCUUAAGAUUGC | 1123 |
| AM13547-AS | usCfsgsucaaUfAfugCfuUfaUfucagsc | 740 | UCGUCAAUAUGCUUAUUCAGC | 1115 |
| AM14662-AS | usUfscsAfuAfaGfcAfcUfcUfuAfaGfaAfsg | 741 | UUCAUAAGCACUCUUAAGAAG | 1124 |
| AM14666-AS | cPrpusCfsusUfaAfgAfuUfgUfcAfaAfgGfuGfsc | 742 | UCUUAAGAUUGUCAAAGGUGC | 1120 |
| AM14667-AS | cPrpusUfsasGfgAfuUfuUfcCfaCfuAfcUfuCfsc | 743 | UUAGGAUUUUCCACUACUUCC | 1062 |
| AM14686-AS | cPrpusUfsasGfaUfaAfgAfcAfuUfgUfcUfaAfsg | 744 | UUAGAUAAGACAUUGUCUAAG | 1117 |
| AM14687-AS | cPrpusGfsusAfgUfaGfaAfaUfaCfcAfuCfuUfsg | 745 | UGUAGUAGAAAUACCAUCUUG | 1108 |
| AM14688-AS | cPrpusUfsusGfuAfaUfcAfgUfuCfcUfuGfuCfsc | 746 | UUUGUAAUCAGUUCCUUGUCC | 1113 |
| AM14689-AS | cPrpusUfsasCfaUfuGfuAfuGfcUfuUfaGfuGfsg | 747 | UUACAUUGUAUGCUUUAGUGG | 1111 |
| AM14690-AS | cPrpusUfsgsUfuUfaAfcUfaGfcAfuUfgUfaUfsg | 748 | UUGUUUAACUAGCAUUGUAUG | 1099 |
| AM14859-AS | cPrpasGfsusAfgAfaAfuAfcCfaUfcUfuGfgAfsc | 749 | AGUAGAAAUACCAUCUUGGAC | 1107 |
| AM14861-AS | cPrpusGfsusAfgAfaAfuAfcCfaUfcUfuGfgAfsc | 750 | UGUAGAAAUACCAUCUUGGAC | 1125 |
| AM15563-AS | cPrpusUfsaGfgauuuucCfaCfuAfcuuscsc | 751 | UUAGGAUUUUCCACUACUUCC | 1062 |
| AM15565-AS | cPrpusUfsaggaUfuuucCfaCfuAfcuuscsc | 752 | UUAGGAUUUUCCACUACUUCC | 1062 |
| AM15566-AS | cPrpusUfsaggauuUfucCfaCfuAfcuuscsc | 753 | UUAGGAUUUUCCACUACUUCC | 1062 |
| AM15568-AS | cPrpusUfsaggauuUfucCfaCfuAfcuuscsg | 754 | UUAGGAUUUUCCACUACUUCG | 1126 |
| AM15569-AS | cPrpuUfaggauuUfucCfaCfuAfcuuscsc | 755 | UUAGGAUUUUCCACUACUUCC | 1062 |
| AM15570-AS | cPrpusUfsgUfuuaacuaGfcAfuUfguasusg | 756 | UUGUUUAACUAGCAUUGUAUG | 1099 |
| AM15572-AS | cPrpuUfgUfuuaacuaGfcAfuUfguasusg | 757 | UUGUUUAACUAGCAUUGUAUG | 1099 |
| AM15574-AS | cPrpusUfsgUfuuaacuaGfcAfuUfguascsg | 758 | UUGUUUAACUAGCAUUGUACG | 1127 |
| AM15575-AS | cPrpusUfsguuuAfacuaGfcAfuUfguasusg | 759 | UUGUUUAACUAGCAUUGUAUG | 1099 |
| AM15576-AS | cPrpasGfsuAfgaaauacCfaUfcUfuggsasc | 760 | AGUAGAAAUACCAUCUUGGAC | 1107 |
| AM15578-AS | cPrpasGfsuagaAfauacCfaUfcUfuggsasc | 761 | AGUAGAAAUACCAUCUUGGAC | 1107 |
| AM15579-AS | cPrpasGfsuagaaaUfacCfaUfcUfuggsasc | 762 | AGUAGAAAUACCAUCUUGGAC | 1107 |
| AM15580-AS | cPrpaGfuagaaaUfacCfaUfcUfuggsasc | 763 | AGUAGAAAUACCAUCUUGGAC | 1107 |
| AM15582-AS | cPrpusGfsuagaaaUfacCfaUfcUfuggsasc | 764 | UGUAGAAAUACCAUCUUGGAC | 1125 |
| AM15795-AS | cPrpusUfsusGfuaaucagUfuCfcUfugucsc | 765 | UUUGUAAUCAGUUCCUUGUCC | 1113 |
| AM15797-AS | cPrpusUfsusGfuaaUfCfagUfuCfcUfugucsc | 766 | UUUGUAAUCAGUUCCUUGUCC | 1113 |
| AM15798-AS | cPrpusUfsuGfuaaucagUfuCfcUfuguscsc | 767 | UUUGUAAUCAGUUCCUUGUCC | 1113 |
| AM15799-AS | cPrpuUfuGfuaaucagUfuCfcUfuguscsc | 768 | UUUGUAAUCAGUUCCUUGUCC | 1113 |
| AM15801-AS | cPrpusUfsusGfuaaucagUfuCfcUfugucsg | 769 | UUUGUAAUCAGUUCCUUGUCG | 1128 |
| AM15803-AS | cPrpusAfsasGfuUfuAfcAfuCfcUfgAfuUfaUfsg | 770 | UAAGUUUACAUCCUGAUUAUG | 1069 |
| AM15804-AS | cPrpusAfsasGfuuuacauCfcUfgAfuuausg | 771 | UAAGUUUACAUCCUGAUUAUG | 1069 |
| AM15807-AS | cPrpusAfsasGfuuuacauCfcUfgAfuuacsg | 772 | UAAGUUUACAUCCUGAUUACG | 1129 |
| AM15808-AS | cPrpusAfsasGfuuuAfCfauCfcUfgAfuuausg | 773 | UAAGUUUACAUCCUGAUUAUG | 1069 |
| AM15810-AS | cPrpusAfsgsCfuAfuGfuAfaGfuUfuAfcAfuCfsc | 774 | UAGCUAUGUAAGUUUACAUCC | 1070 |
| AM15812-AS | cPrpusAfsgcuaugUfaaGfuUfuAfcauscsc | 775 | UAGCUAUGUAAGUUUACAUCC | 1070 |
| AM15813-AS | cPrpusCfsusUfaagauugUfcAfaAfggugsc | 776 | UCUUAAGAUUGUCAAAGGUGC | 1120 |
| AM15815-AS | cPrpusCfsusUfaagAfUfugUfcAfaAfggugsc | 777 | UCUUAAGAUUGUCAAAGGUGC | 1120 |
| AM15816-AS | cPrpusCfsusuaaGfauugUfcAfaAfggugsc | 778 | UCUUAAGAUUGUCAAAGGUGC | 1120 |
| AM15818-AS | cPrpusUfsasGfuAfgGfuAfuAfaCfcAfcAfgCfsa | 779 | UUAGUAGGUAUAACCACAGCA | 1122 |
| AM15820-AS | cPrpasAfsgsAfaGfuGfuCfuUfaAfgAfuUfgUfsc | 780 | AAGAAGUGUCUUAAGAUUGUC | 1130 |
| AM15822-AS | cPrpusCfsasUfaAfgAfaAfgUfgUfgCfcCfaUfsg | 781 | UCAUAAGAAAGUGUGCCCAUG | 1131 |
| AM15824-AS | cPrpusAfsasCfuUfaGfgGfuCfaAfuUfuCfuGfsu | 782 | UAACUUAGGGUCAAUUUCUGU | 1132 |
| AM15826-AS | cPrpasAfsasCfgUfaUfuAfaCfgUfaAfgCfaUfsc | 783 | AAACGUAUUAACGUAAGCAUC | 1119 |
| AM15828-AS | cPrpasUfsasCfaUfuUfgGfgUfcAfuAfgCfuUfsg | 784 | AUACAUUUGGGUCAUAGCUUG | 1064 |
| AM15830-AS | cPrpusCfsasUfuAfaGfaUfcUfgAfaUfcGfaCfsa | 785 | UCAUUAAGAUCUGAAUCGACA | 1133 |
| AM15832-AS | cPrpasAfsgsAfaUfaCfcAfcGfaAfaGfcAfaGfsc | 786 | AAGAAUACCACGAAAGCAAGC | 1084 |
| AM16516-AS | cPrpuUfaGfgauuuucCfaCfuAfcuuscsc | 787 | UUAGGAUUUUCCACUACUUCC | 1062 |
| AM16517-AS | cPrpuUfaGfgauuuucCfaCfuAfcuuscsg | 788 | UUAGGAUUUUCCACUACUUCG | 1126 |
| AM16519-AS | cPrpusUfsuGfuaaucagUfuCfcUfuguscsu | 789 | UUUGUAAUCAGUUCCUUGUCU | 1134 |
| AM16521-AS | cPrpusAfsaGfuuuacauCfcUfgAfuuasusg | 790 | UAAGUUUACAUCCUGAUUAUG | 1069 |
| AM16522-AS | cPrpuAfaGfuuuacauCfcUfgAfuuausg | 791 | UAAGUUUACAUCCUGAUUAUG | 1069 |
| AM16523-AS | cPrpuAfaGfuuuacauCfcUfgAfuuascsg | 792 | UAAGUUUACAUCCUGAUUACG | 1129 |
| AM16966-AS | cPrpusUfsaGfgauuuucCfaCfuAfcuuscsg | 793 | UUAGGAUUUUCCACUACUUCG | 1126 |

TABLE 4
CoV RNAi Agent Sense Strand Sequences
(Shown Without Linkers, Conjugates, or Capping Moieties.)

| | | | Underlying Base | |
|---|---|---|---|---|
| | | | Sequence (5′ → 3′) | |
| | Modified Sense Strand | SEQ ID | (Shown as an Unmodified | SEQ ID |
| Strand ID | (5′ → 3′) | NO. | Nucleotide Sequence) | NO. |
| AM11996-SS-NL | gacacacgUfCfCfaacucaguuu | 794 | GACACACGUCCAACUCAGUUU | 1135 |
| AM11998-SS-NL | ggaagaagUfAfGfuggaaaauca | 795 | GGAAGAAGUAGUGGAAAAUCA | 1136 |
| AM12000-SS-NL | ggaaguagUfGfGfaaaauccuaa | 796 | GGAAGUAGUGGAAAAUCCUAA | 1137 |
| AM12002-SS-NL | gucaugugGfUfAfguguuiguuu | 797 | GUCAUGUGGUAGUGUUIGUUU | 1138 |
| AM12004-SS-NL | caagcuauGfAfCfccaaauguau | 798 | CAAGCUAUGACCCAAAUGUAU | 1139 |
| AM12006-SS-NL | gacaugguAfCfCfacauauauca | 799 | GACAUGGUACCACAUAUAUCA | 1140 |
| AM12008-SS-NL | gcaugguaCfCfAfcauauaucaa | 800 | GCAUGGUACCACAUAUAUCAA | 1141 |
| AM12010-SS-NL | gguacauaAfUfCfaggauguaaa | 801 | GGUACAUAAUCAGGAUGUAAA | 1142 |
| AM12012-SS-NL | gcauaaucAfGfGfauguaaacuu | 802 | GCAUAAUCAGGAUGUAAACUU | 1143 |
| AM12014-SS-NL | ca_2NuaaucaGfGfAfuguaaacuua | 803 | CAUAAUCAGGAUGUAAACUUA | 1144 |
| AM12016-SS-NL | ggauguaaAfCfUfuacauagcua | 804 | GGAUGUAAACUUACAUAGCUA | 1145 |
| AM12018-SS-NL | gguaaggcUfAfGfacuuuauua_2Nu | 805 | GGUAAGGCUAGACUUUAUUAU | 1146 |
| AM12020-SS-NL | uggugucuCfUfAfucuguaguaa | 806 | UGGUGUCUCUAUCUGUAGUAA | 1147 |
| AM12022-SS-NL | gucucuauCfUfGfuaguacuaua | 807 | GUCUCUAUCUGUAGUACUAUA | 1148 |
| AM12024-SS-NL | gcucuaucUfGfUfaguacuauga | 808 | GCUCUAUCUGUAGUACUAUGA | 1149 |
| AM12026-SS-NL | gcuaucugUfAfGfuacuaugaca | 809 | GCUAUCUGUAGUACUAUGACA | 1150 |
| AM12028-SS-NL | gucuagcaAfUfGfuugcaaa_2Nuua | 810 | GUCUAGCAAUGUUGCAAAUUA | 1151 |
| AM12030-SS-NL | ga_2NagacucAfUfCfucuaugauga | 811 | GAAGACUCAUCUCUAUGAUGA | 1152 |
| AM12032-SS-NL | guggagucAfCfAfuuaauuggaa | 812 | GUGGAGUCACAUUAAUUGGAA | 1153 |
| AM12034-SS-NL | ggaugaguAfCfGfaacuuaugua | 813 | GGAUGAGUACGAACUUAUGUA | 1154 |
| AM12036-SS-NL | acgaacuuAfUfGfuacucauuca | 814 | ACGAACUUAUGUACUCAUUCA | 1155 |
| AM12038-SS-NL | cgaacuuaUfGfUfacucauucgu | 815 | CGAACUUAUGUACUCAUUCGU | 1156 |
| AM12040-SS-NL | cuuauguaCfUfCfauucguuuca | 816 | CUUAUGUACUCAUUCGUUUCA | 1157 |
| AM12042-SS-NL | cuuauguaCfUfCfauuciuuuca | 817 | CUUAUGUACUCAUUCIUUUCA | 1158 |
| AM12043-SS-NL | guaguuaaUfAfGfcguacuucuu | 818 | GUAGUUAAUAGCGUACUUCUU | 1159 |
| AM12045-SS-NL | gcuugcuuUfCfGfugguauucuu | 819 | GCUUGCUUUCGUGGUAUUCUU | 1160 |
| AM12047-SS-NL | guacacuaGfCfCfauccuuacua | 820 | GUACACUAGCCAUCCUUACUA | 1161 |
| AM12049-SS-NL | guacugcuGfCfAfauauuguuaa | 821 | GUACUGCUGCAAUAUUGUUAA | 1162 |
| AM12051-SS-NL | cugcugcaAfUfAfuuguuaacgu | 822 | CUGCUGCAAUAUUGUUAACGU | 1163 |
| AM12053-SS-NL | ugcugcaaUfAfUfuguuaacgua | 823 | UGCUGCAAUAUUGUUAACGUA | 1164 |
| AM12055-SS-NL | ugcugcaaUfa_2NUfuguuaacgua | 824 | UGCUGCAAUAUUGUUAACGUA | 1164 |
| AM12056-SS-NL | ccugaucuUfCfUfggucuaaaca | 825 | CCUGAUCUUCUGGUCUAAACA | 1165 |
| AM12058-SS-NL | caucacgaAfCfGfcuuucuuauu | 826 | CAUCACGAACGCUUUCUUAUU | 1166 |
| AM12060-SS-NL | caucacgaAfCfGfcuuucuua_2Nuu | 827 | CAUCACGAACGCUUUCUUAUU | 1166 |
| AM12061-SS-NL | cgaacgcuUfUfCfuuauuacaaa | 828 | CGAACGCUUUCUUAUUACAAA | 1167 |
| AM12063-SS-NL | gacaguaaGfUfGfacaacagaua | 829 | GACAGUAAGUGACAACAGAUA | 1168 |
| AM12065-SS-NL | caguaaguGfAfCfaacagauguu | 830 | CAGUAAGUGACAACAGAUGUU | 1169 |
| AM12067-SS-NL | ca_2NaauuggAfUfGfacaaagauca | 831 | CAAAUUGGAUGACAAAGAUCA | 1170 |
| AM12069-SS-NL | cucacauaGfCfAfaucuuuaa_2Nua | 832 | CUCACAUAGCAAUCUUUAAUA | 1171 |
| AM12468-SS-NL | ccuuuugaUfGfUfuguuagacaa | 833 | CCUUUUGAUGUUGUUAGACAA | 1172 |
| AM12470-SS-NL | ugguaaugCfUfUfuagaucaaga | 834 | UGGUAAUGCUUUAGAUCAAGA | 1173 |
| AM12472-SS-NL | gcauacaaUfGfCfuaguuaaaca | 835 | GCAUACAAUGCUAGUUAAACA | 1174 |
| AM12474-SS-NL | ca_2NuacaauGfCfUfaguuaaacaa | 836 | CAUACAAUGCUAGUUAAACAA | 1175 |
| AM12476-SS-NL | gugcauaaUfGfUfcugauagaga | 837 | GUGCAUAAUGUCUGAUAGAGA | 1176 |
| AM12478-SS-NL | ggucgauuCfAfGfaucuuaauga | 838 | GGUCGAUUCAGAUCUUAAUGA | 1177 |
| AM12480-SS-NL | gguccagaUfGfAfccaaauugga | 839 | GGUCCAGAUGACCAAAUUGGA | 1178 |
| AM12482-SS-NL | gccgcaaaUfUfGfcacaauuuga | 840 | GCCGCAAAUUGCACAAUUUGA | 1179 |
| AM12484-SS-NL | gugacgcaUfAfCfaaaacauuca | 841 | GUGACGCAUACAAAACAUUCA | 1180 |
| AM12562-SS-NL | ca_2NaggaagAfCfCfuuaaauucca | 842 | CAAGGAAGACCUUAAAUUCCA | 1181 |
| AM12564-SS-NL | guccaauuAfAfCfaccaauagca | 843 | GUCCAAUUAACACCAAUAGCA | 1182 |
| AM12566-SS-NL | guccaagaUfGfGfuauuucuacu | 844 | GUCCAAGAUGGUAUUUCUACU | 1183 |
| AM12568-SS-NL | ca_2NagauggUfAfUfuucuacuaca | 845 | CAAGAUGGUAUUUCUACUACA | 1184 |
| AM12570-SS-NL | gugaauacAfCfCfaaaagaucaa | 846 | GUGAAUACACCAAAAGAUCAA | 1185 |
| AM12572-SS-NL | cugucacuAfAfGfaaaucuicua | 847 | CUGUCACUAAGAAAUCUICUA | 1186 |
| AM12574-SS-NL | ccacuaaaGfCfAfuacaauguaa | 848 | CCACUAAAGCAUACAAUGUAA | 1187 |
| AM12576-SS-NL | gcagacaaGfGfAfacugauuaca | 849 | GCAGACAAGGAACUGAUUACA | 1188 |
| AM12578-SS-NL | ggacaaggAfAfCfugauuacaaa | 850 | GGACAAGGAACUGAUUACAAA | 1189 |
| AM12580-SS-NL | ggaacugaUfUfAfcaaacauuga | 851 | GGAACUGAUUACAAACAUUGA | 1190 |
| AM12582-SS-NL | gcugaauaAfGfCfauauugacia | 852 | GCUGAAUAAGCAUAUUGACIA | 1191 |
| AM13119-SS-NL | gscuucuuaAfGfAfgugcuuauga | 853 | GCUUCUUAAGAGUGCUUAUGA | 1192 |
| AM13121-SS-NL | csuuagacaAfUfGfucuuaucuaa | 854 | CUUAGACAAUGUCUUAUCUAA | 1193 |
| AM13123-SS-NL | gscauuaauGfGfCfaaucuucaua | 855 | GCAUUAAUGGCAAUCUUCAUA | 1194 |
| AM13125-SS-NL | gsaugcuuaCfGfUfuaauacguuu | 856 | GAUGCUUACGUUAAUACGUUU | 1195 |
| AM13127-SS-NL | gscaccuuuGfAfCfaaucuuaaga | 857 | GCACCUUUGACAAUCUUAAGA | 1196 |
| AM13129-SS-NL | gsgaaguagUfGfGfaaaauccuaa | 858 | GGAAGUAGUGGAAAAUCCUAA | 1137 |
| AM13130-SS-NL | cscuuuugaUfGfUfuguuagacaa | 859 | CCUUUUGAUGUUGUUAGACAA | 1172 |
| AM13131-SS-NL | usgguaaugCfUfUfuagaucaaga | 860 | UGGUAAUGCUUUAGAUCAAGA | 1173 |
| AM13132-SS-NL | csaagcuauGfAfCfccaaauguau | 861 | CAAGCUAUGACCCAAAUGUAU | 1139 |
| AM13133-SS-NL | gsuccaagaUfGfGfuauuucuacu | 862 | GUCCAAGAUGGUAUUUCUACU | 1183 |
| AM13134-SS-NL | csaagauggUfAfUfuucuacuaca | 863 | CAAGAUGGUAUUUCUACUACA | 1184 |
| AM13135-SS-NL | cscacuaaaGfCfAfuacaauguaa | 864 | CCACUAAAGCAUACAAUGUAA | 1187 |
| AM13136-SS-NL | gsgacaaggAfAfCfugauuacaaa | 865 | GGACAAGGAACUGAUUACAAA | 1189 |
| AM13158-SS-NL | gscaugguaCfCfAfcauauaucaa | 866 | GCAUGGUACCACAUAUAUCAA | 1141 |
| AM13159-SS-NL | gsuacugacAfUfUfagauaaucaa | 867 | GUACUGACAUUAGAUAAUCAA | 1197 |
| AM13161-SS-NL | gsgauguaaAfCfUfuacauagcua | 868 | GGAUGUAAACUUACAUAGCUA | 1145 |
| AM13162-SS-NL | csauacaauGfCfUfaguuaaacaa | 869 | CAUACAAUGCUAGUUAAACAA | 1175 |
| AM13542-SS-NL | usgcuguggUfuAfuAfccuacuaa | 870 | UGCUGUGGUUAUACCUACUAA | 1198 |
| AM13544-SS-NL | gscaaucUfuAfaGfacacuucuuu | 871 | GCAAUCUUAAGACACUUCUUU | 1199 |
| AM13546-SS-NL | gscugaaUfaAfgCfauauugacia | 872 | GCUGAAUAAGCAUAUUGACIA | 1191 |
| AM14660-SS-NL | gcuucuuaAfGfAfgugcuuauga | 873 | GCUUCUUAAGAGUGCUUAUGA | 1192 |
| AM14661-SS-NL | cuucuuaaGfAfGfugcuuaugaa | 874 | CUUCUUAAGAGUGCUUAUGAA | 1200 |
| AM14663-SS-NL | cuuagacaAfUfGfucuuaucuaa | 875 | CUUAGACAAUGUCUUAUCUAA | 1193 |
| AM14664-SS-NL | gcaccuuuGfAfCfaaucuuaaga | 876 | GCACCUUUGACAAUCUUAAGA | 1196 |
| AM14665-SS-NL | ggaaguagUfGfGfaaaauccuaa | 877 | GGAAGUAGUGGAAAAUCCUAA | 1137 |
| AM14691-SS-NL | gsgsaaguagUfGfGfaaaauccuasa | 878 | GGAAGUAGUGGAAAAUCCUAA | 1137 |
| AM14860-SS-NL | gsuccaagaUfGfGfuauuucuaca | 879 | GUCCAAGAUGGUAUUUCUACA | 1201 |
| AM15013-SS-NL | cauacaauGfCfUfaguuaaacaa | 880 | CAUACAAUGCUAGUUAAACAA | 1175 |
| AM15014-SS-NL | caagauggUfAfUfuucuacuaca | 881 | CAAGAUGGUAUUUCUACUACA | 1184 |
| AM15015-SS-NL | ggacaaggAfAfCfugauuacaaa | 882 | GGACAAGGAACUGAUUACAAA | 1189 |
| AM15016-SS-NL | ccacuaaaGfCfAfuacaauguaa | 883 | CCACUAAAGCAUACAAUGUAA | 1187 |
| AM15017-SS-NL | guccaagaUfGfGfuauuucuacu | 884 | GUCCAAGAUGGUAUUUCUACU | 1183 |
| AM15564-SS-NL | ggaaguagUfgGfaAfaauccuaa | 885 | GGAAGUAGUGGAAAAUCCUAA | 1137 |
| AM15567-SS-NL | cgaaguagUfgGfaAfaauccuaa | 886 | CGAAGUAGUGGAAAAUCCUAA | 1202 |
| AM15571-SS-NL | cauacaauGfcUfaGfuuaaacaa | 887 | CAUACAAUGCUAGUUAAACAA | 1175 |
| AM15573-SS-NL | cguacaauGfcUfaGfuuaaacaa | 888 | CGUACAAUGCUAGUUAAACAA | 1203 |
| AM15577-SS-NL | guccaagaUfgGfuAfuuucuacu | 889 | GUCCAAGAUGGUAUUUCUACU | 1183 |
| AM15581-SS-NL | guccaagaUfgGfuAfuuucuaca | 890 | GUCCAAGAUGGUAUUUCUACA | 1201 |
| AM15796-SS-NL | ggacaaggAfaCfuGfauuacaaa | 891 | GGACAAGGAACUGAUUACAAA | 1189 |
| AM15800-SS-NL | cgacaaggAfaCfuGfauuacaaa | 892 | CGACAAGGAACUGAUUACAAA | 1204 |
| AM15802-SS-NL | cauaaucaGfGfAfuguaaacuua | 893 | CAUAAUCAGGAUGUAAACUUA | 1144 |
| AM15805-SS-NL | cauaaucaGfgAfuGfuaaacuua | 894 | CAUAAUCAGGAUGUAAACUUA | 1144 |
| AM15806-SS-NL | cguaaucaGfgAfuGfuaaacuua | 895 | CGUAAUCAGGAUGUAAACUUA | 1205 |
| AM15809-SS-NL | ggauguaaAfCfUfuacauagcua | 896 | GGAUGUAAACUUACAUAGCUA | 1145 |
| AM15811-SS-NL | ggauguaaAfcUfuAfcauagcua | 897 | GGAUGUAAACUUACAUAGCUA | 1145 |
| AM15814-SS-NL | gcaccuuuGfaCfaAfucuuaaga | 898 | GCACCUUUGACAAUCUUAAGA | 1196 |
| AM15817-SS-NL | ugcuguggUfUfAfuaccuacuaa | 899 | UGCUGUGGUUAUACCUACUAA | 1198 |
| AM15819-SS-NL | gacaaucuUfAfAfgacacuucuu | 900 | GACAAUCUUAAGACACUUCUU | 1206 |
| AM15821-SS-NL | caugggcaCfAfCfuuucuuauga | 901 | CAUGGGCACACUUUCUUAUGA | 1207 |
| AM15823-SS-NL | acagaaauUfGfAfcccuaaguua | 902 | ACAGAAAUUGACCCUAAGUUA | 1208 |
| AM15825-SS-NL | gaugcuuaCfGfUfuaauacguuu | 903 | GAUGCUUACGUUAAUACGUUU | 1195 |
| AM15827-SS-NL | caagcuauGfAfCfccaaauguau | 904 | CAAGCUAUGACCCAAAUGUAU | 1139 |
| AM15829-SS-NL | ugucgauuCfAfGfaucuuaauga | 905 | UGUCGAUUCAGAUCUUAAUGA | 1209 |
| AM15831-SS-NL | gcuugcuuUfCfGfugguauucuu | 906 | GCUUGCUUUCGUGGUAUUCUU | 1160 |
| AM16518-SS-NL | agacaaggAfaCfuGfauuacaaa | 907 | AGACAAGGAACUGAUUACAAA | 1210 |
| AM16520-SS-NL | cguaaucaGfGfAfuguaaacuua | 908 | CGUAAUCAGGAUGUAAACUUA | 1205 |
| AM16965-SS-NL | csgaaguagUfgGfaAfaauccuaa | 909 | CGAAGUAGUGGAAAAUCCUAA | 1202 |
| AM16967-SS-NL | gsgacaaggAfaCfuGfauuacaaa | 910 | GGACAAGGAACUGAUUACAAA | 1189 |
| AM16968-SS-NL | usgcuguggUfUfAfuaccuacuaa | 911 | UGCUGUGGUUAUACCUACUAA | 1198 |
| AM16969-SS-NL | csauacaauGfcUfaGfuuaaacaa | 912 | CAUACAAUGCUAGUUAAACAA | 1175 |
| AM16970-SS-NL | csguaaucaGfgAfuGfuaaacuua | 913 | CGUAAUCAGGAUGUAAACUUA | 1205 |

a_2N = 2-aminoadenosine nucleotide;
I = hypoxanthine (inosine) nucleotide

TABLE 5
CoV RNAi Agent Sense Strand Sequences (Shown With (TriAlk14) Linker or
(NAG37) Targeting Ligand (see Table 11 for structure information.))

| | | | Underlying Base | |
|---|---|---|---|---|
| | | | Sequence (5′ → 3′) | |
| | | SEQ ID | (Shown as an Unmodified | SEQ ID |
| Strand ID | Modified Sense Strand (5′ → 3′) | NO. | Nucleotide Sequence) | NO. |
| AM11996-SS | (invAb)sgacacacgUfCfCfaacucaguuus(invAb) | 914 | GACACACGUCCAACUCAGUUU | 1135 |
| AM11998-SS | (invAb)sggaagaagUfAfGfuggaaaaucas(invAb) | 915 | GGAAGAAGUAGUGGAAAAUCA | 1136 |
| AM12000-SS | (invAb)sggaaguagUfGfGfaaaauccuaas(invAb) | 916 | GGAAGUAGUGGAAAAUCCUAA | 1137 |
| AM12002-SS | (invAb)sgucaugugGfUfAfguguuiguuus(invAb) | 917 | GUCAUGUGGUAGUGUUIGUUU | 1138 |
| AM12004-SS | (invAb)scaagcuauGfAfCfccaaauguaus(invAb) | 918 | CAAGCUAUGACCCAAAUGUAU | 1139 |
| AM12006-SS | (invAb)sgacaugguAfCfCfacauauaucas(invAb) | 919 | GACAUGGUACCACAUAUAUCA | 1140 |
| AM12008-SS | (invAb)sgcaugguaCfCfAfcauauaucaas(invAb) | 920 | GCAUGGUACCACAUAUAUCAA | 1141 |
| AM12010-SS | (invAb)sgguacauaAfUfCfaggauguaaas(invAb) | 921 | GGUACAUAAUCAGGAUGUAAA | 1142 |
| AM12012-SS | (invAb)sgcauaaucAfGfGfauguaaacuus(invAb) | 922 | GCAUAAUCAGGAUGUAAACUU | 1143 |
| AM12014-SS | (invAb)sca_2NuaaucaGfGfAfuguaaacuuas(invAb) | 923 | CAUAAUCAGGAUGUAAACUUA | 1144 |
| AM12016-SS | (invAb)sggauguaaAfCfUfuacauagcuas(invAb) | 924 | GGAUGUAAACUUACAUAGCUA | 1145 |
| AM12018-SS | (invAb)sgguaaggcUfAfGfacuuuauua_2Nus(invAb) | 925 | GGUAAGGCUAGACUUUAUUAU | 1146 |
| AM12020-SS | (invAb)suggugucuCfUfAfucuguaguaas(invAb) | 926 | UGGUGUCUCUAUCUGUAGUAA | 1147 |
| AM12022-SS | (invAb)sgucucuauCfUfGfuaguacuauas(invAb) | 927 | GUCUCUAUCUGUAGUACUAUA | 1148 |
| AM12024-SS | (invAb)sgcucuaucUfGfUfaguacuaugas(invAb) | 928 | GCUCUAUCUGUAGUACUAUGA | 1149 |
| AM12026-SS | (invAb)sgcuaucugUfAfGfuacuaugacas(invAb) | 929 | GCUAUCUGUAGUACUAUGACA | 1150 |
| AM12028-SS | (invAb)sgucuagcaAfUfGfuugcaaa_2Nuuas(invAb) | 930 | GUCUAGCAAUGUUGCAAAUUA | 1151 |
| AM12030-SS | (invAb)sga_2NagacucAfUfCfucuaugaugas(invAb) | 931 | GAAGACUCAUCUCUAUGAUGA | 1152 |
| AM12032-SS | (invAb)sguggagucAfCfAfuuaauuggaas(invAb) | 932 | GUGGAGUCACAUUAAUUGGAA | 1153 |
| AM12034-SS | (invAb)sggaugaguAfCfGfaacuuauguas(invAb) | 933 | GGAUGAGUACGAACUUAUGUA | 1154 |
| AM12036-SS | (invAb)sacgaacuuAfUfGfuacucauucas(invAb) | 934 | ACGAACUUAUGUACUCAUUCA | 1155 |
| AM12038-SS | (invAb)scgaacuuaUfGfUfacucauucgus(invAb) | 935 | CGAACUUAUGUACUCAUUCGU | 1156 |
| AM12040-SS | (invAb)scuuauguaCfUfCfauucguuucas(invAb) | 936 | CUUAUGUACUCAUUCGUUUCA | 1157 |
| AM12042-SS | (invAb)scuuauguaCfUfCfauuciuuucas(invAb) | 937 | CUUAUGUACUCAUUCIUUUCA | 1158 |
| AM12043-SS | (invAb)sguaguuaaUfAfGfcguacuucuus(invAb) | 938 | GUAGUUAAUAGCGUACUUCUU | 1159 |
| AM12045-SS | (invAb)sgcuugcuuUfCfGfugguauucuus(invAb) | 939 | GCUUGCUUUCGUGGUAUUCUU | 1160 |
| AM12047-SS | (invAb)sguacacuaGfCfCfauccuuacuas(invAb) | 940 | GUACACUAGCCAUCCUUACUA | 1161 |
| AM12049-SS | (invAb)sguacugcuGfCfAfauauuguuaas(invAb) | 941 | GUACUGCUGCAAUAUUGUUAA | 1162 |
| AM12051-SS | (invAb)scugcugcaAfUfAfuuguuaacgus(invAb) | 942 | CUGCUGCAAUAUUGUUAACGU | 1163 |
| AM12053-SS | (invAb)sugcugcaaUfAfUfuguuaacguas(invAb) | 943 | UGCUGCAAUAUUGUUAACGUA | 1164 |
| AM12055-SS | (invAb)sugcugcaaUfa_2NUfuguuaacguas(invAb) | 944 | UGCUGCAAUAUUGUUAACGUA | 1164 |
| AM12056-SS | (invAb)sccugaucuUfCfUfggucuaaacas(invAb) | 945 | CCUGAUCUUCUGGUCUAAACA | 1165 |
| AM12058-SS | (invAb)scaucacgaAfCfGfcuuucuuauus(invAb) | 946 | CAUCACGAACGCUUUCUUAUU | 1166 |
| AM12060-SS | (invAb)scaucacgaAfCfGfcuuucuua_2Nuus(invAb) | 947 | CAUCACGAACGCUUUCUUAUU | 1166 |
| AM12061-SS | (invAb)scgaacgcuUfUfCfuuauuacaaas(invAb) | 948 | CGAACGCUUUCUUAUUACAAA | 1167 |
| AM12063-SS | (invAb)sgacaguaaGfUfGfacaacagauas(invAb) | 949 | GACAGUAAGUGACAACAGAUA | 1168 |
| AM12065-SS | (invAb)scaguaaguGfAfCfaacagauguus(invAb) | 950 | CAGUAAGUGACAACAGAUGUU | 1169 |
| AM12067-SS | (invAb)sca_2NaauuggAfUfGfacaaagaucas(invAb) | 951 | CAAAUUGGAUGACAAAGAUCA | 1170 |
| AM12069-SS | (invAb)scucacauaGfCfAfaucuuuaa_2Nuas(invAb) | 952 | CUCACAUAGCAAUCUUUAAUA | 1171 |
| AM12468-SS | (invAb)sccuuuugaUfGfUfuguuagacaas(invAb) | 953 | CCUUUUGAUGUUGUUAGACAA | 1172 |
| AM12470-SS | (invAb)sugguaaugCfUfUfuagaucaagas(invAb) | 954 | UGGUAAUGCUUUAGAUCAAGA | 1173 |
| AM12472-SS | (invAb)sgcauacaaUfGfCfuaguuaaacas(invAb) | 955 | GCAUACAAUGCUAGUUAAACA | 1174 |
| AM12474-SS | (invAb)sca_2NuacaauGfCfUfaguuaaacaas(invAb) | 956 | CAUACAAUGCUAGUUAAACAA | 1175 |
| AM12476-SS | (invAb)sgugcauaaUfGfUfcugauagagas(invAb) | 957 | GUGCAUAAUGUCUGAUAGAGA | 1176 |
| AM12478-SS | (invAb)sggucgauuCfAfGfaucuuaaugas(invAb) | 958 | GGUCGAUUCAGAUCUUAAUGA | 1177 |
| AM12480-SS | (invAb)sgguccagaUfGfAfccaaauuggas(invAb) | 959 | GGUCCAGAUGACCAAAUUGGA | 1178 |
| AM12482-SS | (invAb)sgccgcaaaUfUfGfcacaauuugas(invAb) | 960 | GCCGCAAAUUGCACAAUUUGA | 1179 |
| AM12484-SS | (invAb)sgugacgcaUfAfCfaaaacauucas(invAb) | 961 | GUGACGCAUACAAAACAUUCA | 1180 |
| AM12562-SS | (invAb)sca_2NaggaagAfCfCfuuaaauuccas(invAb) | 962 | CAAGGAAGACCUUAAAUUCCA | 1181 |
| AM12564-SS | (invAb)sguccaauuAfAfCfaccaauagcas(invAb) | 963 | GUCCAAUUAACACCAAUAGCA | 1182 |
| AM12566-SS | (invAb)sguccaagaUfGfGfuauuucuacus(invAb) | 964 | GUCCAAGAUGGUAUUUCUACU | 1183 |
| AM12568-SS | (invAb)sca_2NagauggUfAfUfuucuacuacas(invAb) | 965 | CAAGAUGGUAUUUCUACUACA | 1184 |
| AM12570-SS | (invAb)sgugaauacAfCfCfaaaagaucaas(invAb) | 966 | GUGAAUACACCAAAAGAUCAA | 1185 |
| AM12572-SS | (invAb)scugucacuAfAfGfaaaucuicuas(invAb) | 967 | CUGUCACUAAGAAAUCUICUA | 1186 |
| AM12574-SS | (invAb)sccacuaaaGfCfAfuacaauguaas(invAb) | 968 | CCACUAAAGCAUACAAUGUAA | 1187 |
| AM12576-SS | (invAb)sgcagacaaGfGfAfacugauuacas(invAb) | 969 | GCAGACAAGGAACUGAUUACA | 1188 |
| AM12578-SS | (invAb)sggacaaggAfAfCfugauuacaaas(invAb) | 970 | GGACAAGGAACUGAUUACAAA | 1189 |
| AM12580-SS | (invAb)sggaacugaUfUfAfcaaacauugas(invAb) | 971 | GGAACUGAUUACAAACAUUGA | 1190 |
| AM12582-SS | (invAb)sgcugaauaAfGfCfauauugacias(invAb) | 972 | GCUGAAUAAGCAUAUUGACIA | 1191 |
| AM13119-SS | (TriAlk14)gscuucuuaAfGfAfgugcuuaugas(invAb) | 973 | GCUUCUUAAGAGUGCUUAUGA | 1192 |
| AM13121-SS | (TriAlk14)csuuagacaAfUfGfucuuaucuaas(invAb) | 974 | CUUAGACAAUGUCUUAUCUAA | 1193 |
| AM13123-SS | (TriAlk14)gscauuaauGfGfCfaaucuucauas(invAb) | 975 | GCAUUAAUGGCAAUCUUCAUA | 1194 |
| AM13125-SS | (TriAlk14)gsaugcuuaCfGfUfuaauacguuus(invAb) | 976 | GAUGCUUACGUUAAUACGUUU | 1195 |
| AM13127-SS | (TriAlk14)gscaccuuuGfAfCfaaucuuaagas(invAb) | 977 | GCACCUUUGACAAUCUUAAGA | 1196 |
| AM13129-SS | (TriAlk14)gsgaaguagUfGfGfaaaauccuaas(invAb) | 978 | GGAAGUAGUGGAAAAUCCUAA | 1137 |
| AM13130-SS | (TriAlk14)cscuuuugaUfGfUfuguuagacaas(invAb) | 979 | CCUUUUGAUGUUGUUAGACAA | 1172 |
| AM13131-SS | (TriAlk14)usgguaaugCfUfUfuagaucaagas(invAb) | 980 | UGGUAAUGCUUUAGAUCAAGA | 1173 |
| AM13132-SS | (TriAlk14)csaagcuauGfAfCfccaaauguaus(invAb) | 981 | CAAGCUAUGACCCAAAUGUAU | 1139 |
| AM13133-SS | (TriAlk14)gsuccaagaUfGfGfuauuucuacus(invAb) | 982 | GUCCAAGAUGGUAUUUCUACU | 1183 |
| AM13134-SS | (TriAlk14)csaagauggUfAfUfuucuacuacas(invAb) | 983 | CAAGAUGGUAUUUCUACUACA | 1184 |
| AM13135-SS | (TriAlk14)cscacuaaaGfCfAfuacaauguaas(invAb) | 984 | CCACUAAAGCAUACAAUGUAA | 1187 |
| AM13136-SS | (TriAlk14)gsgacaaggAfAfCfugauuacaaas(invAb) | 985 | GGACAAGGAACUGAUUACAAA | 1189 |
| AM13158-SS | (TriAlk14)gscaugguaCfCfAfcauauaucaas(invAb) | 986 | GCAUGGUACCACAUAUAUCAA | 1141 |
| AM13159-SS | (TriAlk14)gsuacugacAfUfUfagauaaucaas(invAb) | 987 | GUACUGACAUUAGAUAAUCAA | 1197 |
| AM13161-SS | (TriAlk14)gsgauguaaAfCfUfuacauagcuas(invAb) | 988 | GGAUGUAAACUUACAUAGCUA | 1145 |
| AM13162-SS | (TriAlk14)csauacaauGfCfUfaguuaaacaas(invAb) | 989 | CAUACAAUGCUAGUUAAACAA | 1175 |
| AM13542-SS | (TriAlk14)usgcuguggUfuAfuAfccuacuaas(invAb) | 990 | UGCUGUGGUUAUACCUACUAA | 1198 |
| AM13544-SS | (TriAlk14)gscaaucUfuAfaGfacacuucuuus(invAb) | 991 | GCAAUCUUAAGACACUUCUUU | 1199 |
| AM13546-SS | (TriAlk14)gscugaaUfaAfgCfauauugacias(invAb) | 992 | GCUGAAUAAGCAUAUUGACIA | 1191 |
| AM14660-SS | (NAG37)s(invAb)sgcuucuuaAfGfAfgugcuuaugas(invAb) | 993 | GCUUCUUAAGAGUGCUUAUGA | 1192 |
| AM14661-SS | (NAG37)s(invAb)scuucuuaaGfAfGfugcuuaugaas(invAb) | 994 | CUUCUUAAGAGUGCUUAUGAA | 1200 |
| AM14663-SS | (NAG37)s(invAb)scuuagacaAfUfGfucuuaucuaas(invAb) | 995 | CUUAGACAAUGUCUUAUCUAA | 1193 |
| AM14664-SS | (NAG37)s(invAb)sgcaccuuuGfAfCfaaucuuaagas(invAb) | 996 | GCACCUUUGACAAUCUUAAGA | 1196 |
| AM14665-SS | (NAG37)s(invAb)sggaaguagUfGfGfaaaauccuaas(invAb) | 997 | GGAAGUAGUGGAAAAUCCUAA | 1137 |
| AM14691-SS | (invAb)sgsgsaaguagUfGfGfaaaauccuasa | 998 | GGAAGUAGUGGAAAAUCCUAA | 1137 |
| AM14860-SS | (TriAlk14)gsuccaagaUfGfGfuauuucuacas(invAb) | 999 | GUCCAAGAUGGUAUUUCUACA | 1201 |
| AM15013-SS | (NAG37)s(invAb)scauacaauGfCfUfaguuaaacaas(invAb) | 1000 | CAUACAAUGCUAGUUAAACAA | 1175 |
| AM15014-SS | (NAG37)s(invAb)scaagauggUfAfUfuucuacuacas(invAb) | 1001 | CAAGAUGGUAUUUCUACUACA | 1184 |
| AM15015-SS | (NAG37)s(invAb)sggacaaggAfAfCfugauuacaaas(invAb) | 1002 | GGACAAGGAACUGAUUACAAA | 1189 |
| AM15016-SS | (NAG37)s(invAb)sccacuaaaGfCfAfuacaauguaas(invAb) | 1003 | CCACUAAAGCAUACAAUGUAA | 1187 |
| AM15017-SS | (NAG37)s(invAb)sguccaagaUfGfGfuauuucuacus(invAb) | 1004 | GUCCAAGAUGGUAUUUCUACU | 1183 |
| AM15564-SS | (NAG37)s(invAb)sggaaguagUfgGfaAfaauccuaas(invAb) | 1005 | GGAAGUAGUGGAAAAUCCUAA | 1137 |
| AM15567-SS | (NAG37)s(invAb)scgaaguagUfgGfaAfaauccuaas(invAb) | 1006 | CGAAGUAGUGGAAAAUCCUAA | 1202 |
| AM15571-SS | (NAG37)s(invAb)scauacaauGfcUfaGfuuaaacaas(invAb) | 1007 | CAUACAAUGCUAGUUAAACAA | 1175 |
| AM15573-SS | (NAG37)s(invAb)scguacaauGfcUfaGfuuaaacaas(invAb) | 1008 | CGUACAAUGCUAGUUAAACAA | 1203 |
| AM15577-SS | (NAG37)s(invAb)sguccaagaUfgGfuAfuuucuacus(invAb) | 1009 | GUCCAAGAUGGUAUUUCUACU | 1183 |
| AM15581-SS | (NAG37)s(invAb)sguccaagaUfgGfuAfuuucuacas(invAb) | 1010 | GUCCAAGAUGGUAUUUCUACA | 1201 |
| AM15796-SS | (NAG37)s(invAb)sggacaaggAfaCfuGfauuacaaas(invAb) | 1011 | GGACAAGGAACUGAUUACAAA | 1189 |
| AM15800-SS | (NAG37)s(invAb)scgacaaggAfaCfuGfauuacaaas(invAb) | 1012 | CGACAAGGAACUGAUUACAAA | 1204 |
| AM15802-SS | (NAG37)s(invAb)scauaaucaGfGfAfuguaaacuuas(invAb) | 1013 | CAUAAUCAGGAUGUAAACUUA | 1144 |
| AM15805-SS | (NAG37)s(invAb)scauaaucaGfgAfuGfuaaacuuas(invAb) | 1014 | CAUAAUCAGGAUGUAAACUUA | 1144 |
| AM15806-SS | (NAG37)s(invAb)scguaaucaGfgAfuGfuaaacuuas(invAb) | 1015 | CGUAAUCAGGAUGUAAACUUA | 1205 |
| AM15809-SS | (NAG37)s(invAb)sggauguaaAfCfUfuacauagcuas(invAb) | 1016 | GGAUGUAAACUUACAUAGCUA | 1145 |
| AM15811-SS | (NAG37)s(invAb)sggauguaaAfcUfuAfcauagcuas(invAb) | 1017 | GGAUGUAAACUUACAUAGCUA | 1145 |
| AM15814-SS | (NAG37)s(invAb)sgcaccuuuGfaCfaAfucuuaagas(invAb) | 1018 | GCACCUUUGACAAUCUUAAGA | 1196 |
| AM15817-SS | (NAG37)s(invAb)sugcuguggUfUfAfuaccuacuaas(invAb) | 1019 | UGCUGUGGUUAUACCUACUAA | 1198 |
| AM15819-SS | (NAG37)s(invAb)sgacaaucuUfAfAfgacacuucuus(invAb) | 1020 | GACAAUCUUAAGACACUUCUU | 1206 |
| AM15821-SS | (NAG37)s(invAb)scaugggcaCfAfCfuuucuuaugas(invAb) | 1021 | CAUGGGCACACUUUCUUAUGA | 1207 |
| AM15823-SS | (NAG37)s(invAb)sacagaaauUfGfAfcccuaaguuas(invAb) | 1022 | ACAGAAAUUGACCCUAAGUUA | 1208 |
| AM15825-SS | (NAG37)s(invAb)sgaugcuuaCfGfUfuaauacguuus(invAb) | 1023 | GAUGCUUACGUUAAUACGUUU | 1195 |
| AM15827-SS | (NAG37)s(invAb)scaagcuauGfAfCfccaaauguaus(invAb) | 1024 | CAAGCUAUGACCCAAAUGUAU | 1139 |
| AM15829-SS | (NAG37)s(invAb)sugucgauuCfAfGfaucuuaaugas(invAb) | 1025 | UGUCGAUUCAGAUCUUAAUGA | 1209 |
| AM15831-SS | (NAG37)s(invAb)sgcuugcuuUfCfGfugguauucuus(invAb) | 1026 | GCUUGCUUUCGUGGUAUUCUU | 1160 |
| AM16518-SS | (NAG37)s(invAb)sagacaaggAfaCfuGfauuacaaas(invAb) | 1027 | AGACAAGGAACUGAUUACAAA | 1210 |
| AM16520-SS | (NAG37)s(invAb)scguaaucaGfGfAfuguaaacuuas(invAb) | 1028 | CGUAAUCAGGAUGUAAACUUA | 1205 |
| AM16965-SS | (TriAlk14)csgaaguagUfgGfaAfaauccuaas(invAb) | 1029 | CGAAGUAGUGGAAAAUCCUAA | 1202 |
| AM16967-SS | (TriAlk14)gsgacaaggAfaCfuGfauuacaaas(invAb) | 1030 | GGACAAGGAACUGAUUACAAA | 1189 |
| AM16968-SS | (TriAlk14)usgcuguggUfUfAfuaccuacuaas(invAb) | 1031 | UGCUGUGGUUAUACCUACUAA | 1198 |
| AM16969-SS | (TriAlk14)csauacaauGfcUfaGfuuaaacaas(invAb) | 1032 | CAUACAAUGCUAGUUAAACAA | 1175 |
| AM16970-SS | (TriAlk14)csguaaucaGfgAfuGfuaaacuuas(invAb) | 1033 | CGUAAUCAGGAUGUAAACUUA | 1205 |

a_2N = 2-aminoadenosine nucleotide;
I = hypoxanthine (inosine) nucleotide

TABLE 6
CoV RNAi Agent Sense Strand Sequences (Shown with Targeting Ligand
Conjugate. The structure of αvβ6-SM6.1 is shown in
Table 11, and the structure of Tri-SM6.1-αvβ6-TA14 is shown in FIG. 1.)

| | | | Corresponding |
|---|---|---|---|
| | | | Sense Strand |
| | | | AM Number |
| | | | Without Linker |
| | | SEQ ID | or Conjugate |
| Strand ID | Modified Sense Strand (5′ → 3′) | NO. | (See Table 4) |
| CS001679 | Tri-SM6.1-avb6-TA14-gscuucuuaAfGfAfgugcuuaugas(invAb) | 1034 | AM13119-SS-NL |
| CS001681 | Tri-SM6.1-avb6-TA14-csuuagacaAfUfGfucuuaucuaas(invAb) | 1035 | AM13121-SS-NL |
| CS001683 | Tri-SM6.1-avb6-TA14-gscauuaauGfGfCfaaucuucauas(invAb) | 1036 | AM13123-SS-NL |
| CS001685 | Tri-SM6.1-avb6-TA14-gsaugcuuaCfGfUfuaauacguuus(invAb) | 1037 | AM13125-SS-NL |
| CS001687 | Tri-SM6.1-avb6-TA14-gscaccuuuGfAfCfaaucuuaagas(invAb) | 1038 | AM13127-SS-NL |
| CS001689 | Tri-SM6.1-avb6-TA14-gsgaaguagUfGfGfaaaauccuaas(invAb) | 1039 | AM13129-SS-NL |
| CS001691 | Tri-SM6.1-avb6-TA14-cscuuuugaUfGfUfuguuagacaas(invAb) | 1040 | AM13130-SS-NL |
| CS001693 | Tri-SM6.1-avb6-TA14-usgguaaugCfUfUfuagaucaagas(invAb) | 1041 | AM13131-SS-NL |
| CS001695 | Tri-SM6.1-avb6-TA14-csaagcuauGfAfCfccaaauguaus(invAb) | 1042 | AM13132-SS-NL |
| CS001697 | Tri-SM6.1-avb6-TA14-gsuccaagaUfGfGfuauuucuacus(invAb) | 1043 | AM13133-SS-NL |
| CS001699 | Tri-SM6.1-avb6-TA14-csaagauggUfAfUfuucuacuacas(invAb) | 1044 | AM13134-SS-NL |
| CS001701 | Tri-SM6.1-avb6-TA14-cscacuaaaGfCfAfuacaauguaas(invAb) | 1045 | AM13135-SS-NL |
| CS001703 | Tri-SM6.1-avb6-TA14-gsgacaaggAfAfCfugauuacaaas(invAb) | 1046 | AM13136-SS-NL |
| CS001705 | Tri-SM6.1-avb6-TA14-gscaugguaCfCfAfcauauaucaas(invAb) | 1047 | AM13158-SS-NL |
| CS001707 | Tri-SM6.1-avb6-TA14-gsuacugacAfUfUfagauaaucaas(invAb) | 1048 | AM13159-SS-NL |
| CS001709 | Tri-SM6.1-avb6-TA14-gsgauguaaAfCfUfuacauagcuas(invAb) | 1049 | AM13161-SS-NL |
| CS001711 | Tri-SM6.1-avb6-TA14-csauacaauGfCfUfaguuaaacaas(invAb) | 1050 | AM13162-SS-NL |
| CS001891 | Tri-SM6.1-avb6-TA14-usgcuguggUfuAfuAfccuacuaas(invAb) | 1051 | AM13542-SS-NL |
| CS001893 | Tri-SM6.1-avb6-TA14-gscaaucUfuAfaGfacacuucuuus(invAb) | 1052 | AM13544-SS-NL |
| CS001895 | Tri-SM6.1-avb6-TA14-gscugaaUfaAfgCfauauugacias(invAb) | 1053 | AM13546-SS-NL |
| CS002495 | Tri-SM6.1-avb6-TA14-gsuccaagaUfGfGfuauuucuacas(invAb) | 1054 | AM14860-SS-NL |
| CS003334 | Tri-SM6.1-avb6-TA14-csgaaguagUfgGfaAfaauccuaas(invAb) | 1055 | AM16965-SS-NL |
| CS003337 | Tri-SM6.1-avb6-TA14-gsgacaaggAfaCfuGfauuacaaas(invAb) | 1056 | AM16967-SS-NL |
| CS003340 | Tri-SM6.1-avb6-TA14-usgcuguggUfUfAfuaccuacuaas(invAb) | 1057 | AM16968-SS-NL |
| CS003342 | Tri-SM6.1-avb6-TA14-csauacaauGfcUfaGfuuaaacaas(invAb) | 1058 | AM16969-SS-NL |
| CS003344 | Tri-SM6.1-avb6-TA14-csguaaucaGfgAfuGfuaaacuuas(invAb) | 1059 | AM16970-SS-NL |

The CoV RNAi agents disclosed herein are formed by annealing an antisense strand with a sense strand. A sense strand containing a sequence listed in Table 2, Table 4, Table 5, or Table 6 can be hybridized to any antisense strand containing a sequence listed in Table 2 or Table 3, provided the two sequences have a region of at least 85% complementarity over a contiguous 16, 17, 18, 19, 20, or 21 nucleotide sequence.

As shown in Table 5 above, certain of the example CoV RNAi agent nucleotide sequences are shown to further include reactive linking groups at one or both of the 5′ terminal end and the 3′ terminal end of the sense strand. For example, many of the CoV RNAi agent sense strand sequences shown in Table 5 above have a (TriAlk14) linking group at the 5′ end of the nucleotide sequence. Other linking groups, such as an (NH2-C6) linking group or a (6-SS-6) or (C6-SS-C6) linking group, may be present as well or alternatively in certain embodiments. Such reactive linking groups are positioned to facilitate the linking of targeting ligands, targeting groups, and/or PK/PD modulators to the CoV RNAi agents disclosed herein. Linking or conjugation reactions are well known in the art and provide for formation of covalent linkages between two molecules or reactants. Suitable conjugation reactions for use in the scope of the inventions herein include, but are not limited to, amide coupling reaction, Michael addition reaction, hydrazone formation reaction, inverse-demand Diels-Alder cycloaddition reaction, oxime ligation, and Copper (I)-catalyzed or strain-promoted azide-alkyne cycloaddition reaction cycloaddition reaction.

In some embodiments, targeting ligands, such as the integrin targeting ligands shown in the examples and figures disclosed herein, can be synthesized as activated esters, such as tetrafluorophenyl (TFP) esters, which can be displaced by a reactive amino group (e.g., NH₂—C₆) to attach the targeting ligand to the CoV RNAi agents disclosed herein. In some embodiments, targeting ligands are synthesized as azides, which can be conjugated to a propargyl (e.g., TriAlk14) or DBCO group, for example, via Copper (I)-catalyzed or strain-promoted azide-alkyne cycloaddition reaction.

Additionally, certain of the nucleotide sequences can be synthesized with a dT nucleotide at the 3′ terminal end of the sense strand, followed by (3′→5′) a linker (e.g., C6-SS-C6). The linker can, in some embodiments, facilitate the linkage to additional components, such as, for example, a PK/PD modulator or one or more targeting ligands. As described herein, the disulfide bond of C6-SS-C6 is first reduced, removing the dT from the molecule, which can then facilitate the conjugation of the desired PK/PD modulator. The terminal dT nucleotide therefore is not a part of the fully conjugated construct.

In some embodiments, the antisense strand of a CoV RNAi agent disclosed herein differs by 0, 1, 2, or 3 nucleotides from any of the antisense strand sequences in Table 3 or Table 10. In some embodiments, the sense strand of a CoV RNAi agent disclosed herein differs by 0, 1, 2, or 3 nucleotides from any of the sense strand sequences in Table 4, Table 5, Table 6, or Table 10.

In some embodiments, a CoV RNAi agent antisense strand comprises a nucleotide sequence of any of the sequences in Table 2 or Table 3. In some embodiments, a CoV RNAi agent antisense strand comprises the sequence of nucleotides (from 5′ end→3′ end) 1-17, 2-17, 1-18, 2-18, 1-19, 2-19, 1-20, 2-20, 1-21, 2-21, 1-22, 2-22, 1-23, 2-23, 1-24, or 2-24 of any of the sequences in Table 2, Table 3, or Table 10. In certain embodiments, a CoV RNAi agent antisense strand comprises or consists of a modified sequence of any one of the modified sequences in Table 3 or Table 10.

In some embodiments, a CoV RNAi agent sense strand comprises the nucleotide sequence of any of the sequences in Table 2 or Table 4. In some embodiments, a CoV RNAi agent sense strand comprises the sequence of nucleotides (from 5′ end→3′ end) 1-17, 2-17, 3-17, 4-17, 1-18, 2-18, 3-18, 4-18, 1-19, 2-19, 3-19, 4-19, 1-20, 2-20, 3-20, 4-20, 1-21, 2-21, 3-21, 4-21, 1-22, 2-22, 3-22, 4-22, 1-23, 2-23, 3-23, 4-23, 1-24, 2-24, 3-24, or 4-24, of any of the sequences in Table 2, Table 4, Table 5, Table 6, or Table 10. In certain embodiments, a CoV RNAi agent sense strand comprises or consists of a modified sequence of any one of the modified sequences in Table 3 or Table 10.

For the RNAi agents disclosed herein, the nucleotide at position 1 of the antisense strand (from 5′ end→3′ end) can be perfectly complementary to a SARS-CoV-2 viral genome, or can be non-complementary to a SARS-CoV-2 viral genome. In some embodiments, the nucleotide at position 1 of the antisense strand (from 5′ end→3′ end) is a U, A, or dT (or a modified version of U, A or dT). In some embodiments, the nucleotide at position 1 of the antisense strand (from 5′ end→3′ end) forms an A:U or U:A base pair with the sense strand.

In some embodiments, a CoV RNAi agent antisense strand comprises the sequence of nucleotides (from 5′ end→3′ end) 2-18 or 2-19 of any of the antisense strand sequences in Table 2, Table 3, or Table 10. In some embodiments, a SARS-CoV-2 RNAi sense strand comprises the sequence of nucleotides (from 5′ end→3′ end) 1-17 or 1-18 of any of the sense strand sequences in Table 2, Table 4, Table 5, Table 6, or Table 10.

In some embodiments, a CoV RNAi agent includes (i) an antisense strand comprising the sequence of nucleotides (from 5′ end→3′ end) 2-18 or 2-19 of any of the antisense strand sequences in Table 2, Table 3, or Table 10, and (ii) a sense strand comprising the sequence of nucleotides (from 5′ end→3′ end) 1-17 or 1-18 of any of the sense strand sequences in Table 2, Table 4, Table 5, Table 6, or Table 10.

A sense strand containing a sequence listed in Table 2 or Table 4 can be hybridized to any antisense strand containing a sequence listed in Table 2 or Table 3 provided the two sequences have a region of at least 85% complementarity over a contiguous 16, 17, 18, 19, 20, or 21 nucleotide sequence. In some embodiments, the CoV RNAi agent has a sense strand consisting of the modified sequence of any of the modified sequences in Table 4, Table 5, Table 6, or Table 10, and an antisense strand consisting of the modified sequence of any of the modified sequences in Table 3 or Table 10. Certain representative sequence pairings are exemplified by the Duplex ID Nos. shown in Tables 7A, 7B, 8, and 9.

In some embodiments, a CoV RNAi agent comprises, consists of, or consists essentially of a duplex represented by any one of the Duplex ID Nos. presented herein. In some embodiments, a CoV RNAi agent consists of any of the Duplex ID Nos. presented herein. In some embodiments, a CoV RNAi agent comprises the sense strand and antisense strand nucleotide sequences of any of the Duplex ID Nos. presented herein. In some embodiments, a CoV RNAi agent comprises the sense strand and antisense strand nucleotide sequences of any of the Duplex ID Nos. presented herein and a targeting group, linking group, and/or other non-nucleotide group wherein the targeting group, linking group, and/or other non-nucleotide group is covalently linked (i.e., conjugated) to the sense strand or the antisense strand. In some embodiments, a CoV RNAi agent includes the sense strand and antisense strand modified nucleotide sequences of any of the Duplex ID Nos. presented herein. In some embodiments, a CoV RNAi agent comprises the sense strand and antisense strand modified nucleotide sequences of any of the Duplex ID Nos. presented herein and a targeting group, linking group, and/or other non-nucleotide group, wherein the targeting group, linking group, and/or other non-nucleotide group is covalently linked to the sense strand or the antisense strand.

In some embodiments, a CoV RNAi agent comprises an antisense strand and a sense strand having the nucleotide sequences of any of the antisense strand/sense strand duplexes of Tables 2, 7A, 7B, 8, 9, or 10, and comprises a targeting group. In some embodiments, a CoV RNAi agent comprises an antisense strand and a sense strand having the nucleotide sequences of any of the antisense strand/sense strand duplexes of Tables 2, 7A, 7B, 8, 9, or 10, and comprises one or more αvβ6 integrin targeting ligands.

In some embodiments, a CoV RNAi agent comprises an antisense strand and a sense strand having the nucleotide sequences of any of the antisense strand/sense strand duplexes of Tables 2, 7A, 7B, 8, 9, or 10, and comprises a targeting group that is an integrin targeting ligand. In some embodiments, a CoV RNAi agent comprises an antisense strand and a sense strand having the nucleotide sequences of any of the antisense strand/sense strand duplexes of Tables 2, 7A, 7B, 8, 9, or 10, and comprises one or more αvβ6 integrin targeting ligands or clusters of αvβ6 integrin targeting ligands (e.g., a tridentate αvβ6 integrin targeting ligand).

In some embodiments, a CoV RNAi agent comprises an antisense strand and a sense strand having the modified nucleotide sequences of any of the antisense strand/sense strand duplexes of Tables 7A, 7B, 8, 9, and 10.

In some embodiments, a CoV RNAi agent comprises an antisense strand and a sense strand having the modified nucleotide sequences of any of the antisense strand/sense strand duplexes of Tables 7A, 7B, 8, 9, and 10, and comprises an integrin targeting ligand.

In some embodiments, a CoV RNAi agent comprises, consists of, or consists essentially of any of the duplexes of Tables 7A, 7B, 8, 9, and 10.

TABLE 7A
CoV RNAi agent Duplexes with Corresponding Sense and Antisense Strand
ID Numbers and Sequence ID numbers for the modified and unmodified
nucleotide sequences. (Shown without Linking Agents or Conjugates)

| | AS modified | AS unmodified | | SS modified | SS unmodified |
|---|---|---|---|---|---|
| AS ID | SEQ ID NO: | SEQ ID NO: | SS ID | SEQ ID NO: | SEQ ID NO: |
| AM11997-AS | 676 | 1060 | AM11996-SS-NL | 794 | 1135 |
| AM11999-AS | 677 | 1061 | AM11998-SS-NL | 795 | 1136 |
| AM12001-AS | 678 | 1062 | AM12000-SS-NL | 796 | 1137 |
| AM12003-AS | 679 | 1063 | AM12002-SS-NL | 797 | 1138 |
| AM12005-AS | 680 | 1064 | AM12004-SS-NL | 798 | 1139 |
| AM12007-AS | 681 | 1065 | AM12006-SS-NL | 799 | 1140 |
| AM12009-AS | 682 | 1066 | AM12008-SS-NL | 800 | 1141 |
| AM12011-AS | 683 | 1067 | AM12010-SS-NL | 801 | 1142 |
| AM12013-AS | 684 | 1068 | AM12012-SS-NL | 802 | 1143 |
| AM12015-AS | 685 | 1069 | AM12014-SS-NL | 803 | 1144 |
| AM12017-AS | 686 | 1070 | AM12016-SS-NL | 804 | 1145 |
| AM12019-AS | 687 | 1071 | AM12018-SS-NL | 805 | 1146 |
| AM12021-AS | 688 | 1072 | AM12020-SS-NL | 806 | 1147 |
| AM12023-AS | 689 | 1073 | AM12022-SS-NL | 807 | 1148 |
| AM12025-AS | 690 | 1074 | AM12024-SS-NL | 808 | 1149 |
| AM12027-AS | 691 | 1075 | AM12026-SS-NL | 809 | 1150 |
| AM12029-AS | 692 | 1076 | AM12028-SS-NL | 810 | 1151 |
| AM12031-AS | 693 | 1077 | AM12030-SS-NL | 811 | 1152 |
| AM12033-AS | 694 | 1078 | AM12032-SS-NL | 812 | 1153 |
| AM12035-AS | 695 | 1079 | AM12034-SS-NL | 813 | 1154 |
| AM12037-AS | 696 | 1080 | AM12036-SS-NL | 814 | 1155 |
| AM12039-AS | 697 | 1081 | AM12038-SS-NL | 815 | 1156 |
| AM12041-AS | 698 | 1082 | AM12040-SS-NL | 816 | 1157 |
| AM12041-AS | 698 | 1082 | AM12042-SS-NL | 817 | 1158 |
| AM12044-AS | 699 | 1083 | AM12043-SS-NL | 818 | 1159 |
| AM12046-AS | 700 | 1084 | AM12045-SS-NL | 819 | 1160 |
| AM12048-AS | 701 | 1085 | AM12047-SS-NL | 820 | 1161 |
| AM12050-AS | 702 | 1086 | AM12049-SS-NL | 821 | 1162 |
| AM12052-AS | 703 | 1087 | AM12051-SS-NL | 822 | 1163 |
| AM12054-AS | 704 | 1088 | AM12053-SS-NL | 823 | 1164 |
| AM12054-AS | 704 | 1088 | AM12055-SS-NL | 824 | 1164 |
| AM12057-AS | 705 | 1089 | AM12056-SS-NL | 825 | 1165 |
| AM12059-AS | 706 | 1090 | AM12058-SS-NL | 826 | 1166 |
| AM12059-AS | 706 | 1090 | AM12060-SS-NL | 827 | 1166 |
| AM12062-AS | 707 | 1091 | AM12061-SS-NL | 828 | 1167 |
| AM12064-AS | 708 | 1092 | AM12063-SS-NL | 829 | 1168 |
| AM12066-AS | 709 | 1093 | AM12065-SS-NL | 830 | 1169 |
| AM12068-AS | 710 | 1094 | AM12067-SS-NL | 831 | 1170 |
| AM12070-AS | 711 | 1095 | AM12069-SS-NL | 832 | 1171 |
| AM12469-AS | 712 | 1096 | AM12468-SS-NL | 833 | 1172 |
| AM12471-AS | 713 | 1097 | AM12470-SS-NL | 834 | 1173 |
| AM12473-AS | 714 | 1098 | AM12472-SS-NL | 835 | 1174 |
| AM12475-AS | 715 | 1099 | AM12474-SS-NL | 836 | 1175 |
| AM12477-AS | 716 | 1100 | AM12476-SS-NL | 837 | 1176 |
| AM12479-AS | 717 | 1101 | AM12478-SS-NL | 838 | 1177 |
| AM12481-AS | 718 | 1102 | AM12480-SS-NL | 839 | 1178 |
| AM12483-AS | 719 | 1103 | AM12482-SS-NL | 840 | 1179 |
| AM12485-AS | 720 | 1104 | AM12484-SS-NL | 841 | 1180 |
| AM12563-AS | 721 | 1105 | AM12562-SS-NL | 842 | 1181 |
| AM12565-AS | 722 | 1106 | AM12564-SS-NL | 843 | 1182 |
| AM12567-AS | 723 | 1107 | AM12566-SS-NL | 844 | 1183 |
| AM12569-AS | 724 | 1108 | AM12568-SS-NL | 845 | 1184 |
| AM12571-AS | 725 | 1109 | AM12570-SS-NL | 846 | 1185 |
| AM12573-AS | 726 | 1110 | AM12572-SS-NL | 847 | 1186 |
| AM12575-AS | 727 | 1111 | AM12574-SS-NL | 848 | 1187 |
| AM12577-AS | 728 | 1112 | AM12576-SS-NL | 849 | 1188 |
| AM12579-AS | 729 | 1113 | AM12578-SS-NL | 850 | 1189 |
| AM12581-AS | 730 | 1114 | AM12580-SS-NL | 851 | 1190 |
| AM12583-AS | 731 | 1115 | AM12582-SS-NL | 852 | 1191 |
| AM13120-AS | 732 | 1116 | AM13119-SS-NL | 853 | 1192 |
| AM13122-AS | 733 | 1117 | AM13121-SS-NL | 854 | 1193 |
| AM13124-AS | 734 | 1118 | AM13123-SS-NL | 855 | 1194 |
| AM13126-AS | 735 | 1119 | AM13125-SS-NL | 856 | 1195 |
| AM13128-AS | 736 | 1120 | AM13127-SS-NL | 857 | 1196 |
| AM12001-AS | 678 | 1062 | AM13129-SS-NL | 858 | 1137 |
| AM12469-AS | 712 | 1096 | AM13130-SS-NL | 859 | 1172 |
| AM12471-AS | 713 | 1097 | AM13131-SS-NL | 860 | 1173 |
| AM12005-AS | 680 | 1064 | AM13132-SS-NL | 861 | 1139 |
| AM12567-AS | 723 | 1107 | AM13133-SS-NL | 862 | 1183 |
| AM12569-AS | 724 | 1108 | AM13134-SS-NL | 863 | 1184 |
| AM12575-AS | 727 | 1111 | AM13135-SS-NL | 864 | 1187 |
| AM12579-AS | 729 | 1113 | AM13136-SS-NL | 865 | 1189 |
| AM12009-AS | 682 | 1066 | AM13158-SS-NL | 866 | 1141 |
| AM13160-AS | 737 | 1121 | AM13159-SS-NL | 867 | 1197 |
| AM12017-AS | 686 | 1070 | AM13161-SS-NL | 868 | 1145 |
| AM12475-AS | 715 | 1099 | AM13162-SS-NL | 869 | 1175 |
| AM13543-AS | 738 | 1122 | AM13542-SS-NL | 870 | 1198 |
| AM13545-AS | 739 | 1123 | AM13544-SS-NL | 871 | 1199 |
| AM13547-AS | 740 | 1115 | AM13546-SS-NL | 872 | 1191 |
| AM13120-AS | 732 | 1116 | AM14660-SS-NL | 873 | 1192 |
| AM14662-AS | 741 | 1124 | AM14661-SS-NL | 874 | 1200 |
| AM13122-AS | 733 | 1117 | AM14663-SS-NL | 875 | 1193 |
| AM13128-AS | 736 | 1120 | AM14664-SS-NL | 876 | 1196 |
| AM12001-AS | 678 | 1062 | AM14665-SS-NL | 877 | 1137 |
| AM14666-AS | 742 | 1120 | AM13127-SS-NL | 857 | 1196 |
| AM14667-AS | 743 | 1062 | AM13129-SS-NL | 858 | 1137 |
| AM14686-AS | 744 | 1117 | AM13121-SS-NL | 854 | 1193 |
| AM14687-AS | 745 | 1108 | AM13134-SS-NL | 863 | 1184 |
| AM14688-AS | 746 | 1113 | AM13136-SS-NL | 865 | 1189 |
| AM14689-AS | 747 | 1111 | AM13135-SS-NL | 864 | 1187 |
| AM14690-AS | 748 | 1099 | AM13162-SS-NL | 869 | 1175 |
| AM14859-AS | 749 | 1107 | AM13133-SS-NL | 862 | 1183 |
| AM14861-AS | 750 | 1125 | AM14860-SS-NL | 879 | 1201 |
| AM12475-AS | 715 | 1099 | AM15013-SS-NL | 880 | 1175 |
| AM12569-AS | 724 | 1108 | AM15014-SS-NL | 881 | 1184 |
| AM12579-AS | 729 | 1113 | AM15015-SS-NL | 882 | 1189 |
| AM12575-AS | 727 | 1111 | AM15016-SS-NL | 883 | 1187 |
| AM12567-AS | 723 | 1107 | AM15017-SS-NL | 884 | 1183 |
| AM14667-AS | 743 | 1062 | AM14665-SS-NL | 877 | 1137 |
| AM15563-AS | 751 | 1062 | AM14665-SS-NL | 877 | 1137 |
| AM15563-AS | 751 | 1062 | AM15564-SS-NL | 885 | 1137 |
| AM15565-AS | 752 | 1062 | AM15564-SS-NL | 885 | 1137 |
| AM15566-AS | 753 | 1062 | AM15564-SS-NL | 885 | 1137 |
| AM15568-AS | 754 | 1126 | AM15567-SS-NL | 886 | 1202 |
| AM15569-AS | 755 | 1062 | AM15564-SS-NL | 885 | 1137 |
| AM14690-AS | 748 | 1099 | AM15013-SS-NL | 880 | 1175 |
| AM15570-AS | 756 | 1099 | AM15013-SS-NL | 880 | 1175 |
| AM15570-AS | 756 | 1099 | AM15571-SS-NL | 887 | 1175 |
| AM15572-AS | 757 | 1099 | AM15571-SS-NL | 887 | 1175 |
| AM15574-AS | 758 | 1127 | AM15573-SS-NL | 888 | 1203 |
| AM15575-AS | 759 | 1099 | AM15571-SS-NL | 887 | 1175 |
| AM14859-AS | 749 | 1107 | AM15017-SS-NL | 884 | 1183 |
| AM15576-AS | 760 | 1107 | AM15017-SS-NL | 884 | 1183 |
| AM15576-AS | 760 | 1107 | AM15577-SS-NL | 889 | 1183 |
| AM15578-AS | 761 | 1107 | AM15577-SS-NL | 889 | 1183 |
| AM15579-AS | 762 | 1107 | AM15577-SS-NL | 889 | 1183 |
| AM15580-AS | 763 | 1107 | AM15577-SS-NL | 889 | 1183 |
| AM15582-AS | 764 | 1125 | AM15581-SS-NL | 890 | 1201 |
| AM14688-AS | 746 | 1113 | AM15015-SS-NL | 882 | 1189 |
| AM15795-AS | 765 | 1113 | AM15015-SS-NL | 882 | 1189 |
| AM15795-AS | 765 | 1113 | AM15796-SS-NL | 891 | 1189 |
| AM15797-AS | 766 | 1113 | AM15796-SS-NL | 891 | 1189 |
| AM15798-AS | 767 | 1113 | AM15796-SS-NL | 891 | 1189 |
| AM15799-AS | 768 | 1113 | AM15796-SS-NL | 891 | 1189 |
| AM15801-AS | 769 | 1128 | AM15800-SS-NL | 892 | 1204 |
| AM12015-AS | 685 | 1069 | AM15802-SS-NL | 893 | 1144 |
| AM15803-AS | 770 | 1069 | AM15802-SS-NL | 893 | 1144 |
| AM15804-AS | 771 | 1069 | AM15802-SS-NL | 893 | 1144 |
| AM15804-AS | 771 | 1069 | AM15805-SS-NL | 894 | 1144 |
| AM15807-AS | 772 | 1129 | AM15806-SS-NL | 895 | 1205 |
| AM15808-AS | 773 | 1069 | AM15805-SS-NL | 894 | 1144 |
| AM12017-AS | 686 | 1070 | AM15809-SS-NL | 896 | 1145 |
| AM15810-AS | 774 | 1070 | AM15809-SS-NL | 896 | 1145 |
| AM15812-AS | 775 | 1070 | AM15811-SS-NL | 897 | 1145 |
| AM14666-AS | 742 | 1120 | AM14664-SS-NL | 876 | 1196 |
| AM15813-AS | 776 | 1120 | AM14664-SS-NL | 876 | 1196 |
| AM15813-AS | 776 | 1120 | AM15814-SS-NL | 898 | 1196 |
| AM15815-AS | 777 | 1120 | AM15814-SS-NL | 898 | 1196 |
| AM15816-AS | 778 | 1120 | AM15814-SS-NL | 898 | 1196 |
| AM15818-AS | 779 | 1122 | AM15817-SS-NL | 899 | 1198 |
| AM15820-AS | 780 | 1130 | AM15819-SS-NL | 900 | 1206 |
| AM15822-AS | 781 | 1131 | AM15821-SS-NL | 901 | 1207 |
| AM15824-AS | 782 | 1132 | AM15823-SS-NL | 902 | 1208 |
| AM15826-AS | 783 | 1119 | AM15825-SS-NL | 903 | 1195 |
| AM15828-AS | 784 | 1064 | AM15827-SS-NL | 904 | 1139 |
| AM15830-AS | 785 | 1133 | AM15829-SS-NL | 905 | 1209 |
| AM15832-AS | 786 | 1084 | AM15831-SS-NL | 906 | 1160 |
| AM16516-AS | 787 | 1062 | AM15564-SS-NL | 885 | 1137 |
| AM16517-AS | 788 | 1126 | AM15567-SS-NL | 886 | 1202 |
| AM16519-AS | 789 | 1134 | AM16518-SS-NL | 907 | 1210 |
| AM15807-AS | 772 | 1129 | AM16520-SS-NL | 908 | 1205 |
| AM16521-AS | 790 | 1069 | AM15802-SS-NL | 893 | 1144 |
| AM16522-AS | 791 | 1069 | AM15802-SS-NL | 893 | 1144 |
| AM16523-AS | 792 | 1129 | AM16520-SS-NL | 908 | 1205 |
| AM16517-AS | 788 | 1126 | AM16965-SS-NL | 909 | 1202 |
| AM16966-AS | 793 | 1126 | AM16965-SS-NL | 909 | 1202 |
| AM15798-AS | 767 | 1113 | AM16967-SS-NL | 910 | 1189 |
| AM15813-AS | 776 | 1120 | AM13127-SS-NL | 857 | 1196 |
| AM15818-AS | 779 | 1122 | AM16968-SS-NL | 911 | 1198 |
| AM15570-AS | 756 | 1099 | AM16969-SS-NL | 912 | 1175 |
| AM15807-AS | 772 | 1129 | AM16970-SS-NL | 913 | 1205 |
| AM16966-AS | 793 | 1126 | AM15567-SS-NL | 886 | 1202 |

TABLE 7B
CoV RNAi agent Duplexes with Corresponding Sense and Antisense Strand ID Numbers
and Sequence ID numbers for the modified and unmodified nucleotide sequences.

| | | AS | AS | | SS | SS |
|---|---|---|---|---|---|---|
| | | modified | unmodified | | modified | unmodified |
| Duplex | AS ID | SEQ ID NO: | SEQ ID NO: | SS ID | SEQ ID NO: | SEQ ID NO: |
| AD08582 | AM11997-AS | 676 | 1060 | AM11996-SS | 914 | 1135 |
| AD08583 | AM11999-AS | 677 | 1061 | AM11998-SS | 915 | 1136 |
| AD08584 | AM12001-AS | 678 | 1062 | AM12000-SS | 916 | 1137 |
| AD08585 | AM12003-AS | 679 | 1063 | AM12002-SS | 917 | 1138 |
| AD08586 | AM12005-AS | 680 | 1064 | AM12004-SS | 918 | 1139 |
| AD08587 | AM12007-AS | 681 | 1065 | AM12006-SS | 919 | 1140 |
| AD08588 | AM12009-AS | 682 | 1066 | AM12008-SS | 920 | 1141 |
| AD08589 | AM12011-AS | 683 | 1067 | AM12010-SS | 921 | 1142 |
| AD08590 | AM12013-AS | 684 | 1068 | AM12012-SS | 922 | 1143 |
| AD08591 | AM12015-AS | 685 | 1069 | AM12014-SS | 923 | 1144 |
| AD08592 | AM12017-AS | 686 | 1070 | AM12016-SS | 924 | 1145 |
| AD08593 | AM12019-AS | 687 | 1071 | AM12018-SS | 925 | 1146 |
| AD08594 | AM12021-AS | 688 | 1072 | AM12020-SS | 926 | 1147 |
| AD08595 | AM12023-AS | 689 | 1073 | AM12022-SS | 927 | 1148 |
| AD08596 | AM12025-AS | 690 | 1074 | AM12024-SS | 928 | 1149 |
| AD08597 | AM12027-AS | 691 | 1075 | AM12026-SS | 929 | 1150 |
| AD08598 | AM12029-AS | 692 | 1076 | AM12028-SS | 930 | 1151 |
| AD08599 | AM12031-AS | 693 | 1077 | AM12030-SS | 931 | 1152 |
| AD08600 | AM12033-AS | 694 | 1078 | AM12032-SS | 932 | 1153 |
| AD08601 | AM12035-AS | 695 | 1079 | AM12034-SS | 933 | 1154 |
| AD08602 | AM12037-AS | 696 | 1080 | AM12036-SS | 934 | 1155 |
| AD08603 | AM12039-AS | 697 | 1081 | AM12038-SS | 935 | 1156 |
| AD08604 | AM12041-AS | 698 | 1082 | AM12040-SS | 936 | 1157 |
| AD08605 | AM12041-AS | 698 | 1082 | AM12042-SS | 937 | 1158 |
| AD08606 | AM12044-AS | 699 | 1083 | AM12043-SS | 938 | 1159 |
| AD08607 | AM12046-AS | 700 | 1084 | AM12045-SS | 939 | 1160 |
| AD08608 | AM12048-AS | 701 | 1085 | AM12047-SS | 940 | 1161 |
| AD08609 | AM12050-AS | 702 | 1086 | AM12049-SS | 941 | 1162 |
| AD08610 | AM12052-AS | 703 | 1087 | AM12051-SS | 942 | 1163 |
| AD08611 | AM12054-AS | 704 | 1088 | AM12053-SS | 943 | 1164 |
| AD08612 | AM12054-AS | 704 | 1088 | AM12055-SS | 944 | 1164 |
| AD08613 | AM12057-AS | 705 | 1089 | AM12056-SS | 945 | 1165 |
| AD08614 | AM12059-AS | 706 | 1090 | AM12058-SS | 946 | 1166 |
| AD08615 | AM12059-AS | 706 | 1090 | AM12060-SS | 947 | 1166 |
| AD08616 | AM12062-AS | 707 | 1091 | AM12061-SS | 948 | 1167 |
| AD08617 | AM12064-AS | 708 | 1092 | AM12063-SS | 949 | 1168 |
| AD08618 | AM12066-AS | 709 | 1093 | AM12065-SS | 950 | 1169 |
| AD08619 | AM12068-AS | 710 | 1094 | AM12067-SS | 951 | 1170 |
| AD08620 | AM12070-AS | 711 | 1095 | AM12069-SS | 952 | 1171 |
| AD08857 | AM12469-AS | 712 | 1096 | AM12468-SS | 953 | 1172 |
| AD08858 | AM12471-AS | 713 | 1097 | AM12470-SS | 954 | 1173 |
| AD08859 | AM12473-AS | 714 | 1098 | AM12472-SS | 955 | 1174 |
| AD08860 | AM12475-AS | 715 | 1099 | AM12474-SS | 956 | 1175 |
| AD08861 | AM12477-AS | 716 | 1100 | AM12476-SS | 957 | 1176 |
| AD08862 | AM12479-AS | 717 | 1101 | AM12478-SS | 958 | 1177 |
| AD08863 | AM12481-AS | 718 | 1102 | AM12480-SS | 959 | 1178 |
| AD08864 | AM12483-AS | 719 | 1103 | AM12482-SS | 960 | 1179 |
| AD08865 | AM12485-AS | 720 | 1104 | AM12484-SS | 961 | 1180 |
| AD08927 | AM12563-AS | 721 | 1105 | AM12562-SS | 962 | 1181 |
| AD08928 | AM12565-AS | 722 | 1106 | AM12564-SS | 963 | 1182 |
| AD08929 | AM12567-AS | 723 | 1107 | AM12566-SS | 964 | 1183 |
| AD08930 | AM12569-AS | 724 | 1108 | AM12568-SS | 965 | 1184 |
| AD08931 | AM12571-AS | 725 | 1109 | AM12570-SS | 966 | 1185 |
| AD08932 | AM12573-AS | 726 | 1110 | AM12572-SS | 967 | 1186 |
| AD08933 | AM12575-AS | 727 | 1111 | AM12574-SS | 968 | 1187 |
| AD08934 | AM12577-AS | 728 | 1112 | AM12576-SS | 969 | 1188 |
| AD08935 | AM12579-AS | 729 | 1113 | AM12578-SS | 970 | 1189 |
| AD08936 | AM12581-AS | 730 | 1114 | AM12580-SS | 971 | 1190 |
| AD08937 | AM12583-AS | 731 | 1115 | AM12582-SS | 972 | 1191 |
| AD09274 | AM13120-AS | 732 | 1116 | AM13119-SS | 973 | 1192 |
| AD09275 | AM13122-AS | 733 | 1117 | AM13121-SS | 974 | 1193 |
| AD09276 | AM13124-AS | 734 | 1118 | AM13123-SS | 975 | 1194 |
| AD09277 | AM13126-AS | 735 | 1119 | AM13125-SS | 976 | 1195 |
| AD09278 | AM13128-AS | 736 | 1120 | AM13127-SS | 977 | 1196 |
| AD09279 | AM12001-AS | 678 | 1062 | AM13129-SS | 978 | 1137 |
| AD09280 | AM12469-AS | 712 | 1096 | AM13130-SS | 979 | 1172 |
| AD09281 | AM12471-AS | 713 | 1097 | AM13131-SS | 980 | 1173 |
| AD09282 | AM12005-AS | 680 | 1064 | AM13132-SS | 981 | 1139 |
| AD09283 | AM12567-AS | 723 | 1107 | AM13133-SS | 982 | 1183 |
| AD09284 | AM12569-AS | 724 | 1108 | AM13134-SS | 983 | 1184 |
| AD09285 | AM12575-AS | 727 | 1111 | AM13135-SS | 984 | 1187 |
| AD09286 | AM12579-AS | 729 | 1113 | AM13136-SS | 985 | 1189 |
| AD09298 | AM12009-AS | 682 | 1066 | AM13158-SS | 986 | 1141 |
| AD09299 | AM13160-AS | 737 | 1121 | AM13159-SS | 987 | 1197 |
| AD09300 | AM12017-AS | 686 | 1070 | AM13161-SS | 988 | 1145 |
| AD09301 | AM12475-AS | 715 | 1099 | AM13162-SS | 989 | 1175 |
| AD09530 | AM13543-AS | 738 | 1122 | AM13542-SS | 990 | 1198 |
| AD09531 | AM13545-AS | 739 | 1123 | AM13544-SS | 991 | 1199 |
| AD09532 | AM13547-AS | 740 | 1115 | AM13546-SS | 992 | 1191 |
| AD10293 | AM13120-AS | 732 | 1116 | AM14660-SS | 993 | 1192 |
| AD10294 | AM14662-AS | 741 | 1124 | AM14661-SS | 994 | 1200 |
| AD10295 | AM13122-AS | 733 | 1117 | AM14663-SS | 995 | 1193 |
| AD10296 | AM13128-AS | 736 | 1120 | AM14664-SS | 996 | 1196 |
| AD10297 | AM12001-AS | 678 | 1062 | AM14665-SS | 997 | 1137 |
| AD10298 | AM14666-AS | 742 | 1120 | AM13127-SS | 977 | 1196 |
| AD10299 | AM14667-AS | 743 | 1062 | AM13129-SS | 978 | 1137 |
| AD10319 | AM14686-AS | 744 | 1117 | AM13121-SS | 974 | 1193 |
| AD10320 | AM14687-AS | 745 | 1108 | AM13134-SS | 983 | 1184 |
| AD10321 | AM14688-AS | 746 | 1113 | AM13136-SS | 985 | 1189 |
| AD10322 | AM14689-AS | 747 | 1111 | AM13135-SS | 984 | 1187 |
| AD10323 | AM14690-AS | 748 | 1099 | AM13162-SS | 989 | 1175 |
| AD10424 | AM14859-AS | 749 | 1107 | AM13133-SS | 982 | 1183 |
| AD10425 | AM14861-AS | 750 | 1125 | AM14860-SS | 999 | 1201 |
| AD10536 | AM12475-AS | 715 | 1099 | AM15013-SS | 1000 | 1175 |
| AD10537 | AM12569-AS | 724 | 1108 | AM15014-SS | 1001 | 1184 |
| AD10538 | AM12579-AS | 729 | 1113 | AM15015-SS | 1002 | 1189 |
| AD10539 | AM12575-AS | 727 | 1111 | AM15016-SS | 1003 | 1187 |
| AD10540 | AM12567-AS | 723 | 1107 | AM15017-SS | 1004 | 1183 |
| AD10912 | AM14667-AS | 743 | 1062 | AM14665-SS | 997 | 1137 |
| AD10913 | AM15563-AS | 751 | 1062 | AM14665-SS | 997 | 1137 |
| AD10914 | AM15563-AS | 751 | 1062 | AM15564-SS | 1005 | 1137 |
| AD10915 | AM15565-AS | 752 | 1062 | AM15564-SS | 1005 | 1137 |
| AD10916 | AM15566-AS | 753 | 1062 | AM15564-SS | 1005 | 1137 |
| AD10917 | AM15568-AS | 754 | 1126 | AM15567-SS | 1006 | 1202 |
| AD10918 | AM15569-AS | 755 | 1062 | AM15564-SS | 1005 | 1137 |
| AD10919 | AM14690-AS | 748 | 1099 | AM15013-SS | 1000 | 1175 |
| AD10920 | AM15570-AS | 756 | 1099 | AM15013-SS | 1000 | 1175 |
| AD10921 | AM15570-AS | 756 | 1099 | AM15571-SS | 1007 | 1175 |
| AD10922 | AM15572-AS | 757 | 1099 | AM15571-SS | 1007 | 1175 |
| AD10923 | AM15574-AS | 758 | 1127 | AM15573-SS | 1008 | 1203 |
| AD10924 | AM15575-AS | 759 | 1099 | AM15571-SS | 1007 | 1175 |
| AD10925 | AM14859-AS | 749 | 1107 | AM15017-SS | 1004 | 1183 |
| AD10926 | AM15576-AS | 760 | 1107 | AM15017-SS | 1004 | 1183 |
| AD10927 | AM15576-AS | 760 | 1107 | AM15577-SS | 1009 | 1183 |
| AD10928 | AM15578-AS | 761 | 1107 | AM15577-SS | 1009 | 1183 |
| AD10929 | AM15579-AS | 762 | 1107 | AM15577-SS | 1009 | 1183 |
| AD10930 | AM15580-AS | 763 | 1107 | AM15577-SS | 1009 | 1183 |
| AD10931 | AM15582-AS | 764 | 1125 | AM15581-SS | 1010 | 1201 |
| AD11101 | AM14688-AS | 746 | 1113 | AM15015-SS | 1002 | 1189 |
| AD11102 | AM15795-AS | 765 | 1113 | AM15015-SS | 1002 | 1189 |
| AD11103 | AM15795-AS | 765 | 1113 | AM15796-SS | 1011 | 1189 |
| AD11104 | AM15797-AS | 766 | 1113 | AM15796-SS | 1011 | 1189 |
| AD11105 | AM15798-AS | 767 | 1113 | AM15796-SS | 1011 | 1189 |
| AD11106 | AM15799-AS | 768 | 1113 | AM15796-SS | 1011 | 1189 |
| AD11107 | AM15801-AS | 769 | 1128 | AM15800-SS | 1012 | 1204 |
| AD11108 | AM12015-AS | 685 | 1069 | AM15802-SS | 1013 | 1144 |
| AD11109 | AM15803-AS | 770 | 1069 | AM15802-SS | 1013 | 1144 |
| AD11110 | AM15804-AS | 771 | 1069 | AM15802-SS | 1013 | 1144 |
| AD11111 | AM15804-AS | 771 | 1069 | AM15805-SS | 1014 | 1144 |
| AD11112 | AM15807-AS | 772 | 1129 | AM15806-SS | 1015 | 1205 |
| AD11113 | AM15808-AS | 773 | 1069 | AM15805-SS | 1014 | 1144 |
| AD11114 | AM12017-AS | 686 | 1070 | AM15809-SS | 1016 | 1145 |
| AD11115 | AM15810-AS | 774 | 1070 | AM15809-SS | 1016 | 1145 |
| AD11116 | AM15812-AS | 775 | 1070 | AM15811-SS | 1017 | 1145 |
| AD11117 | AM14666-AS | 742 | 1120 | AM14664-SS | 996 | 1196 |
| AD11118 | AM15813-AS | 776 | 1120 | AM14664-SS | 996 | 1196 |
| AD11119 | AM15813-AS | 776 | 1120 | AM15814-SS | 1018 | 1196 |
| AD11120 | AM15815-AS | 777 | 1120 | AM15814-SS | 1018 | 1196 |
| AD11121 | AM15816-AS | 778 | 1120 | AM15814-SS | 1018 | 1196 |
| AD11122 | AM15818-AS | 779 | 1122 | AM15817-SS | 1019 | 1198 |
| AD11123 | AM15820-AS | 780 | 1130 | AM15819-SS | 1020 | 1206 |
| AD11124 | AM15822-AS | 781 | 1131 | AM15821-SS | 1021 | 1207 |
| AD11125 | AM15824-AS | 782 | 1132 | AM15823-SS | 1022 | 1208 |
| AD11126 | AM15826-AS | 783 | 1119 | AM15825-SS | 1023 | 1195 |
| AD11127 | AM15828-AS | 784 | 1064 | AM15827-SS | 1024 | 1139 |
| AD11128 | AM15830-AS | 785 | 1133 | AM15829-SS | 1025 | 1209 |
| AD11129 | AM15832-AS | 786 | 1084 | AM15831-SS | 1026 | 1160 |
| AD11610 | AM16516-AS | 787 | 1062 | AM15564-SS | 1005 | 1137 |
| AD11611 | AM16517-AS | 788 | 1126 | AM15567-SS | 1006 | 1202 |
| AD11612 | AM16519-AS | 789 | 1134 | AM16518-SS | 1027 | 1210 |
| AD11613 | AM15807-AS | 772 | 1129 | AM16520-SS | 1028 | 1205 |
| AD11614 | AM16521-AS | 790 | 1069 | AM15802-SS | 1013 | 1144 |
| AD11615 | AM16522-AS | 791 | 1069 | AM15802-SS | 1013 | 1144 |
| AD11616 | AM16523-AS | 792 | 1129 | AM16520-SS | 1028 | 1205 |
| AD11958 | AM16517-AS | 788 | 1126 | AM16965-SS | 1029 | 1202 |
| AD11959 | AM16966-AS | 793 | 1126 | AM16965-SS | 1029 | 1202 |
| AD11960 | AM15798-AS | 767 | 1113 | AM16967-SS | 1030 | 1189 |
| AD11961 | AM15813-AS | 776 | 1120 | AM13127-SS | 977 | 1196 |
| AD11962 | AM15818-AS | 779 | 1122 | AM16968-SS | 1031 | 1198 |
| AD11963 | AM15570-AS | 756 | 1099 | AM16969-SS | 1032 | 1175 |
| AD11964 | AM15807-AS | 772 | 1129 | AM16970-SS | 1033 | 1205 |
| AD12040 | AM16966-AS | 793 | 1126 | AM15567-SS | 1006 | 1202 |

TABLE 8
CoV RNAi agent Duplexes with Corresponding Sense and Antisense Strand
ID Numbers and Sequence ID numbers for the modified and unmodified
nucleotide sequences. (Shown with Targeting Ligand Conjugates)

| | | AS | AS | | SS | SS |
|---|---|---|---|---|---|---|
| | | modified | unmodified | | modified | unmodified |
| Duplex | AS ID | SEQ ID NO: | SEQ ID NO: | SS ID | SEQ ID NO: | SEQ ID NO: |
| AC001334 | AM13120-AS | 732 | 1116 | CS001679 | 1034 | 1192 |
| AC001335 | AM13122-AS | 733 | 1117 | CS001681 | 1035 | 1193 |
| AC001336 | AM13124-AS | 734 | 1118 | CS001683 | 1036 | 1194 |
| AC001337 | AM13126-AS | 735 | 1119 | CS001685 | 1037 | 1195 |
| AC001338 | AM13128-AS | 736 | 1120 | CS001687 | 1038 | 1196 |
| AC001339 | AM12001-AS | 678 | 1062 | CS001689 | 1039 | 1137 |
| AC001340 | AM12469-AS | 712 | 1096 | CS001691 | 1040 | 1172 |
| AC001341 | AM12471-AS | 713 | 1096 | CS001693 | 1041 | 1173 |
| AC001342 | AM12005-AS | 680 | 1064 | CS001695 | 1042 | 1139 |
| AC001343 | AM12567-AS | 723 | 1107 | CS001697 | 1043 | 1183 |
| AC001344 | AM12569-AS | 724 | 1108 | CS001699 | 1044 | 1184 |
| AC001345 | AM12575-AS | 727 | 1111 | CS001701 | 1045 | 1187 |
| AC001346 | AM12579-AS | 729 | 1113 | CS001703 | 1046 | 1189 |
| AC001347 | AM12009-AS | 682 | 1066 | CS001705 | 1047 | 1141 |
| AC001348 | AM13160-AS | 737 | 1121 | CS001707 | 1048 | 1197 |
| AC001349 | AM12017-AS | 686 | 1070 | CS001709 | 1049 | 1145 |
| AC001350 | AM12475-AS | 715 | 1099 | CS001711 | 1050 | 1175 |
| AC001474 | AM13543-AS | 738 | 1122 | CS001891 | 1051 | 1198 |
| AC001475 | AM13545-AS | 739 | 1123 | CS001893 | 1052 | 1199 |
| AC001476 | AM13547-AS | 740 | 1115 | CS001895 | 1053 | 1191 |
| AC001887 | AM14666-AS | 742 | 1120 | CS001687 | 1038 | 1196 |
| AC001888 | AM14667-AS | 743 | 1062 | CS001689 | 1039 | 1137 |
| AC001922 | AM14686-AS | 744 | 1117 | CS001681 | 1035 | 1193 |
| AC001923 | AM14687-AS | 745 | 1108 | CS001699 | 1044 | 1184 |
| AC001924 | AM14688-AS | 746 | 1113 | CS001703 | 1046 | 1189 |
| AC001925 | AM14689-AS | 747 | 1111 | CS001701 | 1045 | 1187 |
| AC001926 | AM14690-AS | 748 | 1099 | CS001711 | 1050 | 1175 |
| AC001961 | AM14859-AS | 749 | 1107 | CS001697 | 1043 | 1183 |
| AC001962 | AM14861-AS | 750 | 1125 | CS002495 | 1054 | 1201 |
| AC002617 | AM16517-AS | 788 | 1126 | CS003334 | 1055 | 1202 |
| AC002618 | AM16966-AS | 793 | 1126 | CS003334 | 1055 | 1202 |
| AC002619 | AM15798-AS | 767 | 1113 | CS003337 | 1056 | 1189 |
| AC002620 | AM15813-AS | 776 | 1120 | CS001687 | 1038 | 1196 |
| AC002621 | AM15818-AS | 779 | 1122 | CS003340 | 1057 | 1198 |
| AC002622 | AM15570-AS | 756 | 1099 | CS003342 | 1058 | 1175 |
| AC002623 | AM15807-AS | 772 | 1129 | CS003344 | 1059 | 1205 |

TABLE 9
Conjugate Duplex ID Numbers Referencing Position
Targeted On SARS-Cov-2 Viral Genome

| | | | | Targeted SARS- |
|---|---|---|---|---|
| | | | | CoV-2 Viral |
| Conjugate | | | Duplex ID | Genome Position |
| Duplex ID | AS ID | SS ID | (Pre-Conjugation) | (Of SEQ ID NO: 1) |
| AC001334 | AM13120-AS | CS001679 | AD09274 | 3652 |
| AC001335 | AM13122-AS | CS001681 | AD09275 | 8140 |
| AC001336 | AM13124-AS | CS001683 | AD09276 | 4038 |
| AC001337 | AM13126-AS | CS001685 | AD09277 | 8039 |
| AC001338 | AM13128-AS | CS001687 | AD09278 | 4917 |
| AC001339 | AM12001-AS | CS001689 | AD09279 | 6412 |
| AC001340 | AM12469-AS | CS001691 | AD09280 | 10931 |
| AC001341 | AM12471-AS | CS001693 | AD09281 | 11434 |
| AC001342 | AM12005-AS | CS001695 | AD09282 | 12284 |
| AC001343 | AM12567-AS | CS001697 | AD09283 | 28587 |
| AC001344 | AM12569-AS | CS001699 | AD09284 | 28590 |
| AC001345 | AM12575-AS | CS001701 | AD09285 | 29064 |
| AC001346 | AM12579-AS | CS001703 | AD09286 | 29150 |
| AC001347 | AM12009-AS | CS001705 | AD09298 | 13766 |
| AC001348 | AM13160-AS | CS001707 | AD09299 | 14050 |
| AC001349 | AM12017-AS | CS001709 | AD09300 | 14511 |
| AC001350 | AM12475-AS | CS001711 | AD09301 | 15886 |
| AC001474 | AM13543-AS | CS001891 | AD09530 | 4156 |
| AC001475 | AM13545-AS | CS001893 | AD09531 | 4926 |
| AC001476 | AM13547-AS | CS001895 | AD09532 | 29329 |
| AC001887 | AM14666-AS | CS001687 | AD10298 | 4917 |
| AC001888 | AM14667-AS | CS001689 | AD10299 | 6412 |
| AC001922 | AM14686-AS | CS001681 | AD10319 | 8140 |
| AC001923 | AM14687-AS | CS001699 | AD10320 | 28590 |
| AC001924 | AM14688-AS | CS001703 | AD10321 | 29150 |
| AC001925 | AM14689-AS | CS001701 | AD10322 | 29064 |
| AC001926 | AM14690-AS | CS001711 | AD10323 | 15886 |
| AC001961 | AM14859-AS | CS001697 | AD10424 | 28587 |
| AC001962 | AM14861-AS | CS002495 | AD10425 | 28587 |
| AC002617 | AM16517-AS | CS003334 | AD11958 | 6412 |
| AC002618 | AM16966-AS | CS003334 | AD11959 | 6412 |
| AC002619 | AM15798-AS | CS003337 | AD11960 | 29150 |
| AC002620 | AM15813-AS | CS001687 | AD11961 | 4917 |
| AC002621 | AM15818-AS | CS003340 | AD11962 | 4156 |
| AC002622 | AM15570-AS | CS003342 | AD11963 | 15886 |
| AC002623 | AM15807-AS | CS003344 | AD11964 | 14503 |

TABLE 10
Conjugate ID Numbers With Chemically Modified Antisense
and Sense Strands (including Linkers and Conjugates)

| | Sense Strand | | | |
|---|---|---|---|---|
| AC ID | (Fully Modified with Conjugated | SEQ ID | | SEQ ID |
| Number | Targeting Ligand)(5′ → 3′) | NO: | Antisense Strand (5′ → 3′) | NO: |
| AC001334 | Tri-SM6.1-avb6-TA14- | 1034 | usCfsasUfaAfgCfaCfuCfuUfaAfgAfaGfsc | 732 |
| | gscuucuuaAfGfAfgugcuuaugas(invAb) | | | |
| AC001335 | Tri-SM6.1-avb6-TA14- | 1035 | usUfsasGfaUfaAfgAfcAfuUfgUfcUfaAfsg | 733 |
| | csuuagacaAfUfGfucuuaucuaas(invAb) | | | |
| AC001336 | Tri-SM6.1-avb6-TA14- | 1036 | usAfsusGfaAfgAfuUfgCfcAfuUfaAfuGfsc | 734 |
| | gscauuaauGfGfCfaaucuucauas(invAb) | | | |
| AC001337 | Tri-SM6.1-avb6-TA14- | 1037 | asAfsasCfgUfaUfuAfaCfgUfaAfgCfaUfsc | 735 |
| | gsaugcuuaCfGfUfuaauacguuus(invAb) | | | |
| AC001338 | Tri-SM6.1-avb6-TA14- | 1038 | usCfsusUfaAfgAfuUfgUfcAfaAfgGfuGfsc | 736 |
| | gscaccuuuGfAfCfaaucuuaagas(invAb) | | | |
| AC001339 | Tri-SM6.1-avb6-TA14- | 1039 | usUfsasGfgAfuUfuUfcCfaCfuAfcUfuCfsc | 678 |
| | gsgaaguagUfGfGfaaaauccuaas(invAb) | | | |
| AC001340 | Tri-SM6.1-avb6-TA14- | 1040 | usUfsgsUfcUfaAfcAfaCfaUfcAfaAfaGfsg | 712 |
| | cscuuuugaUfGfUfuguuagacaas(invAb) | | | |
| AC001341 | Tri-SM6.1-avb6-TA14- | 1041 | usCfsusUfgAfuCfuAfaAfgCfaUfuAfcCfsa | 713 |
| | usgguaaugCfUfUfuagaucaagas(invAb) | | | |
| AC001342 | Tri-SM6.1-avb6-TA14- | 1042 | asUfsasCfaUfuUfgGfgUfcAfuAfgCfuUfsg | 680 |
| | csaagcuauGfAfCfccaaauguaus(invAb) | | | |
| AC001343 | Tri-SM6.1-avb6-TA14- | 1043 | asGfsusAfgAfaAfuAfcCfaUfcUfuGfgAfsc | 723 |
| | gsuccaagaUfGfGfuauuucuacus(invAb) | | | |
| AC001344 | Tri-SM6.1-avb6-TA14- | 1044 | usGfsusAfgUfaGfaAfaUfaCfcAfuCfuUfsg | 724 |
| | csaagauggUfAfUfuucuacuacas(invAb) | | | |
| AC001345 | Tri-SM6.1-avb6-TA14- | 1045 | usUfsasCfaUfuGfuAfuGfcUfuUfaGfuGfsg | 727 |
| | cscacuaaaGfCfAfuacaauguaas(invAb) | | | |
| AC001346 | Tri-SM6.1-avb6-TA14- | 1046 | usUfsusGfuAfaUfcAfgUfuCfcUfuGfuCfsc | 729 |
| | gsgacaaggAfAfCfugauuacaaas(invAb) | | | |
| AC001347 | Tri-SM6.1-avb6-TA14- | 1047 | usUfsgsAfuAfuAfuGfuGfgUfaCfcAfuGfsc | 682 |
| | gscaugguaCfCfAfcauauaucaas(invAb) | | | |
| AC001348 | Tri-SM6.1-avb6-TA14- | 1048 | usUfsgsAfuUfaUfcUfaAfuGfuCfaGfuAfsc | 737 |
| | gsuacugacAfUfUfagauaaucaas(invAb) | | | |
| AC001349 | Tri-SM6.1-avb6-TA14- | 1049 | usAfsgsCfuAfuGfuAfaGfuUfuAfcAfuCfsc | 686 |
| | gsgauguaaAfCfUfuacauagcuas(invAb) | | | |
| AC001350 | Tri-SM6.1-avb6-TA14- | 1050 | usUfsgsUfuUfaAfcUfaGfcAfuUfgUfaUfsg | 715 |
| | csauacaauGfCfUfaguuaaacaas(invAb) | | | |
| AC001474 | Tri-SM6.1-avb6-TA14- | 1051 | usUfsasguagguauAfaCfcAfcagcsa | 738 |
| | usgcuguggUfuAfuAfccuacuaas(invAb) | | | |
| AC001475 | Tri-SM6.1-avb6-TA14- | 1052 | asAfsasGfaagugucUfuAfaGfauugsc | 739 |
| | gscaaucUfuAfaGfacacuucuuus(invAb) | | | |
| AC001476 | Tri-SM6.1-avb6-TA14- | 1053 | usCfsgsucaaUfAfugCfuUfaUfucagsc | 740 |
| | gscugaaUfaAfgCfauauugacias(invAb) | | | |
| AC001887 | Tri-SM6.1-avb6-TA14- | 1038 | cPrpusCfsusUfaAfgAfuUfgUfcAfaAfgGfuGfsc | 742 |
| | gscaccuuuGfAfCfaaucuuaagas(invAb) | | | |
| AC001888 | Tri-SM6.1-avb6-TA14- | 1039 | cPrpusUfsasGfgAfuUfuUfcCfaCfuAfcUfuCfsc | 743 |
| | gsgaaguagUfGfGfaaaauccuaas(invAb) | | | |
| AC001922 | Tri-SM6.1-avb6-TA14- | 1035 | cPrpusUfsasGfaUfaAfgAfcAfuUfgUfcUfaAfsg | 744 |
| | csuuagacaAfUfGfucuuaucuaas(invAb) | | | |
| AC001923 | Tri-SM6.1-avb6-TA14- | 1044 | cPrpusGfsusAfgUfaGfaAfaUfaCfcAfuCfuUfsg | 745 |
| | csaagauggUfAfUfuucuacuacas(invAb) | | | |
| AC001924 | Tri-SM6.1-avb6-TA14- | 1046 | cPrpusUfsusGfuAfaUfcAfgUfuCfcUfuGfuCfsc | 746 |
| | gsgacaaggAfAfCfugauuacaaas(invAb) | | | |
| AC001925 | Tri-SM6.1-avb6-TA14- | 1045 | cPrpusUfsasCfaUfuGfuAfuGfcUfuUfaGfuGfsg | 747 |
| | cscacuaaaGfCfAfuacaauguaas(invAb) | | | |
| AC001926 | Tri-SM6.1-avb6-TA14- | 1050 | cPrpusUfsgsUfuUfaAfcUfaGfcAfuUfgUfaUfsg | 748 |
| | csauacaauGfCfUfaguuaaacaas(invAb) | | | |
| AC001961 | Tri-SM6.1-avb6-TA14- | 1043 | cPrpasGfsusAfgAfaAfuAfcCfaUfcUfuGfgAfsc | 749 |
| | gsuccaagaUfGfGfuauuucuacus(invAb) | | | |
| AC001962 | Tri-SM6.1-avb6-TA14- | 1054 | cPrpusGfsusAfgAfaAfuAfcCfaUfcUfuGfgAfsc | 750 |
| | gsuccaagaUfGfGfuauuucuacas(invAb) | | | |
| AC002617 | Tri-SM6.1-avb6-TA14- | 1055 | cPrpuUfaGfgauuuucCfaCfuAfcuuscsg | 788 |
| | csgaaguagUfgGfaAfaauccuaas(invAb) | | | |
| AC002618 | Tri-SM6.1-avb6-TA14- | 1055 | cPrpusUfsaGfgauuuucCfaCfuAfcuuscsg | 793 |
| | csgaaguagUfgGfaAfaauccuaas(invAb) | | | |
| AC002619 | Tri-SM6.1-avb6-TA14- | 1056 | cPrpusUfsuGfuaaucagUfuCfcUfuguscsc | 767 |
| | gsgacaaggAfaCfuGfauuacaaas(invAb) | | | |
| AC002620 | Tri-SM6.1-avb6-TA14- | 1038 | cPrpusCfsusUfaagauugUfcAfaAfggugsc | 776 |
| | gscaccuuuGfAfCfaaucuuaagas(invAb) | | | |
| AC002621 | Tri-SM6.1-avb6-TA14- | 1057 | cPrpusUfsasGfuAfgGfuAfuAfaCfcAfcAfgCfsa | 779 |
| | usgcuguggUfUfAfuaccuacuaas(invAb) | | | |
| AC002622 | Tri-SM6.1-avb6-TA14- | 1058 | cPrpusUfsgUfuuaacuaGfcAfuUfguasusg | 756 |
| | csauacaauGfcUfaGfuuaaacaas(invAb) | | | |
| AC002623 | Tri-SM6.1-avb6-TA14- | 1059 | cPrpusAfsasGfuuuacauCfcUfgAfuuacsg | 772 |
| | csguaaucaGfgAfuGfuaaacuuas(invAb) | | | |

In some embodiments, a CoV RNAi agent is prepared or provided as a salt, mixed salt, or a free-acid. In some embodiments, a CoV RNAi agent is prepared or provided as a pharmaceutically acceptable salt. In some embodiments, a CoV RNAi agent is prepared or provided as a pharmaceutically acceptable sodium or potassium salt The RNAi agents described herein, upon delivery to a cell expressing a SARS-CoV-2 viral genome, inhibit or knockdown expression of one or more SARS-CoV-2 viral genomes in vivo and/or in vitro.

### Targeting Groups, Linking Groups, Pharmacokinetic/Pharmacodynamic (PK/PD) Modulators, and Delivery Vehicles

In some embodiments, a CoV RNAi agent contains or is conjugated to one or more non-nucleotide groups including, but not limited to, a targeting group, a linking group, a pharmacokinetic/pharmacodynamic (PK/PD) modulator, a delivery polymer, or a delivery vehicle. The non-nucleotide group can enhance targeting, delivery, or attachment of the RNAi agent. The non-nucleotide group can be covalently linked to the 3′ and/or 5′ end of either the sense strand and/or the antisense strand. In some embodiments, a CoV RNAi agent contains a non-nucleotide group linked to the 3′ and/or 5′ end of the sense strand. In some embodiments, a non-nucleotide group is linked to the 5′ end of a CoV RNAi agent sense strand. A non-nucleotide group can be linked directly or indirectly to the RNAi agent via a linker/linking group. In some embodiments, a non-nucleotide group is linked to the RNAi agent via a labile, cleavable, or reversible bond or linker.

In some embodiments, a non-nucleotide group enhances the pharmacokinetic or biodistribution properties of an RNAi agent or conjugate to which it is attached to improve cell- or tissue-specific distribution and cell-specific uptake of the conjugate. In some embodiments, a non-nucleotide group enhances endocytosis of the RNAi agent.

Targeting groups or targeting moieties enhance the pharmacokinetic or biodistribution properties of a conjugate or RNAi agent to which they are attached to improve cell-specific (including, in some cases, organ specific) distribution and cell-specific (or organ specific) uptake of the conjugate or RNAi agent. A targeting group can be monovalent, divalent, trivalent, tetravalent, or have higher valency for the target to which it is directed. Representative targeting groups include, without limitation, compounds with affinity to cell surface molecule, cell receptor ligands, hapten, antibodies, monoclonal antibodies, antibody fragments, and antibody mimics with affinity to cell surface molecules. In some embodiments, a targeting group is linked to an RNAi agent using a linker, such as a PEG linker or one, two, or three abasic and/or ribitol (abasic ribose) residues, which in some instances can serve as linkers.

A targeting group, with or without a linker, can be attached to the 5′ or 3′ end of any of the sense and/or antisense strands disclosed in Tables 2, 3, 4, 5, 6, and 10. A linker, with or without a targeting group, can be attached to the 5′ or 3′ end of any of the sense and/or antisense strands disclosed in Tables 2, 3, 4, 5, 6, and 10.

The CoV RNAi agents described herein can be synthesized having a reactive group, such as an amino group (also referred to herein as an amine), at the 5′-terminus and/or the 3′-terminus. The reactive group can be used subsequently to attach a targeting moiety using methods typical in the art.

For example, in some embodiments, the CoV RNAi agents disclosed herein are synthesized having an NH2-C6 group at the 5′-terminus of the sense strand of the RNAi agent. The terminal amino group subsequently can be reacted to form a conjugate with, for example, a group that includes an αvβ6 integrin targeting ligand. In some embodiments, the CoV RNAi agents disclosed herein are synthesized having one or more alkyne groups at the 5′-terminus of the sense strand of the RNAi agent. The terminal alkyne group(s) can subsequently be reacted to form a conjugate with, for example, a group that includes an αvβ6 integrin targeting ligand.

In some embodiments, a targeting group comprises an integrin targeting ligand. In some embodiments, an integrin targeting ligand is an αvβ6 integrin targeting ligand. The use of an αvβ6 integrin targeting ligand facilitates cell-specific targeting to cells having αvβ6 on its respective surface, and binding of the integrin targeting ligand can facilitate entry of the therapeutic agent, such as an RNAi agent, to which it is linked, into cells such as epithelial cells, including pulmonary epithelial cells and renal epithelial cells. Integrin targeting ligands can be monomeric or monovalent (e.g., having a single integrin targeting moiety) or multimeric or multivalent (e.g., having multiple integrin targeting moieties). The targeting group can be attached to the 3′ and/or 5′ end of the RNAi oligonucleotide using methods known in the art. The preparation of targeting groups, such as αvβ6 integrin targeting ligands, is described, for example, in International Patent Application Publication No. WO 2018/085415 and in International Patent Application Publication No. WO 2019/089765, the contents of each of which are incorporated herein in its entirety.

In some embodiments, targeting groups are linked to the CoV RNAi agents without the use of an additional linker. In some embodiments, the targeting group is designed having a linker readily present to facilitate the linkage to a CoV RNAi agent. In some embodiments, when two or more RNAi agents are included in a composition, the two or more RNAi agents can be linked to their respective targeting groups using the same linkers. In some embodiments, when two or more RNAi agents are included in a composition, the two or more RNAi agents are linked to their respective targeting groups using different linkers.

In some embodiments, a linking group is conjugated to the RNAi agent. The linking group facilitates covalent linkage of the agent to a targeting group, pharmacokinetic modulator, delivery polymer, or delivery vehicle. The linking group can be linked to the 3′ and/or the 5′ end of the RNAi agent sense strand or antisense strand. In some embodiments, the linking group is linked to the RNAi agent sense strand. In some embodiments, the linking group is conjugated to the 5′ or 3′ end of an RNAi agent sense strand. In some embodiments, a linking group is conjugated to the 5′ end of an RNAi agent sense strand. Examples of linking groups, include but are not limited to: C6-SS-C6, 6-SS-6, reactive groups such a primary amines (e.g., NH2-C6) and alkynes, alkyl groups, abasic residues/nucleotides, amino acids, tri-alkyne functionalized groups, ribitol, and/or PEG groups. Examples of certain linking groups are provided in Table 11.

A linker or linking group is a connection between two atoms that links one chemical group (such as an RNAi agent) or segment of interest to another chemical group (such as a targeting group, pharmacokinetic modulator, or delivery polymer) or segment of interest via one or more covalent bonds. A labile linkage contains a labile bond. A linkage can optionally include a spacer that increases the distance between the two joined atoms. A spacer may further add flexibility and/or length to the linkage. Spacers include, but are not limited to, alkyl groups, alkenyl groups, alkynyl groups, aryl groups, aralkyl groups, aralkenyl groups, and aralkynyl groups; each of which can contain one or more heteroatoms, heterocycles, amino acids, nucleotides, and saccharides. Spacer groups are well known in the art and the preceding list is not meant to limit the scope of the description. In some embodiments, a CoV RNAi agent is conjugated to a polyethylene glycol (PEG) moiety, or to a hydrophobic group having 12 or more carbon atoms, such as a cholesterol or palmitoyl group.

In some embodiments, a CoV RNAi agent is linked to one or more pharmacokinetic/pharmacodynamic (PK/PD) modulators. PK/PD modulators can increase circulation time of the conjugated drug and/or increase the activity of the RNAi agent through improved cell receptor binding, improved cellular uptake, and/or other means. Various PK/PD modulators suitable for use with RNAi agents are known in the art. In some embodiments, the PK/PD modulatory can be cholesterol or cholesteryl derivatives, or in some circumstances a PK/PD modulator can be comprised of alkyl groups, alkenyl groups, alkynyl groups, aryl groups, aralkyl groups, aralkenyl groups, or aralkynyl groups, each of which may be linear, branched, cyclic, and/or substituted or unsubstituted. In some embodiments, the location of attachment for these moieties is at the 5′ or 3′ end of the sense strand, at the 2′ position of the ribose ring of any given nucleotide of the sense strand, and/or attached to the phosphate or phosphorothioate backbone at any position of the sense strand.

Any of the CoV RNAi agent nucleotide sequences listed in Tables 2, 3, 4, 5, 6, and 10, whether modified or unmodified, can contain 3′ and/or 5′ targeting group(s), linking group(s), and/or PK/PD modulator(s). Any of the CoV RNAi agent sequences listed in Tables 3, 4, 5, 6, and 10, or are otherwise described herein, which contain a 3′ or 5′ targeting group, linking group, and/or PK/PD modulator can alternatively contain no 3′ or 5′ targeting group, linking group, or PK/PD modulator, or can contain a different 3′ or 5′ targeting group, linking group, or pharmacokinetic modulator including, but not limited to, those depicted in Table 11. Any of the CoV RNAi agent duplexes listed in Tables 7A, 7B, 8, 9, and 10, whether modified or unmodified, can further comprise a targeting group or linking group, including, but not limited to, those depicted in Table 11, and the targeting group or linking group can be attached to the 3′ or 5′ terminus of either the sense strand or the antisense strand of the CoV RNAi agent duplex.

Examples of certain modified nucleotides, capping moieties, and linking groups are provided in Table 11.

```
TABLE 11
Structures Representing Various Modified Nucleotides, Capping Moieties, and Linking Groups (wherein [symbol]     indicates the point of connection)

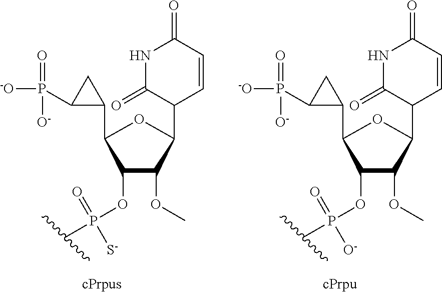

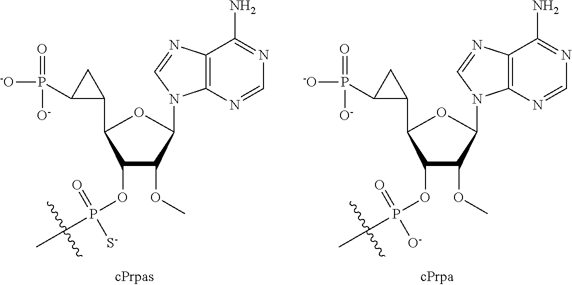

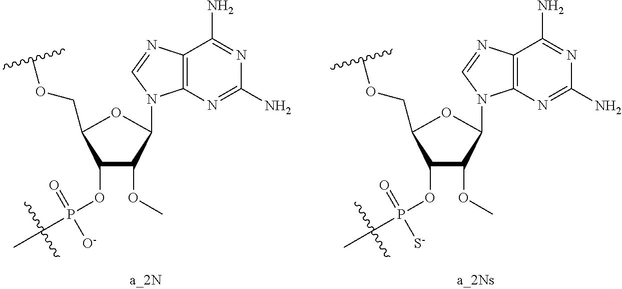

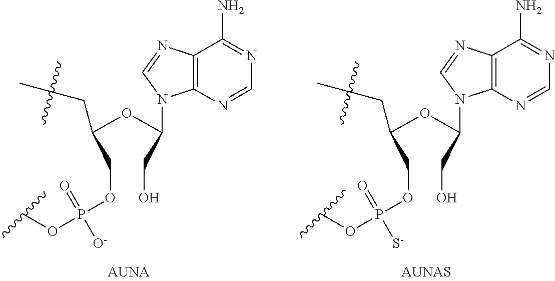

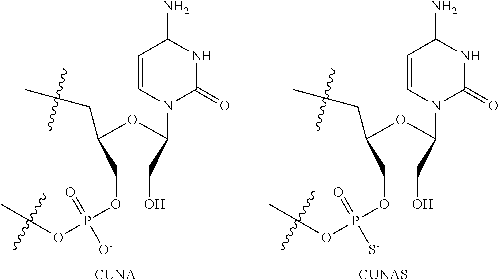

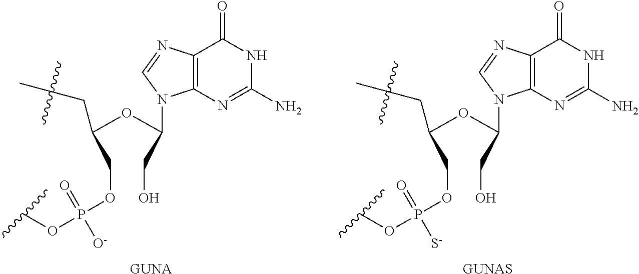

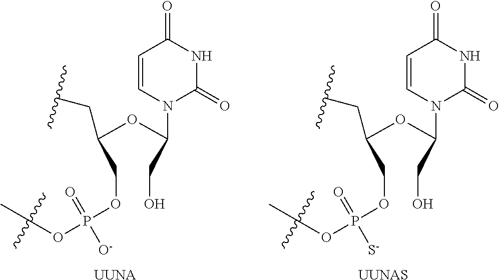







                    When positioned internally:

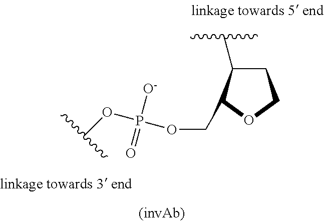

                    When positioned internally:

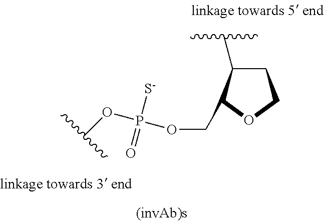

                    When positioned at the 3′ terminal end:

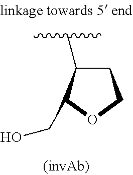

      When positioned at the 3′ terminal end:

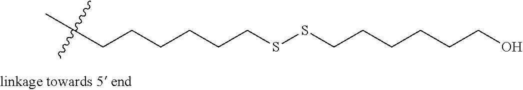 [SMILES: *CCCCCCSSCCCCCCO]

(C6-SS-C6)
      When positioned internally:

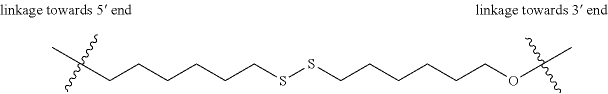 [SMILES: *CCCCCCSSCCCCCCO*]

(C6-SS-C6)
      When positioned at the 3′ terminal end:

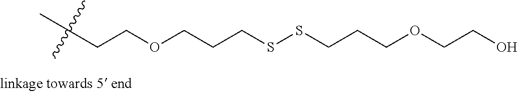 [SMILES: *CCOCCCSSCCCOCCO]

(6-SS-6)
      When positioned internally:

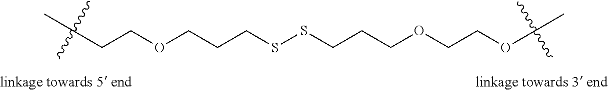 [SMILES: *CCOCCCSSCCCOCCO*]

(6-SS-6)

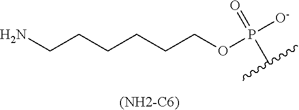 [SMILES: *P(=O)([O-])OCCCCCCN ; "(NH2-C6)"]

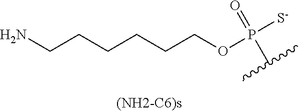 [SMILES: *P(=O)([S-])OCCCCCCN ; "(NH2-C6)s"]

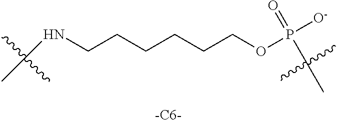 [SMILES: *NCCCCCCOP(*)(=O)[O-] ; "-C6-"]

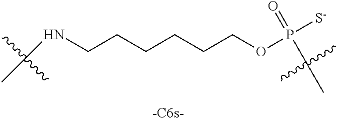 [SMILES: *NCCCCCCOP(*)(=O)[S-] ; "-C6s-"]

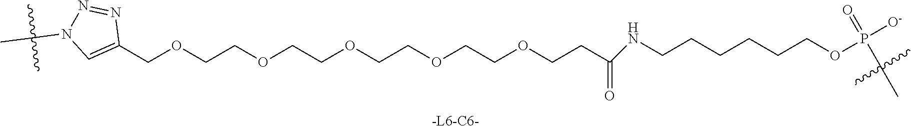 [SMILES: *n1cc(COCCOCCOCCOCCOCCC(=O)NCCCCCCOP(*)(=O)[O-])nn1 ; "-L6-C6-"]

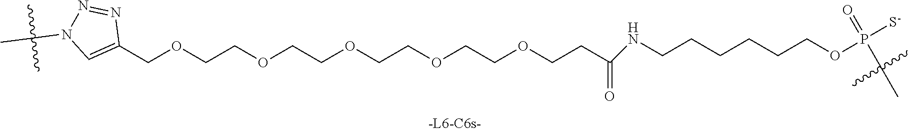 [SMILES: *n1cc(COCCOCCOCCOCCOCCC(=O)NCCCCCCOP(*)(=O)[S-])nn1]

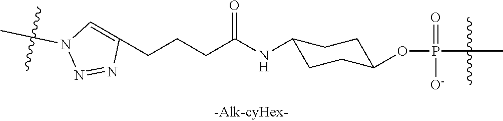 [SMILES: *n1cc(CCCC(=O)NC2CCC(OP(*)(=O)[O-])CC2)nn1]

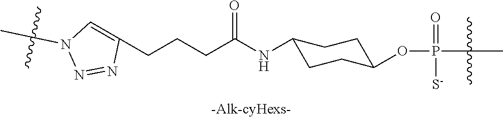 [SMILES: *n1cc(CCCC(=O)NC2CCC(OP(*)(=O)[S-])CC2)nn1]

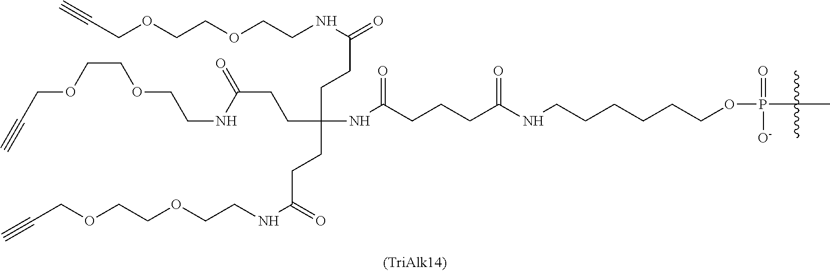 [SMILES: *P(=O)([O-])OCCCCCCNC(=O)CCCC(=O)NC(CCC(=O)NCCOCCOCC#C)(CCC(=O)NCCOCCOCC#C)CCC(=O)NCCOCCOCC#C]

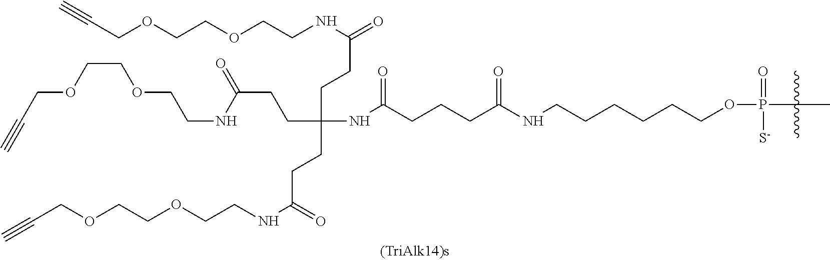 [SMILES: *P(=O)([S-])OCCCCCCNC(=O)CCCC(=O)NC(CCC(=O)NCCOCCOCC#C)(CCC(=O)NCCOCCOCC#C)CCC(=O)NCCOCCOCC#C]

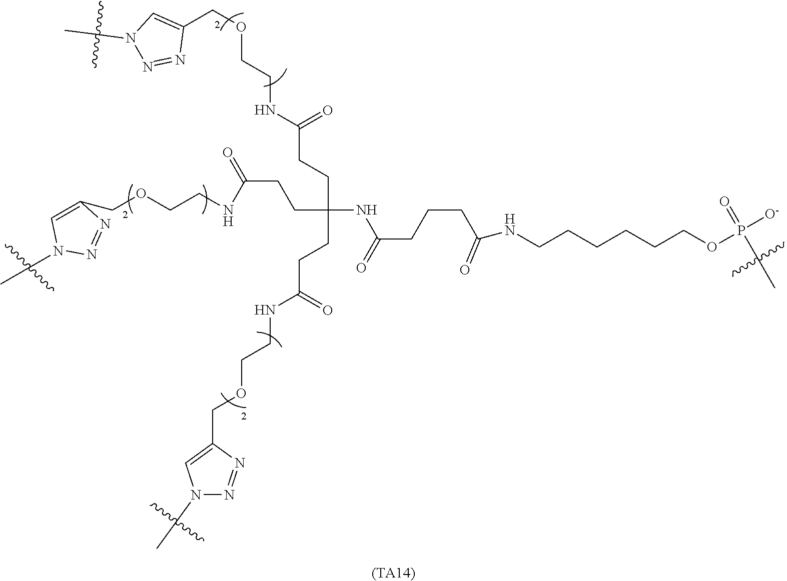

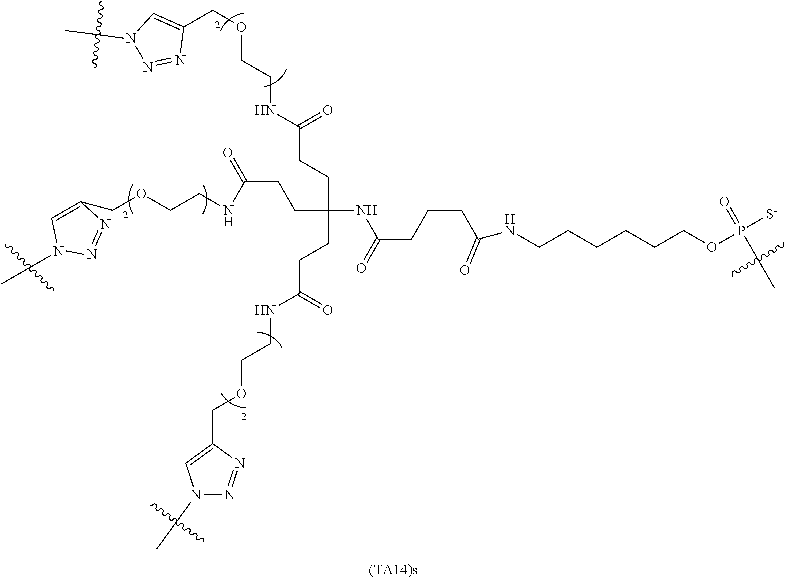

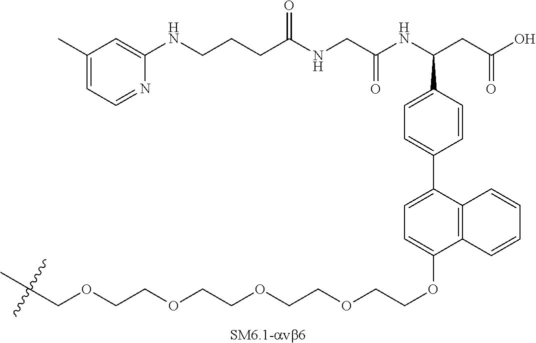 [SMILES: *COCCOCCOCCOCCOc1ccc(-c2ccc([C@H](CC(=O)O)NC(=O)CNC(=O)CCCNc3cc(C)ccn3)cc2)c2ccccc12]

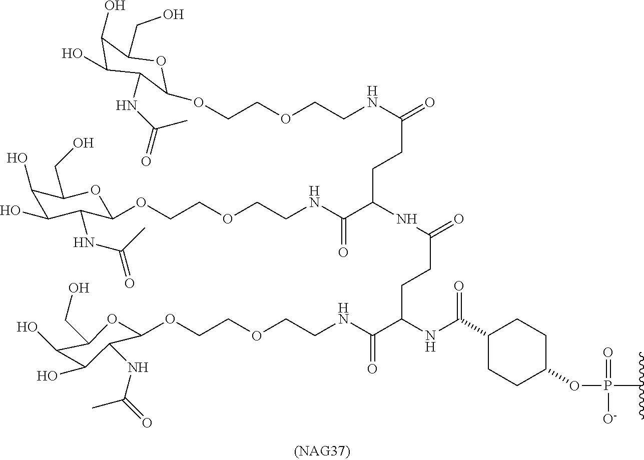

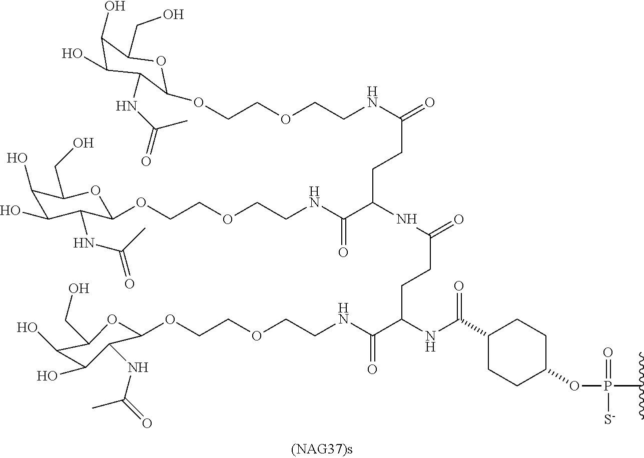















```

Alternatively, other linking groups known in the art may be used. In many instances, linking groups can be commercially acquired or alternatively, are incorporated into commercially available nucleotide phosphoramidites. (See, e.g., International Patent Application Publication No. WO 2019/161213, which is incorporated herein by reference in its entirety).

In some embodiments, a CoV RNAi agent is delivered without being conjugated to a targeting ligand or pharmacokinetic/pharmacodynamic (PK/PD) modulator (referred to as being “naked” or a “naked RNAi agent”).

In some embodiments, a CoV RNAi agent is conjugated to a targeting group, a linking group, a PK modulator, and/or another non-nucleotide group to facilitate delivery of the CoV RNAi agent to the cell or tissue of choice, for example, to an epithelial cell in vivo. In some embodiments, a CoV RNAi agent is conjugated to a targeting group wherein the targeting group includes an integrin targeting ligand. In some embodiments, the integrin targeting ligand is an αvβ6 integrin targeting ligand. In some embodiments, a targeting group includes one or more αvβ6 integrin targeting ligands.

In some embodiments, a delivery vehicle may be used to deliver an RNAi agent to a cell or tissue. A delivery vehicle is a compound that improves delivery of the RNAi agent to a cell or tissue. A delivery vehicle can include, or consist of, but is not limited to: a polymer, such as an amphipathic polymer, a membrane active polymer, a peptide, a melittin peptide, a melittin-like peptide (MLP), a lipid, a reversibly modified polymer or peptide, or a reversibly modified membrane active polyamine.

In some embodiments, the RNAi agents can be combined with lipids, nanoparticles, polymers, liposomes, micelles, DPCs or other delivery systems available in the art for nucleic acid delivery. The RNAi agents can also be chemically conjugated to targeting groups, lipids (including, but not limited to cholesteryl and cholesteryl derivatives), encapsulating in nanoparticles, liposomes, micelles, conjugating to polymers or DPCs (see, for example WO 2000/053722, WO 2008/022309, WO 2011/104169, and WO 2012/083185, WO 2013/032829, WO 2013/158141, each of which is incorporated herein by reference), by iontophoresis, or by incorporation into other delivery vehicles or systems available in the art such as hydrogels, cyclodextrins, biodegradable nanocapsules, bioadhesive microspheres, or proteinaceous vectors. In some embodiments the RNAi agents can be conjugated to antibodies having affinity for pulmonary epithelial cells. In some embodiments, the RNAi agents can be linked to targeting ligands that have affinity for pulmonary epithelial cells or receptors present on pulmonary epithelial cells.

### Pharmaceutical Compositions and Formulations

The CoV RNAi agents disclosed herein can be prepared as pharmaceutical compositions or formulations (also referred to herein as “medicaments”). In some embodiments, pharmaceutical compositions include at least one CoV RNAi agent. These pharmaceutical compositions are particularly useful in the inhibition of the expression of SARS-CoV-2 RNA or another CoV RNA transcript in a target cell, a group of cells, a tissue, or an organism. The pharmaceutical compositions can be used to treat a subject having a disease, disorder, or condition that would benefit from reduction in the level of the target coronavirus mRNA or RNA transcript, or inhibition in expression of the target viral genome. The pharmaceutical compositions can be used to treat a subject at risk of developing a disease or disorder that would benefit from reduction of the level of the target RNA or the target viral genome. In one embodiment, the method includes administering a CoV RNAi agent linked to a targeting ligand as described herein, to a subject to be treated. In some embodiments, one or more pharmaceutically acceptable excipients (including vehicles, carriers, diluents, and/or delivery polymers) are added to the pharmaceutical compositions that include a CoV RNAi agent, thereby forming a pharmaceutical formulation or medicament suitable for in vivo delivery to a subject, including a human.

The pharmaceutical compositions that include a CoV RNAi agent and methods disclosed herein decrease the level of the target coronavirus RNA in a cell, group of cells, group of cells, tissue, organ, or subject, including by administering to the subject a therapeutically effective amount of a herein described CoV RNAi agent, thereby inhibiting the expression of SARS-CoV-2 RNA or another CoV RNA or RNA transcript in the subject. In some embodiments, the subject has been previously identified or diagnosed as having a disease or disorder related to CoV infection, including SARS-CoV-2 infection, such as COVID-19. In some embodiments, the subject has been previously diagnosed with having pulmonary inflammation or other pulmonary symptoms consistent with a CoV infection.

Embodiments of the present disclosure include pharmaceutical compositions for delivering a CoV RNAi agent to a pulmonary epithelial cell in vivo. Such pharmaceutical compositions can include, for example, a CoV RNAi agent conjugated to a targeting group that comprises an integrin targeting ligand. In some embodiments, the integrin targeting ligand is comprised of an αvβ6 integrin ligand.

In some embodiments, the described pharmaceutical compositions including a CoV RNAi agent are used for treating or managing clinical presentations in a subject that would benefit from the inhibition of expression of SARS-CoV-2. In some embodiments, a therapeutically or prophylactically effective amount of one or more of pharmaceutical compositions is administered to a subject in need of such treatment. In some embodiments, administration of any of the disclosed CoV RNAi agents can be used to decrease the number, severity, and/or frequency of symptoms of a disease in a subject.

In some embodiments, the described CoV RNAi agents are optionally combined with one or more additional (i.e., second, third, etc.) therapeutics. A second therapeutic can be another CoV RNAi agent (e.g., a CoV RNAi agent that targets a different sequence within a SARS-CoV-2 viral genome). In some embodiments, a second therapeutic can be an RNAi agent that targets the SARS-CoV-2 viral genome or the genome of a different coronavirus. An additional therapeutic can also be a small molecule drug, antibody, antibody fragment. peptide, vaccine. and/or aptamer. The CoV RNAi agents, with or without the one or more additional therapeutics, can be combined with one or more excipients to form pharmaceutical compositions.

The described pharmaceutical compositions that include a CoV RNAi agent can be used to treat at least one symptom in a subject having a disease or disorder caused by a coronavirus infection. In some embodiments, the subject is administered a therapeutically effective amount of one or more pharmaceutical compositions that include a CoV RNAi agent thereby treating the symptom. In other embodiments, the subject is administered a prophylactically effective amount of one or more CoV RNAi agents, thereby preventing or inhibiting the at least one symptom by preventing the coronavirus from establishing itself and replicating in the cells of the organism.

In some embodiments, one or more of the described CoV RNAi agents are administered to a mammal in a pharmaceutically acceptable carrier or diluent. In some embodiments, the mammal is a human.

The route of administration is the path by which a CoV RNAi agent is brought into contact with the body. In general, methods of administering drugs, oligonucleotides, and nucleic acids, for treatment of a mammal are well known in the art and can be applied to administration of the compositions described herein. The CoV RNAi agents disclosed herein can be administered via any suitable route in a preparation appropriately tailored to the particular route. Thus, in some embodiments, the herein described pharmaceutical compositions are administered via inhalation, intranasal administration, intratracheal administration, or oropharyngeal aspiration administration. In some embodiments, the pharmaceutical compositions can be administered by injection, for example, intravenously, intramuscularly, intracutaneously, subcutaneously, intraarticularly, intraocularly, or intraperitoneally, or topically.

The pharmaceutical compositions including a CoV RNAi agent described herein can be delivered to a cell, group of cells, tissue, or subject using oligonucleotide delivery technologies known in the art. In general, any suitable method recognized in the art for delivering a nucleic acid molecule (in vitro or in vivo) can be adapted for use with the compositions described herein. For example, delivery can be by local administration, (e.g., direct injection, implantation, or topical administering), systemic administration, or subcutaneous, intravenous, intraperitoneal, or parenteral routes, including intracranial (e.g., intraventricular, intraparenchymal and intrathecal), intramuscular, transdermal, airway (aerosol), nasal, oral, rectal, or topical (including buccal and sublingual) administration. In some embodiments, the compositions are administered via inhalation, intranasal administration, oropharyngeal aspiration administration, or intratracheal administration. For example, in some embodiments, it is desired that the CoV RNAi agents described herein inhibit the expression of a SARS-CoV-2 viral genome or the genome of another coronavirus in the pulmonary epithelium, for which administration via inhalation (e.g., by an inhaler device, such as a metered-dose inhaler, or a nebulizer such as a jet or vibrating mesh nebulizer, or a soft mist inhaler) is particularly suitable and advantageous

In some embodiments, the pharmaceutical compositions described herein comprise one or more pharmaceutically acceptable excipients. The pharmaceutical compositions described herein are formulated for administration to a subject.

As used herein, a pharmaceutical composition or medicament includes a pharmacologically effective amount of at least one of the described therapeutic compounds and one or more pharmaceutically acceptable excipients. Pharmaceutically acceptable excipients (excipients) are substances other than the Active Pharmaceutical Ingredient (API, therapeutic product, e.g., CoV RNAi agent) that are intentionally included in the drug delivery system. Excipients do not exert or are not intended to exert a therapeutic effect at the intended dosage. Excipients can act to a) aid in processing of the drug delivery system during manufacture, b) protect, support or enhance stability, bioavailability or patient acceptability of the API, c) assist in product identification, and/or d) enhance any other attribute of the overall safety, effectiveness, of delivery of the API during storage or use. A pharmaceutically acceptable excipient may or may not be an inert substance.

Excipients include, but are not limited to: absorption enhancers, anti-adherents, anti-foaming agents, anti-oxidants, binders, buffering agents, carriers, coating agents, colors, delivery enhancers, delivery polymers, detergents, dextran, dextrose, diluents, disintegrants, emulsifiers, extenders, fillers, flavors, glidants, humectants, lubricants, oils, polymers, preservatives, saline, salts, solvents, sugars, surfactants, suspending agents, sustained release matrices, sweeteners, thickening agents, tonicity agents, vehicles, water-repelling agents, and wetting agents.

Pharmaceutical compositions suitable for injectable use include sterile aqueous solutions (where water-soluble) or dispersions and sterile powders for the extemporaneous preparation of sterile injectable solutions or dispersion. For intravenous administration, suitable carriers include physiological saline, bacteriostatic water, Cremophor® ELTM (BASF, Parsippany, NJ) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying which yields a powder of the active ingredient plus any additional desired ingredient from a previously sterile-filtered solution thereof.

Formulations suitable for intra-articular administration can be in the form of a sterile aqueous preparation of the drug that can be in microcrystalline form, for example, in the form of an aqueous microcrystalline suspension. Liposomal formulations or biodegradable polymer systems can also be used to present the drug for both intra-articular and ophthalmic administration.

Formulations suitable for inhalation administration can be prepared by incorporating the active compound in the desired amount in an appropriate solvent, followed by sterile filtration. In general, formulations for inhalation administration are sterile solutions at physiological pH and have low viscosity (<5 cP). Salts may be added to the formulation to balance tonicity. In some cases, surfactants or co-solvents can be added to increase active compound solubility and improve aerosol characteristics. In some cases, excipients can be added to control viscosity in order to ensure size and distribution of nebulized droplets.

In some embodiments, pharmaceutical formulations that include the CoV RNAi agents disclosed herein suitable for inhalation administration can be prepared in water for injection (sterile water), or an aqueous sodium phosphate buffer (for example, the CoV RNAi agent formulated in 0.5 mM sodium phosphate monobasic, 0.5 mM sodium phosphate dibasic, in water).

The active compounds can be prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

The CoV RNAi agents can be formulated in compositions in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the therapeutic effect to be achieved, and the limitations inherent in the art of compounding such an active compound for the treatment of individuals.

A pharmaceutical composition can contain other additional components commonly found in pharmaceutical compositions. Such additional components include, but are not limited to: anti-prurities, astringents, local anesthetics, or anti-inflammatory agents (e.g., antihistamine, diphenhydramine, etc.). It is also envisioned that cells, tissues, or isolated organs that express or comprise the herein defined RNAi agents may be used as “pharmaceutical compositions.” As used herein, “pharmacologically effective amount,” “therapeutically effective amount,” or simply “effective amount” refers to that amount of an RNAi agent to produce a pharmacological, therapeutic, or preventive result.

In some embodiments, the methods disclosed herein further comprise the step of administering a second therapeutic or treatment in addition to administering an RNAi agent disclosed herein. In some embodiments, the second therapeutic is another CoV RNAi agent (e.g., a CoV RNAi agent that targets a different sequence within the SARS-CoV-2 target). In other embodiments, the second therapeutic can be a small molecule drug, an antibody, an antibody fragment, a peptide, a vaccine, and/or an aptamer.

In some embodiments, described herein are compositions that include a combination or cocktail of at least two CoV RNAi agents having different sequences. In some embodiments, the two or more CoV RNAi agents are each separately and independently linked to targeting groups. In some embodiments, the two or more CoV RNAi agents are each linked to targeting groups that include or consist of integrin targeting ligands. In some embodiments, the two or more CoV RNAi agents are each linked to targeting groups that include or consist of αvβ6 integrin targeting ligands.

In some embodiments, described herein are compositions that include a combination or cocktail of one or more CoV RNAi agents and RNAi agents targeting other genes associated with causing CoV-related diseases. The other genes associated with causing CoV-related diseases can be, but are not limited to, genes that are associated with the severity of CoV-related diseases. In some embodiments, the combination of CoV RNAi agent(s) and RNAi agents targeting other genes associated with causing CoV-related diseases are each linked to targeting groups that include or consist of αvβ6 integrin targeting ligands. In some embodiments, CoV RNAi(s) are used in combination with RNAi agents targeting other genes associated with causing CoV-related diseases. In some embodiments the RNAi agents targeting other genes associated with causing CoV-related diseases are RNAi agents targeting to transmembrane serine protease 2 (TMPRSS2).

Described herein are compositions for delivery of CoV RNAi agents to pulmonary epithelial cells.

Generally, an effective amount of a CoV RNAi agent disclosed herein will be in the range of from about 0.0001 to about 30 mg/kg of body weight/deposited dose, e.g., from about 0.001 to about 5 mg/kg of body weight/deposited dose. In some embodiments, an effective amount of a CoV RNAi agent will be in the range of from about 0.01 mg/kg to about 3.0 mg/kg of body weight per deposited dose. In some embodiments, an effective amount of a CoV RNAi agent will be in the range of from about 0.03 mg/kg to about 2.0 mg/kg of body weight per deposited dose. In some embodiments, an effective amount of a CoV RNAi agent will be in the range of from about 0.01 to about 1.0 mg/kg of deposited dose per body weight. In some embodiments, an effective amount of a CoV RNAi agent will be in the range of from about 0.50 to about 1.0 mg/kg of deposited dose per body weight. The amount administered will also likely depend on such variables as the overall health status of the patient, the relative biological efficacy of the compound delivered, the formulation of the drug, the presence and types of excipients in the formulation, and the route of administration. Also, it is to be understood that the initial dosage administered can be increased beyond the above upper level to rapidly achieve the desired blood-level or tissue level, or the initial dosage can be smaller than the optimum. In some embodiments, a dose is administered daily. In some embodiments, a dose is administered weekly. In further embodiments, a dose is administered bi-weekly, tri-weekly, once monthly, or once quarterly (i.e., once every three months).

For treatment of disease or for formation of a medicament or composition for treatment of a disease, the pharmaceutical compositions described herein including a CoV RNAi agent can be combined with an excipient or with a second therapeutic agent or treatment including, but not limited to: a second or other RNAi agent, a small molecule drug, an antibody, an antibody fragment, a peptide, a vaccine and/or an aptamer.

The described CoV RNAi agents, when added to pharmaceutically acceptable excipients or adjuvants, can be packaged into kits, containers, packs, or dispensers. The pharmaceutical compositions described herein can be packaged in dry powder or aerosol inhalers, other metered-dose inhalers, nebulizers, pre-filled syringes, or vials.

### Methods of Treatment and Inhibition of CoV Viral Genomes

The CoV RNAi agents disclosed herein can be used to treat a subject (e.g., a human or other mammal) having a disease or disorder that would benefit from administration of the RNAi agent. In some embodiments, the RNAi agents disclosed herein can be used to treat a subject (e.g., a human) that would benefit from a reduction and/or inhibition in expression of SARS-CoV-2 mRNA and/or viral transcripts, or a reduction and/or inhibition of another coronavirus that is infecting the subject.

In some embodiments, the RNAi agents disclosed herein can be used to treat a subject (e.g., a human) having a disease or disorder caused by a coronavirus infection, including but not limited to, pulmonary inflammation or COVID-19. Treatment of a subject can include therapeutic and/or prophylactic treatment. The subject is administered a therapeutically effective amount of any one or more CoV RNAi agents described herein. The subject can be a human, patient, or human patient. The subject may be an adult, adolescent, child, or infant. Administration of a pharmaceutical composition described herein can be to a human being or animal.

In certain embodiments, the present disclosure provides methods for treatment of diseases, disorders, conditions, or pathological states mediated at least in part by SARS-CoV-2 viral genome expression, in a patient in need thereof, wherein the methods include administering to the patient any of the CoV RNAi agents described herein.

In some embodiments, the CoV RNAi agents are used to treat or manage a clinical presentation or pathological state in a subject, wherein the clinical presentation or pathological state is caused by a coronavirus infection. The subject is administered a therapeutically effective amount of one or more of the CoV RNAi agents or CoV RNAi agent-containing compositions described herein. In some embodiments, the method comprises administering a composition comprising a CoV RNAi agent described herein to a subject to be treated.

In a further aspect, the disclosure features methods of treatment (including prophylactic or preventative treatment) of diseases or symptoms that may be addressed by a reduction in CoV mRNA or RNA transcripts, including for example a reduction in SARS-CoV-2 mRNA or RNA transcripts, the methods comprising administering to a subject in need thereof a CoV RNAi agent that includes an antisense strand comprising the sequence of any of the sequences in Table 2, Table 3, or Table 10. Also described herein are compositions for use in such methods.

In another aspect, the disclosure provides methods for the treatment (including prophylactic treatment) of a pathological state (such as a condition or disease) caused by a coronavirus infection, such as COVID-19, wherein the methods include administering to a subject a therapeutically effective amount of an RNAi agent that includes an antisense strand comprising the sequence of any of the sequences in Table 2, Table 3, or Table 10.

In some embodiments, methods for inhibiting expression of a SARS-CoV-2 viral genome are disclosed herein, wherein the methods include administering to a cell an RNAi agent that includes an antisense strand comprising the sequence of any of the sequences in Table 2, Table 3, or Table 10.

In some embodiments, methods for the treatment (including prophylactic treatment) of a pathological state mediated at least in part by SARS-CoV-2 viral RNA are disclosed herein, wherein the methods include administering to a subject a therapeutically effective amount of an RNAi agent that includes a sense strand comprising the sequence of any of the sequences in Table 2, Table 4, Table 5, Table 6, or Table 10.

In some embodiments, methods for inhibiting expression of a SARS-CoV-2 viral genome are disclosed herein, wherein the methods comprise administering to a cell an RNAi agent that includes a sense strand comprising the sequence of any of the sequences in Table 2, Table 4, Table 5, Table 6, or Table 10.

In some embodiments, methods for the treatment (including prophylactic treatment) of a pathological state mediated at least in part by SARS-CoV-2 viral RNA are disclosed herein, wherein the methods include administering to a subject a therapeutically effective amount of an RNAi agent that includes a sense strand comprising the sequence of any of the sequences in Table 4, Table 5, Table 6, or Table 10, and an antisense strand comprising the sequence of any of the sequences in Table 3 or Table 10.

In some embodiments, methods for inhibiting expression of a SARS-CoV-2 viral genome are disclosed herein, wherein the methods include administering to a cell an RNAi agent that includes a sense strand comprising the sequence of any of the sequences in Table 4. Table 5, Table 6, or Table 10, and an antisense strand comprising the sequence of any of the sequences in Table 3 or Table 10.

In some embodiments, methods of inhibiting expression of a SARS-CoV-2 viral genome are disclosed herein, wherein the methods include administering to a subject a CoV RNAi agent that includes a sense strand consisting of the nucleobase sequence of any of the sequences in Table 4, Table 5, Table 6, or Table 10, and the antisense strand consisting of the nucleobase sequence of any of the sequences in Table 3 or Table 10. In other embodiments, disclosed herein are methods of inhibiting expression of a SARS-CoV-2 viral genome, wherein the methods include administering to a subject a CoV RNAi agent that includes a sense strand consisting of the modified sequence of any of the modified sequences in Table 4, Table 5, Table 6, or Table 10, and the antisense strand consisting of the modified sequence of any of the modified sequences in Table 3 or Table 10.

In some embodiments, methods for inhibiting expression of a SARS-CoV-2 viral genome in a cell are disclosed herein, wherein the methods include administering one or more CoV RNAi agents comprising a duplex structure of one of the duplexes set forth in Tables 7A, 7B, 8, 9, and 10.

In some embodiments, the SARS-CoV-2 viral RNA level in certain epithelial cells of subject to whom a described CoV RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the CoV RNAi agent or to a subject not receiving the CoV RNAi agent. In some embodiments, the SARS-CoV-2 subgenomic RNA levels in certain epithelial cells of a subject to whom a described CoV RNAi agent is administered is reduced by at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, or greater than 99%, relative to the subject prior to being administered the CoV RNAi agent or to a subject not receiving the CoV RNAi agent. The viral RNA transcript level, mRNA level, and/or subgenomic RNA level in the subject may be reduced in a cell, group of cells, and/or tissue of the subject. In some embodiments, the SARS-CoV-2 mRNA levels in certain epithelial cells subject to whom a described CoV RNAi agent has been administered is reduced by at least about 30%, 35%, 40%, 45%, 50%, 55%, 60%, 65%, 70%, 75%, 80%, 85%, 90%, 95%, or 98% relative to the subject prior to being administered the CoV RNAi agent or to a subject not receiving the CoV RNAi agent.

In some embodiments, methods for the treatment (including prophylactic treatment) of a pathological state mediated at least in part by SARS-CoV-2 viral RNA are disclosed herein, wherein the methods include administering to a subject a therapeutically effective amount of a combination of an RNAi agent that includes a sense strand comprising the sequence of any of the sequences in Table 4, Table 5, Table 6, or Table 10, and an antisense strand comprising the sequence of any of the sequences in Table 3 or Table 10, in addition to RNAi agents targeting other genes associated with causing CoV-related diseases. The other genes associated with causing CoV-related diseases can be, but are not limited to, genes that are associated with the severity of CoV-related diseases. In some embodiments, the combination of CoV RNAi agent(s) and RNAi agents targeting other genes associated with causing CoV-related diseases are each linked to targeting groups that include or consist of αvβ6 integrin targeting ligands. In some embodiments, CoV RNAi(s) are used in combination with RNAi agents targeting other genes associated with causing CoV-related diseases. In some embodiments the RNAi agents targeting other genes associated with causing CoV-related diseases are RNAi agents targeting to transmembrane serine protease 2 (TMPRSS2).

Reductions in viral RNA can be assessed by any methods known in the art and are collectively referred to herein as a decrease in, reduction of, or inhibition of SARS-CoV-2. The Examples set forth herein illustrate known methods for assessing inhibition of SARS-CoV-2 viral RNA.

### Cells, Tissues, Organs, and Non-Human Organisms

Cells, tissues, organs, and non-human organisms that include at least one of the CoV RNAi agents described herein are contemplated. The cell, tissue, organ, or non-human organism is made by delivering the RNAi agent to the cell, tissue, organ, or non-human organism.

### ADDITIONAL ILLUSTRATIVE EMBODIMENTS

Provided here are certain additional illustrative embodiments of the disclosed technology. These embodiments are illustrative only and do not limit the scope of the present disclosure or of the claims attached hereto.

Embodiment 1. An RNAi agent for inhibiting expression of a coronavirus (CoV) genome, comprising:
an antisense strand comprising at least 17 contiguous nucleotides differing by 0 or 1 nucleotides from any one of the sequences provided in Table 2 or Table 3; and
a sense strand comprising a nucleotide sequence that is at least partially complementary to the antisense strand.

Embodiment 2. The RNAi agent of embodiment 1, wherein the antisense strand comprises nucleotides 2-18 of any one of the sequences provided in Table 2 or Table 3.

Embodiment 3. The RNAi agent of embodiment 1 or embodiment 2, wherein the sense strand comprises a nucleotide sequence of at least 17 contiguous nucleotides differing by 0 or 1 nucleotides from any one of the sequences provided in Table 2 or Table 4, and wherein the sense strand has a region of at least 85% complementarity over the 17 contiguous nucleotides to the antisense strand.

Embodiment 4. The RNAi agent of any one of embodiments 1-3, wherein at least one nucleotide of the RNAi agent is a modified nucleotide or includes a modified internucleoside linkage.

Embodiment 5. The RNAi agent of any one of embodiments 1-4, wherein all or substantially all of the nucleotides are modified nucleotides.

Embodiment 6. The RNAi agent of any one of embodiments 4-5, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

Embodiment 7. The RNAi agent of embodiment 5, wherein all or substantially all of the nucleotides are modified with 2′-O-methyl nucleotides, 2′-fluoro nucleotides, or combinations thereof.

Embodiment 8. The RNAi agent of any one of embodiments 1-7, wherein the antisense strand comprises the nucleotide sequence of any one of the modified sequences provided in Table 3.

Embodiment 9. The RNAi agent of any one of embodiments 1-8, wherein the sense strand comprises the nucleotide sequence of any one of the modified sequences provided in Table 4.

Embodiment 10. The RNAi agent of embodiment 1, wherein the antisense strand comprises the nucleotide sequence of any one of the modified sequences provided in Table 3 and the sense strand comprises the nucleotide sequence of any one of the modified sequences provided in Table 4.

Embodiment 11. The RNAi agent of any one of embodiments 1-10, wherein the sense strand is between 18 and 30 nucleotides in length, and the antisense strand is between 18 and 30 nucleotides in length.

Embodiment 12. The RNAi agent of embodiment 11, wherein the sense strand and the antisense strand are each between 18 and 27 nucleotides in length.

Embodiment 13. The RNAi agent of embodiment 12, wherein the sense strand and the antisense strand are each between 18 and 24 nucleotides in length.

Embodiment 14. The RNAi agent of embodiment 13, wherein the sense strand and the antisense strand are each 21 nucleotides in length.

Embodiment 15. The RNAi agent of embodiment 14, wherein the RNAi agent has two blunt ends.

Embodiment 16. The RNAi agent of any one of embodiments 1-15, wherein the sense strand comprises one or two terminal caps.

Embodiment 17. The RNAi agent of any one of embodiments 1-16, wherein the sense strand comprises one or two inverted abasic residues.

Embodiment 18. The RNAi agent of embodiment 1, wherein the RNAi agent is comprised of a sense strand and an antisense strand that form a duplex having the structure of any one of the duplexes in Table 7A, Table 7B, Table 8, Table 9, or Table 10.

Embodiment 19. The RNAi agent of embodiment 18, wherein all or substantially all of the nucleotides are modified nucleotides.

Embodiment 20. The RNAi agent of embodiment 1, comprising an antisense strand that consists of, consists essentially of, or comprises a nucleotide sequence that differs by 0 or 1 nucleotides from one of the following nucleotide sequences (5′→3′):

(SEQ ID NO: 1122)
UUAGUAGGUAUAACCACAGCA;
or
(SEQ ID NO: 1113)
UUUGUAAUCAGUUCCUUGUCC.

Embodiment 21. The RNAi agent of any one of embodiments 1-20, wherein the nucleotides of the antisense strand located at position 2 and position 14 from the 5′-end are 2′-fluoro modified nucleotides.

Embodiment 22. The RNAi agent of embodiment 21, wherein the nucleotide of the antisense strand at position 2 is a 2′-fluoro uridine, and the nucleotide of the antisense strand at position 14 is a 2′-fluoro cytidine, and wherein the antisense strand comprises 3 or 4 phosphorothioate intemucleoside linkages.

Embodiment 23. The RNAi agent of any one of embodiments 1-22, wherein the sense strand consists of, consists essentially of, or comprises a nucleotide sequence that differs by 0 or 1 nucleotides from one of the following nucleotide sequences (5′→3′):

(SEQ ID NO: 1198)
UGCUGUGGUUAUACCUACUAA;
or
(SEQ ID NO: 1189)
GGACAAGGAACUGAUUACAAA.

Embodiment 24. The RNAi agent of any one of embodiments 20-23, wherein all or substantially all of the nucleotides are modified nucleotides.

Embodiment 25. The RNAi agent of embodiment 1, comprising an antisense strand that comprises, consists of, or consists essentially of a modified nucleotide sequence that differs by 0 or 1 nucleotides from one of the following nucleotide sequences (5′→3′):

(SEQ ID NO: 779)
cPrpusUfsasGfuAfgGfuAfuAfaCfcAfcAfgCfsa;
or
(SEQ ID NO: 746)
cPrpusUfsusGfuAfaUfcAfgUfuCfcUfuGfuCfsc.

wherein a represents 2′-O-methyl adenosine, c represents 2′-O-methyl cytidine, g represents 2′-O-methyl guanosine, and u represents 2′-O-methyl uridine; Af, represents 2′-fluoro adenosine, Cf represents 2′-fluoro cytidine, Gf represents 2′-fluoro guanosine, and Uf represents 2′-fluoro uridine; cPrpu represents a 5′-cyclopropyl phosphonate-2′-O-methyl uridine; s represents a phosphorothioate linkage; and wherein all or substantially all of the nucleotides on the sense strand are modified nucleotides.

Embodiment 26. The RNAi agent of embodiment 1, wherein the sense strand comprises, consists of, or consists essentially of a modified nucleotide sequence that differs by 0 or 1 nucleotides from one of the following nucleotide sequences (5′→3′): usgcuguggUfUfAfuaccuacuaas (SEQ ID NO: 1057); or gsgacaaggAfAfCfugauuacaaas (SEQ ID NO: 1046).
wherein a represents 2′-O-methyl adenosine, c represents 2′-O-methyl cytidine, g represents 2′-O-methyl guanosine, and u represents 2′-O-methyl uridine; Af, represents 2′-fluoro adenosine, Cf represents 2′-fluoro cytidine, Gf represents 2′-fluoro guanosine, and Uf represents 2′-fluoro uridine; s represents a phosphorothioate linkage; and wherein all or substantially all of the nucleotides on the antisense strand are modified nucleotides.

Embodiment 27. The RNAi agent of any one of embodiments 20-26, wherein the sense strand further includes inverted abasic residues at the 3′ terminal end of the nucleotide sequence, at the 5′ end of the nucleotide sequence, or at both.

Embodiment 28. The RNAi agent of any one of embodiments 1-27, wherein the RNAi agent is linked to a targeting ligand.

Embodiment 29. The RNAi agent of embodiment 28, wherein the targeting ligand has affinity for a cell receptor expressed on an epithelial cell.

Embodiment 30. The RNAi agent of embodiment 29, wherein the targeting ligand comprises an integrin targeting ligand.

Embodiment 31. The RNAi agent of embodiment 30, wherein the integrin targeting ligand is an αvβ6 integrin targeting ligand.

Embodiment 32. The RNAi agent of embodiment 31, wherein the targeting ligand comprises the structure:

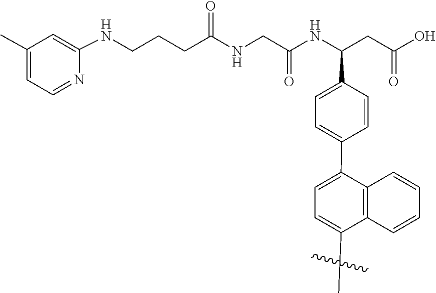 [SMILES: *c1ccc(-c2ccc([C@H](CC(=O)O)NC(=O)CNC(=O)CCCNc3cc(C)ccn3)cc2)c2ccccc12]

or a pharmaceutically acceptable salt thereof, or

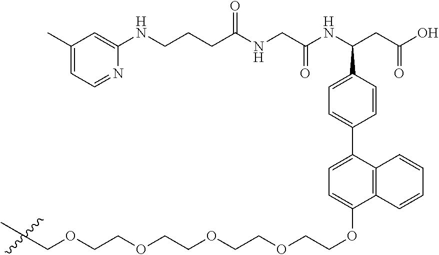 [SMILES: *COCCOCCOCCOCCOc1ccc(-c2ccc([C@H](CC(=O)O)NC(=O)CNC(=O)CCCNc3cc(C)ccn3)cc2)c2ccccc12]

or a pharmaceutically acceptable salt thereof, wherein [symbol] indicates the point of connection to the RNAi agent.

Embodiment 33. The RNAi agent of any one of embodiments 28-31, wherein the targeting ligand has a structure selected from the group consisting of:

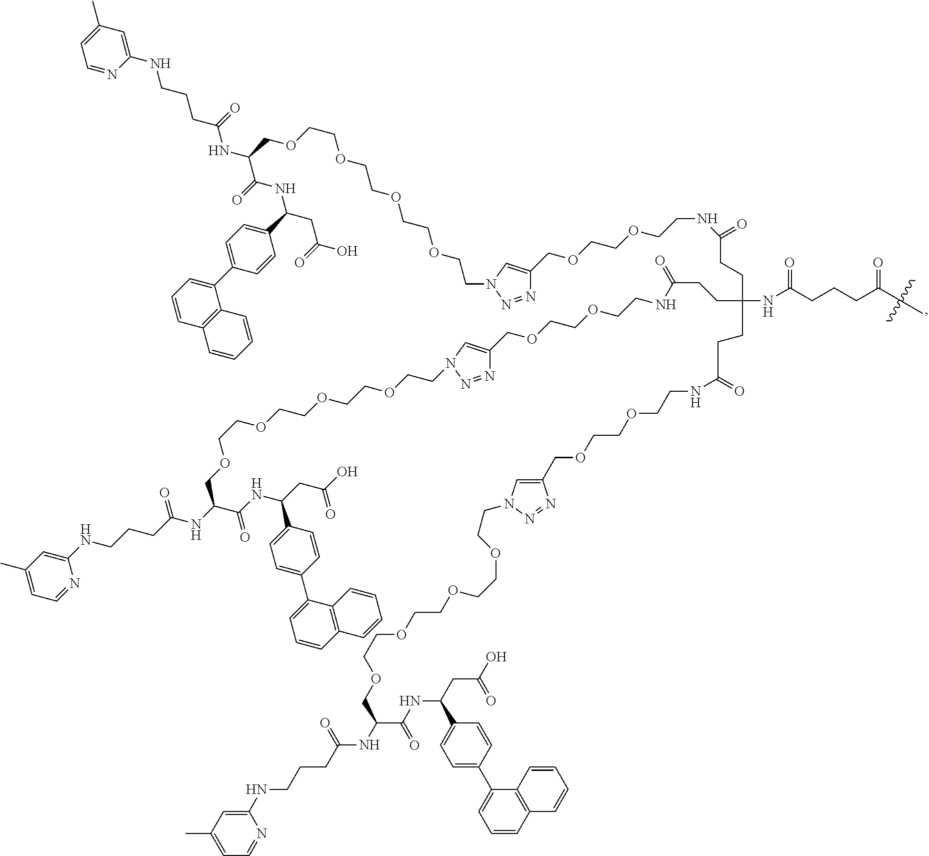 [SMILES: *C(=O)CCCC(=O)NC(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1]

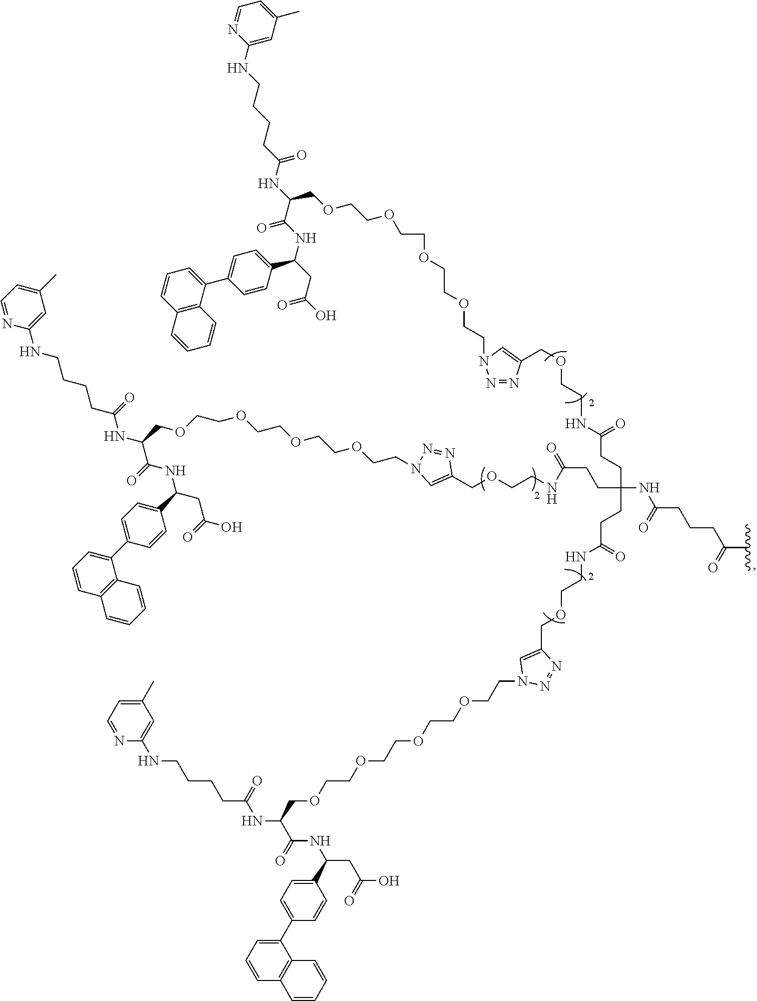

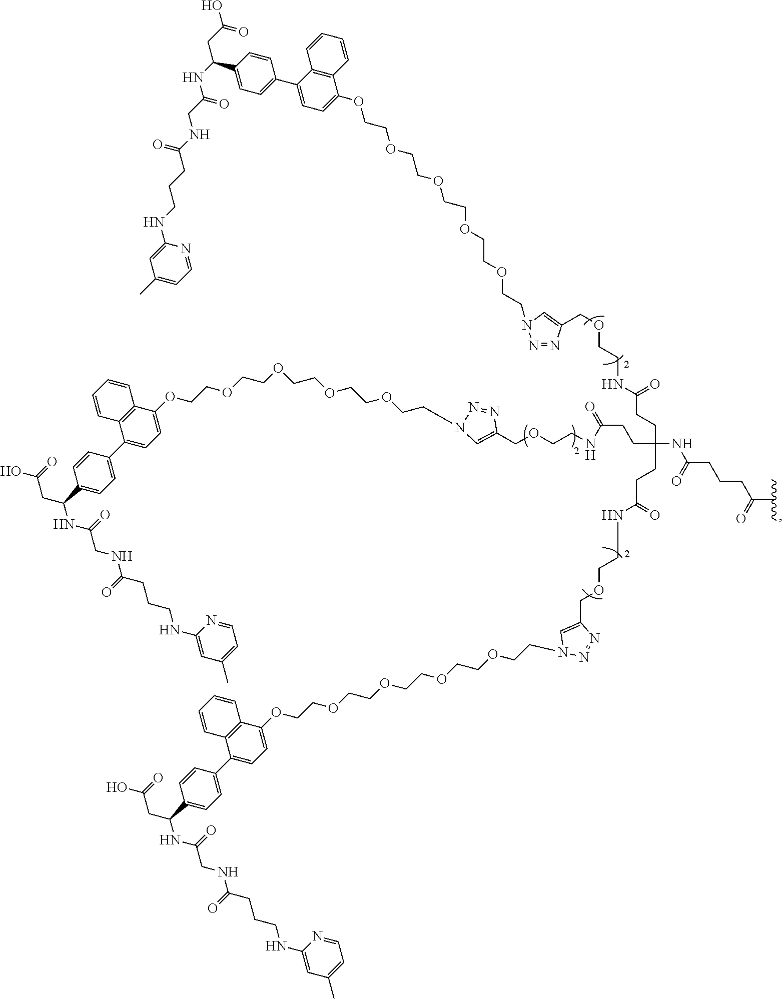

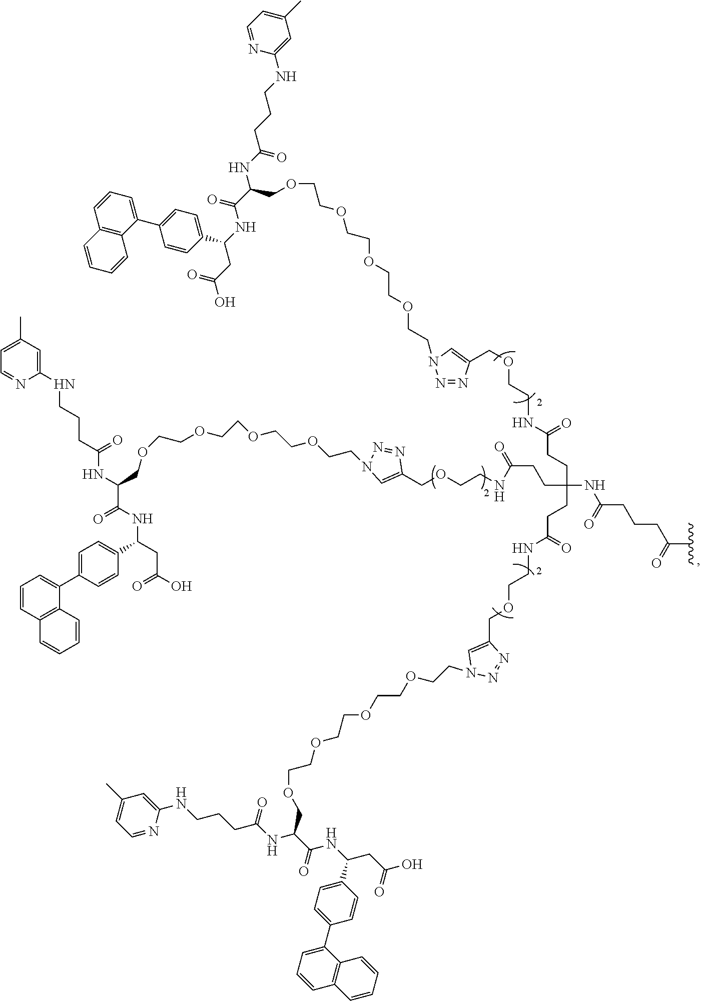

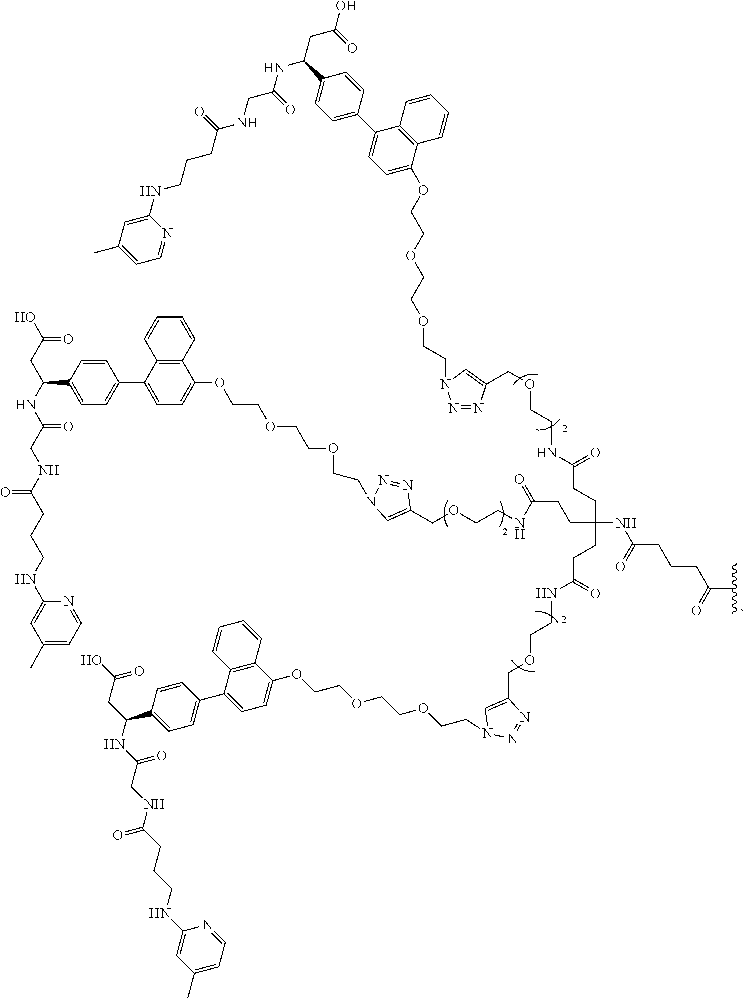

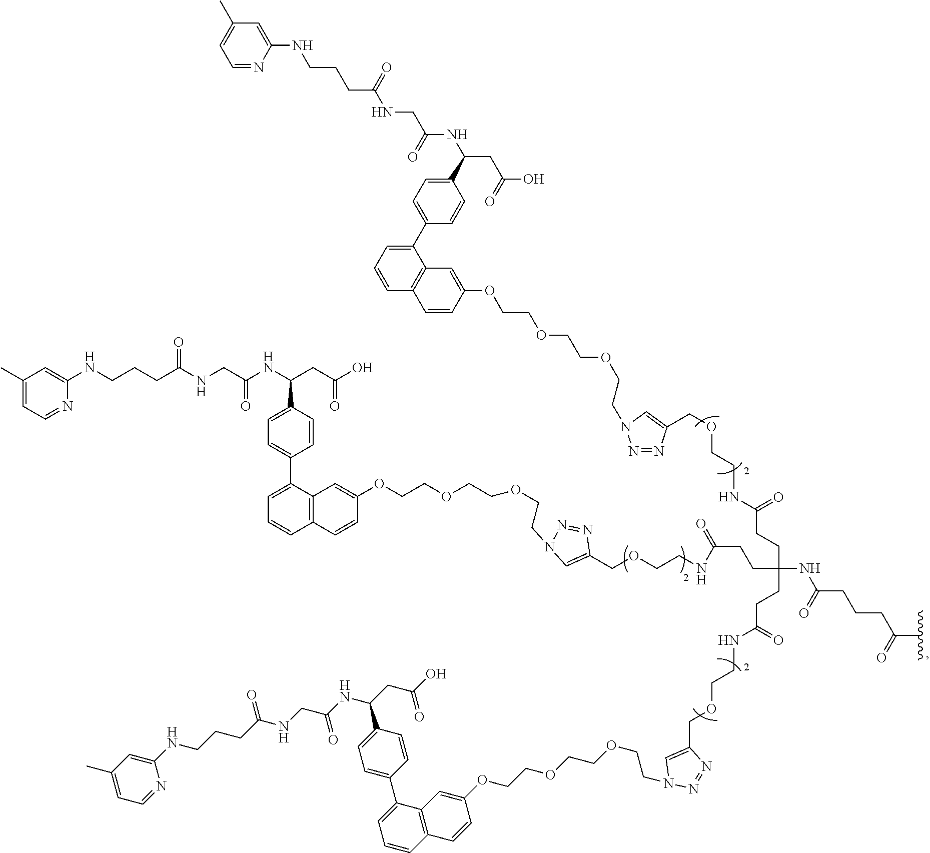

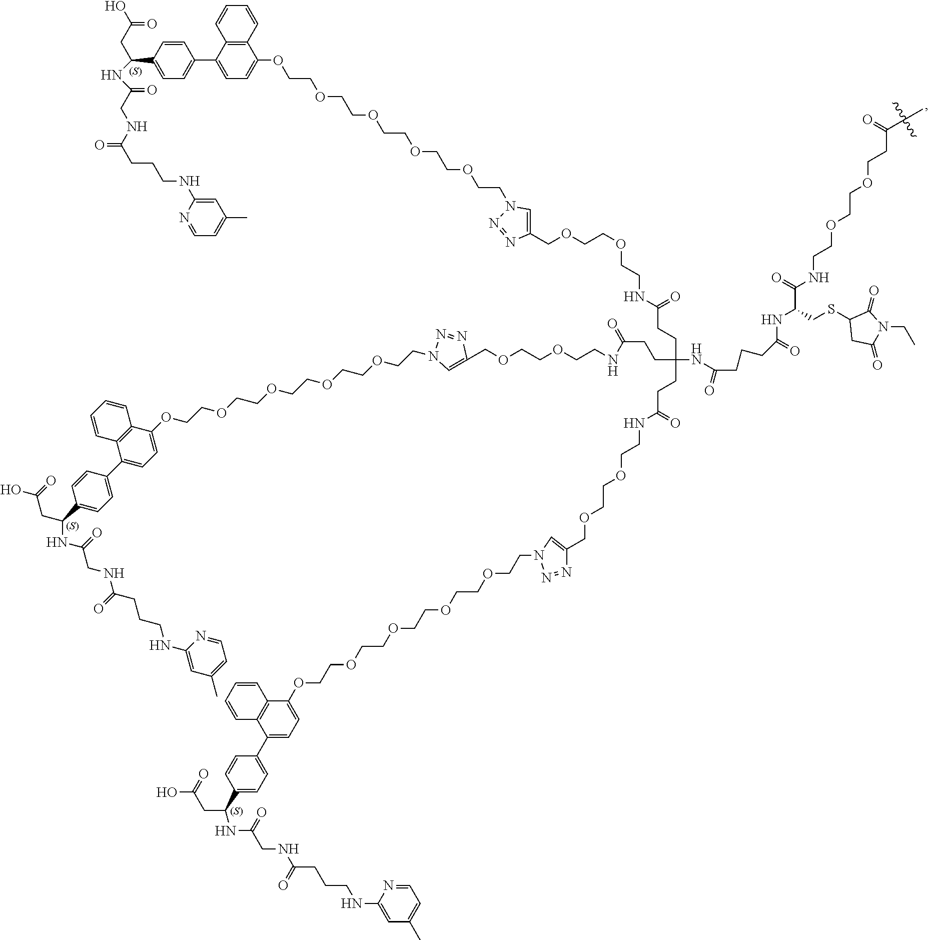

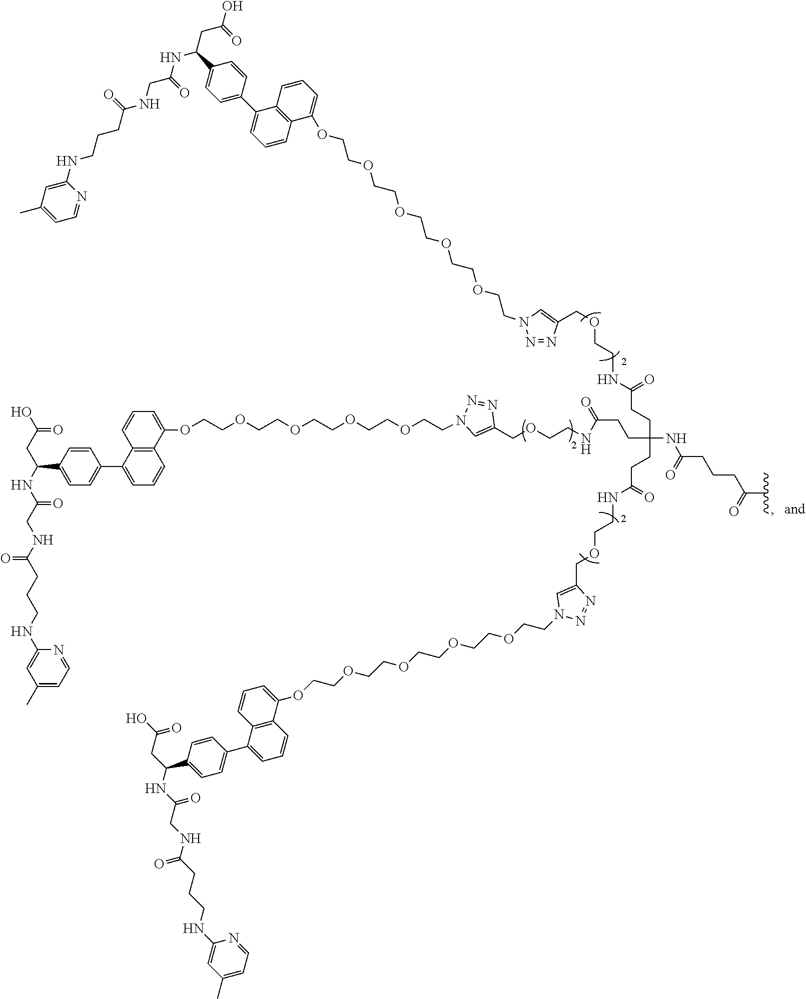

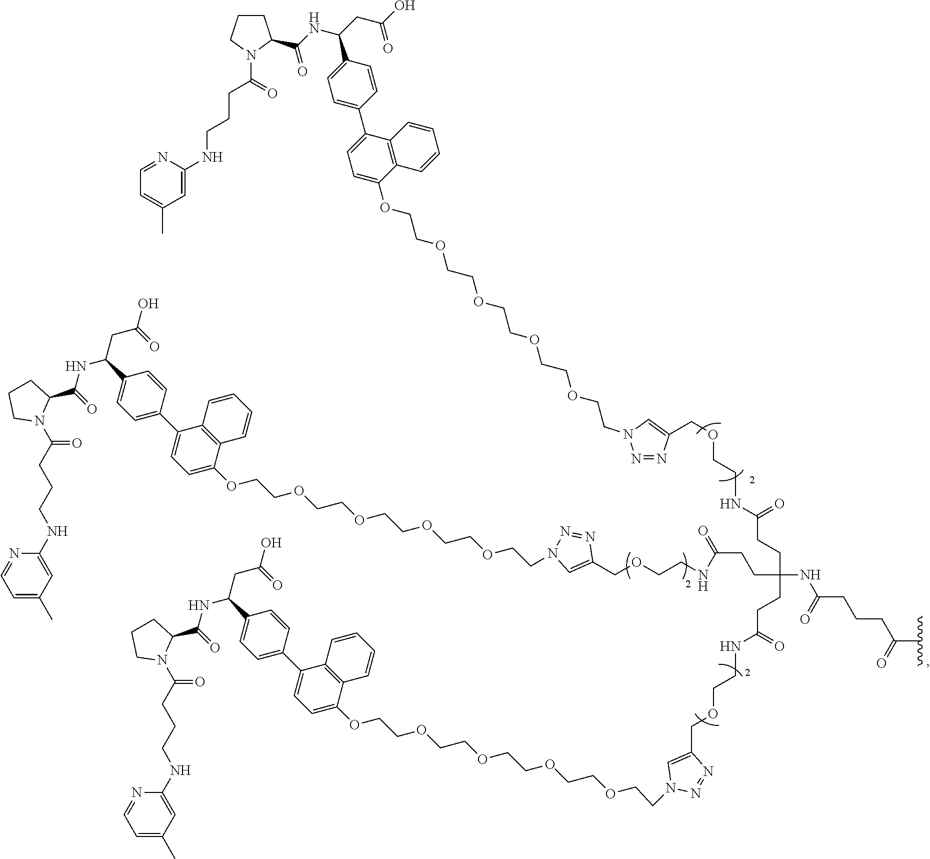

wherein [symbol] indicates the point of connection to the RNAi agent.

Embodiment 34. The RNAi agent of embodiment 33, wherein RNAi agent is conjugated to a targeting ligand having the following structure:

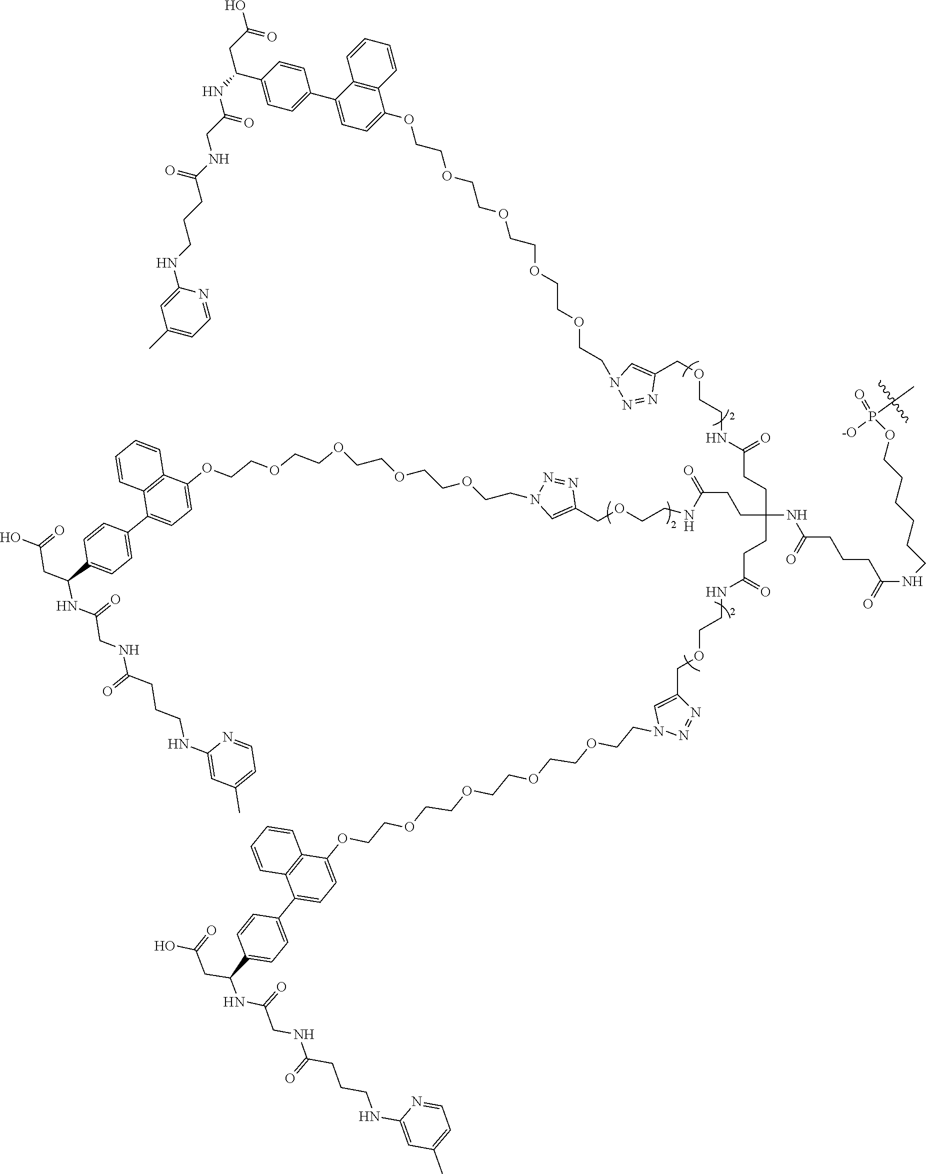

Embodiment 35. The RNAi agent of any one of embodiments 28-31, wherein the targeting ligand has the following structure:

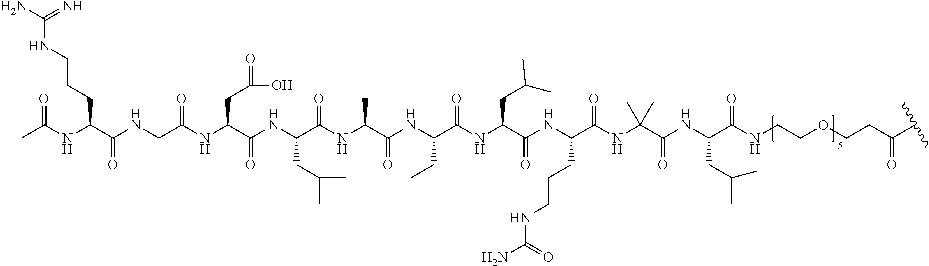

Embodiment 36. The RNAi agent of any one of embodiments 28-35, wherein the targeting ligand is conjugated to the sense strand.

Embodiment 37. The RNAi agent of embodiment 36, wherein the targeting ligand is conjugated to the 5′ terminal end of the sense strand.

Embodiment 38. A composition comprising the RNAi agent of any one of embodiments 1-37, wherein the composition further comprises a pharmaceutically acceptable excipient.

Embodiment 39. The composition of embodiment 38, further comprising a second RNAi agent capable of inhibiting the expression of a coronavirus (CoV) genome.

Embodiment 40. The composition of any one of embodiments 38-39, further comprising one or more additional therapeutics.

Embodiment 41. The composition of any one of embodiments 38-40, wherein the composition is formulated for administration by inhalation.

Embodiment 42. The composition of embodiment 41, wherein the composition is delivered by a metered-dose inhaler, jet nebulizer, vibrating mesh nebulizer, or soft mist inhaler.

Embodiment 43. The composition of any of embodiments 38-42, wherein the RNAi agent is a sodium salt.

Embodiment 44. The composition of any of embodiments 38-43, wherein the pharmaceutically acceptable excipient is water for injection.

Embodiment 45. The composition of any of embodiments 38-43, wherein the pharmaceutically acceptable excipient is a buffered saline solution.

Embodiment 46. A method for inhibiting a coronavirus (CoV) genome in a cell, the method comprising introducing into a cell an effective amount of an RNAi agent of any one of embodiments 1-37 or the composition of any one of embodiments 38-45.

Embodiment 47. The method of embodiment 46, wherein the cell is within a subject.

Embodiment 48. The method of embodiment 47, wherein the subject is a human subject.

Embodiment 49. The method of any one of embodiments 46-48, wherein following the administration of the RNAi agent the CoV genome expression is inhibited by at least about 30%.

Embodiment 50. A method of treating one or more symptoms or diseases associated with coronavirus (CoV) infection, the method comprising administering to a human subject in need thereof a therapeutically effective amount of the composition of any one of embodiments 38-45.

Embodiment 51. The method of embodiment 50, wherein the diseases is a respiratory disease.

Embodiment 52. The method of embodiment 51, wherein the respiratory disease is pulmonary inflammation.

Embodiment 53. The method of embodiment 51, wherein the respiratory disease is COVID-19.

Embodiment 54. The method of embodiment 50, wherein the symptoms are caused by SARS-CoV-2 viral infection.

Embodiment 55. The method of any one of embodiments 46-54, wherein the RNAi agent is administered at a deposited dose of about 0.01 mg/kg to about 5.0 mg/kg of body weight of the subject.

Embodiment 56. The method of any one of embodiments 46-55, wherein the RNAi agent is administered at a deposited dose of about 0.03 mg/kg to about 2.0 mg/kg of body weight of the subject.

Embodiment 57. The method of any one of claims 46-56, wherein the RNAi agent is administered in two or more doses.

Embodiment 58. Use of the RNAi agent of any one of embodiments 1-37, for the treatment of a disease, disorder, or symptom that is caused by coronavirus (CoV) infection, preferably wherein the disease, disorder, or symptom can be mediated at least in part by a reduction in SARS-CoV-2 activity and/or SARS-CoV-2 viral genome expression.

Embodiment 59. Use of the composition according to any one of embodiments 38-45, for the treatment of a disease, disorder, or symptom that is caused by coronavirus (CoV) infection, preferably wherein the disease, disorder, or symptom can be mediated at least in part by a reduction in SARS-CoV-2 activity and/or SARS-CoV-2 viral genome expression.

Embodiment 60. Use of the composition according to any one of embodiments 38-45, for the manufacture of a medicament for the treatment of a disease, disorder, or symptom that is caused by coronavirus (CoV) infection, preferably wherein the disease, disorder, or symptom can be mediated at least in part by a reduction in SARS-CoV-2 activity and/or SARS-CoV-2 viral genome expression.

Embodiment 61. The use of any one of embodiments 58-60, wherein the disease is pulmonary inflammation.

Embodiment 62. A method of making an RNAi agent of any one of embodiments 1-37, comprising annealing a sense strand and an antisense strand to form a double-stranded ribonucleic acid molecule.

Embodiment 63. The method of embodiment 62, wherein the sense strand comprises a targeting ligand.

Embodiment 64. The method of embodiment 63, comprising conjugating a targeting ligand to the sense strand.

The above provided embodiments and items are now illustrated with the following, non-limiting examples.

### EXAMPLES

### Example 1. Synthesis of CoV RNAi Agents

CoV RNAi agent duplexes disclosed herein were synthesized in accordance with the following:

A. Synthesis. The sense and antisense strands of the CoV RNAi agents were synthesized according to phosphoramidite technology on solid phase used in oligonucleotide synthesis. Depending on the scale, a MerMade96E® (Bioautomation), a MerMade12® (Bioautomation), or an OP Pilot 100 (GE Healthcare) was used. Syntheses were performed on a solid support made of controlled pore glass (CPG, 500 Å or 600 Å, obtained from Prime Synthesis, Aston, PA, USA). All RNA and 2′-modified RNA phosphoramidites were purchased from Thermo Fisher Scientific (Milwaukee, WI, USA). Specifically, the 2′-O-methyl phosphoramidites that were used included the following: (5′-O-dimethoxytrityl-N⁶-(benzoyl)-2′-O-methyl-adenosine-3′-O-(2-cyanoethyl-N,N-diisopropylamino) phosphoramidite, 5′-O-dimethoxy-trityl-N⁴-(acetyl)-2′-O-methyl-cytidine-3′-O-(2-cyanoethyl-N,N-diisopropyl-amino) phosphoramidite, (5′-O-dimethoxytrityl-N²-(isobutyryl)-2′-O-methyl-guanosine-3′-O-(2-cyanoethyl-N,N-diisopropylamino) phosphoramidite, and 5′-O-dimethoxytrityl-2′-O-methyl-uridine-3′-O-(2-cyanoethyl-N,N-diisopropylamino) phosphoramidite. The 2′-deoxy-2′-fluoro-phosphoramidites carried the same protecting groups as the 2′-O-methyl RNA amidites. 5′-dimethoxytrityl-2′-O-methyl-inosine-3′-O-(2-cyanoethyl-N,N-diisopropylamino) phosphoramidites were purchased from Glen Research (Virginia). The inverted abasic (3′-O-dimethoxytrityl-2′-deoxyribose-5′-O-(2-cyanoethyl-N,N-diisopropylamino) phosphoramidites were purchased from ChemGenes (Wilmington, MA, USA). The following UNA phosphoramidites were used: 5′-(4,4′-Dimethoxytrityl)-N6-(benzoyl)-2′,3′-seco-adenosine, 2′-benzoyl-3′-[(2-cyanoethyl)-(N,N-diisopropyl)]-phosphoramidite, 5′-(4,4′-Dimethoxytrityl)-N-acetyl-2′,3′-seco-cytosine, 2′-benzoyl-3′-[(2-cyanoethyl)-(N,N-diiso-propyl)]-phosphoramidite, 5′-(4,4′-Dimethoxytrityl)-N-isobutyryl-2′,3′-seco-guanosine, 2′-benzoyl-3′-[(2-cyanoethyl)-(N,N-diisopropyl)]-phosphoramidite, and 5′-(4,4′-Dimethoxy-trityl)-2′,3′-seco-uridine, 2′-benzoyl-3′-[(2-cyanoethyl)-(N,N-diiso-propyl)]-phosphoramidite. TFA aminolink phosphoramidites were also commercially purchased (ThermoFisher). Linker L6 was purchased as propargyl-PEG5-NHS from BroadPharm (catalog #BP-20907) and coupled to the NH2-C6 group from an aminolink phosphoramidite to form -L6-C6-, using standard coupling conditions. The linker Alk-cyHex was similarly commercially purchased from Lumiprobe (alkyne phosphoramidite, 5′-terminal) as a propargyl-containing compound phosphoramidite compound to form the linker -Alk-cyHex-. In each case, phosphorothioate linkages were introduced as specified using the conditions set forth herein. The cyclopropyl phosphonate phosphoramidites were synthesized in accordance with International Patent Application Publication No. WO 2017/214112 (see also Altenhofer et. al., Chem. Communications (Royal Soc. Chem.), 57(55):6808-6811 (July 2021)). The (NAG37)s targeting ligand phosphoramidite compounds used in synthesizing the RNAi agents disclosed herein for performing certain SEAP studies described below were synthesized in accordance with International Patent Application Publication No. WO 2018/044350 to Arrowhead Pharmaceuticals, Inc.; the targeting ligand-containing phosphoramidite compounds were added during the solid phase oligonucleotide synthesis process described herein.

Tri-alkyne-containing phosphoramidites were dissolved in anhydrous dichloromethane or anhydrous acetonitrile (50 mM), while all other amidites were dissolved in anhydrous acetonitrile (50 mM) and molecular sieves (3 Å) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

Alternatively, tri-alkyne moieties were introduced post-synthetically (see section E, below). For this route, the sense strand was functionalized with a 5′ and/or 3′ terminal nucleotide containing a primary amine. TFA aminolink phosphoramidite was dissolved in anhydrous acetonitrile (50 mM) and molecular sieves (3A) were added. 5-Benzylthio-1H-tetrazole (BTT, 250 mM in acetonitrile) or 5-Ethylthio-1H-tetrazole (ETT, 250 mM in acetonitrile) was used as activator solution. Coupling times were 10 minutes (RNA), 90 seconds (2′ O-Me), and 60 seconds (2′ F). In order to introduce phosphorothioate linkages, a 100 mM solution of 3-phenyl 1,2,4-dithiazoline-5-one (POS, obtained from PolyOrg, Inc., Leominster, MA, USA) in anhydrous acetonitrile was employed.

B. Cleavage and deprotection of support bound oligomer. After finalization of the solid phase synthesis, the dried solid support was treated with a 1:1 volume solution of 40 wt. % methylamine in water and 28% to 31% ammonium hydroxide solution (Aldrich) for 1.5 hours at 30° C. The solution was evaporated and the solid residue was reconstituted in water (see below).

C. Purification. Crude oligomers were purified by anionic exchange HPLC using a TSKgel SuperQ-5PW 13 μm column and Shimadzu LC-8 system. Buffer A was 20 mM Tris, 5 mM EDTA, pH 9.0 and contained 20% Acetonitrile and buffer B was the same as buffer A with the addition of 1.5 M sodium chloride. UV traces at 260 nm were recorded. Appropriate fractions were pooled then run on size exclusion IIPLC using a GE Healthcare XK 16/40 column packed with Sephadex G-25 fine with a running buffer of 100 mM ammonium bicarbonate, pH 6.7 and 20% Acetonitrile or filtered water. Alternatively, pooled fractions were desalted and exchanged into an appropriate buffer or solvent system via tangential flow filtration.

D. Annealing. Complementary strands were mixed by combining equimolar RNA solutions (sense and antisense) in 1×PBS (Phosphate-Buffered Saline, 1×, Corning, Cellgro) to form the RNAi agents. Some RNAi agents were lyophilized and stored at −15 to −25° C. Duplex concentration was determined by measuring the solution absorbance on a UV-Vis spectrometer in 1×PBS. The solution absorbance at 260 nm was then multiplied by a conversion factor (0.050 mg/(mL·cm)) and the dilution factor to determine the duplex concentration.

E. Conjugation of Tri-alkyne linker. In some embodiments a tri-alkyne linker is conjugated to the sense strand of the RNAi agent on resin as a phosphoramidite (see Example 1G for the synthesis of an example tri-alkyne linker phosphoramidite and Example TA for the conjugation of the phosphoramidite.). In other embodiments, a tri-alkyne linker may be conjugated to the sense strand following cleavage from the resin, described as follows: either prior to or after annealing, in some embodiments, the 5′ or 3′ amine functionalized sense strand is conjugated to a tri-alkyne linker. An example tri-alkyne linker structure that can be used in forming the constructs disclosed herein is as follows:

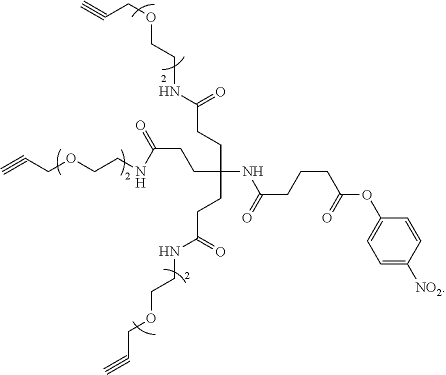

To conjugate the tri-alkyne linker to the annealed duplex, amine-functionalized duplex was dissolved in 90% DMSO/10% H₂O, at ˜50-70 mg/mL. 40 equivalents triethylamine was added, followed by 3 equivalents tri-alkyne-PNP. Once complete, the conjugate was precipitated twice in a solvent system of 1× phosphate buffered saline/acetonitrile (1:14 ratio), and dried.

F. Synthesis of Targeting Ligand SM6.1

### ((S)-3-(4-(4-((14-azido-3,6,9,12-tetraoxatetradecyl)oxy)naphthalen-1-yl)phenyl)-3-(2-(4-((4-methylpyridin-2-yl)amino)butanamido)acetamido)propanoic acid)

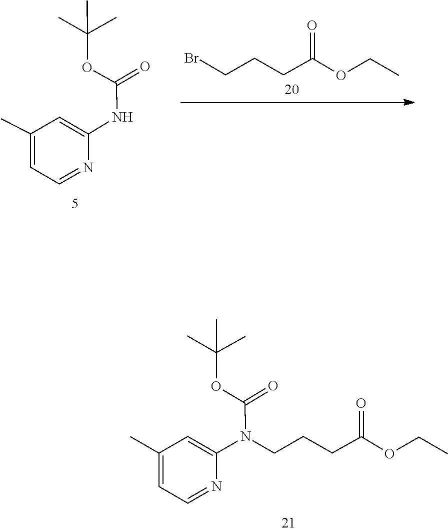

Compound 5 (tert-Butyl(4-methylpyridin-2-yl)carbamate) (0.501 g, 2.406 mmol, 1 equiv.) was dissolved in DMF (17 mL). To the mixture was added NaH (0.116 mg, 3.01 mmol, 1.25 eq, 60% dispersion in oil) The mixture stirred for 10 min before adding Compound 20 (Ethyl 4-Bromobutyrate (0.745 g, 3.82 mmol, 0.547 mL)) (Sigma 167118). After 3 hours the reaction was quenched with ethanol (18 mL) and concentrated. The concentrate was dissolved in DCM (50 mL) and washed with saturated aq. NaCl solution (1×50 mL), dried over Na₂SO₄, filtered and concentrated. The product was purified on silica column, gradient 0-5% Methanol in DCM.

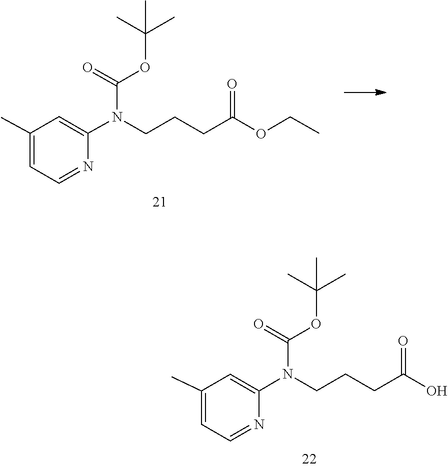

Compound 21 was dissolved (0.80 g, 2.378 mmol) in 100 mL of Acetone: 0.1 M NaOH [1:1]. The reaction was monitored by TLC (5% ethyl acetate in hexane). The organics were concentrated away, and the residue was acidified to pH 3-4 with 0.3 M Citric Acid (40 mL). The product was extracted with DCM (3×75 mL). The organics were pooled, dried over Na₂SO₄, filtered and concentrated. The product was used without further purification.

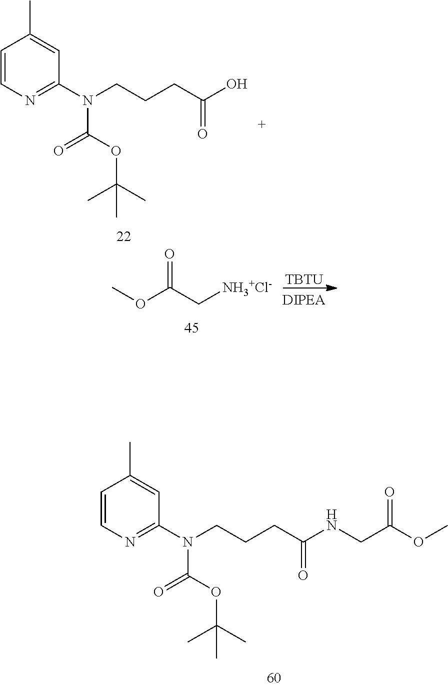

To a solution of Compound 22 (1.1 g, 3.95 mmol, 1 equiv.), Compound 45 (595 mg, 4.74 mmol, 1.2 equiv.), and TBTU (1.52 g, 4.74 mmol, 1.2 equiv.) in anhydrous DMF (10 mL) was added diisopropylethylamine (2.06 mL, 11.85 mmol, 3 equiv.) at 0° C. The reaction mixture was warmed to room temperature and stirred 3 hours. The reaction was quenched by saturated NaHCO₃ solution (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase. LC-MS: calculated [M+H]+ 366.20, found 367.

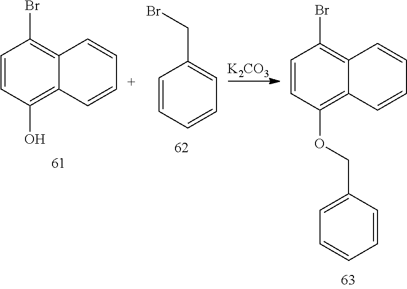

To a solution of compound 61 (2 g, 8.96 mmol, 1 equiv.), and compound 62 (2.13 mL, 17.93 mmol, 2 equiv.) in anhydrous DMF (10 mL) was added K₂CO₃ (2.48 g, 17.93 mmol, 2 equiv.) at 0° C. The reaction mixture was warmed to room temperature and stirred overnight. The reaction was quenched by water (10 mL). The aqueous phase was extracted with ethyl acetate (3×10 mL) and the organic phase was combined, dried over anhydrous Na₂SO₄, and concentrated. The product was separated by CombiFlash® using silica gel as the stationary phase.

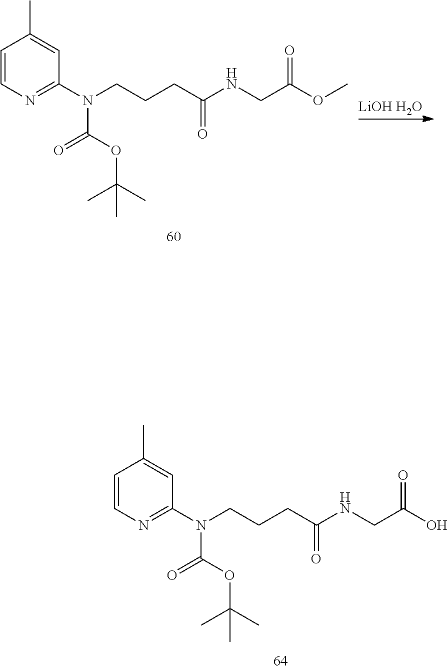

To a solution of compound 60 (1.77 g, 4.84 mmol, 1 equiv.) in THF (5 mL) and H₂O (5 mL) was added lithium hydroxide monohydrate (0.61 g, 14.53 mmol, 3 equiv.) portion-wise at 0° C. The reaction mixture was warmed to room temperature. After stirring at room temperature for 3 hours, the reaction mixture was acidified by HCl (6 N) to pH 3.0. The aqueous phase was extracted with ethyl acetate (3×20 mL) and the organic layer was combined, dried over Na₂SO₄, and concentrated. LC-MS: calculated [M+H]+ 352.18, found 352.

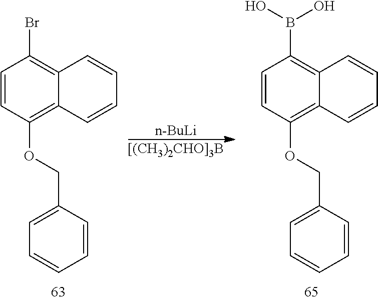

To a solution of compound 63 (1.88 g, 6.0 mmol, 1.0 equiv.) in anhydrous THF (20 mL) was added n-BuLi in hexane (3.6 mL, 9.0 mmol, 1.5 equiv.) drop-wise at −78° C. The reaction was kept at −78° C. for another 1 hour. Triisopropylborate (2.08 mL, 9.0 mmol, 1.5 equiv.) was then added into the mixture at −78° C. The reaction was then warmed up to room temperature and stirred for another 1 hour. The reaction was quenched by saturated NH4Cl solution (20 mL) and the pH was adjusted to 3. The aqueous phase was extracted with EtOAc (3×20 mL) and the organic phase was combined, dried over Na₂SO₄, and concentrated.

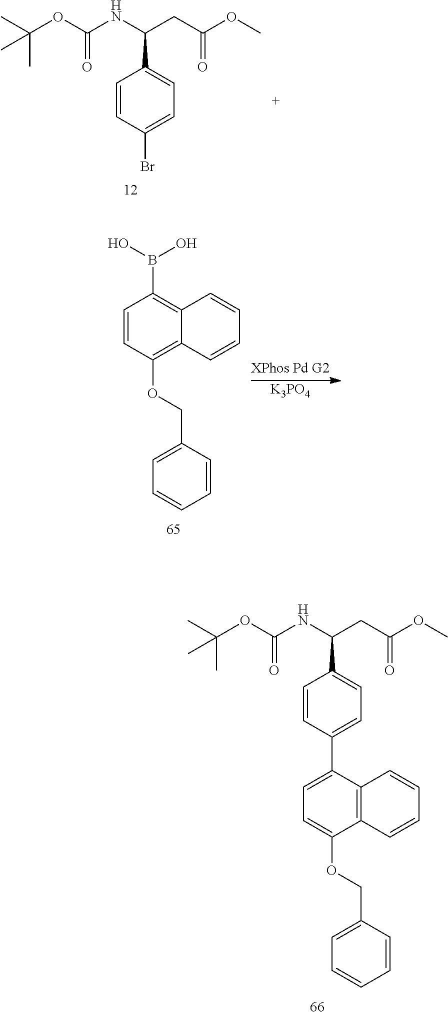

Compound 12 (300 mg, 0.837 mmol, 1.0 equiv.), Compound 65 (349 mg, 1.256 mmol, 1.5 equiv.), XPhos Pd G2 (13 mg, 0.0167 mmol, 0.02 equiv.), and K₃PO₄ (355 mg, 1.675 mmol, 2.0 equiv.) were mixed in a round-bottom flask. The flask was sealed with a screw-cap septum, and then evacuated and backfilled with nitrogen (this process was repeated a total of 3 times). Then, THF (8 mL) and water (2 mL) were added via syringe. The mixture was bubbled with nitrogen for 20 min and the reaction was kept at room temperature for overnight. The reaction was quenched with water (10 mL), and the aqueous phase was extracted with ethyl acetate (3×10 mL). The organic phase was dried over Na₂SO₄, concentrated, and purified via CombiFlash® using silica gel as the stationary phase and was eluted with 15% EtOAc in hexane. LC-MS: calculated [M+H]+ 512.24, found 512.56.

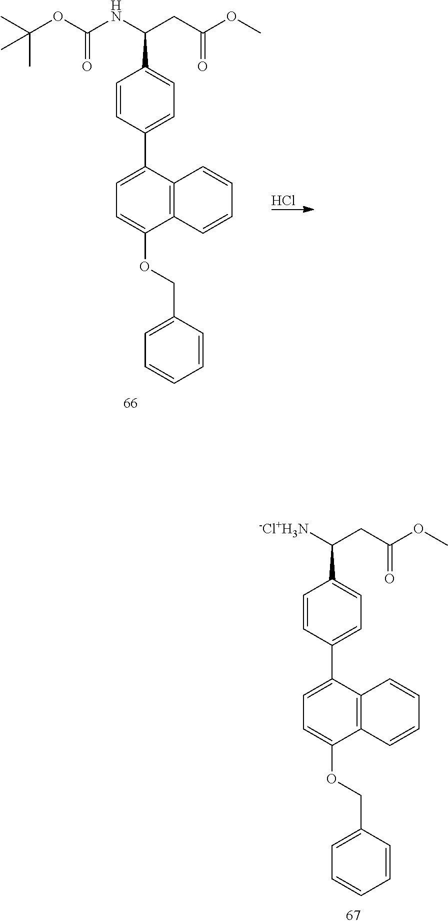

Compound 66 (858 mg, 1.677 mmol, 1.0 equiv.) was cooled by ice bath. HCl in dioxane (8.4 mL, 33.54 mmol, 20 equiv.) was added into the flask. The reaction was warmed to room temperature and stirred for another 1 hr. The solvent was removed by rotary evaporator and the product was directly used without further purification. LC-MS: calculated [M+H]+ 412.18, found 412.46.

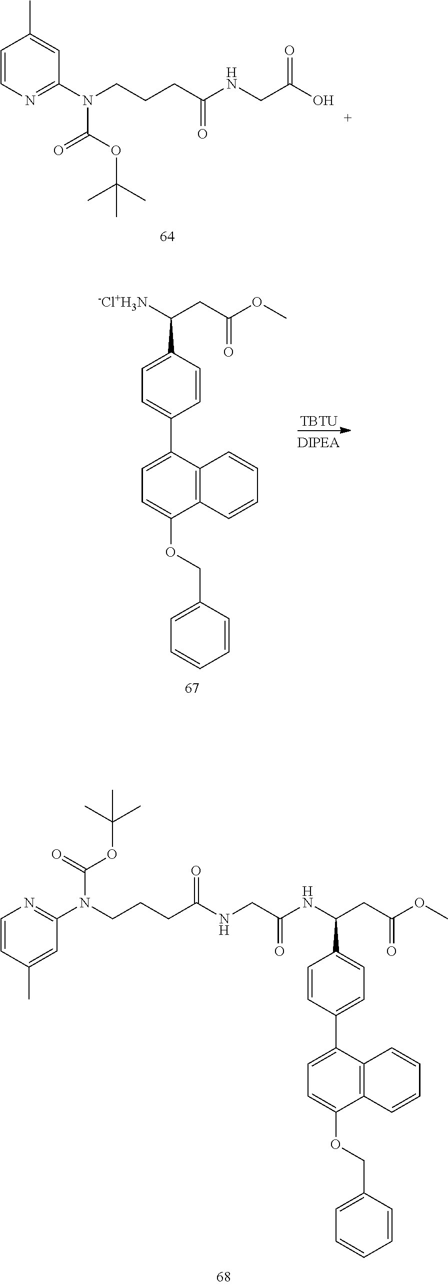

To a solution of compound 64 (500 mg, 1.423 mmol, 1 equiv.), compound 67 (669 mg, 1.494 mmol, 1.05 equiv.), and TBTU (548 mg, 0.492 mmol, 1.2 equiv.) in anhydrous DMF (15 mL) was added diisopropylethylamine (0.744 mL, 4.268 mmol, 3 equiv.) at 0° C. The reaction mixture was warmed to room temperature and stirred for another 1 hr. The reaction was quenched by saturated NaHCO₃ aqueous solution (10 mL) and the product was extracted with ethyl acetate (3×20 mL). The organic phase was combined, dried over Na₂SO₄, and concentrated. The product was purified by CombiFlash® using silica gel as the stationary phase and was eluted with 3-4% methanol in DCM. The yield was 96.23%. LC-MS: calculated [M+H]+ 745.35, found 746.08.

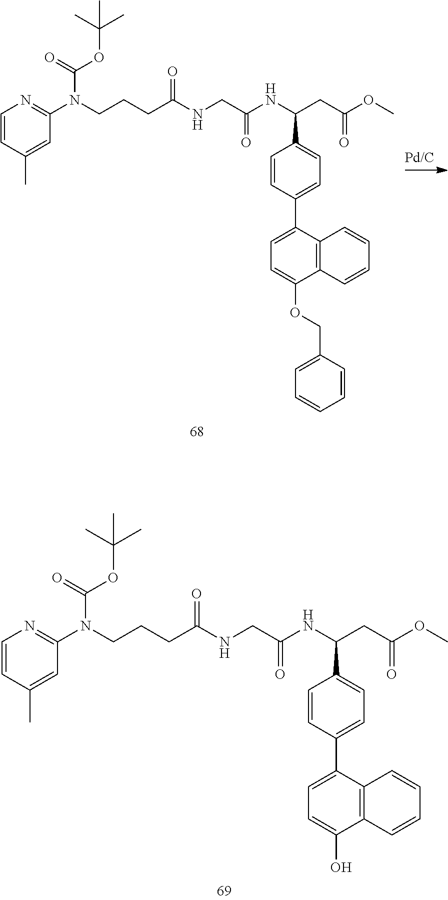 [SMILES: COC(=O)C[C@H](NC(=O)CNC(=O)CCCN(C(=O)OC(C)(C)C)c1cc(C)ccn1)c1ccc(-c2ccc(OCc3ccccc3)c3ccccc23)cc1 ; COC(=O)C[C@H](NC(=O)CNC(=O)CCCN(C(=O)OC(C)(C)C)c1cc(C)ccn1)c1ccc(-c2ccc(O)c3ccccc23)cc1]

To a solution of compound 68 (1.02 g, 1.369 mmol, 1 equiv.) in ethyl acetate (10 mL) was added 100% Pd/C (0.15 g, 50% H₂O) at room temperature. The reaction mixture was warmed to room temperature and the reaction was monitored by LC-MS. The reaction was kept at room temperature overnight. The solids were filtered through Celite® and the solvent was removed by rotary evaporator. The product was directly used without further purification. LC-MS: [M+H]+ 655.31, found 655.87.

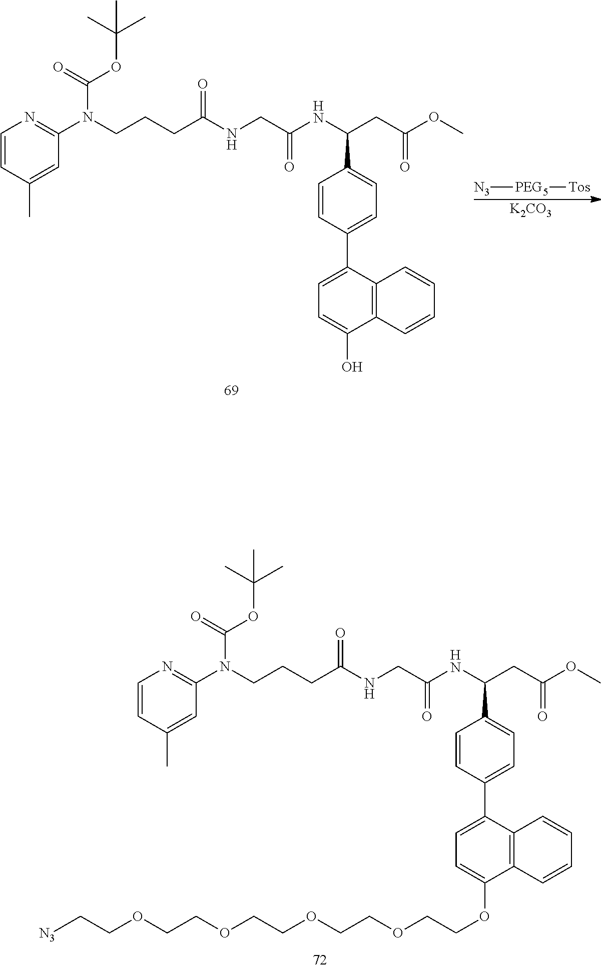

To a solution of compound 69 (100 mg, 0.152 mmol, 1 equiv.) and azido-PEG₅-OTs (128 mg, 0.305 mmol, 2 equiv.) in anhydrous DMF (2 mL) was added K₂CO₃ (42 mg, 0.305 mmol, 2 equiv.) at 0° C. The reaction mixture was stirred for 6 hours at 80° C. The reaction was quenched by saturated NaHCO₃ solution and the aqueous layer was extracted with ethyl acetate (3×10 mL). The organic phase was combined, dried over Na₂SO₄, and concentrated. LC-MS: calculated [M+H]+ 900.40, found 901.46.

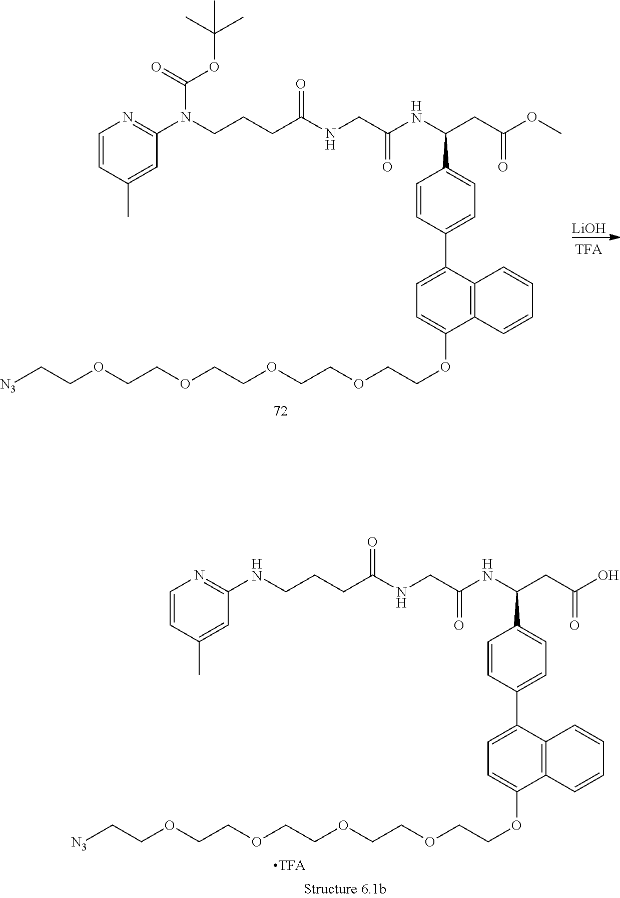 [SMILES: COC(=O)C[C@H](NC(=O)CNC(=O)CCCN(C(=O)OC(C)(C)C)c1cc(C)ccn1)c1ccc(-c2ccc(OCCOCCOCCOCCOCCN=[N+]=[N-])c3ccccc23)cc1 ; "LiOH" ; Cc1ccnc(NCCCC(=O)NCC(=O)N[C@@H](CC(=O)O)c2ccc(-c3ccc(OCCOCCOCCOCCOCCN=[N+]=[N-])c4ccccc34)cc2)c1]

To a solution of compound 72 (59 mg, 0.0656 mmol, 1.0 equiv.) in THF (2 mL) and water (2 mL) was added lithium hydroxide (5 mg, 0.197 mmol, 3.0 equiv.) at room temperature. The mixture was stirred at room temperature for another 1 hr. The pH was adjusted to 3.0 by HCl (6N) and the aqueous phase was extracted with EtOAc (3×10 mL). The organic phase was combined, dried over Na₂SO₄, and concentrated. TFA (0.5 mL) and DCM (0.5 mL) was added into the residue and the mixture was stirred at room temperature for another 3 hr. The solvent was removed by rotary evaporator. LC-MS: calculated [M+H]+ 786.37, found 786.95.

G. Synthesis of TriAlk 14

TriAlk14 and (TriAlkl4)s as shown in Table 11, above, may be synthesized using the synthetic route shown below. Compound 14 may be added to the sense strand as a phosphoramidite using standard oligonucleotide synthesis techniques, or compound 22 may be conjugated to the sense strand comprising an amine in an amide coupling reaction.

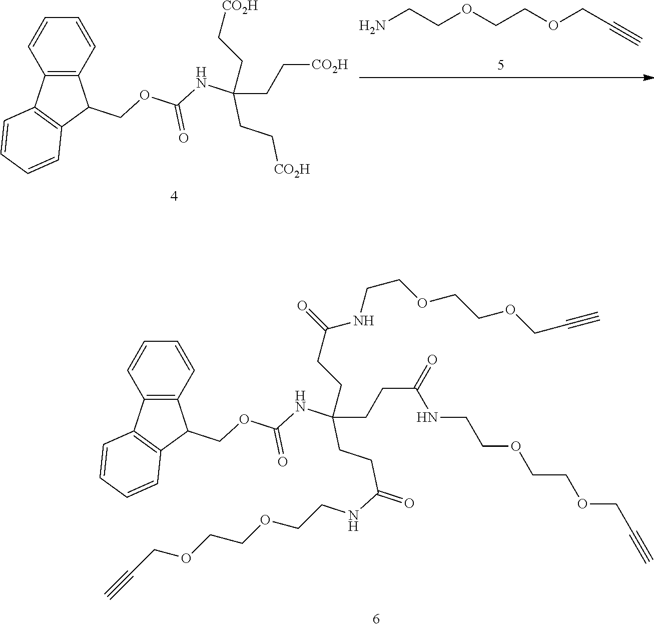

To a 3-L jacketed reactor was added 500 mL DCM and 4 (75.0 g, 0.16 mol). The internal temperature of the reaction was cooled to 0° C. and TBTU (170.0 g, 0.53 mol) was added. The suspension was then treated with the amine 5 (75.5 g, 0.53 mol) dropwise keeping the internal temperature less than 5° C. The reaction was then treated with DIPEA (72.3 g, 0.56 mol) slowly, keeping the internal temperature less than 5° C. After the addition was complete, the reaction was warmed up to 23° C. over 1 hour, and allowed to stir for 3 hours. A 10% kicker charge of all three reagents were added and allowed to stir an additional 3 hours. The reaction was deemed complete when <1% of 4 remained. The reaction mixture was washed with saturated ammonium chloride solution (2×500 mL) and once with saturated sodium bicarbonate solution (500 mL). The organic layer was then dried over sodium sulfate and concentrated to an oil. The mass of the crude oil was 188 g which contained 72% 6 by QNMR. The crude oil was carried to the next step. Calculated mass for C₄₆H₆₀N₄O₁₁=845.0 m/z.

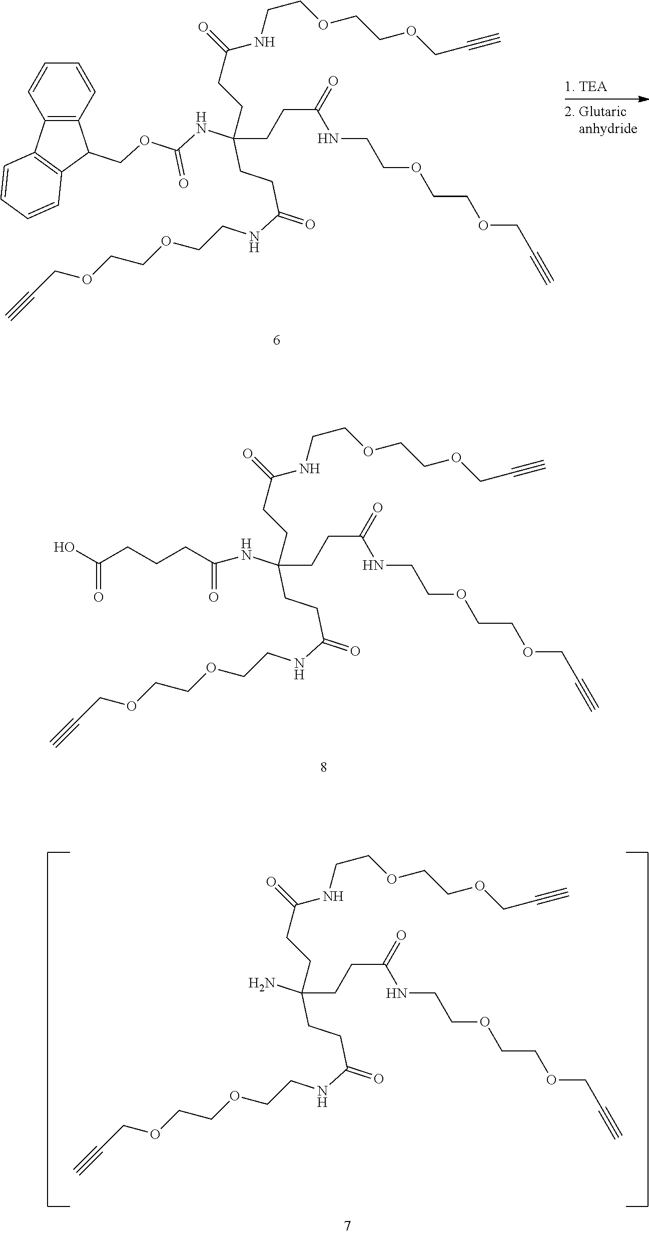

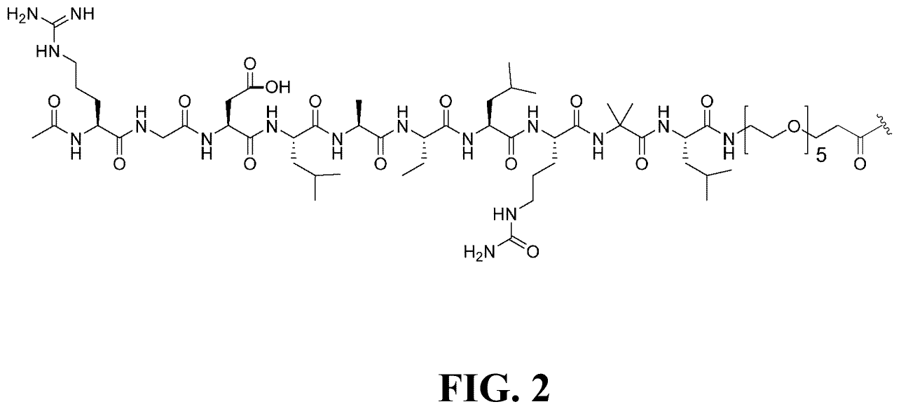

The 121.2 g of crude oil containing 72 wt % compound 6 (86.0 g, 0.10 mol) was dissolved in DMF (344 mL) and treated with TEA (86 mL, 20 v/v %), keeping the internal temperature below 23° C. The formation of dibenzofulvene (DBF) relative to the consumption of Fmoc-amine 6 was monitored via HPLC method 1 (FIG. 2) and the reaction was complete within 10 hours. To the solution was added glutaric anhydride (12.8 g, 0.11 mol) and the intermediate amine 7 was converted to compound 8 within 2 hours. Upon completion, the DMF and TEA were removed at 30° C. under reduced pressure resulting in 100 g of a crude oil. Due to the high solubility of compound 7 in water, an aqueous workup could not be used, and chromatography is the only way to remove DBF, TMU, and glutaric anhydride. The crude oil (75 g) was purified on a Teledyne ISCO Combi-Flash® purification system in three portions. The crude oil (25 g) was loaded onto a 330 g silica column and eluted from 0-20% methanol/DCM over 30 minutes resulting in 42 g of compound 8 (54% yield over 3 steps). Calculated mass for C₃₆H₅₅N₄O₁₂=736.4 m/z. Found [M+H]=737.0.

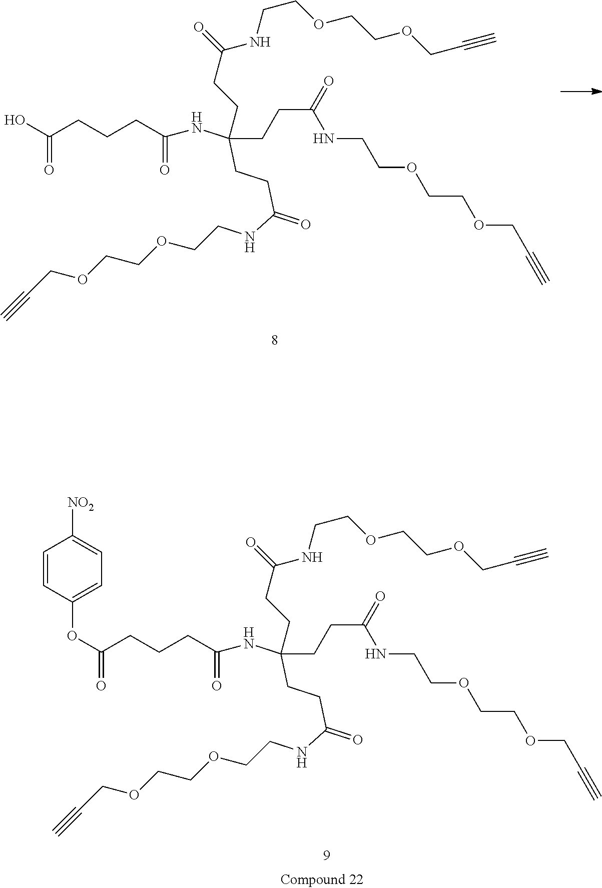 [SMILES: C#CCOCCOCCNC(=O)CCC(CCC(=O)NCCOCCOCC#C)(CCC(=O)NCCOCCOCC#C)NC(=O)CCCC(=O)O ; C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1]

Compound 8 (42.0 g, 0.057 mol) was co-stripped with 10 volumes of acetonitrile prior to use to remove any residual methanol from chromatography solvents. The oil was redissolved in DMF (210 mL) and cooled to 0° C. The solution was treated with 4-nitrophenol (8.7 g, 0.063 mL) followed by EDC-hydrochloride (12.0 g, 0.063 mol) and found to reach completion within 10 hours. The solution was cooled to 0° C. and 10 volumes ethyl acetate was added followed by 10 volumes saturated ammonium chloride solution, keeping the internal temperature below 15° C. The layers were allowed to separate and the ethyl acetate layer was washed with brine. The combined aqueous layers were extracted twice with 5 volumes ethyl acetate. The combined organic layers were dried over sodium sulfate and concentrated to an oil. The crude oil (55 g) was purified on a Teledyne ISCO Combi-Flash® purification system in three portions. The crude oil (25 g) was loaded onto a 330 g silica column and eluted from 0-10% methanol/DCM over 30 minutes resulting in 22 g of pure 9 (Compound 22) (50% yield). Calculated mass for C₄₂H₅₉N₅O₁₄=857.4 m/z. Found [M+H]=858.0.

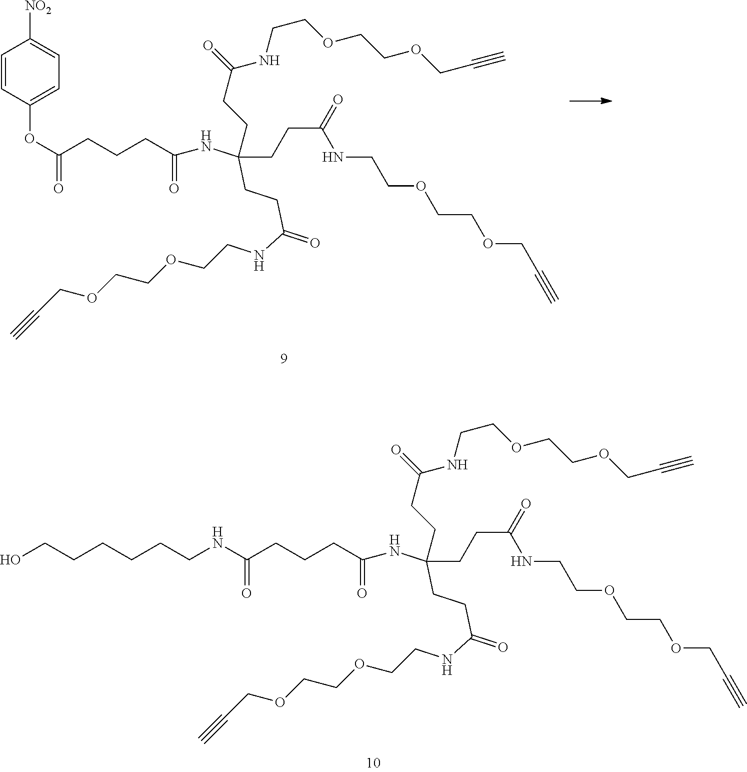 [SMILES: C#CCOCCOCCNC(=O)CCC(CCC(=O)NCCOCCOCC#C)(CCC(=O)NCCOCCOCC#C)NC(=O)CCCC(=O)Oc1ccc([N+](=O)[O-])cc1 ; C#CCOCCOCCNC(=O)CCC(CCC(=O)NCCOCCOCC#C)(CCC(=O)NCCOCCOCC#C)NC(=O)CCCC(=O)NCCCCCCO]

A solution of ester 9 (49.0 g, 57.1 mmol) and 6-amino-1-hexanol (7.36 g, 6.28 mmol) in dichloromethane (3 volumes) was treated with triethylamine (11.56 g, 111.4 mmol) dropwise. The reaction was monitored by observing the disappearance of compound 9 on HPLC Method 1 and was found to be complete in 10 minutes. The crude reaction mixture was diluted with 5 volumes dichloromethane and washed with saturated ammonium chloride (5 volumes) and brine (5 volumes). The organic layer was dried over sodium sulfate and concentrated to an oil. The crude oil was purified on a Teledyne ISCO Combi-Flash® purification system using a 330 g silica column. The 4-nitrophenol was eluted with 100% ethyl acetate and 10 was flushed from the column using 20% methanol/DCM resulting in a colorless oil (39 g, 81% yield). Calculated mass for C₄₂H₆₉N₅O₁₂=836.0 m/z. Found [M+H]=837.0.

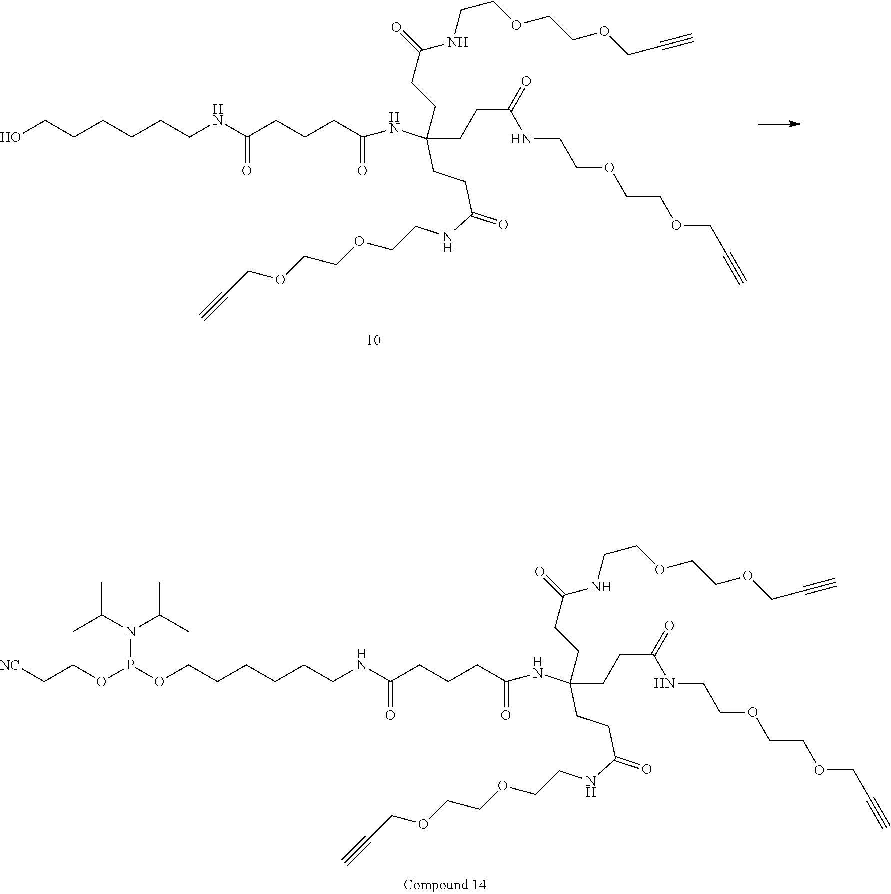 [SMILES: C#CCOCCOCCNC(=O)CCC(CCC(=O)NCCOCCOCC#C)(CCC(=O)NCCOCCOCC#C)NC(=O)CCCC(=O)NCCCCCCO ; C#CCOCCOCCNC(=O)CCC(CCC(=O)NCCOCCOCC#C)(CCC(=O)NCCOCCOCC#C)NC(=O)CCCC(=O)NCCCCCCOP(OCCC#N)N(C(C)C)C(C)C]

Alcohol 10 was co-stripped twice with 10 volumes of acetonitrile to remove any residual methanol from chromatography solvents and once more with dry dichloromethane (KF <60 ppm) to remove trace water. The alcohol 10 (2.30 g, 2.8 mmol) was dissolved in 5 volumes dry dichloromethane (KF<50 ppm) and treated with diisopropylammonium tetrazolide (188 mg, 1.1 mmol). The solution was cooled to 0° C. and treated with 2-cyanoethyl N,N,N′,N′-tetraisopropylphosphoramidite (1.00 g, 3.3 mmol) dropwise. The solution was removed from ice-bath and stirred at 20° C. The reaction was found to be complete within 3-6 hours. The reaction mixture was cooled to 0° C. and treated with 10 volumes of a 1:1 solution of saturated ammonium bicarbonate/brine and then warmed to ambient over 1 minute and allowed to stir an additional 3 minutes at 20° C. The biphasic mixture was transferred to a separatory funnel and 10 volumes of dichloromethane was added. The organic layer was separated and washed with 10 volumes of saturated sodium bicarbonate solution to hydrolyze unreacted bis-phosphorous reagent. The organic layer was dried over sodium sulfate and concentrated to an oil resulting in 3.08 g of 94 wt % Compound 14. Calculated mass for C₅₁H₈₆N₇O₁₃P=1035.6 m/z. Found [M+H]=1036.

H. Conjugation of Targeting Ligands. Either prior to or after annealing, the 5′ or 3′ tridentate alkyne functionalized sense strand is conjugated to targeting ligands. The following example describes the conjugation of targeting ligands to the annealed duplex: Stock solutions of 0.5M Tris(3-hydroxypropyltriazolylmethyl)amine (THPTA), 0.5M of Cu(II) sulfate pentahydrate (Cu(II)SO₄·5H₂O) and 2M solution of sodium ascorbate were prepared in deionized water. A 75 mg/mL solution in DMSO of targeting ligand was made. In a 1.5 mL centrifuge tube containing tri-alkyne functionalized duplex (3 mg, 75 μL, 40 mg/mL in deionized water, ˜15,000 g/mol), 25 μL of 1M Hepes pH 8.5 buffer is added. After vortexing, 35 μL of DMSO was added and the solution is vortexed. Targeting ligand was added to the reaction (6 equivalents/duplex, 2 equivalents/alkyne, ˜15 μL) and the solution is vortexed. Using pH paper, pH was checked and confirmed to be pH ˜8. In a separate 1.5 mL centrifuge tube, 50 μL of 0.5M THPTA was mixed with 10 uL of 0.5M Cu(II)SO₄·5H₂O, vortexed, and incubated at room temp for 5 min. After 5 min, THPTA/Cu solution (7.2 μL, 6 equivalents 5:1 THPTA:Cu) was added to the reaction vial, and vortexed. Immediately afterwards, 2M ascorbate (5 μL, 50 equivalents per duplex, 16.7 per alkyne) was added to the reaction vial and vortexed. Once the reaction was complete (typically complete in 0.5-1 h), the reaction was immediately purified by non-denaturing anion exchange chromatography.

### Example 2. SARS-CoV-2-SEAP Mouse Model

To assess the potency of the RNAi agents, a SARS-CoV-2-SEAP mouse model was used. Six to eight week old female C57BL/6 albino mice were transiently transfected in vivo with plasmid by hydrodynamic tail vein injection, administered at least 15 days prior to administration of an CoV RNAi agent or control. The plasmid contains segments of the SARS-CoV-2 genome sequence (GenBank NC_045512.2 (SEQ ID NO: 1)) inserted into the 3′ UTR of the SEAP (secreted human placental alkaline phosphatase) reporter gene. 10 μg to 50 μg of the plasmid containing the SARS-CoV-2 genome sequence in Ringer's Solution in a total volume of 10% of the animal's body weight was injected into mice via the tail vein to create SARS-CoV-2-SEAP model mice. The solution was injected through a 27-gauge needle in 5-7 seconds as previously described (Zhang G et al., “High levels of foreign gene expression in hepatocytes after tail vein injection of naked plasmid DNA.” Human Gene Therapy 1999 Vol. 10, p 1735-1737.). Inhibition of expression of SARS-CoV-2 sequences by a CoV RNAi agent results in concomitant inhibition of SEAP expression, which is measured by the Phospha-Light™ SEAP Reporter Gene Assay System (Invitrogen). Prior to treatment, SEAP expression levels in serum were measured and the mice were grouped according to average SEAP levels.

Analyses: SEAP levels may be measured at various times, both before and after administration of CoV RNAi agents.
i) Serum collection: Mice were anesthetized with 2-3% isoflurane and blood samples were collected from the submandibular area into serum separation tubes (Sarstedt AG & Co., Nümbrecht, Germany). Blood was allowed to coagulate at ambient temperature for 20 min. The tubes were centrifuged at 8,000×g for 3 min to separate the serum and stored at 4° C.
ii) Serum SEAP levels: Serum was collected and measured by the Phospha-Light™ SEAP Reporter Gene Assay System (Invitrogen) according to the manufacturer's instructions. Serum SEAP levels for each animal was normalized to the control group of mice injected with saline in order to account for the non-treatment related decline in SARS-CoV-2 sequence expression with this model. First, the SEAP level for each animal at a time point was divided by the pre-treatment level of expression in that animal (“pre-treatment”) in order to determine the ratio of expression “normalized to pre-treatment”. Expression at a specific time point was then normalized to the control group by dividing the “normalized to pre-treatment” ratio for an individual animal by the average “normalized to pre-treatment” ratio of all mice in the normal saline control group. Alternatively, in some Examples set forth herein, the serum SEAP levels for each animal were assessed by normalizing to pre-treatment levels only.

### Example 3. In Vivo Testing of CoV RNAi Agents in SARS-CoV-2-SEAP Mice

The SARS-CoV-2-SEAP mouse model described in Example 2, above, was used. At day 1, four (n=4) female C57bl/6 albino mice were given a single subcutaneous (SQ) injection of 200 μl per 20 g body weight containing either 2.0 mg/kg (mpk) of an CoV RNAi agent or saline without an CoV RNAi agent to be used as a control, according to the following Table 12.

TABLE 12
CoV RNAi agent and Dosing for Example 3

| Group ID | Dosing Regimen |
|---|---|
| Group 1 (isotonic saline) | Single SQ injection on day 1 |
| Group 2 (2.0 mg/kg AD10297) | Single SQ injection on day 1 |
| Group 3 (2.0 mg/kg AD10295) | Single SQ injection on day 1 |
| Group 4 (2.0 mg/kg AD10293) | Single SQ injection on day 1 |
| Group 5 (2.0 mg/kg AD10294) | Single SQ injection on day 1 |
| Group 6 (2.0 mg/kg AD10536) | Single SQ injection on day 1 |
| Group 7 (2.0 mg/kg AD10537) | Single SQ injection on day 1 |
| Group 8 (2.0 mg/kg AD10538) | Single SQ injection on day 1 |
| Group 9 (2.0 mg/kg AD10539) | Single SQ injection on day 1 |
| Group 10 (2.0 mg/kg AD10540) | Single SQ injection on day 1 |
| Group 11 (2.0 mg/kg AD10296) | Single SQ injection on day 1 |

Each of the CoV RNAi agents included N-acetyl-galactosamine targeting ligands ((NAG37)s) conjugated to the 5′-terminal end of the sense strand, as shown in Tables 5, 7A, and 11 and were added as phosphoramidite compounds during the oligonucleotide synthesis process described above in Example 1.

The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 13, with Average SEAP reflecting the normalized average value of SEAP.

TABLE 13
Average SEAP normalized to pre-treatment and saline
control in SARS-CoV-2-SEAP mice from Example 3.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (isotonic saline) | 1.000 | 0.156 | 1.000 | 0.143 |
| Group 2 (2.0 mg/kg AD10297) | 0.375 | 0.067 | 0.233 | 0.037 |
| Group 3 (2.0 mg/kg AD10295) | 0.416 | 0.193 | 0.295 | 0.111 |
| Group 4 (2.0 mg/kg AD10293) | 0.497 | 0.161 | 0.420 | 0.092 |
| Group 5 (2.0 mg/kg AD10294) | 0.461 | 0.099 | 0.395 | 0.142 |
| Group 6 (2.0 mg/kg AD10536) | 0.458 | 0.173 | 0.358 | 0.133 |
| Group 7 (2.0 mg/kg AD10537) | 0.385 | 0.087 | 0.229 | 0.039 |
| Group 8 (2.0 mg/kg AD10538) | 0.403 | 0.095 | 0.296 | 0.065 |
| Group 9 (2.0 mg/kg AD10539) | 0.436 | 0.015 | 0.313 | 0.081 |
| Group 10 (2.0 mg/kg AD10540) | 0.397 | 0.102 | 0.240 | 0.071 |
| Group 11 (2.0 mg/kg AD10296) | 0.378 | 0.089 | 0.270 | 0.079 |
| | Day 22 | | Day 29 | |
| | Avg | Std Dev | Avg | Std Dev |
| Group ID | SEAP | (+/−) | SEAP | (+/−) |
| Group 1 (isotonic saline) | 1.000 | 0.233 | 1.000 | 0.206 |
| Group 2 (2.0 mg/kg AD10297) | 0.235 | 0.072 | 0.365 | 0.133 |
| Group 3 (2.0 mg/kg AD10295) | 0.355 | 0.240 | 0.421 | 0.220 |
| Group 4 (2.0 mg/kg AD10293) | 0.508 | 0.164 | 0.770 | 0.184 |
| Group 5 (2.0 mg/kg AD10294) | 0.480 | 0.181 | 0.713 | 0.273 |
| Group 6 (2.0 mg/kg AD10536) | 0.402 | 0.152 | 0.473 | 0.207 |
| Group 7 (2.0 mg/kg AD10537) | 0.228 | 0.045 | 0.324 | 0.072 |
| Group 8 (2.0 mg/kg AD10538) | 0.273 | 0.070 | 0.437 | 0.163 |
| Group 9 (2.0 mg/kg AD10539) | 0.378 | 0.129 | 0.593 | 0.310 |
| Group 10 (2.0 mg/kg AD10540) | 0.263 | 0.088 | 0.378 | 0.113 |
| Group 11 (2.0 mg/kg AD10296) | 0.279 | 0.122 | 0.341 | 0.146 |

Each of the CoV RNAi agents in each of the dosing groups (i.e., Groups 2 through 11) showed reduction in SEAP as compared to the saline control (Group 1) across all measured time points, which as described herein, indicates inhibition of SARS-CoV-2 RNA in the SARS-CoV-2-SEAP mouse model. For example, at Day 22 the CoV RNAi agent of Group 7 (AD10537) showed reductions in SEAP of approximately 770 (0.228).

### Example 4. In Vivo testing of CoV RNAi agents in SARS-CoV-2-SEAP Mice

The SARS-CoV-2-SEAP mouse model described in Example 2, above, was used. At day 1. four (n=4) female C57bl/6 albino mice were given a single subcutaneous (SQ) injection of 200 per 20 g body weight containing either 2.0 mg/kg (mpk) of an CoV RNAi agent or saline without an CoV RNAi agent to be used as a control, according to the following Table 14.

TABLE 14
CoV RNAi agent and Dosing for Example 4

| Group ID | Dosing Regimen |
|---|---|
| Group 1 (isotonic saline) | Single SQ injection on day 1 |
| Group 2 (2.0 mg/kg AD10297) | Single SQ injection on day 1 |
| Group 3 (2.0 mg/kg AD10912) | Single SQ injection on day 1 |
| Group 4 (2.0 mg/kg AD10913) | Single SQ injection on day 1 |
| Group 5 (2.0 mg/kg AD10914) | Single SQ injection on day 1 |
| Group 6 (2.0 mg/kg AD10915) | Single SQ injection on day 1 |
| Group 7 (2.0 mg/kg AD10916) | Single SQ injection on day 1 |
| Group 8 (2.0 mg/kg AD10917) | Single SQ injection on day 1 |
| Group 9 (2.0 mg/kg AD10918) | Single SQ injection on day 1 |
| Group 10 (2.0 mg/kg AD10536) | Single SQ injection on day 1 |
| Group 11 (2.0 mg/kg AD10919) | Single SQ injection on day 1 |
| Group 12 (2.0 mg/kg AD10920) | Single SQ injection on day 1 |
| Group 13 (2.0 mg/kg AD10921) | Single SQ injection on day 1 |
| Group 14 (2.0 mg/kg AD10922) | Single SQ injection on day 1 |
| Group 15 (2.0 mg/kg AD10923) | Single SQ injection on day 1 |
| Group 16 (2.0 mg/kg AD10540) | Single SQ injection on day 1 |
| Group 17 (2.0 mg/kg AD10925) | Single SQ injection on day 1 |
| Group 18 (2.0 mg/kg AD10926) | Single SQ injection on day 1 |
| Group 19 (2.0 mg/kg AD10927) | Single SQ injection on day 1 |
| Group 20 (2.0 mg/kg AD10928) | Single SQ injection on day 1 |
| Group 21 (2.0 mg/kg AD10929) | Single SQ injection on day 1 |
| Group 22 (2.0 mg/kg AD10930) | Single SQ injection on day 1 |
| Group 23 (2.0 mg/kg AD10931) | Single SQ injection on day 1 |

Each of the CoV RNAi agents included N-acetyl-galactosamine targeting ligands ((NAG37)s) conjugated to the 5′-terminal end of the sense strand, as shown in Tables 5, 7A, and 11 and were added as phosphoramidite compounds during the oligonucleotide synthesis process described above in Example 1.

The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 15, with Average SEAP reflecting the normalized average value of SEAP.

TABLE 15
Average SEAP normalized to pre-treatment and saline
control in SARS-CoV-2-SEAP mice from Example 4.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (isotonic saline) | 1.000 | 0.205 | 1.000 | 0.183 |
| Group 2 (2.0 mg/kg AD10297) | 0.204 | 0.035 | 0.177 | 0.096 |
| Group 3 (2.0 mg/kg AD10912) | 0.193 | 0.020 | 0.151 | 0.039 |
| Group 4 (2.0 mg/kg AD10913) | 0.145 | 0.021 | 0.104 | 0.045 |
| Group 5 (2.0 mg/kg AD10914) | 0.164 | 0.043 | 0.100 | 0.045 |
| Group 6 (2.0 mg/kg AD10915) | 0.218 | 0.059 | 0.209 | 0.048 |
| Group 7 (2.0 mg/kg AD10916) | 0.184 | 0.029 | 0.141 | 0.030 |
| Group 8 (2.0 mg/kg AD10917) | 0.165 | 0.041 | 0.115 | 0.018 |
| Group 9 (2.0 mg/kg AD10918) | 0.137 | 0.021 | 0.076 | 0.025 |
| Group 10 (2.0 mg/kg AD10536) | 0.222 | 0.065 | 0.113 | 0.050 |
| Group 11 (2.0 mg/kg AD10919) | 0.194 | 0.060 | 0.124 | 0.015 |
| Group 12 (2.0 mg/kg AD10920) | 0.175 | 0.048 | 0.118 | 0.022 |
| Group 13 (2.0 mg/kg AD10921) | 0.174 | 0.029 | 0.066 | 0.032 |
| Group 14 (2.0 mg/kg AD10922) | 0.180 | 0.031 | 0.091 | 0.041 |
| Group 15 (2.0 mg/kg AD10923) | 0.147 | 0.040 | 0.150 | 0.070 |
| Group 16 (2.0 mg/kg AD10540) | 0.125 | 0.017 | 0.110 | 0.036 |
| Group 17 (2.0 mg/kg AD10925) | 0.228 | 0.063 | 0.192 | 0.044 |
| Group 18 (2.0 mg/kg AD10926) | 0.315 | 0.146 | 0.243 | 0.110 |
| Group 19 (2.0 mg/kg AD10927) | 0.293 | 0.022 | 0.221 | 0.070 |
| Group 20 (2.0 mg/kg AD10928) | 0.320 | 0.038 | 0.272 | 0.107 |
| Group 21 (2.0 mg/kg AD10929) | 0.428 | 0.035 | 0.301 | 0.070 |
| Group 22 (2.0 mg/kg AD10930) | 0.332 | 0.017 | 0.273 | 0.133 |
| Group 23 (2.0 mg/kg AD10931) | 0.248 | 0.069 | 0.229 | 0.026 |
| | Day 22 | | Day 29 | |
| | Avg | Std Dev | Avg | Std Dev |
| Group ID | SEAP | (+/−) | SEAP | (+/−) |
| Group 1 (isotonic saline) | 1.000 | 0.162 | 1.000 | 0.104 |
| Group 2 (2.0 mg/kg AD10297) | 0.149 | 0.061 | 0.177 | 0.016 |
| Group 3 (2.0 mg/kg AD10912) | 0.163 | 0.077 | 0.182 | 0.160 |
| Group 4 (2.0 mg/kg AD10913) | 0.067 | 0.025 | 0.077 | 0.022 |
| Group 5 (2.0 mg/kg AD10914) | 0.032 | 0.004 | 0.036 | 0.018 |
| Group 6 (2.0 mg/kg AD10915) | 0.126 | 0.022 | 0.146 | 0.030 |
| Group 7 (2.0 mg/kg AD10916) | 0.098 | 0.025 | 0.116 | 0.007 |
| Group 8 (2.0 mg/kg AD10917) | 0.050 | 0.022 | 0.073 | 0.023 |
| Group 9 (2.0 mg/kg AD10918) | 0.064 | 0.023 | 0.073 | 0.035 |
| Group 10 (2.0 mg/kg AD10536) | 0.123 | N/A | 0.153 | N/A |
| Group 11 (2.0 mg/kg AD10919) | 0.112 | N/A | 0.057 | 0.049 |
| Group 12 (2.0 mg/kg AD10920) | 0.073 | 0.003 | 0.048 | 0.003 |
| Group 13 (2.0 mg/kg AD10921) | 0.072 | 0.045 | 0.024 | 0.019 |
| Group 14 (2.0 mg/kg AD10922) | 0.170 | 0.105 | 0.055 | 0.040 |
| Group 15 (2.0 mg/kg AD10923) | 0.115 | 0.065 | 0.047 | 0.014 |
| Group 16 (2.0 mg/kg AD10540) | 0.084 | 0.044 | 0.059 | 0.017 |
| Group 17 (2.0 mg/kg AD10925) | 0.181 | 0.075 | 0.203 | 0.101 |
| Group 18 (2.0 mg/kg AD10926) | 0.301 | 0.091 | 0.254 | 0.062 |
| Group 19 (2.0 mg/kg AD10927) | 0.192 | 0.064 | 0.181 | 0.084 |
| Group 20 (2.0 mg/kg AD10928) | 0.249 | 0.062 | 0.238 | 0.079 |
| Group 21 (2.0 mg/kg AD10929) | 0.314 | 0.069 | 0.272 | 0.083 |
| Group 22 (2.0 mg/kg AD10930) | 0.215 | 0.066 | 0.249 | 0.118 |
| Group 23 (2.0 mg/kg AD10931) | 0.181 | 0.026 | 0.153 | 0.039 |

Groups 2-23 showed reduction in SEAP as compared to the saline control (Group 1) across all measured time points, which as described herein, indicates inhibition of SARS-CoV-2 RNA in the SARS-CoV-2-SEAP mouse model.

### Example 5. In Vivo Testing of CoV RNAi Agents in SARS-CoV-2-SEAP Mice

The SARS-CoV-2-SEAP mouse model described in Example 2, above, was used. At day 1, four (n=4) female C57bl/6 albino mice were given a single subcutaneous (SQ) injection of 200 d per 20 g body weight containing either 2.0 mg/kg (mpk) of an CoV RNAi agent or saline without an CoV RNAi agent to be used as a control, according to the following Table 16.

TABLE 16
CoV RNAi agent and Dosing for Example 5

| Group ID | Dosing Regimen |
|---|---|
| Group 1 (isotonic saline) | Single SQ injection on day 1 |
| Group 2 (2.0 mg/kg AD10536) | Single SQ injection on day 1 |
| Group 3 (2.0 mg/kg AD10538) | Single SQ injection on day 1 |
| Group 4 (2.0 mg/kg AD11101) | Single SQ injection on day 1 |
| Group 5 (2.0 mg/kg AD11102) | Single SQ injection on day 1 |
| Group 6 (2.0 mg/kg AD11103) | Single SQ injection on day 1 |
| Group 7 (2.0 mg/kg AD11104) | Single SQ injection on day 1 |
| Group 8 (2.0 mg/kg AD11105) | Single SQ injection on day 1 |
| Group 9 (2.0 mg/kg AD11106) | Single SQ injection on day 1 |
| Group 10 (2.0 mg/kg AD11107) | Single SQ injection on day 1 |
| Group 11 (2.0 mg/kg AD11108) | Single SQ injection on day 1 |
| Group 12 (2.0 mg/kg AD11109) | Single SQ injection on day 1 |
| Group 13 (2.0 mg/kg AD11110) | Single SQ injection on day 1 |
| Group 14 (2.0 mg/kg AD11111) | Single SQ injection on day 1 |
| Group 15 (2.0 mg/kg AD11112) | Single SQ injection on day 1 |
| Group 16 (2.0 mg/kg AD11113) | Single SQ injection on day 1 |
| Group 17 (2.0 mg/kg AD11114) | Single SQ injection on day 1 |
| Group 18 (2.0 mg/kg AD11115) | Single SQ injection on day 1 |
| Group 19 (2.0 mg/kg AD11116) | Single SQ injection on day 1 |
| Group 20 (2.0 mg/kg AD10296) | Single SQ injection on day 1 |
| Group 21 (2.0 mg/kg AD11117) | Single SQ injection on day 1 |
| Group 22 (2.0 mg/kg AD11118) | Single SQ injection on day 1 |
| Group 23 (2.0 mg/kg AD11119) | Single SQ injection on day 1 |
| Group 24 (2.0 mg/kg AD11120) | Single SQ injection on day 1 |

Each of the CoV RNAi agents included N-acetyl-galactosamine targeting ligands ((NAG37)s) conjugated to the 5′-terminal end of the sense strand, as shown in Tables 5, 7A, and 11 and were added as phosphoramidite compounds during the oligonucleotide synthesis process described above in Example 1.

The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 17, with Average SEAP reflecting the normalized average value of SEAP:

TABLE 17
Average SEAP normalized to pre-treatment and saline
control in SARS-CoV-2-SEAP mice from Example 5.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (isotonic saline) | 1.000 | 0.184 | 1.000 | 0.259 |
| Group 2 (2.0 mg/kg AD10536) | 0.345 | 0.111 | 0.286 | 0.104 |
| Group 3 (2.0 mg/kg AD10538) | 0.353 | 0.250 | 0.290 | 0.117 |
| Group 4 (2.0 mg/kg AD11101) | 0.219 | 0.135 | 0.153 | 0.074 |
| Group 5 (2.0 mg/kg AD11102) | 0.224 | 0.080 | 0.145 | 0.023 |
| Group 6 (2.0 mg/kg AD11103) | 0.231 | 0.043 | 0.161 | 0.033 |
| Group 7 (2.0 mg/kg AD11104) | 0.266 | 0.056 | 0.178 | 0.055 |
| Group 8 (2.0 mg/kg AD11105) | 0.227 | 0.032 | 0.104 | 0.042 |
| Group 9 (2.0 mg/kg AD11106) | 0.263 | 0.058 | 0.111 | 0.046 |
| Group 10 (2.0 mg/kg AD11107) | 0.234 | 0.030 | 0.122 | 0.004 |
| Group 11 (2.0 mg/kg AD11108) | 0.349 | 0.100 | 0.286 | 0.051 |
| Group 12 (2.0 mg/kg AD11109) | 0.200 | 0.030 | 0.143 | 0.017 |
| Group 13 (2.0 mg/kg AD11110) | 0.175 | 0.027 | 0.053 | 0.012 |
| Group 14 (2.0 mg/kg AD11111) | 0.220 | 0.013 | 0.089 | 0.013 |
| Group 15 (2.0 mg/kg AD11112) | 0.223 | 0.051 | 0.076 | 0.023 |
| Group 16 (2.0 mg/kg AD11113) | 0.196 | 0.035 | 0.134 | 0.041 |
| Group 17 (2.0 mg/kg AD11114) | 0.598 | 0.197 | 0.311 | 0.065 |
| Group 18 (2.0 mg/kg AD11115) | 0.555 | 0.029 | 0.334 | 0.031 |
| Group 19 (2.0 mg/kg AD11116) | 0.335 | 0.062 | 0.159 | 0.053 |
| Group 20 (2.0 mg/kg AD10296) | 0.451 | 0.145 | 0.242 | 0.051 |
| Group 21 (2.0 mg/kg AD11117) | 0.234 | 0.039 | 0.234 | 0.054 |
| Group 22 (2.0 mg/kg AD11118) | 0.225 | 0.078 | 0.105 | 0.040 |
| Group 23 (2.0 mg/kg AD11119) | 0.978 | 0.268 | 0.726 | 0.219 |
| Group 24 (2.0 mg/kg AD11120) | 1.118 | 0.201 | 0.804 | 0.336 |
| | Day 22 | | Day 29 | |
| | Avg | Std Dev | Avg | Std Dev |
| Group ID | SEAP | (+/−) | SEAP | (+/−) |
| Group 1 (isotonic saline) | 1.000 | 0.228 | 1.000 | 0.923 |
| Group 2 (2.0 mg/kg AD10536) | 0.319 | 0.128 | 0.397 | 0.098 |
| Group 3 (2.0 mg/kg AD10538) | 0.353 | 0.106 | 0.328 | 0.056 |
| Group 4 (2.0 mg/kg AD11101) | 0.226 | 0.039 | 0.219 | 0.082 |
| Group 5 (2.0 mg/kg AD11102) | 0.148 | 0.045 | 0.192 | 0.091 |
| Group 6 (2.0 mg/kg AD11103) | 0.167 | 0.064 | 0.185 | 0.111 |
| Group 7 (2.0 mg/kg AD11104) | 0.171 | 0.046 | 0.189 | 0.090 |
| Group 8 (2.0 mg/kg AD11105) | 0.098 | 0.054 | 0.137 | 0.079 |
| Group 9 (2.0 mg/kg AD11106) | 0.110 | 0.069 | 0.122 | 0.040 |
| Group 10 (2.0 mg/kg AD11107) | 0.118 | 0.009 | 0.089 | 0.047 |
| Group 11 (2.0 mg/kg AD11108) | 0.333 | 0.078 | 0.445 | 0.147 |
| Group 12 (2.0 mg/kg AD11109) | 0.168 | 0.020 | 0.285 | 0.235 |
| Group 13 (2.0 mg/kg AD11110) | 0.092 | 0.032 | 0.095 | 0.030 |
| Group 14 (2.0 mg/kg AD11111) | 0.192 | 0.025 | 0.124 | 0.027 |
| Group 15 (2.0 mg/kg AD11112) | 0.104 | 0.019 | 0.115 | 0.034 |
| Group 16 (2.0 mg/kg AD11113) | 0.202 | 0.090 | 0.132 | 0.060 |
| Group 17 (2.0 mg/kg AD11114) | 0.389 | 0.099 | 0.196 | 0.056 |
| Group 18 (2.0 mg/kg AD11115) | 0.448 | 0.088 | 0.261 | 0.036 |
| Group 19 (2.0 mg/kg AD11116) | 0.149 | 0.085 | 0.149 | 0.094 |
| Group 20 (2.0 mg/kg AD10296) | 0.369 | 0.107 | 0.243 | 0.068 |
| Group 21 (2.0 mg/kg AD11117) | 0.235 | 0.126 | 0.216 | 0.057 |
| Group 22 (2.0 mg/kg AD11118) | 0.122 | 0.054 | 0.135 | 0.067 |
| Group 23 (2.0 mg/kg AD11119) | 0.990 | 0.344 | 0.517 | 0.192 |
| Group 24 (2.0 mg/kg AD11120) | 0.831 | 0.251 | 0.487 | 0.198 |

Groups 2-22 showed reductions in SEAP as compared to the saline control (Group 1), which as described herein, indicates inhibition of SARS-CoV-2 RNA in the SARS-CoV-2-SEAP mouse model.

### Example 6. In Vivo Testing of CoVRNAi Agents in SARS-CoV-2-SEAP Mice

The SARS-CoV-2-SEAP mouse model described in Example 2, above, was used. At day 1, four (n=4) female C57bl/6 albino mice were given a single subcutaneous (SQ) injection of 250 μl per 25 g body weight containing either 2.0 mg/kg (mpk) of an CoV RNAi agent or saline without an CoV RNAi agent to be used as a control, according to the following Table 18.

TABLE 18
CoV RNAi agent and Dosing for Example 6

| Group ID | Dosing Regimen |
|---|---|
| Group 1 (isotonic saline) | Single SQ injection on day 1 |
| Group 2 (2.0 mg/kg AD10912) | Single SQ injection on day 1 |
| Group 3 (2.0 mg/kg AD11122) | Single SQ injection on day 1 |
| Group 4 (2.0 mg/kg AD11123) | Single SQ injection on day 1 |
| Group 5 (2.0 mg/kg AD11124) | Single SQ injection on day 1 |
| Group 6 (2.0 mg/kg AD11125) | Single SQ injection on day 1 |
| Group 7 (2.0 mg/kg AD11126) | Single SQ injection on day 1 |
| Group 8 (2.0 mg/kg AD11127) | Single SQ injection on day 1 |
| Group 9 (2.0 mg/kg AD11128) | Single SQ injection on day 1 |
| Group 10 (2.0 mg/kg AD11129) | Single SQ injection on day 1 |

Each of the CoV RNAi agents included N-acetyl-galactosamine targeting ligands ((NAG37)s) conjugated to the 5′-terminal end of the sense strand, as shown in Tables 5, 7A, and 11 and were added as phosphoramidite compounds during the oligonucleotide synthesis process described above in Example 1.

The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 19, with Average SEAP reflecting the normalized average value of SEAP.

TABLE 19
Average SEAP normalized to pre-treatment and saline
control in SARS-CoV-2-SEAP mice from Example 6.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (isotonic saline) | 1.000 | 0.303 | 1.000 | 0.415 |
| Group 2 (2.0 mg/kg AD10912) | 0.478 | 0.174 | 0.540 | 0.230 |
| Group 3 (2.0 mg/kg AD11122) | 0.208 | 0.069 | 0.142 | 0.035 |
| Group 4 (2.0 mg/kg AD11123) | 0.494 | 0.077 | 0.559 | 0.098 |
| Group 5 (2.0 mg/kg AD11124) | 0.913 | 0.234 | 0.953 | 0.215 |
| Group 6 (2.0 mg/kg AD11125) | 0.351 | 0.084 | 0.398 | 0.085 |
| Group 7 (2.0 mg/kg AD11126) | 0.810 | 0.148 | 0.874 | 0.188 |
| Group 8 (2.0 mg/kg AD11127) | 0.530 | 0.113 | 0.561 | 0.131 |
| Group 9 (2.0 mg/kg AD11128) | 1.152 | 0.279 | 1.126 | 0.342 |
| Group 10 (2.0 mg/kg AD11129) | 0.976 | 0.180 | 1.000 | 0.248 |
| | Day 22 | | Day 29 | |
| | Avg | Std Dev | Avg | Std Dev |
| Group ID | SEAP | (+/−) | SEAP | (+/−) |
| Group 1 (isotonic saline) | 1.000 | 0.459 | 1.000 | 0.539 |
| Group 2 (2.0 mg/kg AD10912) | 0.634 | 0.285 | 0.748 | 0.422 |
| Group 3 (2.0 mg/kg AD11122) | 0.158 | 0.032 | 0.170 | 0.029 |
| Group 4 (2.0 mg/kg AD11123) | 0.531 | 0.096 | 0.657 | 0.183 |
| Group 5 (2.0 mg/kg AD11124) | 0.891 | 0.193 | 1.109 | 0.292 |
| Group 6 (2.0 mg/kg AD11125) | 0.398 | 0.104 | 0.606 | 0.238 |
| Group 7 (2.0 mg/kg AD11126) | 0.901 | 0.181 | 1.047 | 0.121 |
| Group 8 (2.0 mg/kg AD11127) | 0.588 | 0.195 | 0.768 | 0.377 |
| Group 9 (2.0 mg/kg AD11128) | 1.068 | 0.345 | 1.159 | 0.495 |
| Group 10 (2.0 mg/kg AD11129) | 0.960 | 0.310 | 1.236 | 0.715 |

Groups 2-4, 6, and 8 showed reduction in SEAP as compared to the saline control (Group 1), which as described herein, indicates inhibition of SARS-CoV-2 RNA in the SARS-CoV-2-SEAP mouse model.

### Example 7. In Vivo Testing of CoV RNAi Agents in SARS-Co V-2-SEAP Mice

The SARS-CoV-2-SEAP mouse model described in Example 2, above, was used. At day 1, four (n=4) female C57bl/6 albino mice were given a single subcutaneous (SQ) injection of 200 μl per 20 g body weight containing either 2.0 mg/kg (mpk), 1.0 mg/kg (mpk) or 0.5 mg/kg (mpk) of an CoV RNAi agent or saline without an CoV RNAi agent to be used as a control, according to the following Table 20.

TABLE 20
CoV RNAi agent and Dosing for Example 7

| Group ID | Dosing Regimen |
|---|---|
| Group 1 (isotonic saline) | Single SQ injection on day 1 |
| Group 2 (1.0 mg/kg AD10297) | Single SQ injection on day 1 |
| Group 3 (2.0 mg/kg AD10914) | Single SQ injection on day 1 |
| Group 4 (1.0 mg/kg AD10914) | Single SQ injection on day 1 |
| Group 5 (0.5 mg/kg AD10914) | Single SQ injection on day 1 |
| Group 6 (1.0 mg/kg AD11610) | Single SQ injection on day 1 |
| Group 7 (1.0 mg/kg AD11611) | Single SQ injection on day 1 |
| Group 8 (1.0 mg/kg AD10538) | Single SQ injection on day 1 |
| Group 9 (1.0 mg/kg AD11105) | Single SQ injection on day 1 |
| Group 10 (1.0 mg/kg AD11612) | Single SQ injection on day 1 |
| Group 11 (1.0 mg/kg AD11118) | Single SQ injection on day 1 |

Each of the CoV RNAi agents included N-acetyl-galactosamine targeting ligands ((NAG37)s) conjugated to the 5′-terminal end of the sense strand, as shown in Tables 5, 7A, and 11 and were added as phosphoramidite compounds during the oligonucleotide synthesis process described above in Example 1.

The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 21, with Average SEAP reflecting the normalized average value of SEAP:

TABLE 21
Average SEAP normalized to pre-treatment and saline
control in SARS-CoV-2-SEAP mice from Example 7.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (isotonic saline) | 1.000 | 0.112 | 1.000 | 0.252 |
| Group 2 (1.0 mg/kg AD10297) | 0.533 | 0.109 | 0.420 | 0.230 |
| Group 3 (2.0 mg/kg AD10914) | 0.224 | 0.076 | 0.143 | 0.022 |
| Group 4 (1.0 mg/kg AD10914) | 0.468 | 0.158 | 0.426 | 0.102 |
| Group 5 (0.5 mg/kg AD10914) | 0.558 | 0.229 | 0.397 | 0.205 |
| Group 6 (1.0 mg/kg AD11610) | 0.476 | 0.098 | 0.286 | 0.102 |
| Group 7 (1.0 mg/kg AD11611) | 0.362 | 0.073 | 0.221 | 0.024 |
| Group 8 (1.0 mg/kg AD10538) | 0.600 | 0.147 | 0.526 | 0.151 |
| Group 9 (1.0 mg/kg AD11105) | 0.337 | 0.040 | 0.137 | 0.069 |
| Group 10 (1.0 mg/kg AD11612) | 0.282 | 0.062 | 0.212 | 0.086 |
| Group 11 (1.0 mg/kg AD11118) | 0.401 | 0.083 | 0.324 | 0.100 |
| | Day 22 | | Day 29 | |
| | Avg | Std Dev | Avg | Std Dev |
| Group ID | SEAP | (+/−) | SEAP | (+/−) |
| Group 1 (isotonic saline) | 1.000 | 0.517 | 1.000 | 0.888 |
| Group 2 (1.0 mg/kg AD10297) | 0.576 | 0.188 | 0.505 | 0.177 |
| Group 3 (2.0 mg/kg AD10914) | 0.175 | 0.021 | 0.248 | 0.073 |
| Group 4 (1.0 mg/kg AD10914) | 0.491 | 0.395 | 0.547 | 0.552 |
| Group 5 (0.5 mg/kg AD10914) | 0.490 | 0.189 | 0.444 | 0.225 |
| Group 6 (1.0 mg/kg AD11610) | 0.463 | 0.120 | 0.431 | 0.124 |
| Group 7 (1.0 mg/kg AD11611) | 0.282 | 0.106 | 0.348 | 0.174 |
| Group 8 (1.0 mg/kg AD10538) | 0.521 | 0.248 | 0.410 | 0.128 |
| Group 9 (1.0 mg/kg AD11105) | 0.285 | 0.076 | 0.259 | 0.056 |
| Group 10 (1.0 mg/kg AD11612) | 0.261 | 0.147 | 0.243 | 0.108 |
| Group 11 (1.0 mg/kg AD11118) | 0.410 | 0.157 | 0.535 | 0.235 |

Groups 2-11 (at all time points) showed reduction in SEAP as compared to the saline control (Group 1), which as described herein, indicates inhibition of SARS-CoV-2 RNA in the SARS-CoV-2-SEAP mouse model.

### Example 8. In Vivo Testing of CoV RNAi Agents in SARS-CoV-2-SEAP Mice

The SARS-CoV-2-SEAP mouse model described in Example 2, above, was used. At day 1, four (n=4) female C57bl/6 albino mice were given a single subcutaneous (SQ) injection of 250 μl per 25 g body weight containing either 1.0 mg/kg (mpk), 2.0 mg/kg (mpk) or 0.5 mg/kg (mpk) of a CoV RNAi agent or saline without an CoV RNAi agent to be used as a control, according to the following Table 22.

TABLE 22
CoV RNAi agent and Dosing for Example 8

| Group ID | Dosing Regimen |
|---|---|
| Group 1 (isotonic saline) | Single SQ injection on day 1 |
| Group 2 (1.0 mg/kg AD10536) | Single SQ injection on day 1 |
| Group 3 (2.0 mg/kg AD10921) | Single SQ injection on day 1 |
| Group 4 (1.0 mg/kg AD10921) | Single SQ injection on day 1 |
| Group 5 (0.5 mg/kg AD10921) | Single SQ injection on day 1 |
| Group 6 (1.0 mg/kg AD11108) | Single SQ injection on day 1 |
| Group 7 (1.0 mg/kg AD11110) | Single SQ injection on day 1 |
| Group 8 (1.0 mg/kg AD11613) | Single SQ injection on day 1 |
| Group 9 (1.0 mg/kg AD11112) | Single SQ injection on day 1 |
| Group 10 (1.0 mg/kg AD11614) | Single SQ injection on day 1 |
| Group 11 (1.0 mg/kg AD11615) | Single SQ injection on day 1 |
| Group 12 (1.0 mg/kg AD11616) | Single SQ injection on day 1 |

Each of the CoV RNAi agents included N-acetyl-galactosamine targeting ligands ((NAG37)s) conjugated to the 5′-terminal end of the sense strand, as shown in Tables 5, 7A, and 11 and were added as phosphoramidite compounds during the oligonucleotide synthesis process described above in Example 1.

The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 23, with Average SEAP reflecting the normalized average value of SEAP.

TABLE 23
Average SEAP normalized to pre-treatment and saline
control in SARS-CoV-2-SEAP mice from Example 8.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (isotonic saline) | 1.000 | 0.248 | 1.000 | 0.266 |
| Group 2 (1.0 mg/kg AD10536) | 0.418 | 0.054 | 0.381 | 0.054 |
| Group 3 (2.0 mg/kg AD10921) | 0.151 | 0.024 | 0.122 | 0.009 |
| Group 4 (1.0 mg/kg AD10921) | 0.236 | 0.076 | 0.124 | 0.073 |
| Group 5 (0.5 mg/kg AD10921) | 0.380 | 0.161 | 0.230 | 0.042 |
| Group 6 (1.0 mg/kg AD11108) | 0.263 | 0.060 | 0.254 | 0.092 |
| Group 7 (1.0 mg/kg AD11110) | 0.218 | 0.057 | 0.128 | 0.041 |
| Group 8 (1.0 mg/kg AD11613) | 0.195 | 0.047 | 0.168 | 0.058 |
| Group 9 (1.0 mg/kg AD11112) | 0.212 | 0.042 | 0.149 | 0.035 |
| Group 10 (1.0 mg/kg AD11614) | 0.196 | 0.059 | 0.140 | 0.062 |
| Group 11 (1.0 mg/kg AD11615) | 0.190 | 0.033 | 0.160 | 0.079 |
| Group 12 (1.0 mg/kg AD11616) | 0.183 | 0.060 | 0.141 | 0.048 |
| | Day 22 | | Day 29 | |
| | Avg | Std Dev | Avg | Std Dev |
| Group ID | SEAP | (+/−) | SEAP | (+/−) |
| Group 1 (isotonic saline) | 1.000 | 0.350 | 1.000 | 0.341 |
| Group 2 (1.0 mg/kg AD10536) | 0.364 | 0.094 | 0.539 | 0.203 |
| Group 3 (2.0 mg/kg AD10921) | 0.121 | 0.027 | 0.172 | 0.083 |
| Group 4 (1.0 mg/kg AD10921) | 0.159 | 0.054 | 0.184 | 0.047 |
| Group 5 (0.5 mg/kg AD10921) | 0.207 | 0.050 | 0.321 | 0.140 |
| Group 6 (1.0 mg/kg AD11108) | 0.301 | 0.160 | 0.512 | 0.352 |
| Group 7 (1.0 mg/kg AD11110) | 0.149 | 0.080 | 0.137 | 0.039 |
| Group 8 (1.0 mg/kg AD11613) | 0.140 | 0.061 | 0.176 | 0.084 |
| Group 9 (1.0 mg/kg AD11112) | 0.170 | 0.048 | 0.178 | 0.027 |
| Group 10 (1.0 mg/kg AD11614) | 0.124 | 0.065 | 0.182 | 0.096 |
| Group 11 (1.0 mg/kg AD11615) | 0.142 | 0.094 | 0.155 | 0.101 |
| Group 12 (1.0 mg/kg AD11616) | 0.171 | 0.068 | 0.286 | 0.265 |

Each of the CoV RNAi agents in each of the dosing groups (i.e., Groups 2 through 12) showed reduction in SEAP as compared to the saline control (Group 1) across all measured time points, which as described herein, indicates inhibition of SARS-CoV-2 in the SARS-CoV-2-SEAP mouse model.

### Example 9. In Vivo Testing of CoV RNAi Agents in SARS-CoV-2-SEAP Mice

The SARS-CoV-2-SEAP mouse model described in Example 2, above, was used. At day 1, four (n=4) female C57bl/6 albino mice were given a single subcutaneous (SQ) injection of 200 μl per 20 g body weight containing either 2.0 mg/kg (mpk), 1.0 mg/kg (mpk) or 0.5 mg/kg (mpk) of an CoV RNAi agent or saline without an CoV RNAi agent to be used as a control, according to the following Table 24.

TABLE 24
CoV RNAi agent and Dosing for Example 9

| Group ID | Dosing Regimen |
|---|---|
| Group 1 (isotonic saline) | Single SQ injection on day 1 |
| Group 2 (2.0 mg/kg AD11611) | Single SQ injection on day 1 |
| Group 3 (1.0 mg/kg AD11611) | Single SQ injection on day 1 |
| Group 4 (0.5 mg/kg AD11611) | Single SQ injection on day 1 |
| Group 5 (2.0 mg/kg AD11122) | Single SQ injection on day 1 |
| Group 6 (1.0 mg/kg AD11122) | Single SQ injection on day 1 |
| Group 7 (0.5 mg/kg AD11122) | Single SQ injection on day 1 |
| Group 8 (2.0 mg/kg AD11105) | Single SQ injection on day 1 |
| Group 9 (1.0 mg/kg AD11105) | Single SQ injection on day 1 |
| Group 10 (0.5 mg/kg AD11105) | Single SQ injection on day 1 |

Each of the CoV RNAi agents included N-acetyl-galactosamine targeting ligands ((NAG37)s) conjugated to the 5′-terminal end of the sense strand, as shown in Tables 5, 7A, and 11 and were added as phosphoramidite compounds during the oligonucleotide synthesis process described above in Example 1.

These CoV RNAi agents were selected for inclusion in this study based upon data from previous studies that identified each of them as being the most highly potent at inhibiting expression. AD11611 includes an antisense strand nucleotide sequence targeting position 6412 of the SARS-CoV-2 genome; AD11122 includes an antisense strand nucleotide sequence targeting position 4156 of the SARS-CoV-2 genome; and AD11105 includes an antisense strand nucleotide sequence targeting position 29150 of the SARS-CoV-2 genome.

The injections were performed between the skin and muscle (i.e. subcutaneous injections) into the loose skin over the neck and shoulder area. Four (4) mice in each group were tested (n=4). Serum was collected on day 8, day 15, day 22, and day 29, and SEAP expression levels were determined pursuant to the procedure set forth in Example 2, above. Data from the experiment are shown in the following Table 25, with Average SEAP reflecting the normalized average value of SEAP

TABLE 25
Average SEAP normalized to pre-treatment and saline
control in SARS-CoV-2-SEAP mice from Example 9.

| | Day 8 | | Day 15 | |
|---|---|---|---|---|
| | Avg | Std Dev | Avg | Std Dev |
| Group ID | SEAP | (+/−) | SEAP | (+/−) |
| Group 1 (isotonic saline) | 1.000 | 0.308 | 1.000 | 0.189 |
| Group 2 (2.0 mg/kg AD11611) | 0.243 | 0.081 | 0.213 | 0.088 |
| Group 3 (1.0 mg/kg AD11611) | 0.377 | 0.129 | 0.345 | 0.206 |
| Group 4 (0.5 mg/kg AD11611) | 0.438 | 0.136 | 0.365 | 0.114 |
| Group 5 (2.0 mg/kg AD11122) | 0.246 | 0.097 | 0.147 | 0.080 |
| Group 6 (1.0 mg/kg AD11122) | 0.422 | 0.086 | 0.330 | 0.090 |
| Group 7 (0.5 mg/kg AD11122) | 0.496 | 0.187 | 0.536 | 0.204 |
| Group 8 (2.0 mg/kg AD11105) | 0.238 | 0.043 | 0.164 | 0.050 |
| Group 9 (1.0 mg/kg AD11105) | 0.427 | 0.360 | 0.339 | 0.336 |
| Group 10 (0.5 mg/kg AD11105) | 0.319 | 0.102 | 0.230 | 0.058 |
| | Day 22 | | Day 29 | |
| | Avg | Std Dev | Avg | Std Dev |
| Group ID | SEAP | (+/−) | SEAP | (+/−) |
| Group 1 (isotonic saline) | 1.000 | 0.185 | 1.000 | 0.243 |
| Group 2 (2.0 mg/kg AD11611) | 0.231 | 0.115 | 0.231 | 0.095 |
| Group 3 (1.0 mg/kg AD11611) | 0.382 | 0.112 | 0.343 | 0.109 |
| Group 4 (0.5 mg/kg AD11611) | 0.464 | 0.151 | 0.329 | 0.103 |
| Group 5 (2.0 mg/kg AD11122) | 0.146 | 0.079 | 0.170 | 0.081 |
| Group 6 (1.0 mg/kg AD11122) | 0.409 | 0.121 | 0.483 | 0.179 |
| Group 7 (0.5 mg/kg AD11122) | 0.420 | 0.125 | 0.380 | 0.157 |
| Group 8 (2.0 mg/kg AD11105) | 0.153 | 0.038 | 0.143 | 0.040 |
| Group 9 (1.0 mg/kg AD11105) | 0.335 | 0.340 | 0.262 | 0.266 |
| Group 10 (0.5 mg/kg AD11105) | 0.225 | 0.076 | 0.216 | 0.092 |

As shown in the tables above, each of the CoV RNAi agents in each of the dosing groups (i.e., Groups 2 through 10) showed substantial reductions in SEAP as compared to the saline control (Group 1) across all measured time points, which as described herein, indicates inhibition of SARS-CoV-2 in the SARS-CoV-2-SEAP mouse model.

### Example 10. Testing of RNAi Triggers in a Hamster Model of SARS-CoV-2 Infection

To assess the potency of the RNAi agents, a hamster model of SARS-CoV-2 infection was also used. Six to eight week old male Syrian golden hamsters were divided into 9 groups according to Table 26 below. Hamsters were pre-treated with RNAi agent or saline on study days −8 and −6 via intratracheal instillation (IT) prior to SARS-CoV-2 challenge delivered intranasally (IN) on study day 0. Groups were euthanized on either study day 3 or day 7 post SARS-CoV-2 challenge. Group 1 was a control group administered saline on day −8 and day −6 pre-challenge. Groups 2-4 were administered AC001924 and AC001926 individually or in combination on day −8 and day −6 at single 5 mg/kg IT doses and euthanized on study day 3 post-SARS-CoV-2 challenge. Groups 5 and 6 were administered saline on day −8 and day −6 Groups 7-9 were administered AC0001924 and AC001926 individually or in combination on day −8 and day −6 at single 5 mg/kg IT doses and euthanized on study day 7 post-SARS-CoV-2 challenge. For animals receiving a combination of AC0001924 and AC001926, the two RNAi agents were combined, and the dose indicated in Table 26 is the total dose of the two duplexes. AC001924 includes an antisense strand nucleotide sequence targeting position 29150 of the SARS-CoV-2 genome, and AC001926 includes an antisense strand nucleotide sequence targeting position 15886 of the SARS-CoV-2 genome. Intratracheal instillation was administered at a volume of 2 mL/kg based on body weight. For SARS-CoV-2 challenge intranasally, administration of 9×10³ plaque-forming units (PFU) of WA01 isolate was given at a volume of 50 μL volume in each nostril. Body weights were determined daily from day −4 until terminal collection for all groups. At euthanization, left lung lobes were collected, with one half snap frozen for analysis of PFU in tissue homogenate, and one half for viral RNA qPCR. Right lungs were inflated with 10% neutral buffered formalin (NBF), transferred to 70% ethanol, and processed into paraffin blocks for H&E staining.

TABLE 26
Experimental Design

| | | | | Dose | | | |
|---|---|---|---|---|---|---|---|
| | | | | Level per | Treatment | SARS-CoV-2 | Terminal |
| Group | N | Treatment | Route | Treatment | Days | challenge | Collection |
| 1 | 8 | Saline | IT | 0 mg/kg | D -8, D -6 | D 0 | D 3 |
| 2 | 8 | AC001924 | IT | 5 mg/kg | D -8, D -6 | D 0 | D 3 |
| | | (pos 29150) | | | | | |
| 3 | 8 | AC001926 | IT | 5 mg/kg | D -8, D -6 | D 0 | D 3 |
| | | (pos 15886) | | | | | |
| 4 | 8 | AC001924 | IT | 5 mg/kg | D -8, D -6 | D 0 | D 3 |
| | | (pos 29150) + | | | | | |
| | | AC001926 | | | | | |
| | | (pos 15886) | | | | | |
| 5 | 3 | Saline | IT | 0 mg/kg | D -8, D -6 | No challenge | D 7 |
| 6 | 8 | Saline | IT | 0 mg/kg | D -8, D -6 | D 0 | D 7 |
| 7 | 8 | AC001924 | IT | 5 mg/kg | D -8, D -6 | D 0 | D 7 |
| | | (pos 29150) | | | | | |
| 8 | 8 | AC001926 | IT | 5 mg/kg | D -8, D -6 | D 0 | D 7 |
| | | (pos 15886) | | | | | |
| 9 | 8 | AC001924 | IT | 5 mg/kg | D -8, D -6 | D 0 | D 7 |
| | | (pos 29150) + | | | | | |
| | | AC001926 | | | | | |
| | | (pos 15886) | | | | | |

### Results

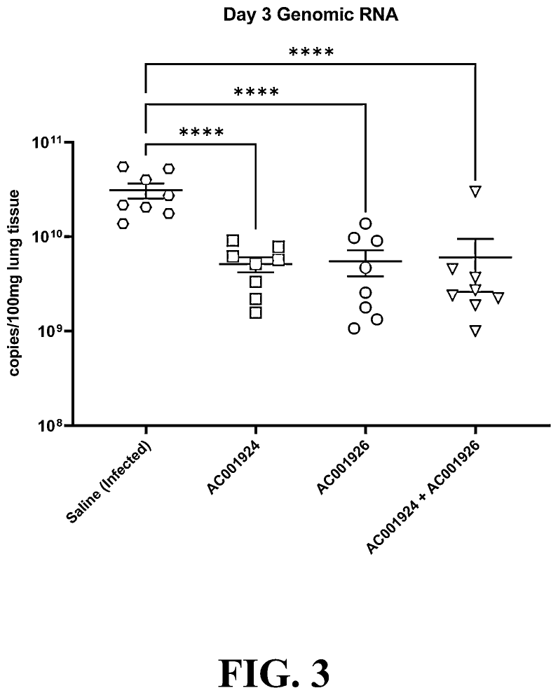

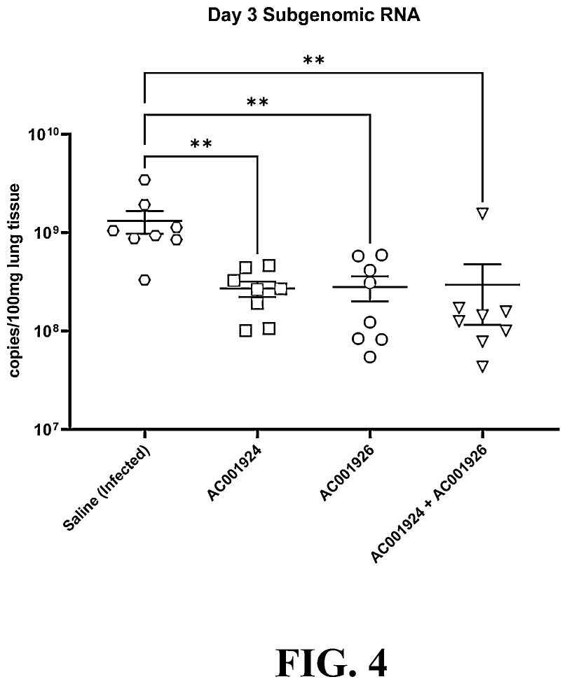

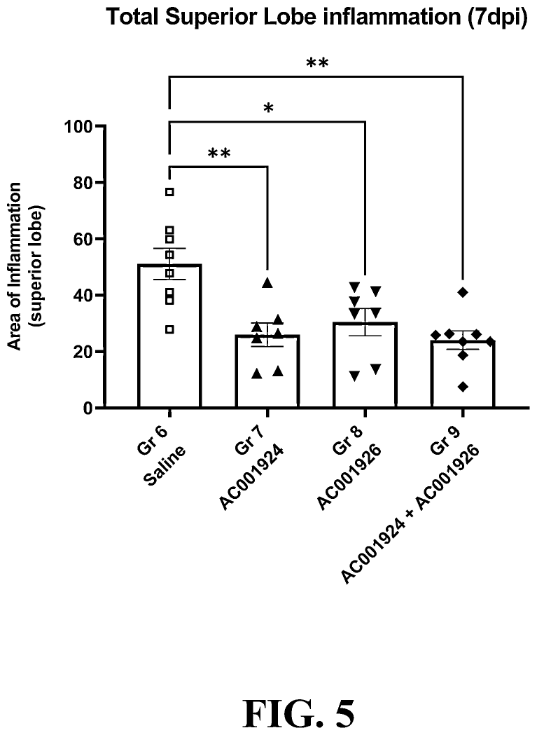

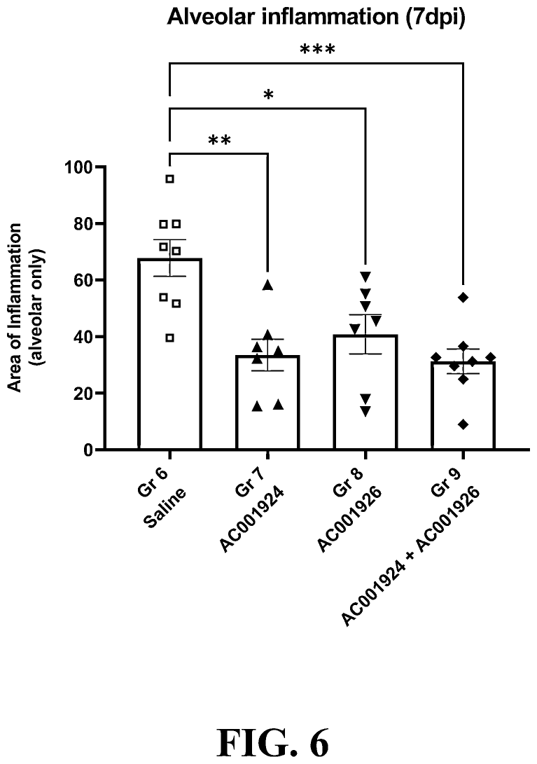

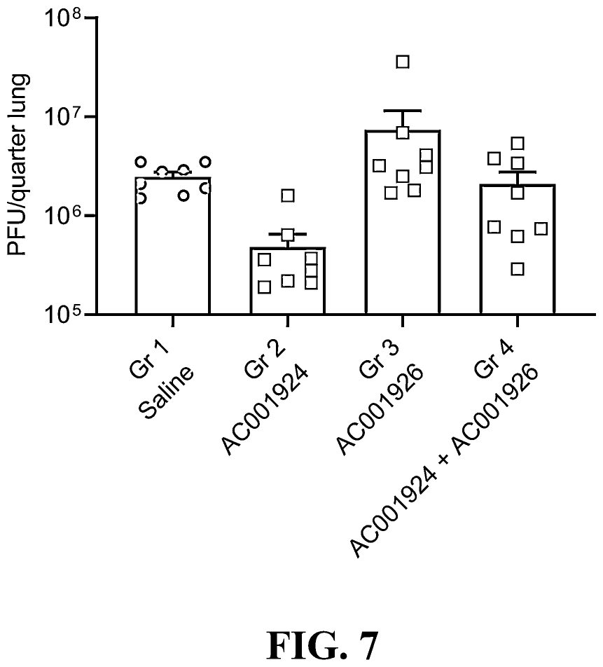

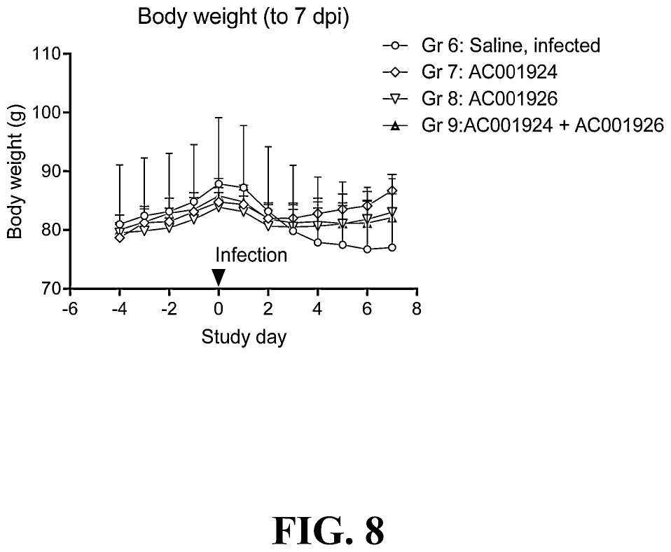

The results shown in FIGS. 3 through 8 demonstrate that RNAi agents AC001924 and AC001926 delivered individually or in combination reduce SARS-CoV-2 genomic and subgenomic RNA, reduce total inflammation and alveolar inflammation, reduce the number of PFUs in tissue homogenate, and allows body weight restoration. Specifically, and for example, RNAi agent AC001924 (position 29150) reduced genomic RNA and subgenomic RNA by 83% and 79%, respectively, relative to the SARS-CoV-2 infected saline control group on day 3 post-challenge as shown in FIG. 3 and FIG. 4. Hamsters treated with AC001924 also reduced total lung tissue inflammation and alveolar inflammation (as quantified by HALO) by 49% and 51%, respectively, relative to the SARS-CoV-2 infected saline control group on day 7 post-challenge as shown in FIG. 5 and FIG. 6. Further, AC001924 resulted in an 80% reduction in tissue homogenate PFU on day 3 dpi, as shown in FIG. 7. Lastly, AC001924 treatment resulted in the greatest restoration of body weights following SARS-CoV-2 infection over the course of the study, as reported in FIG. 8.

### Example 11. SARS-COV-2 Delta and Omicron Variants In-Silico Analysis

In late 2020, the Delta variant (B.1.617.2) of SARS-CoV-2 was first detected in India, and rapidly spread to become the dominant global strain of SARS-CoV-2. An in silico assessment was conducted to determine whether the six identified targeted sequence positions in Table 2 (i.e., CoV RNAi agents targeting the SARS-CoV-2 genome at positions 29150, 6412, 4156, 4917, 14503, and 15886) were conserved across the Delta variant transcripts reported in the NCBI database. A total of 7,794 SARS-CoV-2 transcripts from a human host that had Pango lineage (B.1.617.1, B.1.617.2, and B.1.617.3) were identified, and all six of the identified candidate sequence positions reported in Table 2 were conserved across at least 98% of the reported Delta variant transcripts in the NCBI database. This indicates that CoV RNAi agents designed to target the SARS-CoV-2 RNA at these positions would be expected to inhibit the SARS-CoV-2 Delta variant in the vast majority of infected subjects.

In November 2021, the Omicron variant (B.1.1.529) of SARS-CoV-2 was reported in South Africa, which was identified as being capable of multiplying approximately 70 times faster than the previously most prominent variant, the Delta variant. Shortly thereafter, the Omicron variant became the most prominent variant across the world. An in silico assessment was conducted to determine whether the six identified targeted sequence positions in Table 2 (i.e., CoV RNAi agents targeting the SARS-CoV-2 genome at positions 29150, 6412, 4156, 4917, 14503, and 15886) were conserved across the reported Omicron gene variant sequences reported in the NCBI database. As of Jan. 31, 2022, there were 820 different Omicron variant genome sequences reported in the NCBI database, and all six of the identified candidate sequence positions reported in Table 2 had sequences that were conserved across 99% of the reported Omicron sequences, indicating that CoV RNAi agents disclosed herein having sequences designed to inhibit expression of SARS-CoV-2 at positions 29150, 6412, 4156, 4917, 14503, and 15886 would be expected to inhibit the SARS-CoV-2 Omicron variant in the vast majority of infected subjects.

### Example 12. In Vitro Testing of CoV RNAi Agents in Vero E6 Cells. Texas

CoV RNAi agents were evaluated for their effectiveness (individually and in combination) to reduce SARS-CoV-2 virions, genomic and subgenomic RNA. SARS-CoV-2 (BEI Resources, 2019-nCoV/USA-WA1/2020 strain) was obtained, and infected onto Vero E6 cells at a multiplicity of infection (MOI) of 0.001 to create working viral stocks. Viral titers were determined by plaque assay using Vero E6 cells.

Transfection conditions were characterized for Vero E6 cells. Positive and negative siRNA construct controls were selected. Vero E6 cells were transfected with Lipofectamine RNAiMAX in 96-well plates with 0.1 nM, 1 nM, and 10 nM siRNA. At time points 24 hour (hr), 48 hr, and 72 hr post-transfection, RNA analysis was performed using Invitrogen TaqMan™ Gene Expression Cells to CT™ kit (Invitrogen Catalog No. 4399002). RT-qPCR measurement of positive control mRNA normalized to hPPIA; the hPPIA endogenous control for normalization (cyclophilin A, Thermo Fisher catalog #4326316E).

SARS-CoV-2 RNAi agents were transfected onto Vero E6 cells. At 48 hr post transfection, the Vero E6 cells (transfected with CoV RNAi agents) were then infected with SARS-CoV-2. Transfection was performed at 5000 cells/well via RNAiMax, MOI 0.01 (200-300 PFU/ml)-96-well-format. The plaque assay immunostained for SARS-NP. Percent % virus inhibition was calculated by the following equation:

$$\frac{[ ( plaques in NC siRNA ) - ( plaques in test siRNA ) ]}{plaques in NC siRNA} \times 100$$

The CoV RNAi agents tested are listed in the following Table 27. The in vitro screen results are shown in the following Table 28, from two separate experiments.

TABLE 27
CoV RNAi agents screened for Example 12.

| | Targeted Viral | Region of |
|---|---|---|
| AD Duplex ID | Genome Position | CoV Genome |
| AD08584 | 6412 | NSP3 |
| AD08586 | 12284 | NSP8 |
| AD08588 | 13766 | RDRP |
| AD08591 | 14503 | RDRP |
| AD08592 | 14511 | RDRP |
| AD08607 | 26304 | E |
| AD08608 | 26330 | E |
| AD08609 | 26367 | E |
| AD08610 | 26370 | E |
| AD08611 | 26371 | E |
| AD08617 | 27184 | M |
| AD08857 | 10931 | 3CP |
| AD08858 | 11434 | NSP6 |
| AD08859 | 15885 | RDRP |
| AD08860 | 15886 | RDRP |
| AD08862 | 20943 | 2′ORMT |
| AD08930 | 28590 | N |
| AD08933 | 29064 | N |
| AD08935 | 29150 | N |
| AD08929 | 28587 | N |

TABLE 28A
In vitro CoV RNAi agent screening,
percent % CoV virus inhibition.

| AD Duplex ID | % CoV Virus Inhibition | Standard Deviation |
|---|---|---|
| AD08584 | 98.5491 | 0.9135 |
| AD08586 | 100.0000 | 0.0000 |
| AD08588 | 79.1295 | 6.0258 |
| AD08591 | 99.6652 | 0.3702 |
| AD08592 | 99.6652 | 0.5799 |
| AD08607 | 99.4420 | 0.9665 |
| AD08608 | 89.0625 | 1.4293 |
| AD08609 | 85.7143 | 4.0672 |
| AD08610 | 93.8616 | 1.4595 |
| AD08611 | 98.1027 | 3.0340 |
| AD08617 | 58.8170 | 6.2050 |
| AD08857 | 90.9598 | 2.8476 |
| AD08858 | 92.6339 | 1.4974 |
| AD08859 | 97.6563 | 4.0595 |
| AD08860 | 98.5491 | 1.3895 |
| AD08862 | 99.2188 | 0.9135 |
| AD08930 | 98.9955 | 0.7970 |
| AD08933 | 99.6652 | 0.5799 |
| AD08935 | 100.0000 | 0.0000 |
| AD08929 | 99.5536 | 0.3157 |

TABLE 28B
In vitro CoV RNAi agent screening,
percent % CoV virus inhibition.

| AD Duplex ID | % CoV Virus Inhibition | Standard Deviation |
|---|---|---|
| AD08584 | 98.0000 | 1.3237 |
| AD08586 | 99.7273 | 0.4724 |
| AD08588 | 69.0000 | 2.8561 |
| AD08591 | 99.9091 | 0.1575 |
| AD08592 | 99.9091 | 0.1575 |
| AD08607 | 99.7273 | 0.4724 |
| AD08608 | 88.0000 | 1.6262 |
| AD08609 | 83.4545 | 3.3575 |
| AD08610 | 92.2727 | 2.2471 |
| AD08611 | 89.6364 | 1.1923 |
| AD08617 | 45.4545 | 4.6355 |
| AD08857 | 77.6364 | 4.5455 |
| AD08858 | 89.0909 | 1.8363 |
| AD08859 | 99.3636 | 0.3963 |
| AD08860 | 99.1818 | 0.4724 |
| AD08862 | 99.5455 | 0.5961 |
| AD08930 | 99.0909 | 0.7497 |
| AD08933 | 99.6364 | 0.3636 |
| AD08935 | 99.8182 | 0.3149 |
| AD08929 | 99.6364 | 0.2571 |

As shown in Tables 28A and B, CoV RNAi agents showed inhibition activity, up to 10000 inhibition of the CoV virus inhibition.

### Example 13. Testing of CoV RNAi Agents in Golden Syrian Hamsters Against SARS-CoV-2 Infection

Golden Syrian hamsters are described as a suitable model to test vaccines and therapeutics for the treatment of SARS-CoV-2 infection. The hamster model of SARS-CoV-2 infection shows signs of weight loss (morbidity), viral replication in the lungs and nasal turbinate, and significant histopathology changes including immune cell infiltration into the lungs. SARS-CoV-2 infection in the hamster model mimics mild SARS-CoV-2 infections reported in humans and, therefore, represents an excellent tool to test anti-SARS-CoV-2 agents (Chen et al, 2020; Imai et al, 2020).

Vero E6 cells obtained from the American Type Culture Collection (ATCC, CRL-1586) were cultured in Dulbecco's Modified Eagle Medium (DMEM) supplemented with 10% heat inactivated fetal bovine serum (FBS), penicillin (P; 100 IU/ml), streptomycin (S; 100 μg/ml) and L-glutamine (G; 292 μg/ml)) at 37° C. in a 5% CO₂ atmosphere.

SARS-CoV-2 WA-1/US 2020 strain (Genbank accession MT020880) was obtained from the Biodefense and Emerging Infections Research Resources Repository (BEI Resources, NR-52281). This SARS-CoV-2 WA-1/US 2020 strain was isolated from an oropharyngeal swab from a middle-age patient with a respiratory illness in January 2020 in the state of Washington, US. The virus stock received from BEI Resources was a passage (P4) stock. BEI Resources P4 stock was used to generate a master P5 seed stock. The P5 stock was further used to generate a P6 working stock. Both P5 and P6 SARS-CoV-2 WA-1/US 2020 stocks were generated by infecting Vero E6 cells at low multiplicity of infection (MOI, 0.01) for 72 h. At 72 h post-infection, tissue culture supernatants were collected, clarified, aliquoted, and stored at −80° C. A standard plaque assay (plaque forming units, PFU/ml) in Vero E6 cells was used to titrate P6 viral stock (2.5×10⁶ PFU/ml). Both P5 seed and P6 working stocks were sequenced, using next generation sequencing, and were identical to the BEI Resources original stock compromising virus infectivity.

Five-week-old male golden Syrian hamsters (n=70 and n=5 spare) were purchased from Charles River Laboratories (Wilmington, MA). Hamsters were provided sterile water and chow ad libitum and acclimatized for at least one week prior to experimental manipulation. All hamsters were healthy at the start of the experiment and ear tagged for identification.

Animals were distributed to the experimental groups as shown in Table 29. Animals were administered with either saline or test article (5 mg/kg in 2 ml/kg) via the intra-tracheal route on days −7 and −5. AC001888 includes an antisense strand nucleotide sequence targeting position 6412 of the SARS-CoV-2 genome, and AC001961 includes an antisense strand nucleotide sequence targeting position 28587 of the SARS-CoV-2 genome. The hamsters were challenged on day 7 post first administration of test articles, with 2×10⁵ PFU of SARS-CoV-2 (day 0). Hamsters were weighed daily and dosing volume was calculated and adjusted as weight changed for individual hamster. Animals were monitored for morbidity and mortality during the study and were euthanized on days 3 and 7 post infection by intraperitoneal injection of pentobarbital overdose (Fatal plus).

TABLE 29
CoV RNAi agent dosing for animal test groups.

| | Number of | Treatment | | Challenge |
|---|---|---|---|---|
| Experimental Group | Animals | Day | Route | (Intranasal) |
| Saline | 6 | Day −7, −5 | Intratracheal | SARS-CoV-2 |
| Saline + SARS-CoV-2 | 16 | Day −7, −5 | Intratracheal | SARS-CoV-2 |
| AC001888 + SARS- | 16 | Day −7, −5 | Intratracheal | SARS-CoV-2 |
| CoV-2 | | | | |
| AC001961 + SARS- | 16 | Day −7, −5 | Intratracheal | SARS-CoV-2 |
| CoV-2 | | | | |
| AC001888 + AC001961 + | 16 | Day −7, −5 | Intratracheal | SARS-CoV-2 |
| SARS-CoV-2 | | | | |

During necropsy, the trachea was cannulated and secured with a 2-0 suture, lungs were harvested and rinsed with PBS, and blot dried to avoid PBS getting into the airways. The right bronchus was clamped and ligated and right lung lobes were cut in half and weighed. One half of the right lung lobes were homogenized in Trizol for RNA isolation. The other half was homogenized in 1 ml of sterile PBS using Precellys tissue homogenizer (Bertin Instruments, Rockville, MD). Lung homogenates were centrifuged at 8,000×g for 15 min at 4° C. and supernatants were collected in aliquots and stored at −80° C. Left lungs were inflated (gravity instillation method) with 3 mL of 10% neutral buffered formalin fixative maintaining 23-25 cmH₂O pressure with fixative for 5 mins to prevent collapse and were submerged in over 10× volumes of 10% formalin (about 35 ml) for 7 days at room temperature. Ligature was removed seven days later, tissue rinsed with PBS, and transferred into 70% ethanol for further processing into paraffin blocks.

Vero E6 cells were seeded at a density of 2×10⁵ cells/well in flat bottom 24-well tissue culture plates. The following day, media was removed and replaced with 100 μl of ten-fold serial dilutions of the lung homogenate. Virus was adsorbed for 1 h at 37° C. in a humidified 5% CO₂ incubator. After viral adsorption, post infection media containing 0.9% agarose overlay (Sigma-Aldrich) was added and cells were incubated in a humidified 5% CO₂ incubator at 37° C. for 48 h. After 48 h, plates were inactivated in 10% neutral buffered formalin (NBF, Thermo-Fisher Scientific) for 12 h. For immunostaining, cells were washed three times with PBS and permeabilized with 0.5% Triton X-100 for 10 min at room temperature. Cells were immuno-stained with 1 μg/ml of a SARS-CoV-1/-2 nucleocapsid protein (NP) cross-reactive monoclonal antibody (Mab; Sigma-Aldrich) 1C7, diluted in 1% BSA for 1 h at 37° C. After incubation with the primary NP Mab, cells were washed three times with PBS, and developed with the Vectastain ABC kit and DAB Peroxidase Substrate kit (Vector 580 Laboratory, Inc., CA, USA) according to manufacturers' instructions. Viral determinations were counted and viral titers were calculated by number of counted plaques for a given dilution, and results were presented as PFU/ml.

One half of the right lung lobes was weighed and Trizol was calculated and added corresponding to lung tissue weight (1 ml Trizol/100 mg tissue). The tissues were homogenized using Precellys tissue homogenizer (Bertin Instruments, Rockville, MD) and the homogenate was stored at −80 C until RNA extraction. The frozen samples were thawed and 200 μl of chloroform was added to 1 ml lung homogenate. The tubes were then centrifuged and the aqueous layer transferred to a fresh tube. The subsequent steps were performed using on the KingFisher Flex System (Thermo Fisher) with NucleoMag Pathogen kit (Macherey-Nagel 744210.4).

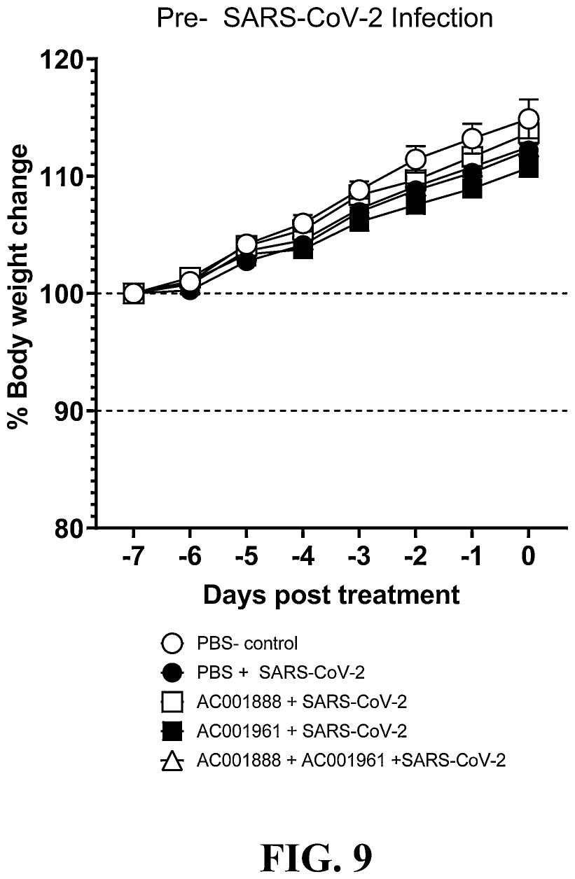

Hamsters were daily weighed just before the saline/test article treatment i.e. 7 days before SARS-CoV-2 infection (day 0) until the end of the study. Body weight at day −7 was used to calculate % body weight gain/loss in the pre-infection phase. Hamsters in all experimental groups continued to gain weight and showed no signs of morbidity post saline or test articles treatment. All hamsters remained healthy throughout the duration treatment (up to the day of virus challenge). As shown in FIG. 9, the groups receiving saline (n=22) had an average weight gain of 12.9%, whereas groups receiving AC001888 (n=16), AC001961 (n=16) and AC001888+AC001961 (n=16) had an average weight gain of 13.7%, 10.7% and 12.5% respectively on day 7 post first treatment.

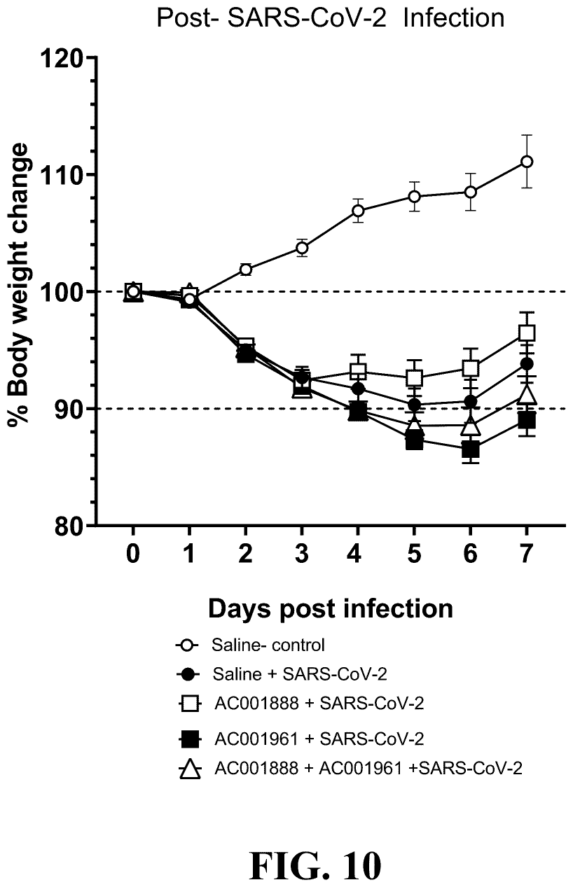

After SARS-CoV-2 infection, hamsters in saline group showed an average body weight loss of 7.3%, whereas hamsters in AC00188, AC001961 and AC00188+AC001961 showed an average body weight loss of 7.5%, 8.06% and 8.22% by day 3 post infection, respectively (n=16/group). On day 6 post infection, hamsters in saline, AC001888, AC001961 and AC001888+AC001961 showed an average body weight loss of 9.7%, 7.4%, 13.4% and 11.4% respectively (n=8/group; FIG. 10). Body weight at day 0 was used to calculate % body weight gain/loss in post infection phase.

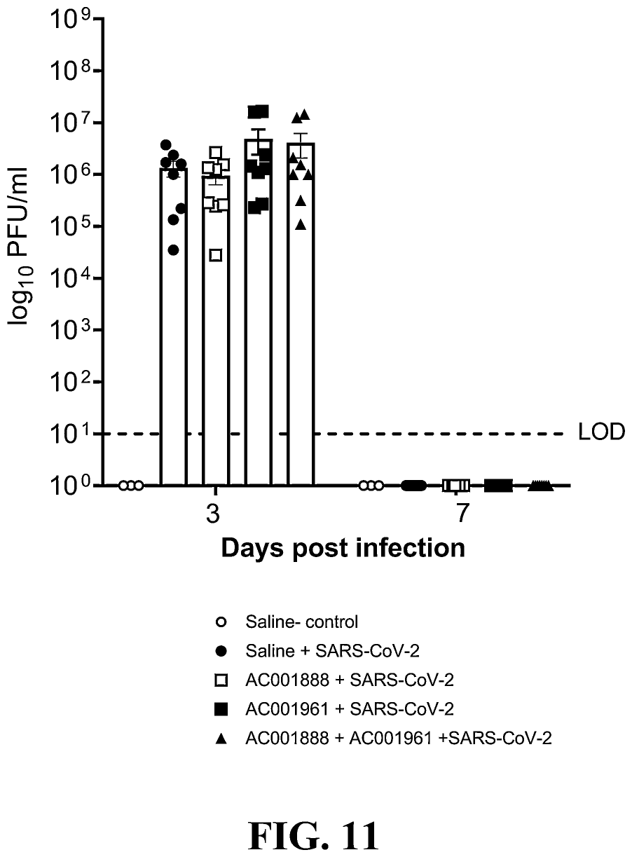

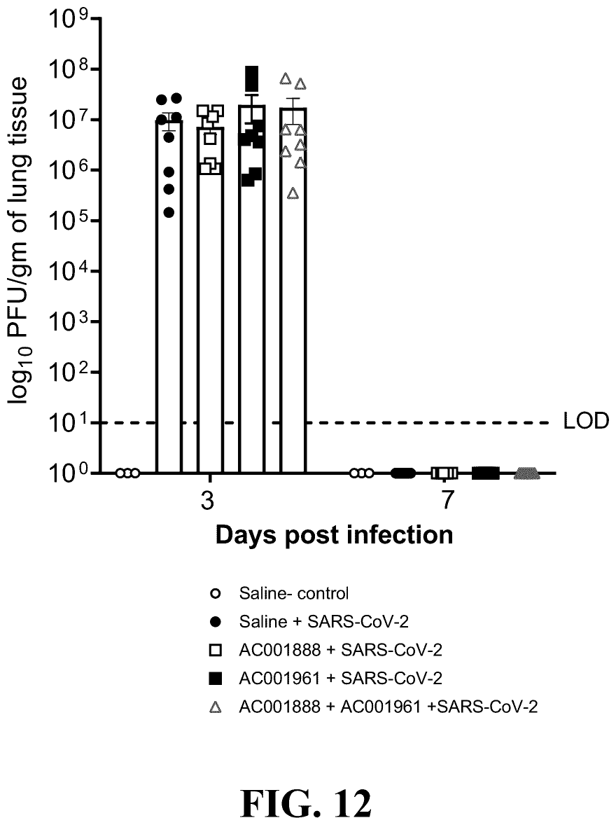

To determine the anti-SARS-CoV-2 effect of test articles, we performed plaque assay to quantitate viral titers in the lungs. Eight hamsters from each group were euthanized at days 3 and 7 post-infection and lungs were collected as described above. The viral titers are shown in FIGS. 11 and 12. At day 3 post infection, the average viral titers in the control group (Saline+SARS-CoV-2) was 1.3×10⁶ PFU/ml; whereas for the groups receiving the test articles, the average viral titers were; 9.5×10⁵ (AC001888); 4.9×10⁶ (AC001961) and 4.1×10⁶ (AC001888+AC001961) PFU/ml. No virus was detected at day 7 post infection in any of the groups. FIG. 12 represents the viral titer normalized to the weight of the tissue and expressed as PFU/gram of lung tissue. Viral load in lungs of saline and test article treated and SARS-CoV-2 infected hamsters showed comparable viral load (FIGS. 11 and 12).

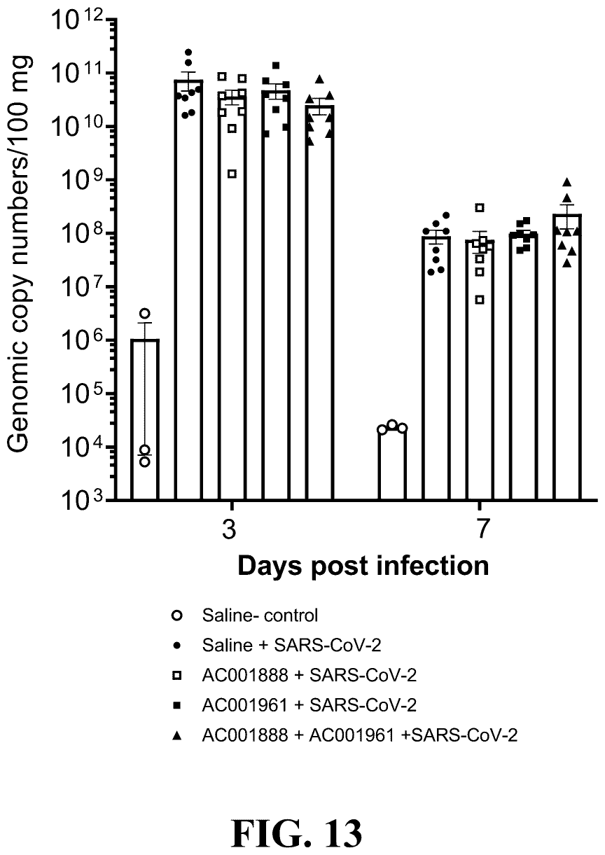

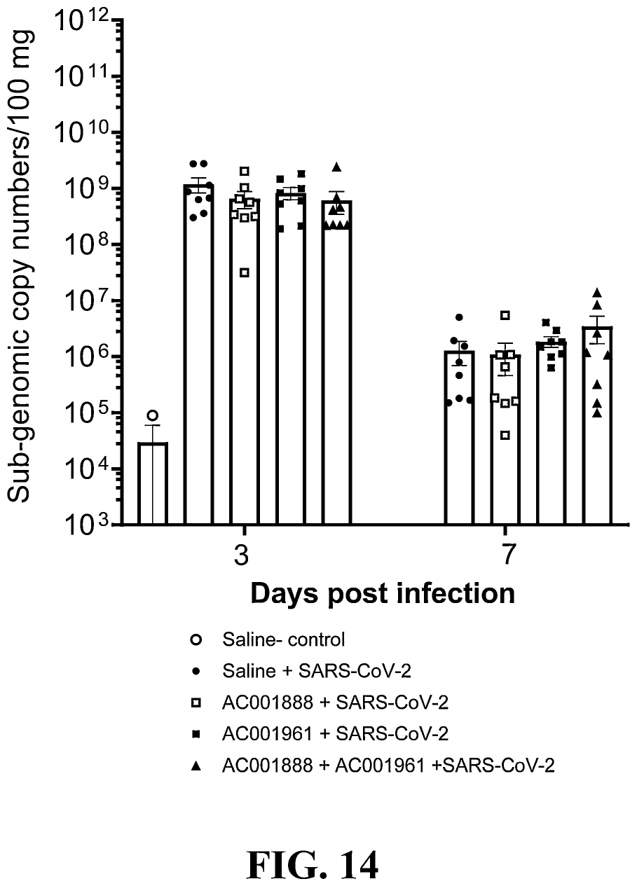

Viral genomic and subgenomic RNA copies were quantitated by RT-PCR using CDC recommended primers and probes set in the lung homogenate at day 3 and 7 post infection (FIGS. 13 and 14). The primer and probe set amplified the nucleoprotein (N) region of SARS-CoV-2 for genomic RNA copies, whereas the primer and probe set amplified the envelope (E) region for subgenomic RNA copies.

At day 3 post infection, the average genomic copies in the control group (Saline+SARS-CoV-2) was 10.9 logs/100 mg of lung tissue, whereas, among the test article groups, the average genomic copies measured were 10.6 (AC001888); 10.7 (AC001961) and 10.4 (AC001888+AC001961) logs/100 mg of lung tissue. The subgenomic RNA copies were approximately 2 logs lower than the genomic copies. The average subgenomic copies in the control group was 9.1 logs/100 mg of lung tissue, whereas, among the test article groups, the average subgenomic copies measured were 8.8 (AC001888); 8.9 (AC001961) and 8.8 (AC001888+AC001961) logs/100 mg of lung tissue.

The genomic and subgenomic viral RNA copies were also detected in lung tissues obtained at day 7 post infection. The levels were 2 to 3 logs lower than that observed on day 3 post infection. The average genomic copies in the control group were 7.9 logs/100 mg of lung tissue, whereas, among the test article groups, the average genomic copies measured were 7.9 (AC001888); 8 (AC0001961) and 8.4 (AC001888+AC001961) logs/100 mg of lung tissue. The average subgenomic copies in the control group at day 7 post infection was 6.1 logs/100 mg of lung tissue, whereas, among the test article groups, the average subgenomic copies measured were 6 (AC001888); 6.3 (AC001961) and 6.5 (AC001888+AC001961) logs/100 mg of lung tissue.

### Example 14. Testing of CoV RNAi Agents in Golden Syrian Hamsters Against SARS-CoV-2 Infection

SARS-CoV-2, USA-WA1/2020 strain (Gen Bank: MN985325.1) was obtained from BEI Resources (NR-52281). Passage 6 (P6) of SARS-CoV-2 was generated by infecting Vero E6 cells obtained from the American Type Culture Collection (ATCC, CRL-1586) for 72 h. At 72 h post-infection, tissue culture supernatants were collected, clarified, aliquoted, and stored at −80° C. A standard plaque assay (plaque forming units, PFU/ml) in Vero E6 cells was be used to titrate P6 viral stocks. P6 working stock was sequenced and was compared to the original stock for deletions or mutations compromising virus infectivity as provided by BEI Resources.

Six-eight weeks old male golden Syrian hamsters were purchased from Charles River Laboratories (Wilmington, MA.). Hamsters were provided sterile water and chow ad libitum and acclimatized for at least one week prior to experimental manipulation. Baseline body weights were measured before infection. Hamsters were infected intranasally (i.n., 50 μl per nostril) with 1×10⁴ PFU of SARS-CoV-2 in a final volume of 100 μl following isoflurane sedation.

Hamsters were housed in micro-isolator cages at the ABSL3. Hamsters were provided sterile water and chow ad libitum and acclimatized for at least one week prior to experimental manipulation. Baseline body weights were measured before treatment for RNAi dose calculations. Hamsters were treated by intra-tracheal route on day (−7) and (−5) before infection following isoflurane sedation. Hamsters were monitored and body weight recorded. On study day 0 (5 days post second RNAi treatment) hamsters were infected intranasally (i.n., 50 μl per nostril) with 1×10⁴ PFU of SARS-CoV-2 in a final volume of 100 μl following isoflurane sedation. Hamsters were monitored and body weight recorded up to day 7 post infection. On day 3 and 7 post infection, hamsters were euthanized by intra-peritoneal injection of pentobarbital overdose (Fatal plus).

TABLE 30
CoV RNAi agent dosing for animal test groups.

| | RNAi Agent | Number of | Treatment | | |
|---|---|---|---|---|---|
| Experimental Group | Dose | Animals | Day | Route | Challenge (Intranasal), at Day 0 |
| 1. Naïve, no infection | N/A | 3 | Day −7, −5 | Intratracheal | N/A |
| 2. Saline, no infection | N/A | 3 | Day −7, −5 | Intratracheal | N/A |
| 3. Saline, SARS-CoV-2 | N/A | 16 | Day −7, −5 | Intratracheal | SARS-CoV-2, 1 × 10⁴ PFU |
| 4. AC002623, SARS-CoV-2 | 5 mg/kg | 16 | Day −7, −5 | Intratracheal | SARS-CoV-2, 1 × 10⁴ PFU |
| 5. AC002622, SARS-CoV-2 | 5 mg/kg | 16 | Day −7, −5 | Intratracheal | SARS-CoV-2, 1 × 10⁴ PFU |
| 6. AC002619, SARS-CoV-2 | 5 mg/kg | 16 | Day −7, −5 | Intratracheal | SARS-CoV-2, 1 × 10⁴ PFU |
| 7. AC001927 (RISC-blocked), | 5 mg/kg | 16 | Day −7, −5 | Intratracheal | SARS-CoV-2, 1 × 10⁴ PFU |
| SARS-CoV-2 | | | | | |
| 8. AC002617, SARS-CoV-2 | 5 mg/kg | 16 | Day −7, −5 | Intratracheal | SARS-CoV-2, 1 × 10⁴ PFU |
| 9. AC002618, SARS-CoV-2 | 5 mg/kg | 16 | Day −7, −5 | Intratracheal | SARS-CoV-2, 1 × 10⁴ PFU |
| 10. AC002620, SARS-CoV-2 | 5 mg/kg | 16 | Day −7, −5 | Intratracheal | SARS-CoV-2, 1 × 10⁴ PFU |
| 11. AC002621, SARS-CoV-2 | 5 mg/kg | 16 | Day −7, −5 | Intratracheal | SARS-CoV-2, 1 × 10⁴ PFU |

AC002617 and AC002618 each include an antisense strand nucleotide sequence targeting position 6412 of the SARS-CoV-2 genome; AC002619 includes an antisense strand nucleotide sequence targeting position 29150 of the SARS-CoV-2 genome; AC002620 includes an antisense strand nucleotide sequence targeting position 4917 of the SARS-CoV-2 genome; AC002621 includes an antisense strand nucleotide sequence targeting position 4156 of the SARS-CoV-2 genome; AC002622 includes an antisense strand nucleotide sequence targeting position 15886 of the SARS-CoV-2 genome; and AC002623 includes an antisense strand nucleotide sequence targeting position 14503 of the SARS-CoV-2 genome.

Trachea were cannulated and secured with 2-0 or 1-0 suture. The lung was harvested in monobloc without heart, lobes rinsed with PBS and blotted dry, while αvoiding getting PBS into airways. Left bronchus was clamped with mosquito, ligated and left lung lobe was cut longitudinally and both halves were weighed. One half of the lobe was collected in a cryovial for RNA isolation using Trizol homogenization. SARS-CoV-2 RNA was measured with CDC recommended N1 probe for genomic copies and probe in E for subgenomic RNA copies, by real-time reverse transcriptase qPCR (RT-qPCR). The other half of the left lung lobe was collected in PBS and homogenized aliquots were frozen at −80 C for PFU measurement.

Right lungs were inflated with gravity instillation of 10% neutral buffered formalin fixative maintaining 23-25 cm H₂O pressure with fixative for 5 minutes to prevent collapse, ligated to keep fixative in lung, and submerged in over 10× volumes of fixative for 7 days at room temperature. Ligatures were removed and tissue rinsed with PBS. Further processing into paraffin blocks was to measure inflammation, perform immunohistochemistry of viral proteins (TBD), and for RNA scope detection of viral RNA.

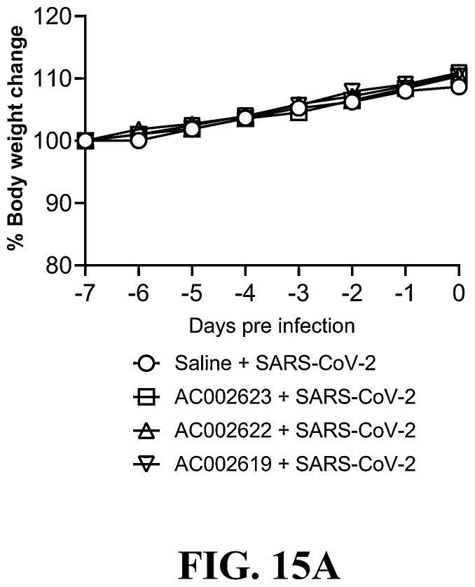

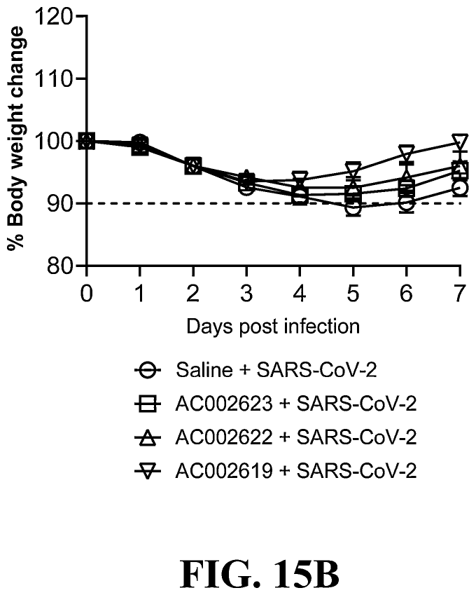

The hamsters in groups 3-6 were treated as one cohort. Hamsters were daily weighed just before the saline/test article treatment i.e. 7 days before SARS-CoV-2 infection (day 0) until the end of the study. Body weight at day −7 was used to calculate % body weight gain/loss in the pre-infection phase. Hamsters in all experimental groups continued to gain weight and showed no signs of morbidity post saline or test articles treatment. All hamsters remained healthy throughout the duration treatment (up to the day of virus challenge). As shown in FIG. 15A, the groups receiving saline (n=16) had an average weight gain of 8.6%, whereas groups receiving AC002623 (n=16), AC002622 (n=16) and AC002619 (n=16) had an average weight gain of 10.3, 10.5 and 10.9% respectively on day 7 post first treatment.

After SARS-CoV-2 infection, hamsters in saline group showed an average body weight loss of 7.4%, whereas hamsters in AC002623, AC002622 and AC002619 showed an average body weight loss of 6.6%, 5.8% and 5.9% by day 3 post infection respectively (n=16/group). On day 6 post infection, hamsters in saline, AC002623, AC002622 and AC002619 showed an average body weight loss of 7.5%, 4.75%, 2.8% and 1.7% respectively (n=8/group; FIG. 15B). Body weight at day 0 was used to calculate % body weight gain/loss in post infection phase.

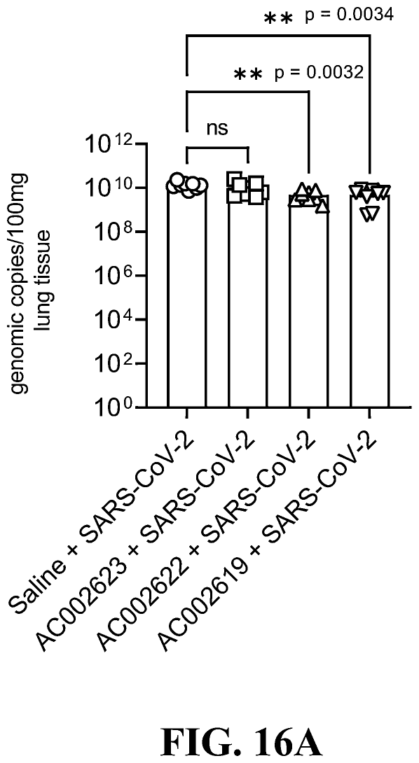

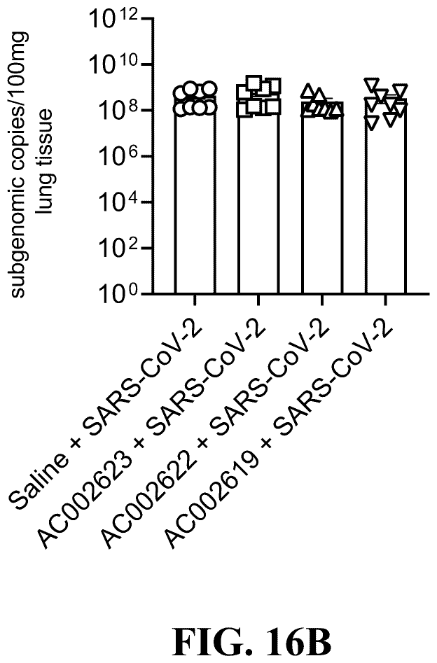

The genomic and subgenomic CoV viral copy levels 3 days post infection are shown in FIGS. 16A and 16B, respectively. As shown in FIG. 16A, CoV RNAi agents AC002622 and AC002619 both demonstrate significant reduction of CoV genomic viral RNA 3 days post CoV infection. At day 3 post infection, the average genomic copies in the control group (Saline+SARS-CoV-2) was 10.0 logs/100 mg of lung tissue, whereas, among the test article groups, the average genomic copies measured were 9.9 (AC002623); 9.6 (AC002622) and 9.4 (AC002619) logs/100 mg of lung tissue. The subgenomic RNA copies were approximately 2 logs lower than the genomic copies. The average subgenomic copies in the control group was 8.4 logs/100 mg of lung tissue, whereas, among the test article groups, the average subgenomic copies measured were 8.5 (AC002623); 8.2 (AC002622) and 8.1 (AC002619) logs/100 mg of lung tissue.

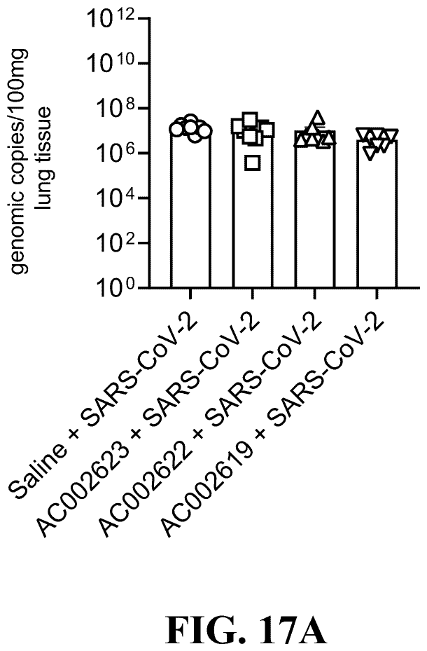

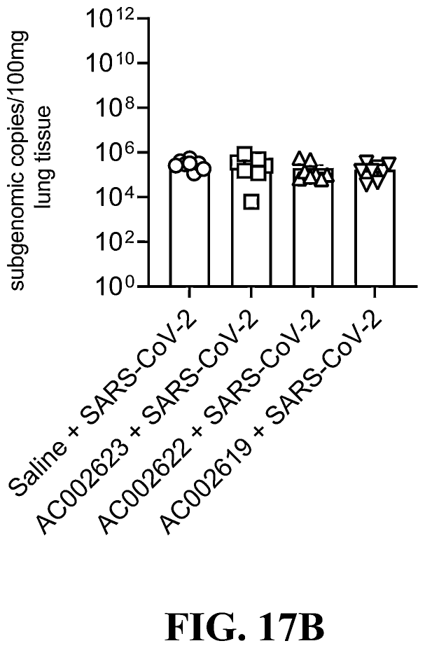

The genomic and subgenomic CoV viral copy levels 7 days post infection are shown in FIGS. 17A and 17B, respectively. The average genomic copies in the control group were 8.1 logs/100 mg of lung tissue, whereas, among the test article groups, the average genomic copies measured were 7.8 (AC002623), 7.8 (AC002622) and 7.5 (AC002619) logs/100 mg of lung tissue. The average subgenomic copies in the control group at day 7 post infection was 6.4 logs/100 mg of lung tissue, whereas, among the test article groups, the average subgenomic copies measured were 6.2 (AC002623), 6.1 (AC002622) and 6.0 (AC002619) logs/100 mg of lung tissue.

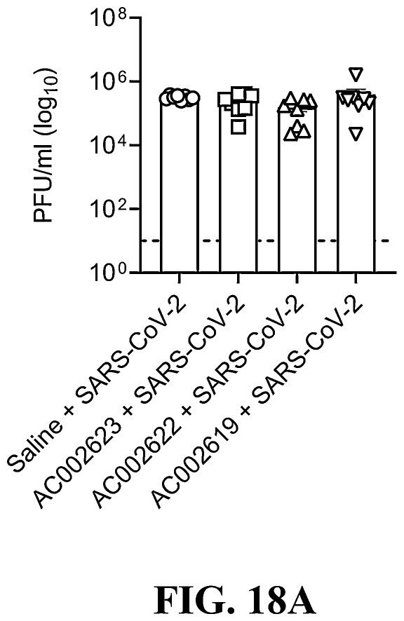

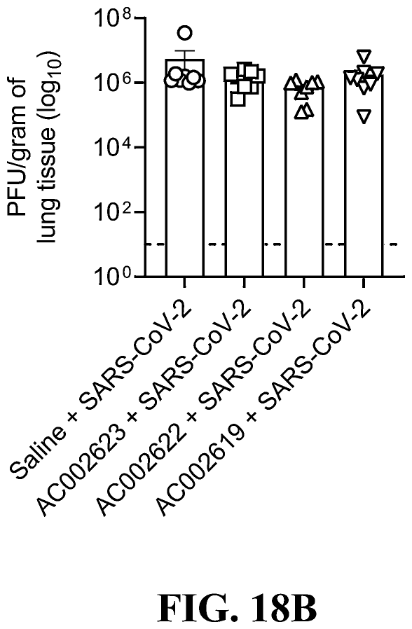

The viral titers determined by plaque assay in PFU/ml as described in Example 13 are shown in FIG. 18A. At day 3 post infection, the average viral titers in the control group (Saline+SARS-CoV-2) was 5.5 log 10 PFU/ml; whereas for the groups receiving the test articles, the average viral titers were 5.4 log 10 (AC002623); 5.2 log 10 (AC002622) and 5.3 log 10 (AC002619) PFU/ml. FIG. 18B represents the viral titer normalized to the weight of the tissue and expressed as PFU/gram of lung tissue. The average viral load in lungs of the control group infected hamsters (Saline+SARS-CoV-2) at day 3 post infection was 6.2 log 10 PFU/g and in the groups receiving the test articles were 6.1 log 10 (AC002623); 5.8 log 10 (AC002622) and 6.2 log 10 (AC002619) PFU/g. On Day 7 post infection, no virus was detected by plaque assay.

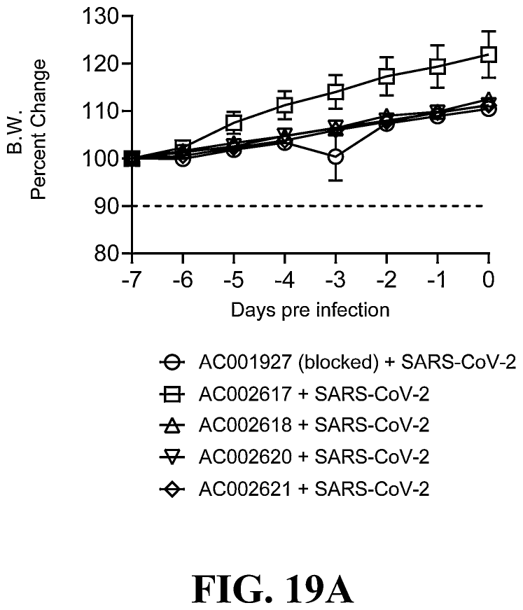

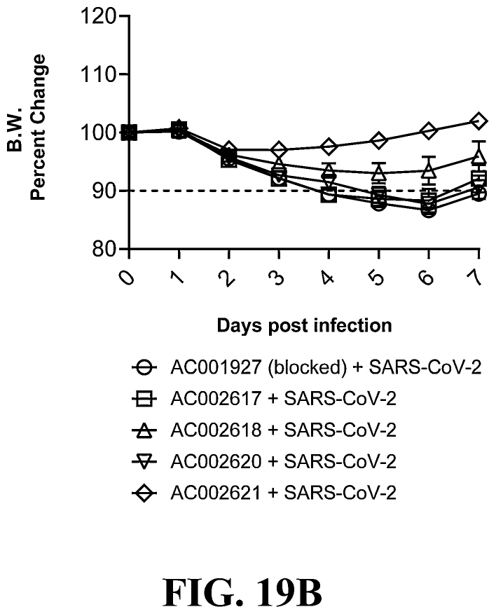

The hamsters in groups 7-11 were treated as a separate cohort. In this cohort the trigger employed specific chemical modifications to block the antisense strand from RISC-loading (AC001927), which served as a control. As such, AC001927 was unable to initiate RISC and RNAi-mediated gene expression silencing. Hamsters were daily weighed just before the saline/test article treatment i.e. 7 days before SARS-CoV-2 infection (day 0) until the end of the study. Body weight at day −7 was used to calculate % body weight gain/loss in the pre-infection phase. Hamsters in all experimental groups continued to gain weight and showed no signs of morbidity post control trigger or test articles treatment. All hamsters remained healthy throughout the duration treatment (up to the day of virus challenge). As shown in FIG. 19A, the groups receiving blocked control AC001927 (n=16) had an average weight gain of 17.3%, whereas groups receiving AC002617 (n=16), AC002618 (n=16), AC002620 (n=16) and AC002621 (n=16) had an average weight gain of 28.8, 19.8, 18.4 and 17.9, respectively, on day 7 post first treatment.

After SARS-CoV-2 infection, hamsters in AC001927 control group showed an average body weight loss of 2.2%, whereas hamsters treated with AC002617, AC002618, AC002620 and AC002621 showed an average body weight loss of 2.3%, −0.6% (Gaining weight), 1.4% and −3.1% (Gaining weight) by day 3 post infection, respectively (n=16/group). On day 7 post infection, hamsters receiving AC001927, AC002617, AC002618, AC002620 and AC002621 showed an average body weight loss of 10.2%, 9.4%, 6.1%, 8.6% and −1.1% (Gaining weight), respectively (n=8/group; FIG. 19B). Body weight at day 0 was used to calculate % body weight gain/loss in post infection phase.

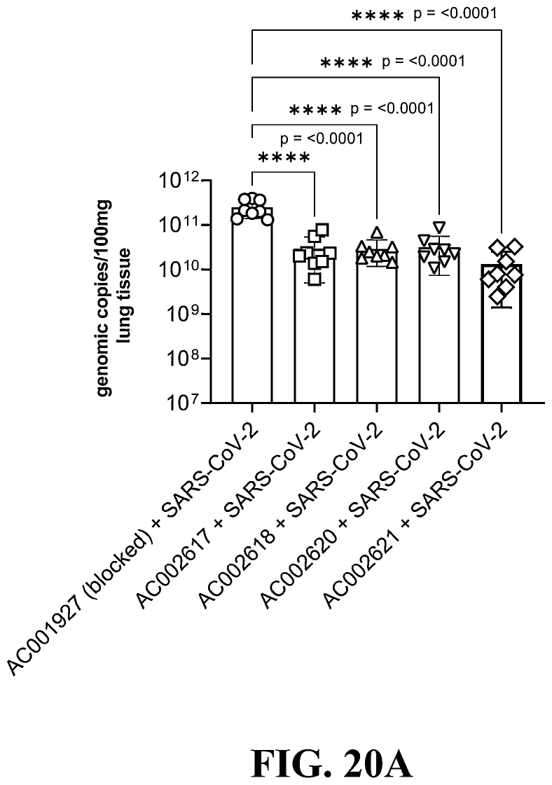

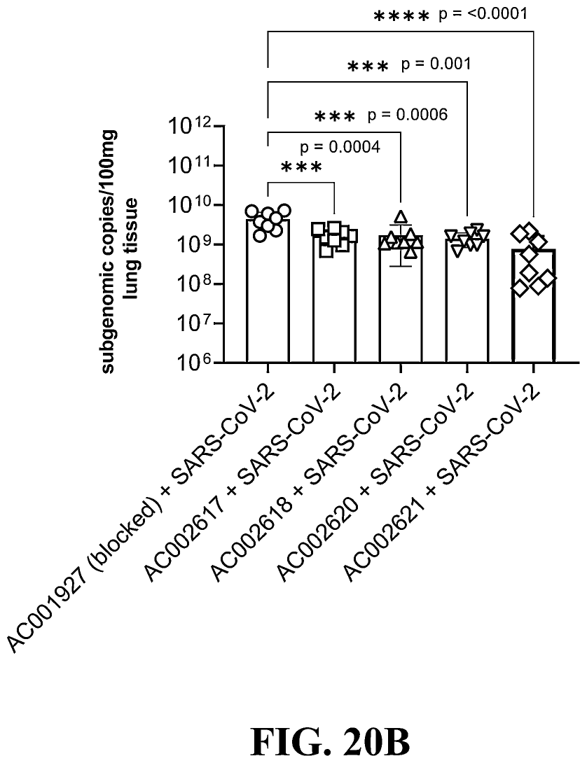

The genomic and subgenomic CoV viral copy levels 3 days post infection are shown in FIGS. 20A and 20B, respectively. CoV RNAi agents AC002617, AC002618, AC002620, and AC002621 all demonstrate significant reduction of both CoV genomic and subgenomic viral RNA 3 days post CoV infection. At day 3 post infection, the average genomic copies in the control group (AC001927+SARS-CoV-2) was 11.4 logs/100 mg of lung tissue, whereas, among the test article groups, the average genomic copies measured were 10.3 (AC002617), 10.4 (AC002618), 10.4 (AC002620) and 10.0 (AC002621) logs/100 mg of lung tissue. The subgenomic RNA copies were approximately 2 logs lower than the genomic copies. The average subgenomic copies in the control AC001927 group was 9.6 logs/100 mg of lung tissue, whereas, among the test article groups, the average subgenomic copies measured were 9.2 (AC002617), 9.1 (AC002618), 9.1 (AC002620) and 8.6 (AC002621) logs/100 mg of lung tissue.

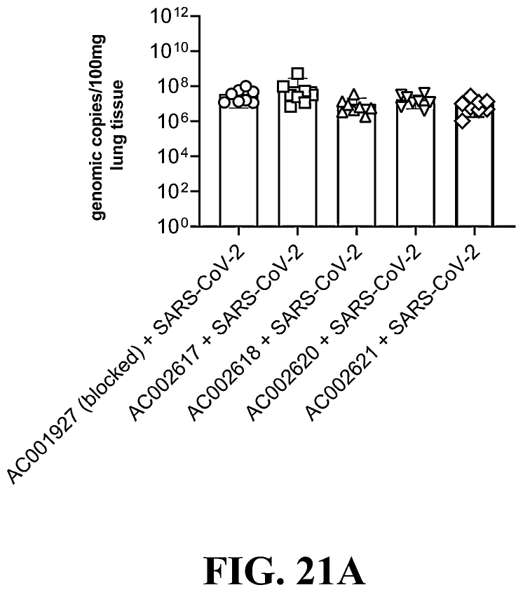

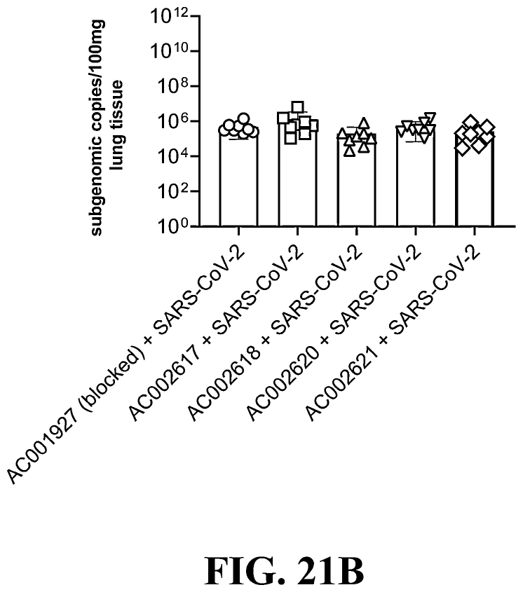

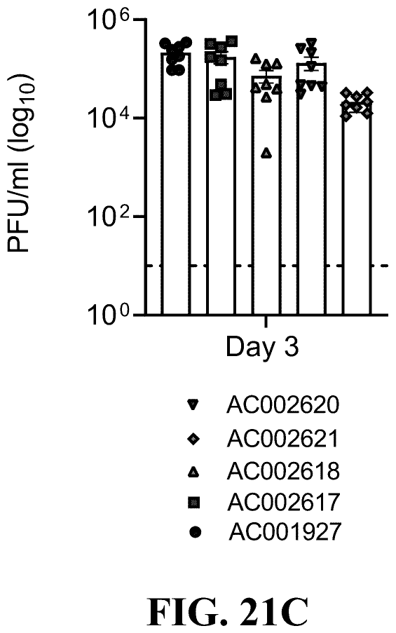

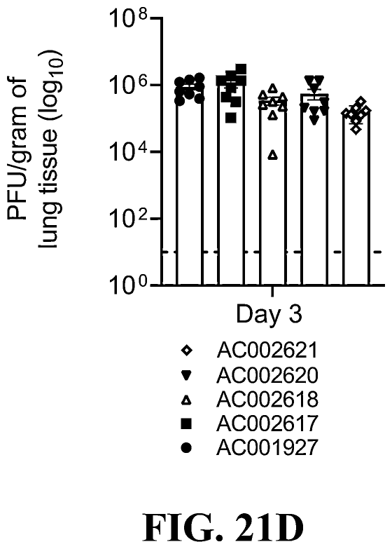

The genomic and subgenomic CoV viral copy levels 7 days post infection are shown in FIGS. 21A and 21B, respectively. The average genomic copies in the control group were 8.4 logs/100 mg of lung tissue, whereas, among the test article groups, the average genomic copies measured were 8.6 (AC002617), 7.8 (AC002618), 8.1 (AC002620) and 7.9 (AC002621) logs/100 mg of lung tissue. The average subgenomic copies in the control group at day 7 post infection was 6.6 logs/100 mg of lung tissue, whereas, among the test article groups, the average subgenomic copies measured were 6.8 (AC002617), 6.0 (AC002618), 6.6 (AC002620) and 6.2 (AC002621) logs/100 mg of lung tissue.

The viral titers determined by plaque assay in PFU/ml as described in Example 13 are shown in FIG. 21C. At day 3 post infection, the average viral titers in the control AC001927 group was 5.3 log 10 PFU/ml; whereas for the groups receiving the test articles, the average viral titers were 5.2 log 10 (AC002617); 4.8 log 10 (AC002618); 5.1 log 10 (AC002620) and 4.3 log 10 (AC002621) PFU/ml. FIG. 21D represents the viral titer normalized to the weight of the tissue and expressed as PFU/gram of lung tissue. The average viral load in lungs of the control AC001927 group infected hamsters at day 3 post infection was 5.87 log 10 PFU/g and in the groups receiving the test articles were 6 log 10 (AC002617); 5.5 log 10 (AC002618); 5.7 log 10 (AC002620) and 5.1 log 10 (AC002621) PFU/g. On Day 7 post infection, no virus was detected by plaque assay.

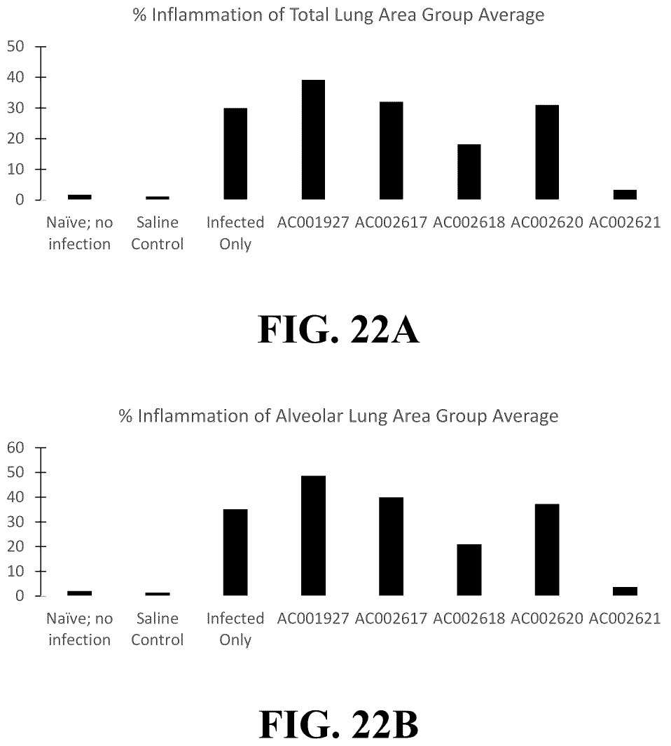

Inflammation in hamster lung tissue was measured from hematoxylin and eosin (H&E) staining of right superior lobe tissue sections followed by HALO quantitation. FIG. 22A shows group averages of the total pulmonary inflammation as a percentage of the tissue on Day 7 after infection in hamsters that were naïve and uninfected (1.9%), uninfected saline controls (1.3%), infected saline controls (30.2%), treated with RISC-blocked negative control AC001927 and infected (39.3%), and groups treated with CoV RNAi agents AC002617 (32.2%), AC002618 (18.4%), AC002620 (31.2%), and AC002621 (3.5%). FIG. 22B shows the percentage of the alveolar lung area with inflammation in naïve and uninfected (2.1%), uninfected saline controls (1.4%), infected saline controls (35.2%), treated with RISC-blocked negative control AC001927 and infected (48.7%), and groups treated with CoV RNAi agents AC002617 (39.9%), AC002618 (20.9%), AC002620 (37.2%), and AC002621 (3.7%). Syrian golden hamsters infected with SARS-CoV-2, upon treatment with the CoV RNAi agent AC002621, showed significant reduction in pulmonary inflammation in both total area and alveolar area.

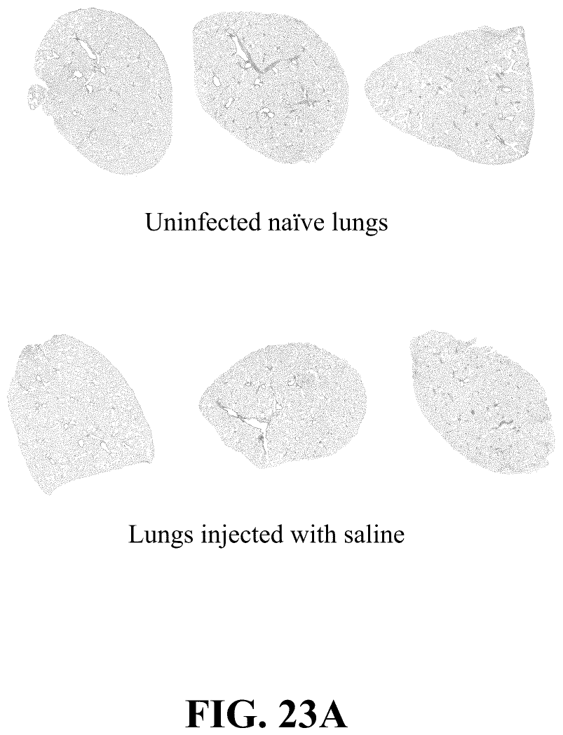

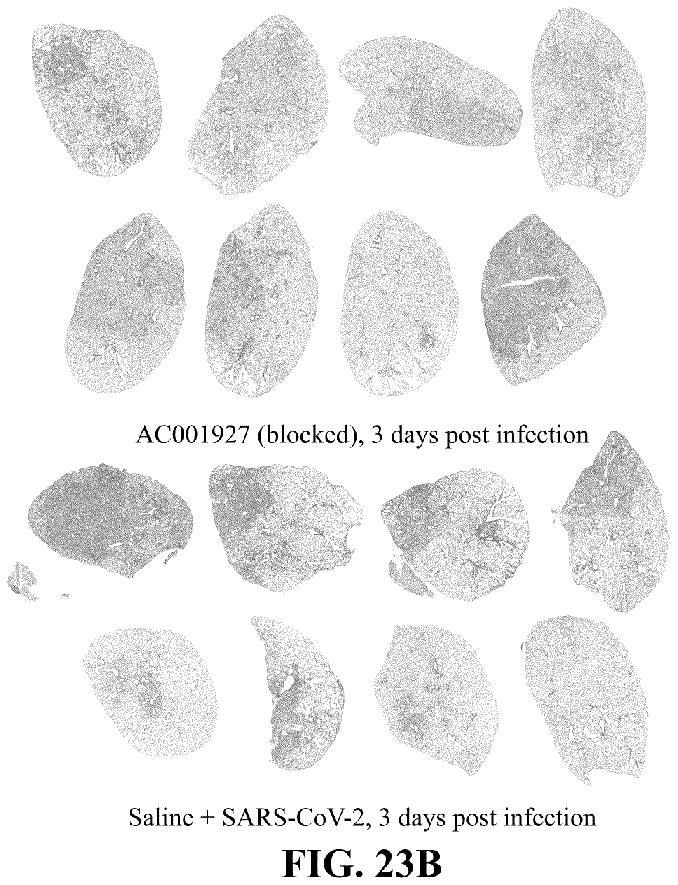

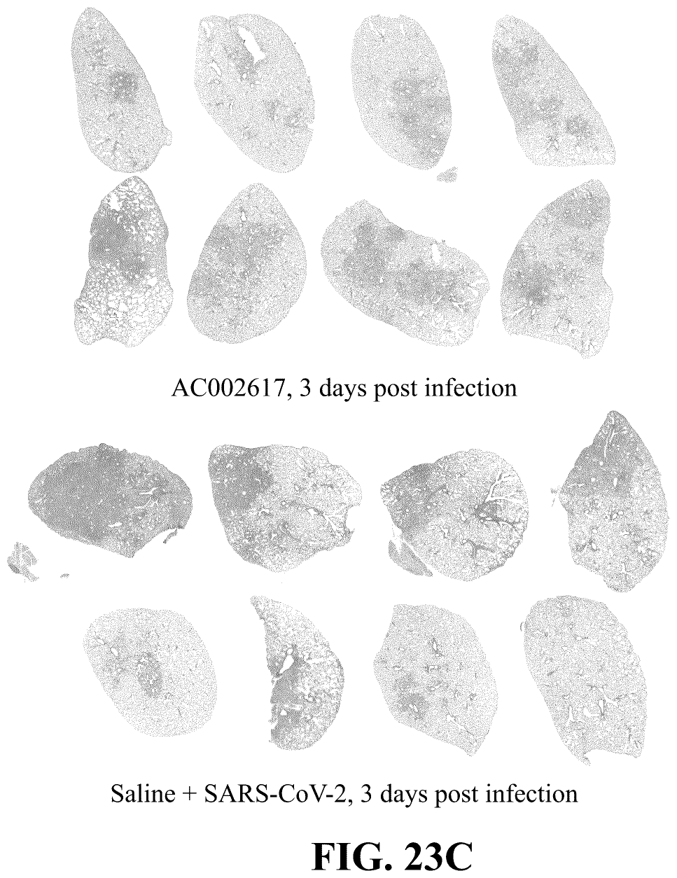

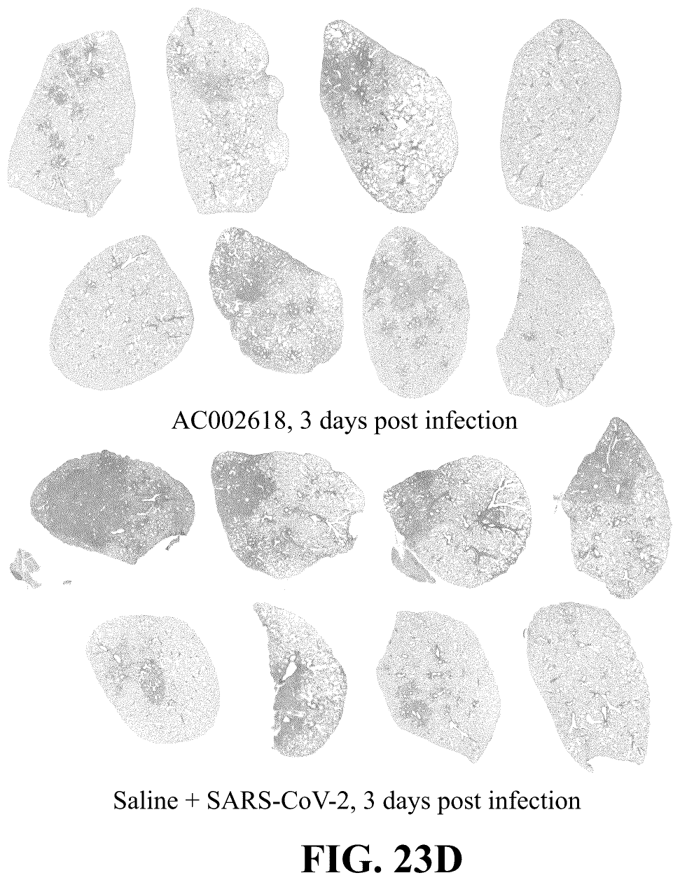

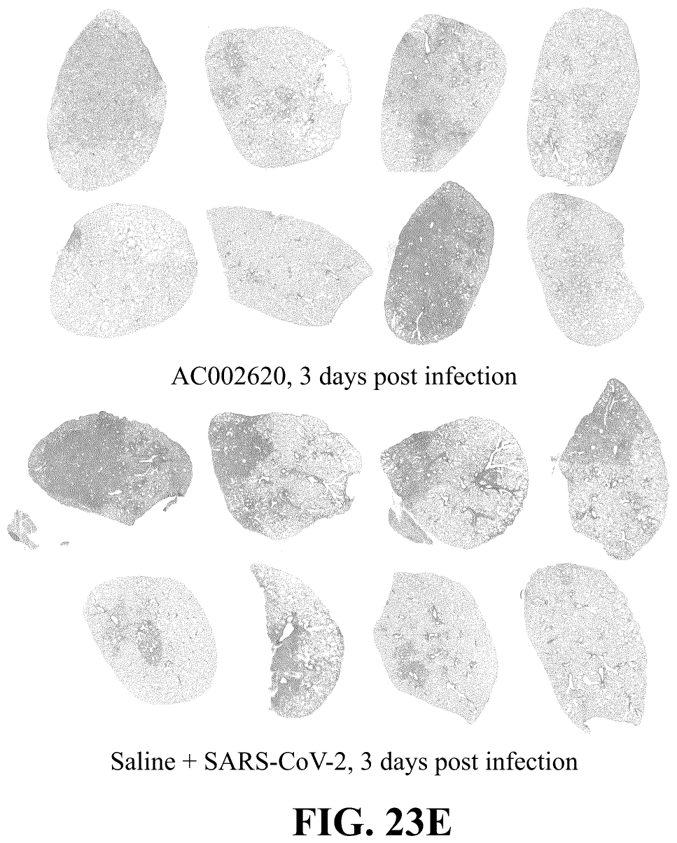

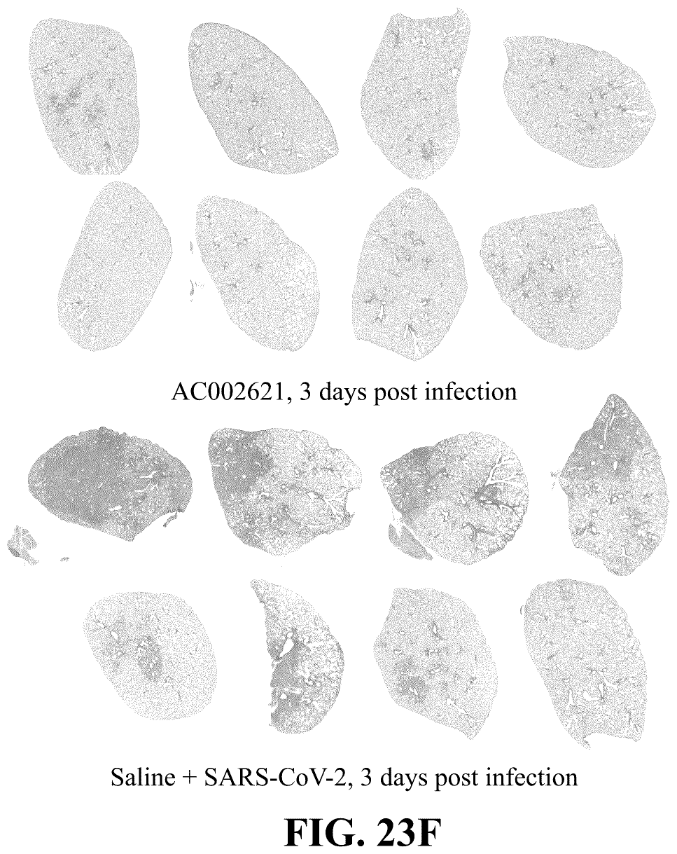

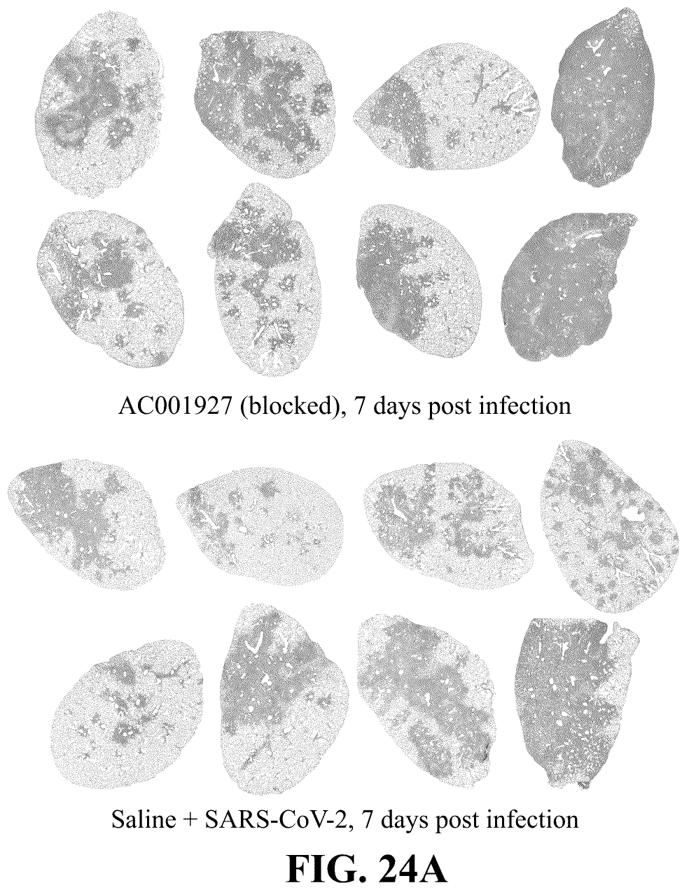

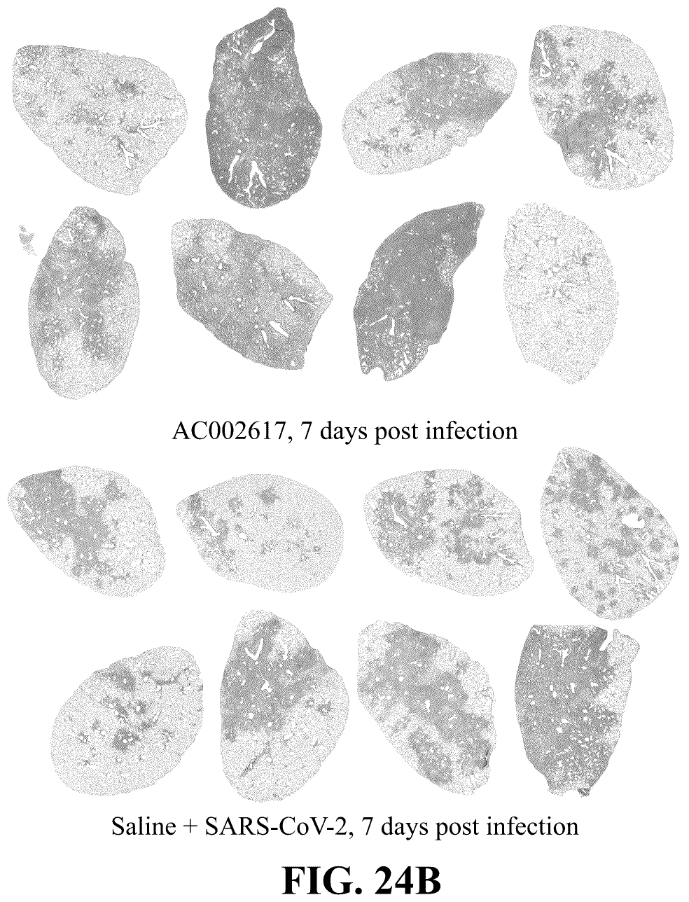

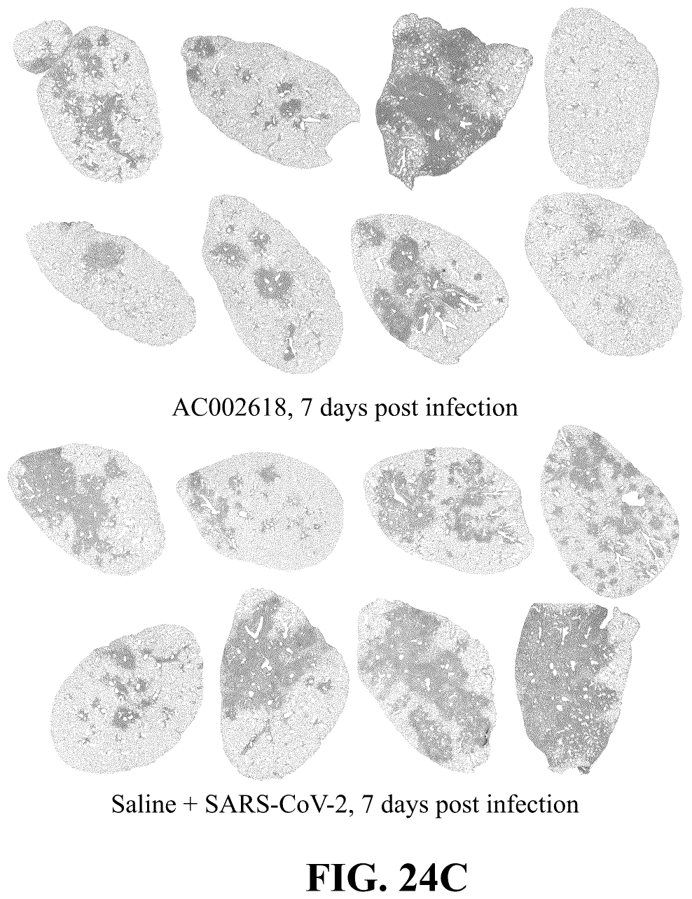

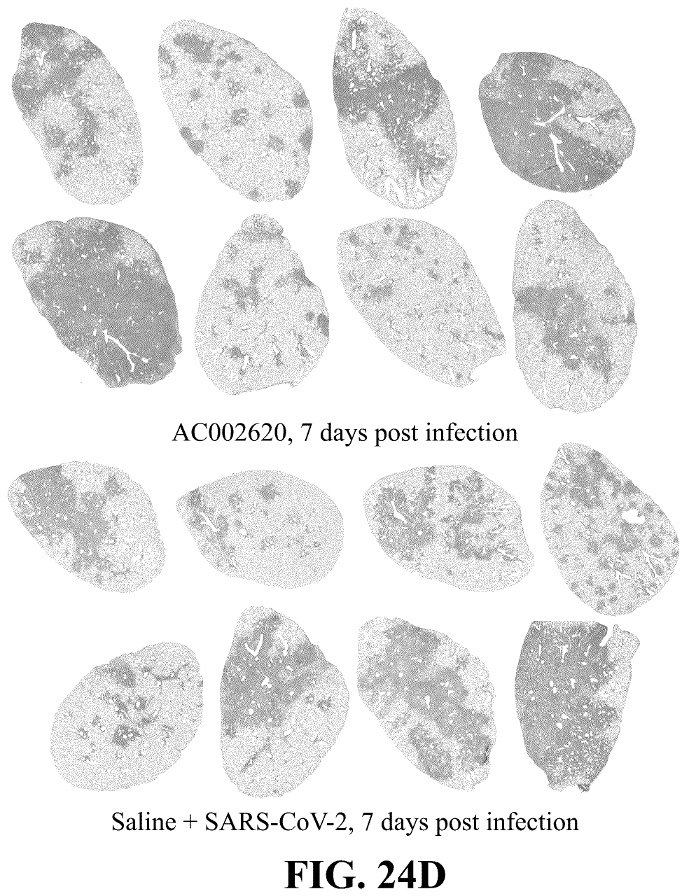

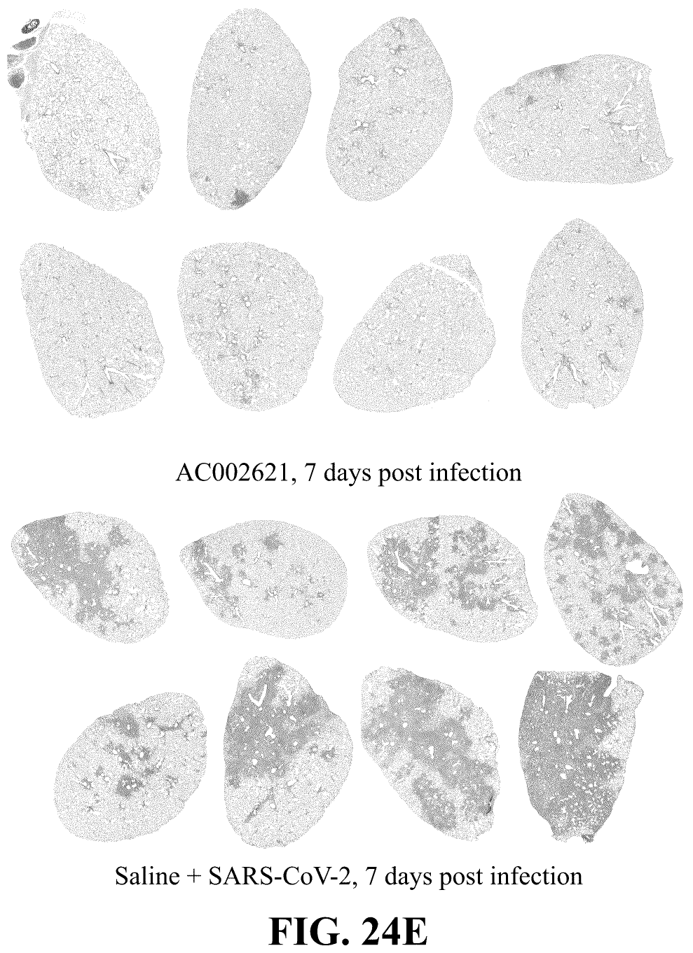

Superior lobe tissue sections stained with H&E are shown in FIGS. 23 and 24. FIG. 23A shows pulmonary tissue of the uninfected naïve and saline-injected hamsters. FIG. 23B demonstrates that three days post-infection the lungs of hamsters injected with RISC-blocked AC001927 were similarly inflamed to those of saline-injected and infected hamsters. FIGS. 23C, 23D, 23E and 23F show the superior lobe of the hamster lungs infected with SARS-CoV-2, subsequent treatment with the CoV RNAi agents, at 3 days post infection, compared to the saline control treated with SARS-CoV-2. The CoV RNAi agent AC002621 (FIG. 23F) achieved significant reduction in lung inflammation. More specifically, AC002621 demonstrates marked reduction in lung inflammation compared to the other CoV RNAi agents in this study.

FIGS. 24A, 24B, 24C, 24D, and 24E show the superior lobe of the hamster lungs infected with SARS-CoV-2, subsequent treatment with RISC-blocked negative control AC0001927 (FIG. 24A) or the CoV RNAi agents, at 7 days post infection, compared to the saline control treated with SARS-CoV-2. Inflammation in hamsters treated with the negative control AC001927 was similar to that in hamsters injected with saline (FIG. 24A). As shown these five Figures, the CoV RNAi agent AC002621 (FIG. 24E) demonstrates marked reduction in lung inflammation compared to the other CoV RNAi agents.

### Example 15. Testing of CoV RNAi Agents in Golden Syrian Hamsters Against SARS-CoV-2 Infection

Golden Syrian hamsters are described as a suitable model to test vaccines and therapeutics for the treatment of SARS-CoV-2 infection. The hamster model of SARS-CoV-2 infection shows signs of weight loss (morbidity), viral replication in the lungs and nasal turbinate, and significant histopathology changes including immune cell infiltration into the lungs. SARS-CoV-2 infection in the hamster model mimics mild SARS-CoV-2 infections reported in humans, and, therefore represents an excellent tool to test anti-SARS-CoV-2 agents (Chen et al, 2020; Imai et al, 2020).

Thirteen (13) week old male Syrian golden hamsters were selected for this study. Animals were distributed to the experimental groups as shown in Tables 31 and 32. Animals were administered with either saline or test article (2 ml/kg) via the intra-tracheal route on days −14 and −12. The hamsters were challenged 14 days post first administration of test articles, with 1×10⁵′ PFU of SARS-CoV-2 (day 0). Hamsters were weighed daily and dosing volume was calculated and adjusted as weight changed for individual hamster. Animals were monitored for morbidity and mortality during the study and were euthanized on days 3 and 7 post infection.

Upon euthanasia, lung tissue was harvested. Right lung lobes were separated from the main bronchus, cut in half, and further processed for RNA isolation and viral RNA qPCR analysis. Left lung was fixed processed for RNA scope and immunohistochemistry.

TABLE 31
CoV RNAi agent dosing for animal test groups, euthanized at 3 days post-infection.

| | | Number of | Treatment | | |
|---|---|---|---|---|---|
| Experimental Group | RNAi Agent Dose | Animals | Day | Route | Challenge (Intranasal) |
| Saline + SARS-CoV-2 | N/A | 8 | Day −14, −12 | Intratracheal | SARS-CoV-2, 5 × 10⁴ PFU/nostril |
| AC000234 + SARS- | 5 mg/kg | 8 | Day −14, −12 | Intratracheal | SARS-CoV-2, 5 × 10⁴ PFU/nostril |
| CoV-2 | | | | | |
| AC000234 + AC001888 + | 2.5 mg/kg AC000234, | 8 | Day −14, −12 | Intratracheal | SARS-CoV-2, 5 × 10⁴ PFU/nostril |
| SARS-CoV-2 | 2.5 mg/kg AC001888 | | | | |
| AC001888 + SARS- | 5 mg/kg | 8 | Day −14, −12 | Intratracheal | SARS-CoV-2, 5 × 10⁴ PFU/nostril |
| CoV-2 | | | | | |

TABLE 32
CoV RNAi agent dosing for animal test groups, euthanized at 7 days post-infection.

| | | Number of | Treatment | | |
|---|---|---|---|---|---|
| Experimental Group | RNAi Agent Dose | Animals | Day | Route | Challenge (Intranasal) |
| Saline* | N/A | 1 | Day −14, −12 | Intratracheal | N/A |
| Saline + SARS-CoV-2 | N/A | 8 | Day −14, −12 | Intratracheal | SARS-CoV-2, 5 × 10⁴ PFU/nostril |
| AC000234 + SARS- | 5 mg/kg | 8 | Day −14, −12 | Intratracheal | SARS-CoV-2, 5 × 10⁴ PFU/nostril |
| CoV-2 | | | | | |
| AC000234 + AC001888 + | 2.5 mg/kg AC000234, | 8 | Day −14, −12 | Intratracheal | SARS-CoV-2, 5 × 10⁴ PFU/nostril |
| SARS-CoV-2 | 2.5 mg/kg AC001888 | | | | |
| AC001888 + SARS- | 5 mg/kg | 8 | Day −14, −12 | Intratracheal | SARS-CoV-2, 5 × 10⁴ PFU/nostril |
| CoV-2 | | | | | |

*Euthanized on post infection Day 6.

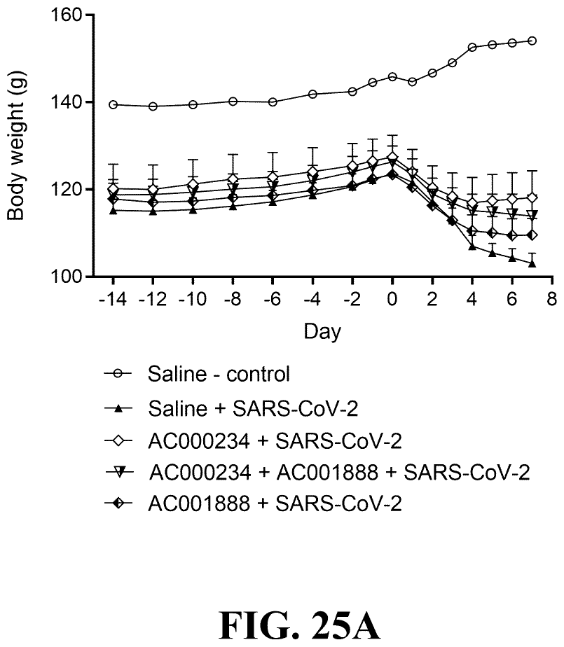

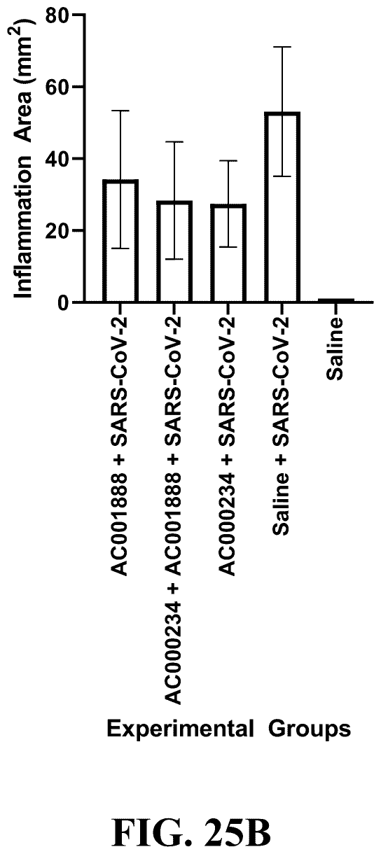

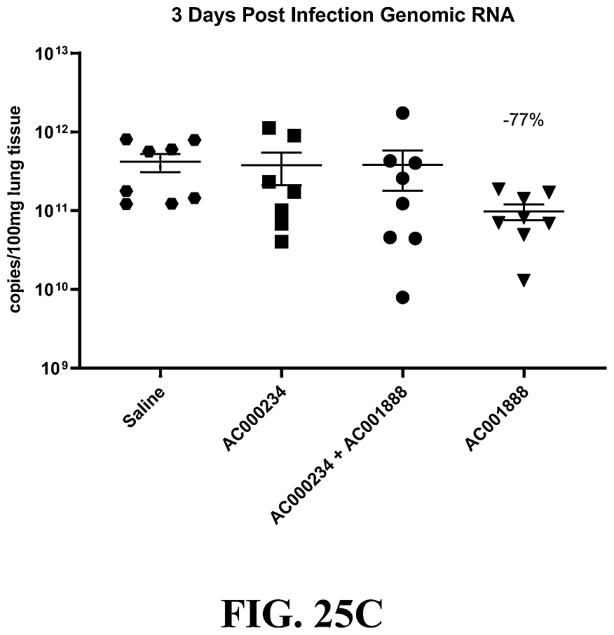

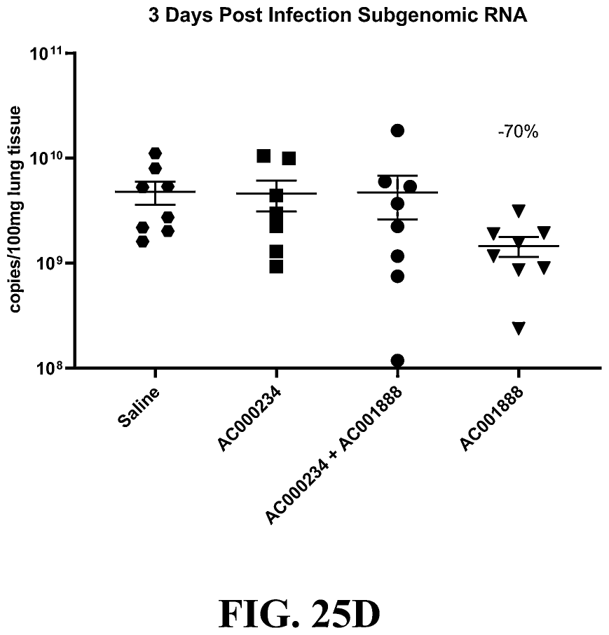

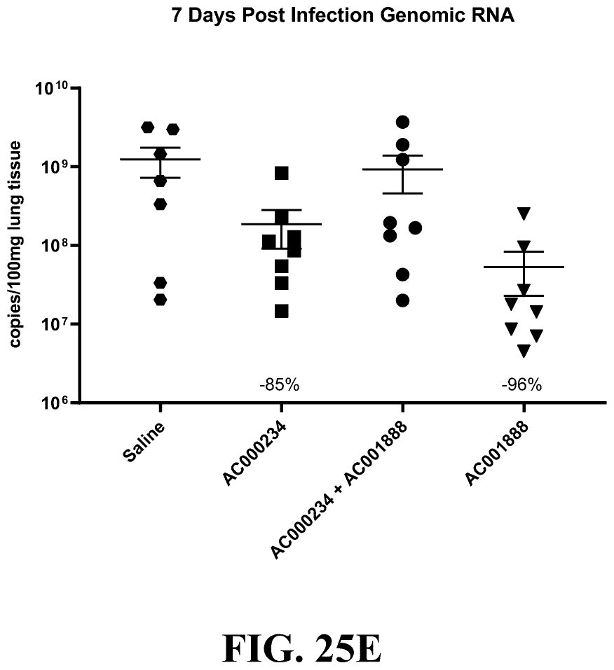

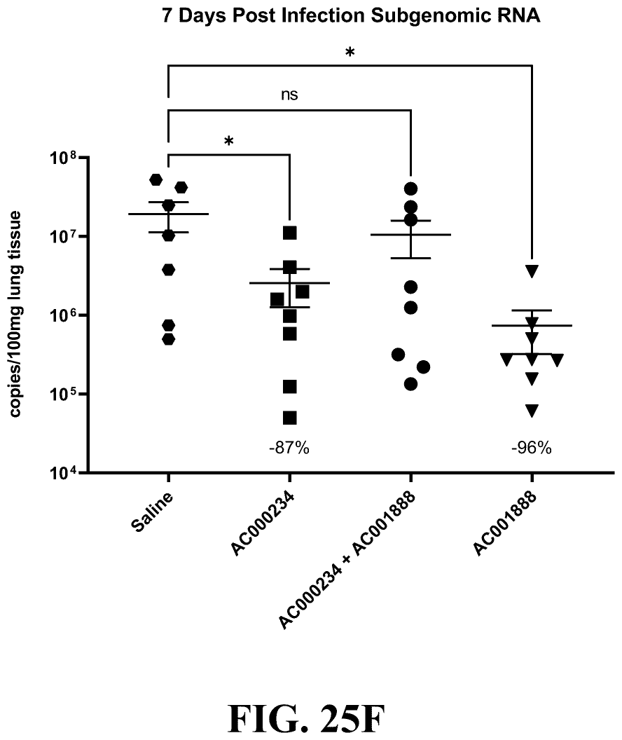

From Day −14 to +7, body weight measurements were collected for all of the experimental groups as well as the saline group. The body weights over time for all of the experimental groups are shown in FIG. 25A. When compared to the saline group infected with SARS-CoV-2, all of the experimental groups dosed with the CoV RNAi agents showed improved body weight recovery. Body weight recovered more quickly in groups treated with AC000234 COV RNAi agent than from AC001888 CoV RNAi agent alone.

FIG. 25B shows the total pulmonary inflammation area of the experimental groups treated with the CoV RNAi agents, in comparison with the non-treated saline. As shown in FIG. 25B, Syrian golden hamsters infected with SARS-CoV-2, upon treatment with the CoV RNAi agents, showed reduction in total area of pulmonary inflammation for all of the experimental groups treated with the CoV RNAi agents.

FIGS. 25C, 25D, 25E, and 25F show the genomic and subgenomic RNA levels at 3 and 7 days post infection by SARS-CoV-2. At Day 3 post infection, AC001888 achieved roughly 77% reduction in genomic RNA and 70% reduction in subgenomic RNA. At Day 7 post infection, AC000234 achieved roughly 85% reduction in genomic RNA and 87% reduction in subgenomic RNA, and AC001888 achieved roughly 96% reduction in both genomic and subgenomic RNA.

AC000234 is an RNAi agent designed to initiate RISC and RNAi in transmembrane serine protease 2 (TMPRSS2), and is not targeted to the SARS-CoV-2 viral genome.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An RNAi agent for inhibiting expression of a coronavirus (CoV) genome, comprising:
an antisense strand comprising at least 17 contiguous nucleotides differing by 0 or 1 nucleotides from any one of the sequences provided in Table 2 or Table 3; and
a sense strand comprising a nucleotide sequence that is at least partially complementary to the antisense strand.

2. The RNAi agent of claim 1, wherein the antisense strand comprises nucleotides 2-18 of any one of the sequences provided in Table 2 or Table 3.

3. The RNAi agent of claim 1 or claim 2, wherein the sense strand comprises a nucleotide sequence of at least 17 contiguous nucleotides differing by 0 or 1 nucleotides from any one of the sequences provided in Table 2 or Table 4, and wherein the sense strand has a region of at least 85% complementarity over the 17 contiguous nucleotides to the antisense strand.

4. The RNAi agent of any one of claims 1-3, wherein at least one nucleotide of the RNAi agent is a modified nucleotide or includes a modified intemucleoside linkage.

5. The RNAi agent of any one of claims 1-4, wherein all or substantially all of the nucleotides are modified nucleotides.

6. The RNAi agent of any one of claims 4-5, wherein the modified nucleotide is selected from the group consisting of: 2′-O-methyl nucleotide, 2′-fluoro nucleotide, 2′-deoxy nucleotide, 2′,3′-seco nucleotide mimic, locked nucleotide, 2′-F-arabino nucleotide, 2′-methoxyethyl nucleotide, abasic nucleotide, ribitol, inverted nucleotide, inverted 2′-O-methyl nucleotide, inverted 2′-deoxy nucleotide, 2′-amino-modified nucleotide, 2′-alkyl-modified nucleotide, morpholino nucleotide, vinyl phosphonate-containing nucleotide, cyclopropyl phosphonate-containing nucleotide, and 3′-O-methyl nucleotide.

7. The RNAi agent of claim 5, wherein all or substantially all of the nucleotides are modified with 2′-O-methyl nucleotides, 2′-fluoro nucleotides, or combinations thereof.

8. The RNAi agent of any one of claims 1-7, wherein the antisense strand comprises the nucleotide sequence of any one of the modified sequences provided in Table 3.

9. The RNAi agent of any one of claims 1-8, wherein the sense strand comprises the nucleotide sequence of any one of the modified sequences provided in Table 4.

10. The RNAi agent of claim 1, wherein the antisense strand comprises the nucleotide sequence of any one of the modified sequences provided in Table 3 and the sense strand comprises the nucleotide sequence of any one of the modified sequences provided in Table 4.

11. The RNAi agent of any one of claims 1-10, wherein the sense strand is between 18 and 30 nucleotides in length, and the antisense strand is between 18 and 30 nucleotides in length.

12. The RNAi agent of claim 11, wherein the sense strand and the antisense strand are each between 18 and 27 nucleotides in length.

13. The RNAi agent of claim 12, wherein the sense strand and the antisense strand are each between 18 and 24 nucleotides in length.

14. The RNAi agent of claim 13, wherein the sense strand and the antisense strand are each 21 nucleotides in length.

15. The RNAi agent of claim 14, wherein the RNAi agent has two blunt ends.

16. The RNAi agent of any one of claims 1-15, wherein the sense strand comprises one or two terminal caps.

17. The RNAi agent of any one of claims 1-16, wherein the sense strand comprises one or two inverted abasic residues.

18. The RNAi agent of claim 1, wherein the RNAi agent is comprised of a sense strand and an antisense strand that form a duplex having the structure of any one of the duplexes in Table 7A, Table 7B, Table 8, Table 9, or Table 10.

19. The RNAi agent of claim 18, wherein all or substantially all of the nucleotides are modified nucleotides.

20. The RNAi agent of claim 1, comprising an antisense strand that consists of, consists essentially of, or comprises a nucleotide sequence that differs by 0 or 1 nucleotides from one of the following nucleotide sequences (5′→3′):
(SEQ ID NO: 1122)
UUAGUAGGUAUAACCACAGCA;
or
(SEQ ID NO: 1113)
UUUGUAAUCAGUUCCUUGUCC.

21. The RNAi agent of any one of claims 1-20, wherein the nucleotides of the antisense strand located at position 2 and position 14 from the 5′-end are 2′-fluoro modified nucleotides.

22. The RNAi agent of claim 21, wherein the nucleotide of the antisense strand at position 2 is a 2′-fluoro uridine, and the nucleotide of the antisense strand at position 14 is a 2′-fluoro cytidine, and wherein the antisense strand comprises 3 or 4 phosphorothioate internucleoside linkages.

23. The RNAi agent of any one of claims 1-22, wherein the sense strand consists of, consists essentially of, or comprises a nucleotide sequence that differs by 0 or 1 nucleotides from one of the following nucleotide sequences (5′→3′):
(SEQ ID NO: 1198)
UGCUGUGGUUAUACCUACUAA;
or
(SEQ ID NO: 1189)
GGACAAGGAACUGAUUACAAA.

24. The RNAi agent of any one of claims 20-23, wherein all or substantially all of the nucleotides are modified nucleotides.

25. The RNAi agent of claim 1, comprising an antisense strand that comprises, consists of, or consists essentially of a modified nucleotide sequence that differs by 0 or 1 nucleotides from one of the following nucleotide sequences (5′→3′):
(SEQ ID NO: 779)
cPrpusUfsasGfuAfgGfuAfuAfaCfcAfcAfgCfsa;
or
(SEQ ID NO: 746)
cPrpusUfsusGfuAfaUfcAfgUfuCfcUfuGfuCfsc.
wherein a represents 2′-O-methyl adenosine, c represents 2′-O-methyl cytidine, g represents 2′-O-methyl guanosine, and u represents 2′-O-methyl uridine; Af, represents 2′-fluoro adenosine, Cf represents 2′-fluoro cytidine, Gf represents 2′-fluoro guanosine, and Uf represents 2′-fluoro uridine; cPrpu represents a 5′-cyclopropyl phosphonate-2′-O-methyl uridine; s represents a phosphorothioate linkage; and wherein all or substantially all of the nucleotides on the sense strand are modified nucleotides.

26. The RNAi agent of claim 1, wherein the sense strand comprises, consists of, or consists essentially of a modified nucleotide sequence that differs by 0 or 1 nucleotides from one of the following nucleotide sequences (5′→3′):
(SEQ ID NO: 1057)
usgcuguggUfUfAfuaccuacuaas;
or
(SEQ ID NO: 1046)
gsgacaaggAfAfCfugauuacaaas.
wherein a represents 2′-O-methyl adenosine, c represents 2′-O-methyl cytidine, g represents 2′-O-methyl guanosine, and u represents 2′-O-methyl uridine; Af, represents 2′-fluoro adenosine, Cf represents 2′-fluoro cytidine, Gf represents 2′-fluoro guanosine, and Uf represents 2′-fluoro uridine; s represents a phosphorothioate linkage; and wherein all or substantially all of the nucleotides on the antisense strand are modified nucleotides.

27. The RNAi agent of any one of claims 20-26, wherein the sense strand further includes inverted abasic residues at the 3′ terminal end of the nucleotide sequence, at the 5′ end of the nucleotide sequence, or at both.

28. The RNAi agent of any one of claims 1-27, wherein the RNAi agent is linked to a targeting ligand.

29. The RNAi agent of claim 28, wherein the targeting ligand has affinity for a cell receptor expressed on an epithelial cell.

30. The RNAi agent of claim 29, wherein the targeting ligand comprises an integrin targeting ligand.

31. The RNAi agent of claim 30, wherein the integrin targeting ligand is an αvβ6 integrin targeting ligand.

32. The RNAi agent of claim 31, wherein the targeting ligand comprises the structure: 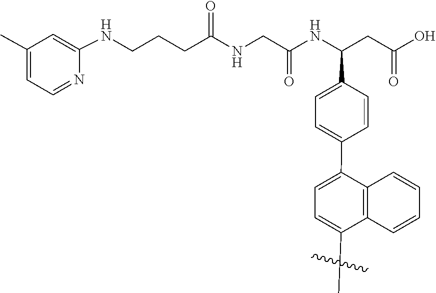 [SMILES: *c1ccc(-c2ccc([C@H](CC(=O)O)NC(=O)CNC(=O)CCCNc3cc(C)ccn3)cc2)c2ccccc12]
or a pharmaceutically acceptable salt thereof, or 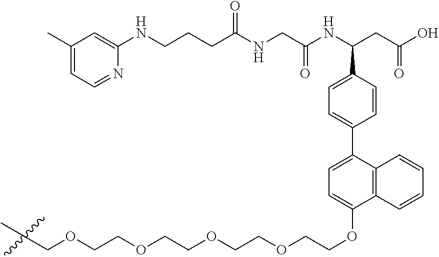 [SMILES: *COCCOCCOCCOCCOc1ccc(-c2ccc([C@H](CC(=O)O)NC(=O)CNC(=O)CCCNc3cc(C)ccn3)cc2)c2ccccc12]
or a pharmaceutically acceptable salt thereof,
wherein [symbol] indicates the point of connection to the RNAi agent.

33. The RNAi agent of any one of claims 28-31, wherein the targeting ligand has a structure selected from the group consisting of 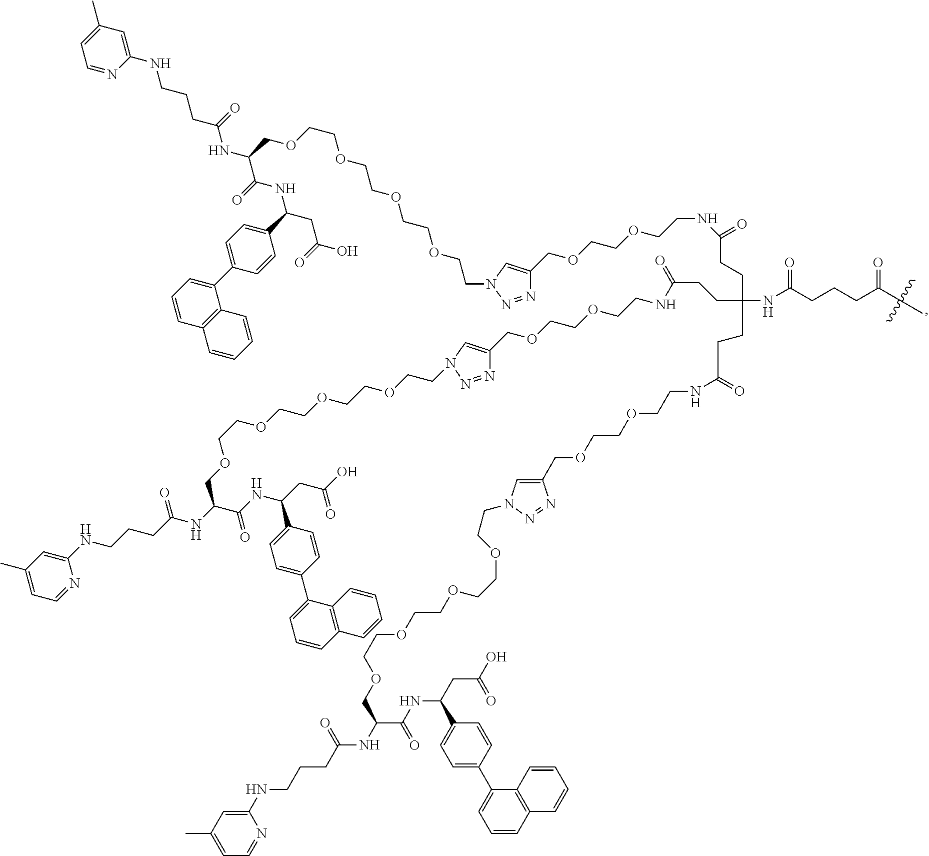 [SMILES: *C(=O)CCCC(=O)NC(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)(CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1)CCC(=O)NCCOCCOCc1cn(CCOCCOCCOCCOC[C@H](NC(=O)CCCNc2cc(C)ccn2)C(=O)N[C@@H](CC(=O)O)c2ccc(-c3cccc4ccccc34)cc2)nn1] 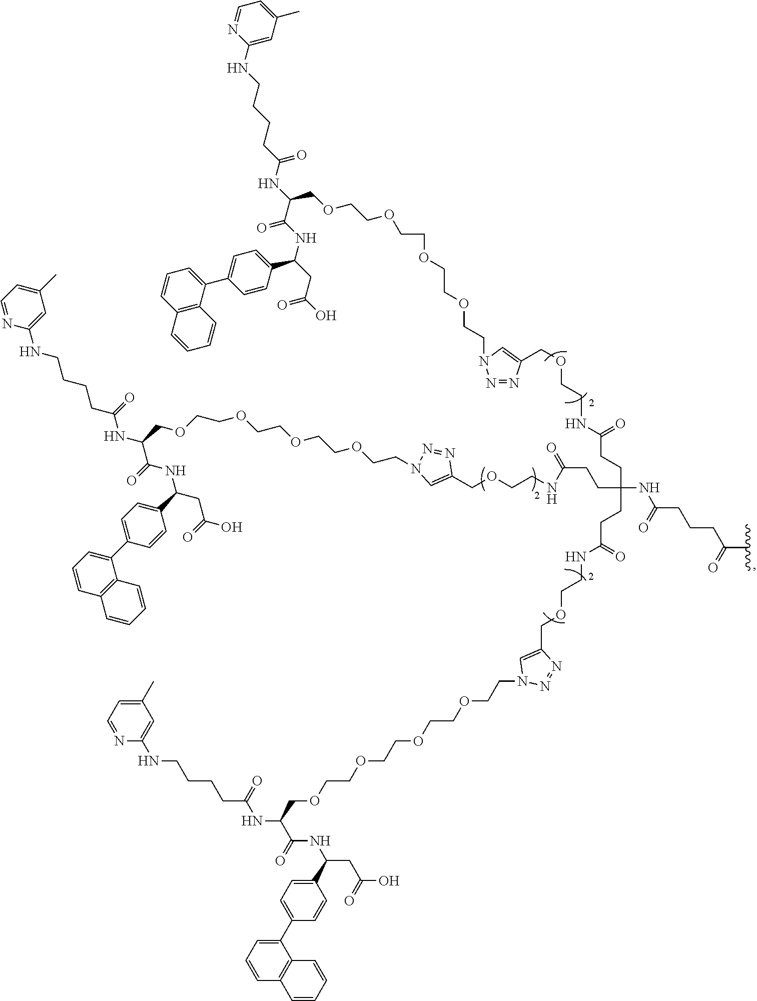 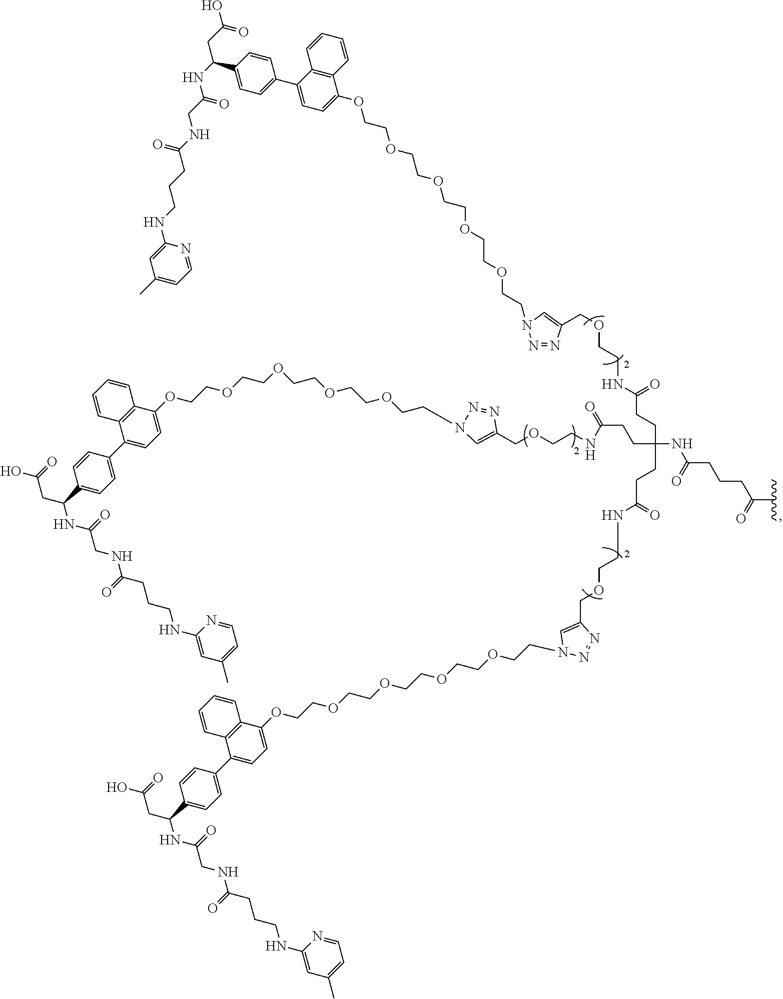 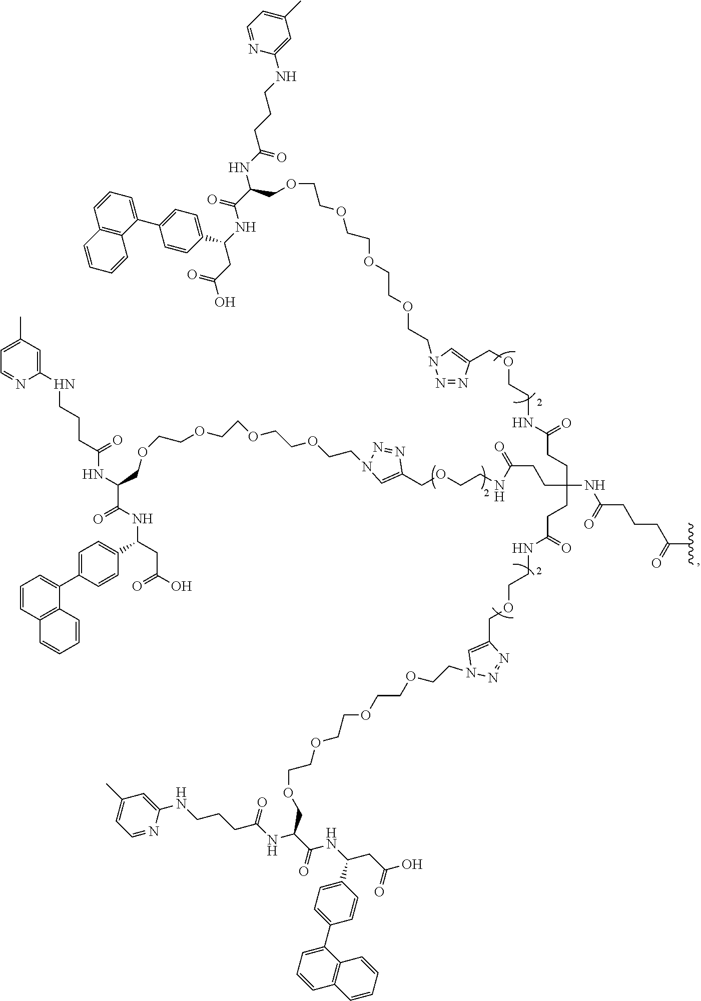 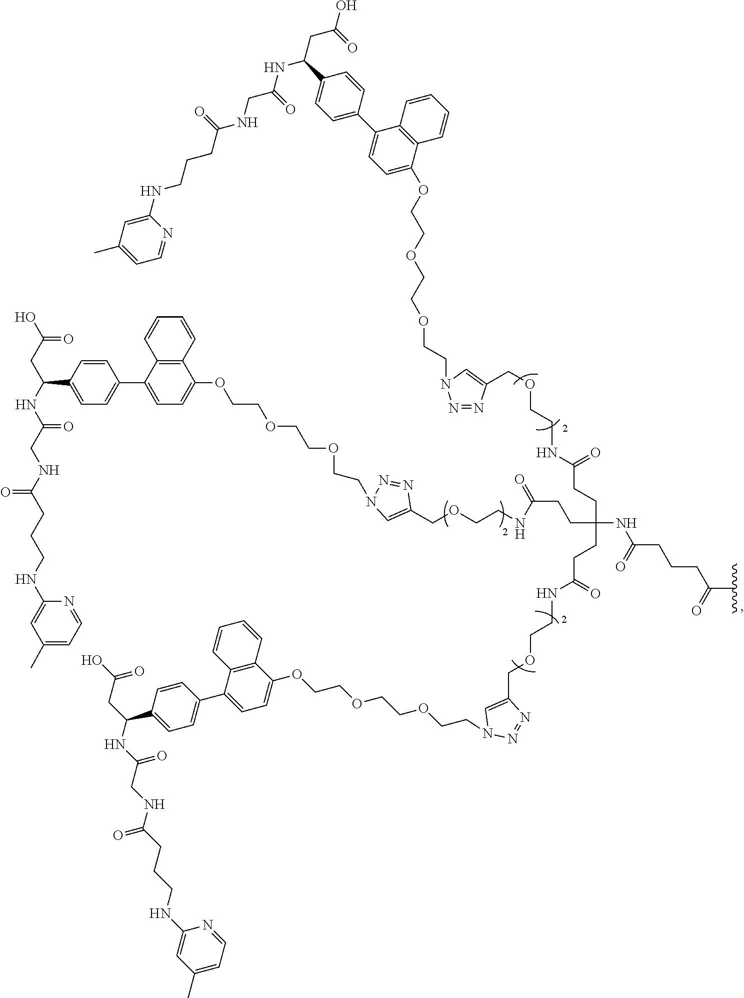 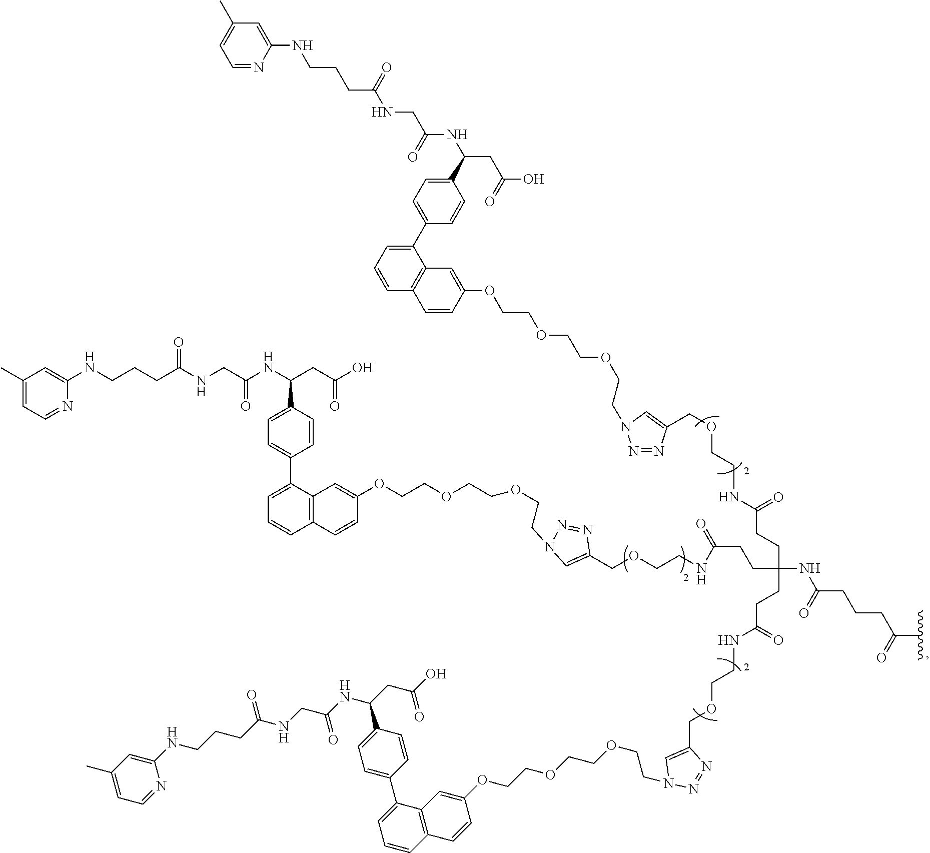 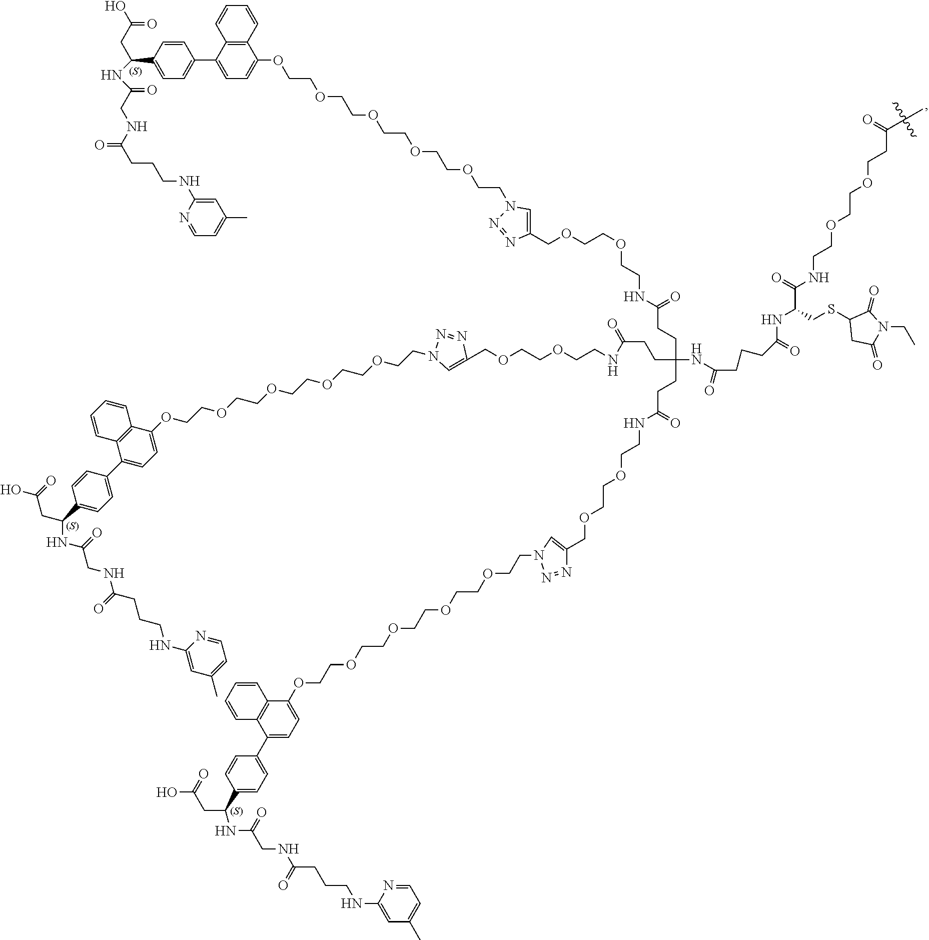 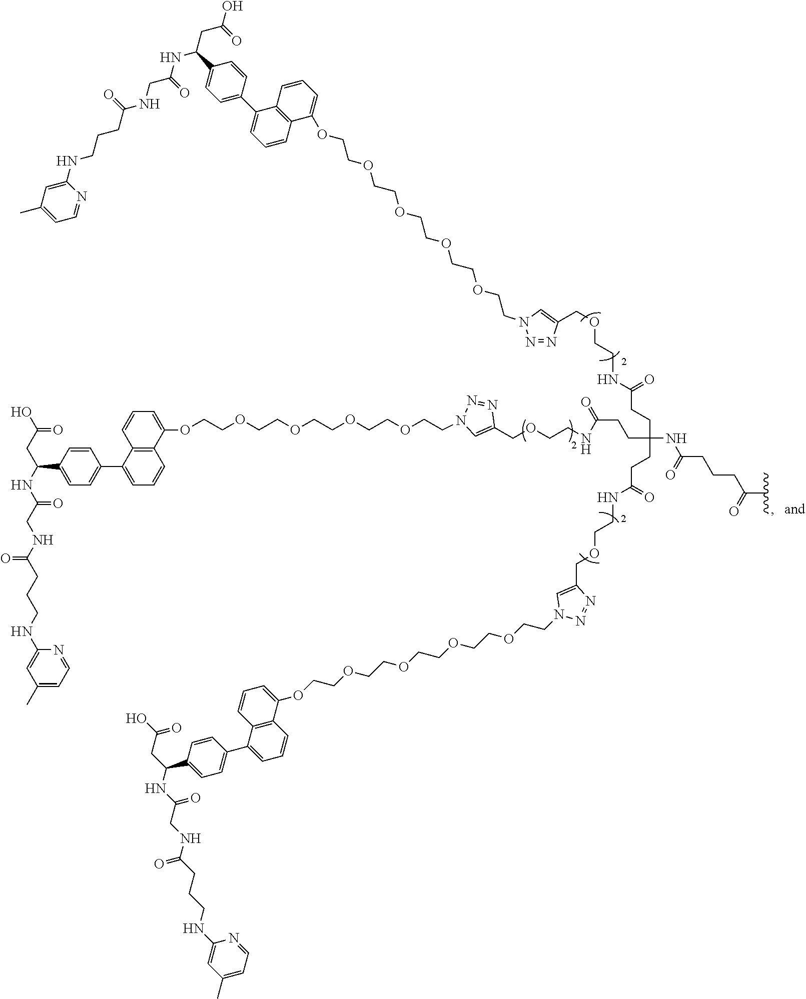 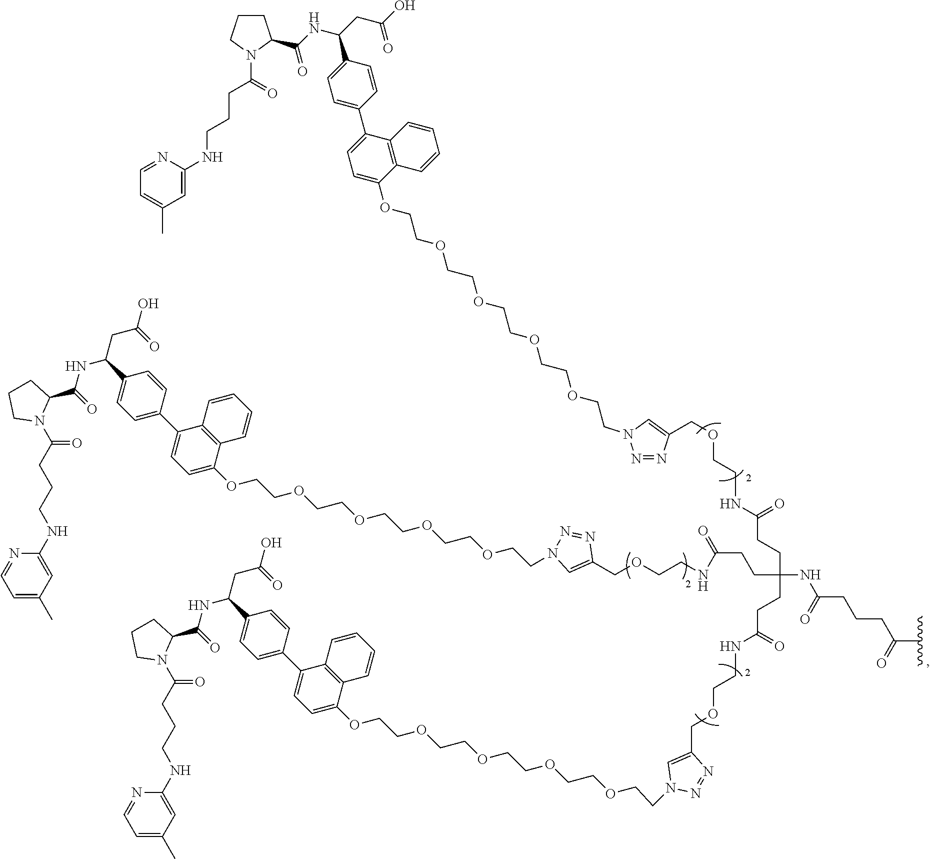
wherein [symbol] indicates the point of connection to the RNAi agent.

34. The RNAi agent of claim 33, wherein RNAi agent is conjugated to a targeting ligand having the following structure:

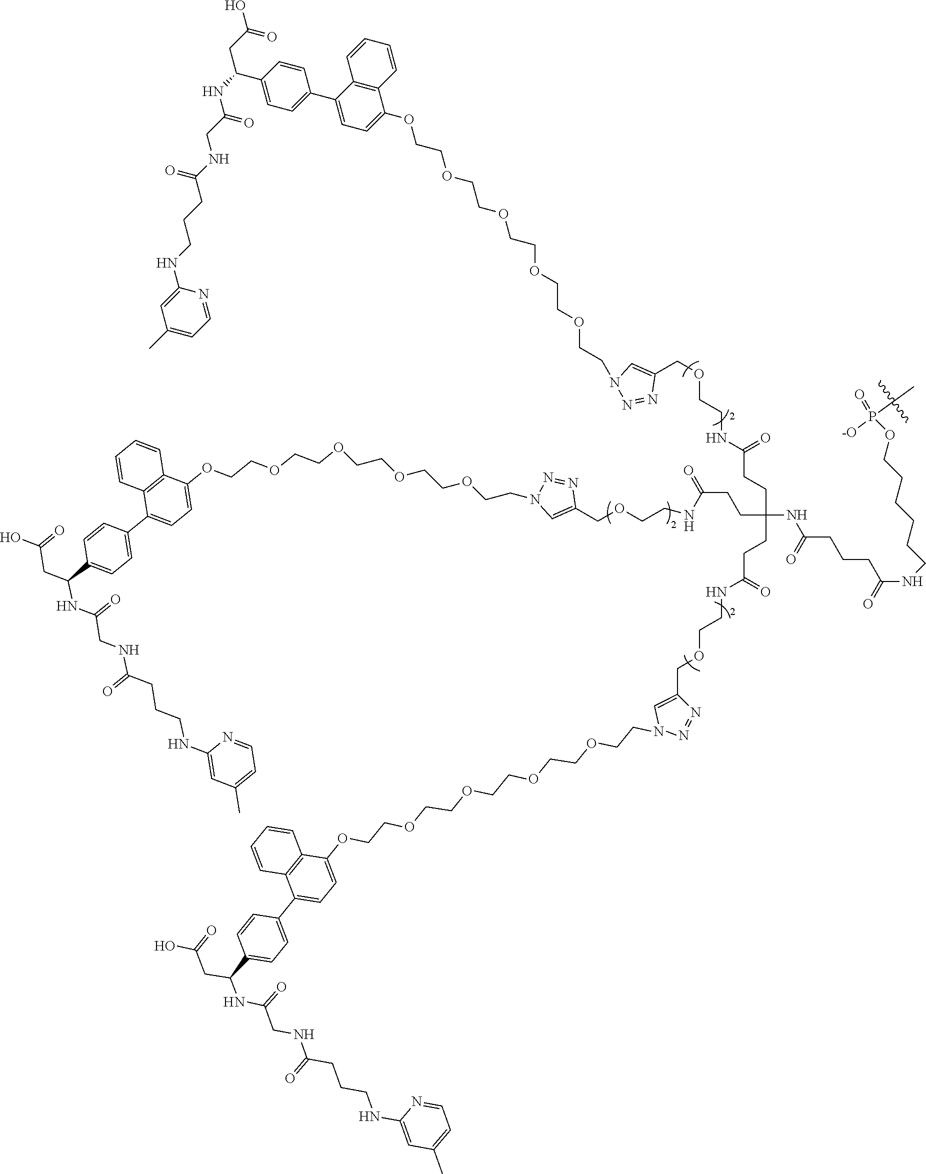

35. The RNAi agent of any one of claims 28-31, wherein the targeting ligand has the following structure:

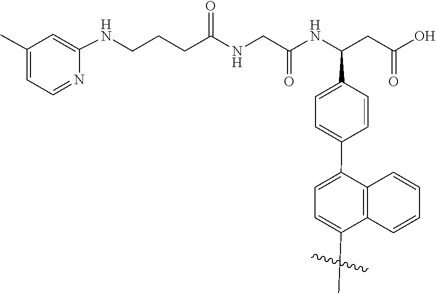 [SMILES: *c1ccc(-c2ccc([C@H](CC(=O)O)NC(=O)CNC(=O)CCCNc3cc(C)ccn3)cc2)c2ccccc12]

36. The RNAi agent of any one of claims 28-35, wherein the targeting ligand is conjugated to the sense strand.

37. The RNAi agent of claim 36, wherein the targeting ligand is conjugated to the 5′ terminal end of the sense strand.

38. The RNAi agent of claim 37, wherein the RNAi agent is a pharmaceutically acceptable salt.

39. The RNAi agent of claim 38, wherein the RNAi agent is a sodium salt.

40. A composition comprising the RNAi agent of any one of claims 1-39, wherein the composition further comprises a pharmaceutically acceptable excipient.

41. The composition of claim 40, further comprising a second RNAi agent capable of inhibiting the expression of a coronavirus (CoV) genome.

42. The composition of any one of claims 40-41, further comprising one or more additional therapeutics.

43. The composition of any one of claims 40-42, wherein the composition is formulated for administration by inhalation.

44. The composition of claim 43, wherein the composition is delivered by a metered-dose inhaler, jet nebulizer, vibrating mesh nebulizer, or soft mist inhaler.

45. The composition of any of claims 40-44, wherein the RNAi agent is a sodium salt.

46. The composition of any of claims 40-45, wherein the pharmaceutically acceptable excipient is water for injection.

47. The composition of any of claims 40-45, wherein the pharmaceutically acceptable excipient is a buffered saline solution.

48. A method for inhibiting a coronavirus (CoV) genome in a cell, the method comprising introducing into a cell an effective amount of an RNAi agent of any one of claims 1-39 or the composition of any one of claims 40-47.

49. The method of claim 48, wherein the cell is within a subject.

50. The method of claim 49, wherein the subject is a human subject.

51. The method of any one of claims 48-50, wherein following the administration of the RNAi agent the CoV genome expression is inhibited by at least about 30%.

52. A method of treating one or more symptoms or diseases associated with coronavirus (CoV) infection, the method comprising administering to a human subject in need thereof a therapeutically effective amount of the composition of any one of claims 40-47.

53. The method of claim 52, wherein the diseases is a respiratory disease.

54. The method of claim 53, wherein the respiratory disease is pulmonary inflammation.

55. The method of claim 54, wherein the respiratory disease is COVID-19.

56. The method of claim 52, wherein the symptoms are caused by SARS-CoV-2 viral infection.

57. The method of any one of claims 48-56, wherein the RNAi agent is administered at a deposited dose of about 0.01 mg/kg to about 5.0 mg/kg of body weight of the subject.

58. The method of any one of claims 48-57, wherein the RNAi agent is administered at a deposited dose of about 0.03 mg/kg to about 2.0 mg/kg of body weight of the subject.

59. The method of any one of claims 48-58, wherein the RNAi agent is administered in two or more doses.

60. Use of the RNAi agent of any one of claims 1-39, for the treatment of a disease, disorder, or symptom that is caused by coronavirus (CoV) infection, preferably wherein the disease, disorder, or symptom can be mediated at least in part by a reduction in SARS-CoV-2 activity and/or SARS-CoV-2 viral genome expression.

61. Use of the composition according to any one of claims 40-47, for the treatment of a disease, disorder, or symptom that is caused by coronavirus (CoV) infection, preferably wherein the disease, disorder, or symptom can be mediated at least in part by a reduction in SARS-CoV-2 activity and/or SARS-CoV-2 viral genome expression.

62. Use of the composition according to any one of claims 40-47, for the manufacture of a medicament for the treatment of a disease, disorder, or symptom that is caused by coronavirus (CoV) infection, preferably wherein the disease, disorder, or symptom can be mediated at least in part by a reduction in SARS-CoV-2 activity and/or SARS-CoV-2 viral genome expression.

63. The use of any one of claims 60-62, wherein the disease is pulmonary inflammation.

64. A method of making an RNAi agent of any one of claims 1-39, comprising annealing a sense strand and an antisense strand to form a double-stranded ribonucleic acid molecule.

65. The method of claim 64, wherein the sense strand comprises a targeting ligand.

66. The method of claim 65, comprising conjugating a targeting ligand to the sense strand.
